(19) United States
(12) Patent Application Publication
(10) Pub. No.: US 2023/0313249 A1
(43) Pub. Date: Oct. 5, 2023

(54) MICROBIAL PRODUCTION OF ARTEMISINIC ACID AND DERIVATIVES

(71) Applicant: Manus Bio Inc., Cambridge, MA (US)

(72) Inventors: Ajikumar PARAYIL KUMARAN, Lexington, MA (US); Christine Nicole S. SANTOS, Boston, MA (US); Stephen SARRIA, Watertown, MA (US); Jason Eric DONALD, Newton, MA (US); Yiying ZHENG, Windham, NH (US); Liwei LI, Arlington, MA (US); Eric NIEMINEN, Brookline, MA (US); Michelle N. GOETTGE, Cambridge, MA (US)

(21) Appl. No.: 18/023,912

(22) PCT Filed: Aug. 26, 2021
(86) PCT No.: PCT/US2021/047694

§ 371 Date: Feb. 28, 2023

Related U.S. Application Data
(60) Provisional application No. 63/071,630, filed on Aug. 28, 2020.

(51) Int. Cl. C12P 17/18; C12N 15/52; C12N 9/02; C12N 9/88

(52) U.S. Cl. CPC C12P 17/181; C12N 15/52; C12N 9/001; C12Y 103/01092; C12N 9/0042; C12N 9/88; C12Y 402/03024

(57) ABSTRACT

The present disclosure provides methods and compositions for producing artemisinic acid, dihydroartemisinic acid or artemisinin. In various aspects, the present disclosure provides enzymes, polynucleotides encoding said enzymes, and recombinant microbial host cells (or microbial host strains) for the production of artemisinic acid, dihydroartemisinic acid or artemisinin. The present disclosure further provides methods of making pharmaceutical products containing artemisinic acid, dihydroartemisinic acid or artemisinin.

### BACKGROUND

Artemisinin was identified as the principal compound in Artemisia annua extract with anti-malarial activity. Its structure was determined to be a sesquiterpene lactone with an endoperoxide bridge. In addition to their anti-malarial properties, artemisinin and its derivatives can be cytotoxic for cancer cells. Artemisinin levels in A. annua are usually in the range of 0.01 to 1% of total dry weight, which together with the fact that complete chemical synthesis of artemisinin is complex and inefficient at least partially accounts for the drug's high price. While methods for chemical synthesis of artemisinin are known, they are not an economical alternative for isolation of artemisinin from the plant. Given the commercial value of artemisinin, cost effective, scalable, and/or sustainable processes for its production are needed.

### SUMMARY OF THE INVENTION

In various aspects, the present disclosure provides methods and compositions for producing artemisinin, as well as dihydroartemisinic acid (DHAA) and artemisinic acid (AA) (immediate precursors for artemisinin). In various aspects, the present disclosure provides enzymes, polynucleotides encoding said enzymes, and recombinant microbial host cells (or microbial host strains) for the production of artemisinin, DHAA, or AA. The present disclosure further provides methods of making products containing artemisinin.

In some embodiments, the present disclosure provides a microbial host cell expressing an enzyme pathway catalyzing the conversion of farnesyl diphosphate (FPP) to artemisinin, DHAA, or AA, the enzymatic pathway comprising an amorphadiene synthase enzyme, an amorphadiene oxidase enzyme, and a double bond reductase. In some embodiments, the enzymatic pathway further comprises a peroxidase or an alpha-ketoglutarate-dependent dioxygenase enzyme capable of catalyzing conversion of artemisinic acid or dihydroartemisinic acid to artemisinin. In these embodiments, the microbial cells can synthesize artemisinin, DHAA, or AA product from any suitable carbon source. In some embodiments, the enzymes described herein enable high yield production of artemisinin, DHAA, or AA.

In some embodiments, the microbial host cell is prokaryotic or eukaryotic, and may be a bacterium or yeast. In some embodiments, the microbial host cell further expresses or overexpresses one or more enzymes in the methylerythritol phosphate (MEP) and/or the mevalonic acid (MVA) pathway to catalyze the conversion of glucose or other carbon sources to isopentenyl pyrophosphate (IPP) and/or dimethylallyl pyrophosphate (DMAPP). In some embodiments, the microbial host cell further expresses an enzyme catalyzing the conversion of IPP and/or DMAPP to farnesyl diphosphate (FPP), allowing for artemisinin, DHAA or AA to be produced from sugar or other carbon sources (carbon substrates such as C1, C2, C3, C4, C5, and/or C6 carbon substrates). In some embodiments, the host cell is a bacterium engineered to increase carbon flux through the MEP pathway.

In some embodiments, the microbial host cell expresses an amorphadiene oxidase, which may be a P450 enzyme providing for oxygenation of amorphadiene substrate. In some embodiments, the amorphadiene oxidase converts amorphadiene to artemisinic acid through three oxygenation events with artemisinic alcohol and artemisinic aldehyde as intermediates. In some embodiments, the cell further expresses at least one cytochrome P450 reductase to support P450 enzyme activity.

In some embodiments, the microbial host cell further expresses one or more alcohol dehydrogenases that convert artemisinic alcohol to artemisinic aldehyde. In some embodiments, the microbial host cell further expresses one or more aldehyde dehydrogenases that convert artemisinic aldehyde to artemisinic acid. In some embodiments, AA is recovered from the culture.

In some embodiments, the microbial host cell further expresses one or more double bond reductases converting artemisinic acid to dihydroartemisinic acid (DHAA). In some embodiments, DHAA is recovered from the culture, which can be converted to artemisinin. In still other embodiments, artemisinin is recovered from the culture.

In further embodiments, the microbial cell comprises or further comprises one or more peroxidase or alpha ketoglutarate-dependent dioxygenases (e.g., from A. annua or a derivative enzyme thereof) capable of converting artemisinic acid or dihydroartemisinic acid to artemisinin. For example, in some embodiments, the host cell expresses the entire enzymatic pathway for biosynthesis of artemisinin from C1-C6 carbon substrates, such as glucose or glycerol. In still other embodiments, the host cell expresses the one or more peroxidase and alpha ketoglutarate-dependent dioxygenase enzyme to allow for whole cell or cell lysate bioconversion of fed substrate (e.g., AA or DHAA). In still other embodiments, the one or more peroxidase and alpha ketoglutarate-dependent dioxygenase enzymes described herein are used in purified recombinant form for conversion of AA or DHAA to artemisinin in an in vitro reaction system.

Other aspects and embodiments of the invention will be apparent from the following detailed disclosure.

### DESCRIPTION OF THE FIGURES

FIG. 1 shows the chemical structure for artemisinin.

FIG. 2 shows an artemisinic acid pathway. Farnesyl diphosphate (FPP) is cyclized by amorphadiene synthase (ADS) into amorphadiene (AD). AD is then hydroxylated at position 12 by an amorphadiene oxidase (AO) (e.g., CYP71AV1/CPR) to form artemisinic alcohol (A-OH). A-OH is then oxidized by an alcohol dehydrogenase (e.g., AaADH1) and the resulting artemisinic aldehyde intermediate (A-CHO) is further oxidized by an aldehyde dehydrogenase (e.g., AaALDH1) to form artemisinic acid (AA). AA undergoes hydrogenation to dihydroartemisinic acid (DHAA) by a double bond reductase (e.g., AaDBR2 or catalyst) prior to being converted (e.g., enzymatically or photochemically) into artemisinin. Enzymatic DHAA production can be achieved by potentially two routes: one branches from artemisinic aldehyde (A-CHO) and goes through an dihydroartemisinic aldehyde (DHA-CHO) intermediate. The other route proceeds directly via AA. Enzymatic artemisinin production can be achieved from AA or DHAA with either a peroxidase or dioxygenase.

FIG. 3 shows results for ADS mutation screening for improvement of amorphadiene production. Mutants were screened by fermentation in 96-well plates for 48 hours.

FIG. 4 shows results for amorphadiene production with combined mutations from round 1 screening. The top mutant (ADS1), having the amino acid substitutions T118S, D162E, I173S, S322D, G363A, V396A, and Y474E, was tested alongside the wild type ADS. Fermentation was performed in a 96-well plate for 48 hours.

FIG. 5 shows results for AO screening to identify mutations (round 1) that would result in oxygenation of amorphadiene to A-OH and A-CHO by a single AO enzyme. Mutants were screened by fermentation in 96-well plates for 48 hours.

FIG. 6 shows amorphadiene oxygenation with WT AO and AO1 (V64L). AO1 shows significant production of the alcohol (the first oxygenation event).

FIG. 7 shows AO mutant screening (round 2). Mutants were screened for production of artemisinic alcohol, artemisinic aldehyde, and artemisinic acid. Fermentation was performed in a 96-well plate for 48 hours.

FIG. 8 shows production of artemisinic alcohol, artemisinic aldehyde and artemisinic acid with AO2, a variant having the following mutations: V64L, S73P, L155I, C320N, K322R, and V369L. AO2 produced substantial amounts of artemisinic acid as the major oxygenation product.

FIG. 9 shows screening of AaADH mutants for conversion of artemisinic alcohol to artemisinic aldehyde. Screening was conducted by fermentation in 96-well plates for 48 hours.

FIG. 10 shows production of artemisinic aldehyde with a single AaADH1 point mutant (A82V) as compared to wild type.

FIG. 11 shows microbial production of DHAA by co-expression of artemisinic acid pathway enzymes with a double bond reductase. Fermentation was performed in 96-well plates for 48 hours. Co-expression of A. annua DBR2 with AO2, AaADH1, and AaALDH, resulted in significant production of DHAA.

FIG. 12 shows improvements in production of DHAA by engineering of A. annua DBR2 for activity in E. coli. DBR2 mutants were screened for their ability to improve production of DHAA. Mutants were screened in two separate 96-well plate fermentation assays for 48 hours. Assay 1 and assay 2 differ by the organic solvent used as a culture overlay. FIG. 11A shows several mutants (i.e., amino acid substitutions) in either assay 1 or assay 2 and the associated beneficial fold-improvement in DHAA. FIG. 11B shows that DBR2 mutant with T241N substitution (DBR2_1) shows a significantly improved titer of DHAA as compared to wild-type.

FIG. 13 shows screening of candidate non-heme Fe(II) α-ketoglutarate-dependent dioxygenase enzymes from Artemisia annua in an AA-producing E. coli strain (co-expressing ADS, AO2, AaCPR, AaADH1, and ALDH) or a DHAA-producing E. coli strain (co-expressing ADS, AO2, AaCPR, AaADH1, ALDH, and DBR2). Fermentation was performed in 96-well plates for 48 hours. FIG. 13A shows a plot of DHAA with and without overexpression of dioxygenase candidate A0A2U1M3G2. FIG. 13B shows a summary of dioxygenase candidates that caused DHAA depletion.

### DETAILED DESCRIPTION

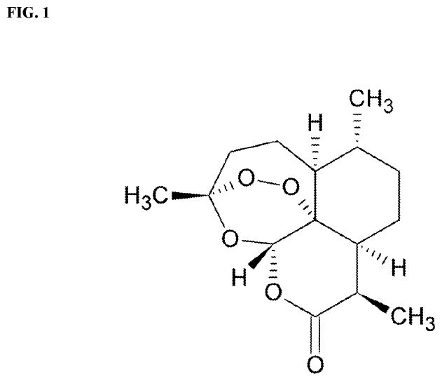

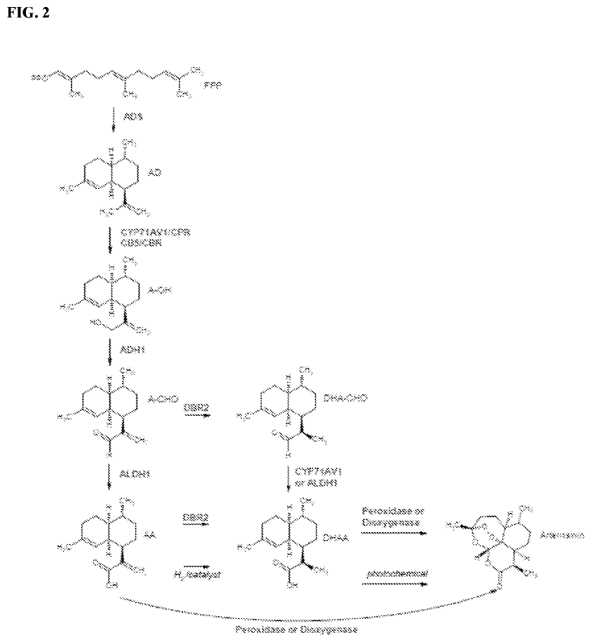

Artemisinin (FIG. 1) is an endoperoxide sesquiterpene lactone and a product of the isoprene pathway in plants. As illustrated in FIG. 2, farnesyl diphosphate (FPP) is a precursor of amorphadiene and the first specific substrate in the biosynthesis of artemisinin. FPP is produced by the condensation of two molecules of isopentenyl diphosphate (IPP) and its isomer dimethylallyl diphosphate (DMAPP). In higher plants, these two compounds can be derived from either the plastid-localized deoxyxylulose-5-phosphate (DXP) pathway or the cytosolic mevalonate pathway (MVA).

A biosynthetic pathway for artemisinin is shown in FIG. 2. The biosynthesis of artemisinin involves cyclization of the C15 sesquiterpene precursor substrate farnesyl diphosphate (FPP) to amorphadiene (AD) by amorphadiene synthase (ADS). AD is then hydroxylated at position 12 by an amorphadiene oxidase (AO), such as CYP7TAV1/CPR while CB5 and CBR assist in electron donation to make artemisinic alcohol (A-OH). A-OH is then oxidized by alcohol dehydrogenase (e.g., AaADH1) and the resulting artemisinic aldehyde intermediate (A-CHO) is further oxidized by an aldehyde dehydrogenase (e.g., AaALDH1) to form artemisinic acid (AA). Dehydrogenation of AA can produce DHAA, which can be converted to artemisinin.

In various aspects, the present disclosure provides enzymes (including variants thereof) related to biosynthesis of artemisinin (or its intermediate precursors DHAA or AA), microbial host cells (or microbial host strains) comprising such enzymes, methods for producing dihydroartemisinic acid (DHAA), AA or artemisinin, and methods of making pharmaceutical products containing these compounds. In some aspects, the present invention provides polynucleotides encoding for the enzymes disclosed herein.

In embodiments, the present disclosure provides a microbial host cell expressing a biosynthetic pathway catalyzing the conversion of farnesyl diphosphate (FPP) to AA, DHAA, or artemisinin. In various embodiments, the biosynthetic pathway comprises an amorphadiene synthase (ADS), an amorphadiene oxidase (AO), and a double bond reductase enzyme. In some embodiments, the microbial host cell may further comprise one or more alcohol dehydrogenase enzymes (ADH) and aldehyde dehydrogenase (ALDH) enzymes. In some embodiments, the microbial host cell may further comprise one or more peroxidases capable of converting artemisinic acid (AA) or dihydroartemisinic acid (DHAA) to artemisinin. In some embodiments, the microbial host cell may further comprise one or more alpha-ketoglutarate-dependent dioxygenases capable of converting artemisinic acid (AA) or dihydroartemisinic acid (DHAA) to artemisinin. In some embodiments, one or more enzymes are engineered for productivity, stability, and or expression in the microbial cell resulting in a high production of AA, DHAA, or artemisinin. In these embodiments, the microbial cells can synthesize AA, DHAA or artemisinin product from any suitable carbon source.

One aspect of the present invention is related to a microbial host cell for producing AA, DHAA, or artemisinin, the microbial cell expressing a biosynthetic pathway comprising: a heterologous enzyme having an amorphadiene synthase activity (ADS), a heterologous enzyme having an amorphadiene oxidase activity (AO), and a heterologous enzyme having a double bond reductase activity (DBR). DHAA is the immediate precursor of artemisinin, and its transformation to artemisinin has been shown to occur spontaneously through photo-oxidation, without enzyme intervention (Sy et al., The mechanism of the spontaneous autoxidation of dihydroartemisinic acid, Tetrahedron, vol. 58, 2002, 897-908 and Wallart et al., Isolation and identification of dihydroartemisinic acid from Artemisia annua and its possible role in the biosynthesis of artemisinin, J. Nat. Prod., vol. 62, 1999, 430-433, which are hereby incorporated by reference in their entirety).

Amorphadiene synthase refers to a terpene synthase that catalyzes formation of amorphadiene from farnesyl diphosphate (FPP), as shown in FIG. 2. In some embodiments, modifications to ADS enzyme can be informed by construction of a homology model. A homology model of the ADS from Artemisia annua is described by Eslami, Habib, et al., Journal of Molecular Modeling 23.7 (2017): 202. The structure disclosed by Eslami is based on the available crystal structure of the 5-epiaristolochene synthase (TEAS) and shows residues forming the substrate recognition pocket. The structural coordinates from Eslami or any other structure available for ADS can be used for constructing a homology model of ADS enzyme, which can be useful for guiding the engineering of ADS enzymes with improved specificity and productivity. See, U.S. Pat. Nos. 6,645,762; 6,495,354; and 6,645,762, which are hereby incorporated by reference in their entireties.

In some embodiments, the ADS comprises the amino acid sequence of SEQ ID NO: 1, 2, or a variant thereof. In some embodiments, the ADS enzyme comprises an amino acid sequence that has 50% or more sequence identity with SEQ ID NO: 1 or 2. In some embodiments, the ADS enzyme comprises an amino acid sequence that has at least about 50%, at least about 55%, at least about 60%, at least about 65%, at least about 70%, at least about 75%, at least about 80%, at least about 85%, at least about 90%, at least about 95%, at least about 96%, at least about 97%, or at least about 98%, or at least about 99% sequence identity with the amino acid sequence of SEQ ID NO: 1 or 2. In some embodiments, the ADS enzyme includes from 1 to about 20, or from 1 to about 10, or from 1 to about 5 amino acid modifications independently selected from substitutions, deletions, and insertions to the amino acid sequence of SEQ ID NO: 1 or 2.

In some embodiments, the ADS enzyme comprises a substitution to one or more of the substrate binding site or active site, as compared to the wild type enzyme. In some embodiments, the amino acid modifications can be selected to improve one or more of the following properties in the microbial cell: enzyme productivity, selectivity for the desired substrate and/or product, stability, temperature tolerance, and expression.

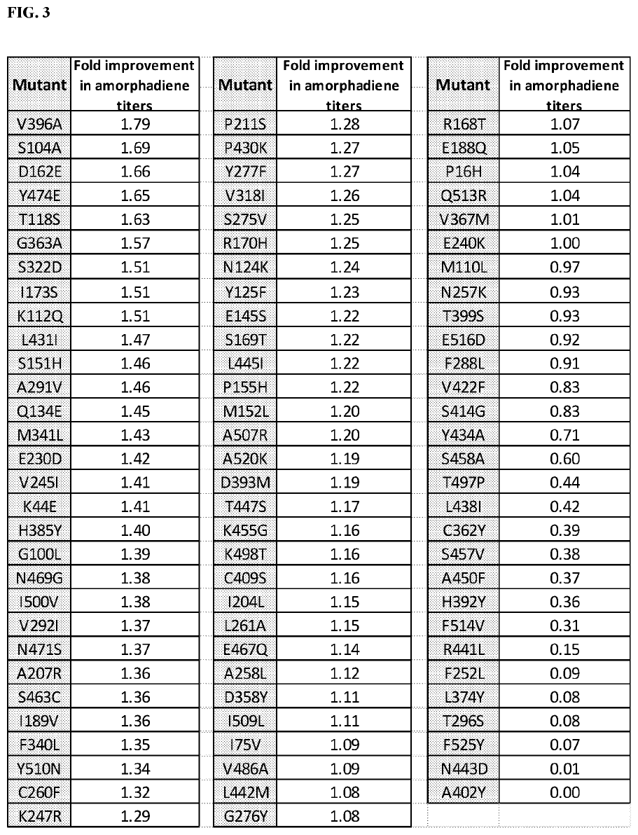

In some embodiments, the ADS enzyme comprises one or more amino acid substitutions at positions shown in FIG. 3 relative to SEQ ID NO: 1. In embodiments, the ADS enzyme comprises one or more amino acid substitutions as shown in FIG. 3 relative to SEQ ID NO: 1.

In some embodiments, the ADS enzyme comprises an amino acid substitution at one or more positions (e.g., from 1 to 20, or from 1 to 10, or from 1 to 5) corresponding to the following positions of SEQ ID NO: 1: 396, 104, 162, 474, 118, 363, 322, 173, 112, 431, 151, 291, 134, 341, 230, 245, 44, 385, 100, 469, 500, 292, 471, 207, 463, 189, 340, 510, 260, 247, 211, 430, 277, 318, 275, 170, 124, 125, 145, 169, 445, 155, 152, 507, 520, 393, 447, 455, 498, 409, 204, and 261. In some embodiments, the ADS enzyme comprises one or more substitutions (e.g., from 1 to 20, or from 1 to 10, or from 1 to 5) selected from the following substitutions numbered according to SEQ ID NO: 1: V396A, S104A, D162E, Y474E, T118S, G363A, S322D, I173S, K112Q, L431I, S151H, A291V, Q134E, M341L, E230D, V245I, K44E, H385Y, G100L, N469G, I500V, V292I, N471S, A207R, S463C, I189V, F340L, Y510N, C260F, K247R, P211S, P430K, Y277F, V318I, S275V, R170H, N124K, Y125F, E145S, S169T, L445I, P155H, M152L, A507R, A520K, D393M, T447S, K455G, K498T, C409S, I204L, and L261A. In some embodiments, the ADS enzyme comprises at least two, at least three, at least four, or at least five amino acid substitutions selected from V396A, S104A, D162E, Y474E, T118S, G363A, S322D, I173S, K112Q, L431I, S151H, A291V, Q134E, M341L, E230D, V245I, K44E, H385Y, G100L, N469G, I500V, V292I, N471S, A207R, S463C, I189V, F340L, Y510N, C260F, K247R, P211S, P430K, Y277F, V318I, S275V, R170H, N124K, Y125F, E145S, S169T, L445I, P155H, M152L, A507R, A520K, D393M, T447S, K455G. K498T, C409S, I204L, and L261A numbered according to SEQ ID NO: 1. In some embodiments, the ADS enzyme comprises the substitutions T118S, D162E, I173S, S322D, G363A, V396A, and Y474E numbered according to SEQ ID NO: 1.

In some embodiments, the ADS enzyme exhibits increased production of amorphadiene in a microbial cell producing farnesyl diphosphate as compared to ADS comprising the amino acid sequence of SEQ ID NO: 1. In some embodiments, the ADS enzyme is isolated or partially purified, or is heterologously expressed in a host cell. Some embodiments of the present invention are related to recombinant nucleic acid molecules comprising a nucleotide sequence encoding the ADS enzyme described herein. The recombinant nucleic acid could be a part of extrachromosomal elements such as plasmids, or bacterial artificial chromosomes, or may be chromosomally integrated.

In some embodiments, the present invention is related to amorphadiene oxidase (AO) or a variant thereof. In other embodiments, the present invention is related to microbial cells that express AO or its variants as described herein. Artemisia annua Amorphadiene oxidase, also known as CYP71AV1, is a cytochrome P450 (CYP450) that can provide for three-step oxidation of amorphadiene, along with its cognate reductase, to artemisinic acid.

CYP450 enzymes are involved in the formation (synthesis) and breakdown (metabolism) of various molecules and chemicals within cells. CYP450 enzymes have been identified in all kingdoms of life (i.e., animals, plants, fungi, protists, bacteria, archaea, and even in viruses). Illustrative structure and function of CYP450 enzymes are described in Uracher et al., TRENDS in Biotechnology, 24(7): 324-330 (2006). In some embodiments, the P450 enzymes are engineered to have a deletion of all or part of the wild type N-terminal transmembrane region, and the addition of a transmembrane domain derived from a bacterial inner membrane cytoplasmic C-terminus protein. In various embodiments, the transmembrane domain is a single-pass transmembrane domain. See U.S. Patent Publication No. 2018/0251738, which is hereby incorporated by reference in its entirety. In various embodiments, the transmembrane domain (or “N-terminal anchor”) is derived from an E. coli gene (or ortholog thereof) selected from waaA, ypfN, yhcB, yhbM, yhhm, zipA, ycgG, djlA, sohB, lpxK, F11O, motA, htpx, pgaC, ygdD, hemr, and ycls. These genes were identified as inner membrane cytoplasmic C-terminus proteins through bioinformatics prediction as well as experimental validation. The AO enzyme may employ an N-terminal anchor sequence that is a derivative of the E. coli wild-type transmembrane domain that has one or more (e.g., one, two, or three) mutations (e.g., amino acid substitutions) with respect to the wild-type sequence. Engineered P450 enzymes in accordance with these embodiments are described in U.S. Patent Publication No. 2018/0251738, which is hereby incorporated by reference in its entirety.

In some embodiments, the AO enzyme comprises an amino acid sequence of SEQ ID NO: 3 or a variant thereof. In embodiments, the AO enzyme comprises the amino acid sequence of SEQ ID NO: 4 or a variant thereof. In some embodiments, the AO enzyme comprises an amino acid sequence that has 50% or more sequence identity with SEQ ID NO: 3 or 4. In some embodiments, the AO enzyme comprises an amino acid sequence that has at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least or about 99% sequence identity with the amino acid sequence of SEQ ID NO: 3 or 4. In some embodiments, the AO enzyme comprises an amino acid sequence that includes from 1 to about 20, or from 1 to about 10, or from 1 to about 5 amino acid modifications independently selected from substitutions, deletions, and insertions to an amino acid sequence of SEQ ID NO: 3 or 4.

In some embodiments, the AO enzyme comprises a substitution to one or more of the substrate binding site or active site. In some embodiments, modifications to enzymes can be informed by available structures including construction of a homology model.

In some embodiments, the amino acid modifications can be selected to improve one or more properties upon expression in the microbial cell, such as those selected from: enzyme productivity, selectivity for the desired substrate and/or product, stability, temperature tolerance, and expression. In some embodiments, selection and modification of enzymes is informed by assaying activity on AD substrate or by expression in microbial cells producing AD.

In some embodiments, the AO enzyme comprises one or more amino acid substitutions at positions shown in FIG. 5 or FIG. 7 relative to SEQ ID NO: 3. In some embodiments, the AO variant comprises one or more amino acid substitutions as shown in FIG. 5 or FIG. 7 relative to SEQ ID NO: 3.

In some embodiments, the AO enzyme comprises an amino acid substitution at one or more positions (e.g., from 1 to 20, or from 1 to 10, or from 1 to 5) corresponding to the following positions of SEQ ID NO: 3: 239, 257, 408, 410, 421, 320, 130, 489, 198, 119, 102, 186, 252, 294, 314, 457, 474, 319, 322, 390, 125, 155, 251, 445, 424, 250, 387, 462, 153, 151, 243, 308, 495, 73, 103, 59, 123, 124, 146, 256, 261, 369, 469, and 64. In some embodiments, the AO enzyme comprises one or more (e.g., from 1 to 20, or from 1 to 10, or from 1 to 5) substitutions selected from the following substitutions numbered according to SEQ ID NO:3: A239R, A257D, A408P, A410E, A421I, C320N, E130D, E489D, G198K, H119G, I102L, I186T, I252L, I294V, I314M, I457L, I474L, K319R, K322R, K390R, L125F, L155I, L25II, L445F, M424K, N250R, N387A, N462D, Q153R, S151Q, S243K, S308T, S495T, S73P, T103A, T59L, V123I, V124A, V146T, V256I, V261E, V369L, V469M, and V64L. In some embodiments, the AO enzyme comprises at least two, at least three, at least four, or at least five amino acid substitutions selected from A239R, A257D, A408P, A410E, A421I, C320N, E130D, E489D, G198K, H119G, I102L, I186T, I252L, I294V, I314M, I457L, I474L, K319R, K322R, K390R, L125F, L155I, L25II, L445F, M424K, N250R, N387A, N462D, Q153R, S151Q, S243K, S308T, S495T, S73P, T103A, T59L, V123I, V124A, V146T, V256I, V261E, V369L, V469M, and V64L numbered according to SEQ ID NO: 3. In some embodiments, the AO enzyme comprises V64L, S73P, L155I, C320N, K322R, and V369L substitutions numbered according to SEQ ID NO: 3.

In some embodiments, the AO enzyme exhibits increased production of artemisinic alcohol (A-OH) as compared to AO comprising the amino acid sequence of SEQ ID NO: 3. In some embodiments, the AO enzyme exhibits increased production of artemisinic aldehyde (A-CHO) as compared to AO comprising the amino acid sequence of SEQ ID NO: 3. In some embodiments, the AO enzyme exhibits increased production of artemisinic acid (AA) as compared to AO comprising the amino acid sequence of SEQ ID NO: 3.

In some embodiments, the AO or variant thereof, as described herein, is isolated or partially purified, or is heterologously expressed in a host cell. Some embodiments of the present invention are related to recombinant nucleic acid molecules comprising a nucleotide sequence encoding the AO or variant thereof described herein. The recombinant nucleic acid could be a part of extrachromosomal elements such as plasmids, or bacterial artificial chromosomes, or may be chromosomally integrated.

In some embodiments, the AO or its variant comprises a leader sequence that supports expression and activity in E. coli, a linker sequence, or a CPR or derivative thereof sufficient to regenerate the AO variant. In embodiments, the AO or its variant requires the presence of an electron transfer protein capable of transferring electrons to the CYP450 protein. In some embodiments, this electron transfer protein is a cytochrome P450 reductase (CPR), which can be expressed by the microbial host cell. Various reductases that may be used are described in U.S. Patent Publication No. 2018/0135081, which is hereby incorporated by reference in its entirety.

Exemplary cytochrome P450 reductase enzymes (CPR), which may be used in the present invention, include that shown herein as SEQ ID NO: 5, or a variant thereof. Variants generally include enzymes comprising an amino acid sequence that has 50% or more sequence identity with of SEQ ID NO: 5. In some embodiments, the P450 reductase enzyme comprises an amino acid sequence that has at least about 60% sequence identity, or at least about 70% sequence identity, or at least about 80% sequence identity, or at least about 90% sequence identity, or at least about 95% sequence identity, or at least about 98% sequence identity, or at least about 99% sequence identity with the amino acid sequence of SEQ ID NO: 5. In some embodiments, the P450 reductase enzyme comprises an amino acid sequence that includes from 1 to about 20, or from 1 to about 10, or from 1 to about 5 amino acid modifications independently selected from substitutions, deletions, and insertions to the amino acid sequence of SEQ ID NO: 5.

In some embodiments, the alcohol intermediate (i.e., artemisinic alcohol) is converted to artemisinic aldehyde by an alcohol dehydrogenase (ADH). In embodiments, the microbial host cell expresses one or more ADHs. In some embodiments, modifications to enzymes can be informed by construction of a homology model. Structural coordinates from known structures of alcohol dehydrogenases can be used for constructing homology models of ADH enzymes, which are useful for guiding the engineering of ADH enzymes with improved specificity and productivity.

In some embodiments, the ADH enzyme comprises an amino acid sequence selected from SEQ ID NOs: 6, 7, or a variant thereof. Variants generally include enzymes comprising an amino acid sequence that has 50% or more sequence identity with any one of SEQ ID NOs: 6 and 7. In some embodiments, the ADH enzyme comprises an amino acid sequence that has at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity with the amino acid sequence of SEQ ID NO: 6 or 7. In some embodiments, the ADH enzyme comprises an amino acid sequence that includes from 1 to about 20, or from 1 to about 10, or from 1 to about 5 amino acid modifications independently selected from substitutions, deletions, and insertions to an amino acid sequence of SEQ ID NO: 6 or 7.

In some embodiments, the ADH enzyme comprises a substitution to one or more of the substrate binding site or active site. In some embodiments, the amino acid modifications can be selected to improve one or more of the following properties in microbial cells: enzyme productivity, selectivity for the desired substrate and/or product, stability, temperature tolerance, and expression.

In some embodiments, the ADH enzyme comprises one or more amino acid substitutions at positions shown in FIG. 9 relative to SEQ ID NO: 6. In embodiments, the ADH enzyme comprises one or more amino acid substitutions as shown in FIG. 9 relative to SEQ ID NO: 6.

In some embodiments, the ADH enzyme comprises an amino acid substitution at one or more positions (e.g., from 1 to 20, or from 1 to 10, or from 1 to 5) corresponding to the following positions of SEQ ID NO: 6: 82, 302, 155, 360, 299, 258, 304, 19, 107, 193, 263, 168, 78, 20, 253, 75, 191, 302, 80, 153, 203, 169, 229, 221, 329, 150, 6, 305, 60, 25, 310, 92, 233, 257, and 170. In embodiments, the ADH enzyme comprises one or more substitutions (e.g., from 1 to 20, from 1 to 10, or from 1 to 5) selected from the following substitutions numbered according to SEQ ID NO: 6: A82V, E302G, A155V, Q360N, A299V, V258I, S304E, S19A, T107S, S193E, H263D, S168P, 178V, S20G, S253P, 175V, Q191K, E302G, K80E, A153T, I203L, Y169L, Q229P, K221R, Q329R, T150S, P6G, V305I, I60E, L25I, L310F, M92I, A233E, L257V, and P170D. In some embodiments, the ADH enzyme comprises at least two, at least three, at least four, or at least five amino acid substitutions selected from A82V, E302G, A155V, Q360N, A299V, V258I, S304E, S19A, T107S, S193E, H263D, S168P, 178V, S20G, S253P, 175V, Q191K, E302G, K80E, A153T, I203L, Y169L, Q229P, K221R, Q329R, T150S, P6G, V305I, I60E, L25I, L310F, M92I, A233E, L257V, and P170D numbered according to SEQ ID NO: 6. In embodiments, the ADH comprises the substitution A82V numbered according to SEQ ID NO: 6.

In some embodiments, the ADH enzyme exhibits increased production of artemisinic aldehyde (A-CHO) in microbial cells producing artemisinic alcohol as compared to ADH comprising the amino acid sequence of SEQ ID NO: 6. In some embodiments, the ADH enzyme described herein is isolated or partially purified, or is heterologously expressed in a host cell. Some embodiments of the present invention are related to recombinant nucleic acid molecules comprising a nucleotide sequence encoding the ADH enzymes described herein. The recombinant nucleic acid could be a part of extrachromosomal elements such as plasmids, or bacterial artificial chromosomes, or may be chromosomally integrated.

In an artemisinin biosynthetic pathway, aldehyde dehydrogenase (ALDH) can catalyze conversion of artemisinic aldehyde to artemisinic acid (FIG. 2). In some embodiments, the microbial host cell expresses one or more heterologous aldehyde dehydrogenases. In some embodiments, modifications to enzymes can be informed by construction of a homology model.

By way of example, in some embodiments, the ALDH enzyme comprises an amino acid sequence of SEQ ID NO: 8 or a variant thereof. ALDH variants generally include enzymes comprising an amino acid sequence that has 50% or more sequence identity with SEQ ID NO: 8. In some embodiments, the ALDH enzyme comprises an amino acid sequence that has at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity with the amino acid sequence of SEQ ID NO: 8. In some embodiments, the ALDH enzyme comprises an amino acid sequence that includes from 1 to about 20, or from 1 to about 10, or from 1 to about 5 amino acid modifications independently selected from substitutions, deletions, and insertions to an amino acid sequence of SEQ ID NO: 8.

In some embodiments, the ALDH enzyme comprises a substitution to one or more of the substrate binding site or active site. In some embodiments, the amino acid modifications can be selected to improve one or more of the following properties in microbial cells: enzyme productivity, selectivity for the desired substrate and/or product, stability, temperature tolerance, and expression. In some embodiments, selection and modification of enzymes is informed by assaying activity on artemisinic alcohol or assaying activity in microbial cells producing artemisinic alcohol. In embodiments, the ALDH has increased production of artemisinic acid (AA) as compared to ALDH comprising the amino acid sequence of SEQ ID NO: 8 (AaALDH).

In some embodiments, the ALDH enzyme described herein, is isolated or partially purified, or is heterologously expressed in a host cell. Some embodiments of the present invention are related to recombinant nucleic acid molecules comprising a nucleotide sequence encoding the ALDH enzyme. The recombinant nucleic acid could be a part of extrachromosomal elements such as plasmids, or bacterial artificial chromosomes, or may be chromosomally integrated.

In some embodiments, the microbial host cell expresses one or more double bond reductases (DBRs). As used herein, a double-bond reductase is an enzyme that enhances production of DHAA in the microbial cells, by action on artemisinic acid and/or artemisinic aldehyde. In some embodiments, modifications to enzymes can be informed by construction of a homology model.

In some embodiments, the DBR comprises an amino acid sequence of SEQ ID NOs: 9, 10, or a variant thereof. In some embodiments, the variants of DBR include enzymes comprising an amino acid sequence that has 50% or more sequence identity with SEQ ID NO: 9 or 10. In some embodiments, the DBR enzyme comprises an amino acid sequence that has at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity with the amino acid sequence of SEQ ID NO: 9 or 10. In some embodiments, the DBR enzyme comprises an amino acid sequence that includes from 1 to about 20, or from 1 to about 10, or from 1 to about 5 amino acid modifications independently selected from substitutions, deletions, and insertions to an amino acid sequence of SEQ ID NO: 9 or 10.

In some embodiments, the DBR enzyme comprises a substitution to one or more of the substrate binding site or active site. In some embodiments, the amino acid modifications can be selected to improve one or more of the following properties in microbial cells: enzyme productivity, selectivity for the desired substrate and/or product, stability, temperature tolerance, and expression. In some embodiments, the DBR enzyme comprises an amino acid sequence that has at least about 80% or at least about 90%, or at least about 95% sequence identity to SEQ ID NO: 9, with an amino acid substitution at position 241 with respect to SEQ ID NO: 9. In some embodiments, the DBR enzyme comprises a substitution of asparagine at position 241 with respect to SEQ ID NO: 9.

In some embodiments, the DBR variant exhibits increased production of DHAA as compared to DBR comprising the amino acid sequence of SEQ ID NO: 9, when coexpressed with ADS, AO, CPR, ADH, and ALDH (as described). In some embodiments, the DBR variant exhibits increased production of DHAA as compared to DBR comprising the amino acid sequence of SEQ ID NO: 10, when coexpressed with ADS, AO, CPR, ADH, and ALDH.

In some embodiments, the DBR or variant thereof, as described herein, is isolated or partially purified, or is heterologously expressed in a host cell. Some embodiments of the present invention are related to recombinant nucleic acid molecules comprising a nucleotide sequence encoding the DBR or variant thereof as described herein. The recombinant nucleic acid could be a part of extrachromosomal elements such as plasmids, or bacterial artificial chromosomes, or may be chromosomally integrated.

In some embodiments, the microbial host cell may further comprise one or more peroxidases capable of converting artemisinic acid (AA) or dihydroartemisinic acid (DHAA) to artemisinin. In some embodiments, the peroxidase comprises any one of the amino acid sequences of SEQ ID NOs: 11-200, or a variant thereof. In some embodiments, the peroxidase comprises an amino acid sequence that has 50% or more sequence identity with any one of SEQ ID NOs: 11-200. In some embodiments, the peroxidase comprises an amino acid sequence that has at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity with the amino acid sequence of any one of SEQ ID NOs: 11-200. In some embodiments, the peroxidase comprises an amino acid sequence that includes from 1 to about 20, or from 1 to about 10, or from 1 to about 5 amino acid modifications independently selected from substitutions, deletions, and insertions to an amino acid sequence of any one of SEQ ID NOs: 11-200.

In some embodiments, the microbial host cell may further comprise one or more alpha-ketoglutarate-dependent dioxygenases capable of converting artemisinic acid (AA) or dihydroartemisinic acid (DHAA) to artemisinin. In some embodiments, the alpha-ketoglutarate-dependent dioxygenase comprises any one of the amino acid sequences of SEQ ID NOs: 201-488, or a variant thereof. In some embodiments, the alpha-ketoglutarate-dependent dioxygenase comprises an amino acid sequence that has 50% or more sequence identity with any one of SEQ ID NOs: 201-488. In some embodiments, the alpha-ketoglutarate-dependent dioxygenase comprises an amino acid sequence that has at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity with the amino acid sequence of any one of SEQ ID NOs: 201-488. In some embodiments, the alpha-ketoglutarate-dependent dioxygenase comprises an amino acid sequence that includes from 1 to about 20, or from 1 to about 10, or from 1 to about 5 amino acid modifications independently selected from substitutions, deletions, and insertions to an amino acid sequence of any one of SEQ ID NOs: 201-488.

In various embodiments, any one of the enzyme variants described herein including, but not limited to, ADS (variants of SEQ ID NO: 1), AO (variants of SEQ ID NO: 3), ADH (variants of SEQ ID NO: 6), ALDH (variants of SEQ ID NO: 8), DBR (variants of SEQ ID NO: 9 or 10), peroxidase (variants of SEQ ID NOs: 11-200), and alpha-ketoglutarate-dependent dioxygenase (variants of SEQ ID NOs: 201-488) result in higher product yields and higher overall productivity of their corresponding product in microbial cells. For example, in various embodiments, the microbial cell expresses an enzyme variant that results in at least 1.5-fold, or at least 2-fold, or at least 4-fold, or at least 10-fold higher titers for the corresponding product in microbial cells, as compared to its wild type counterpart.

The amino acid substitutions, for any one of the enzyme variants described herein including, but not limited to, ADS, AO, CPR, ADH, ALDH, DBR, peroxidase, and alpha-ketoglutarate-dependent dioxygenase may be (independently) conservative or non-conservative substitutions. “Conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups:
(1) hydrophobic: Met, Ala, Val, Leu, Ile;
(2) neutral hydrophilic: Cys, Ser, Thr; Asn, Gln;
(3) acidic: Asp, Glu;
(4) basic: His, Lys, Arg;
(5) residues that influence chain orientation: Gly, Pro; and
(6) aromatic: Trp, Tyr, Phe.

As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices. Some preferred conservative substitutions within the above six groups are exchanges within the following sub-groups: (i) Ala, Val, Leu and Ile; (ii) Ser and Thr; (ii) Asn and Gln; (iv) Lys and Arg; and (v) Tyr and Phe.

As used herein, “non-conservative substitutions” or “non-conservative amino acid exchanges” are defined as exchanges of an amino acid by another amino acid listed in a different group of the six standard amino acid groups (1) to (6) shown above.

The similarity of nucleotide and amino acid sequences can be determined via sequence alignments. Such alignments can be carried out with several art-known algorithms, such as with the mathematical algorithm of Karlin and Altschul (Karlin & Altschul (1993) Proc. Natl. Acad. Sci. USA 90: 5873-5877), with hmmalign (HMMER package, http://hmmer.wustl.edu/) or with the CLUSTAL algorithm (Thompson, J. D., Higgins, D. G. & Gibson, T. J. (1994) Nucleic Acids Res. 22, 4673-80). The grade of sequence identity (sequence matching) may be calculated using e.g. BLAST, BLAT or BlastZ (or BlastX). A similar algorithm is incorporated into the BLASTN and BLASTP programs of Altschul et al (1990) J. Mol. Biol. 215: 403-410. BLAST protein searches may be performed with the BLASTP program, score=50, word length=3. To obtain gapped alignments for comparative purposes, Gapped BLAST is utilized as described in Altschul et al (1997) Nucleic Acids Res. 25: 3389-3402. When utilizing BLAST and Gapped BLAST programs, the default parameters of the respective programs are used. Sequence matching analysis may be supplemented by established homology mapping techniques like Shuffle-LAGAN (Brudno M., Bioinformatics 2003b, 19 Suppl 1:154-162) or Markov random fields.

In various embodiments, the one or more heterologous enzymes selected from ADS, AO, CPR, ADH, ALDH, DBR, peroxidase, and alpha-ketoglutarate-dependent dioxygenase are expressed together in one or more operons, or are expressed individually in a host cell. The enzymes may be expressed from extrachromosomal elements such as plasmids, or bacterial artificial chromosomes, or may be chromosomally integrated.

In some embodiments, the microbial host cell is also engineered to express or overexpress one or more enzymes in the methylerythritol phosphate (MEP) and/or the mevalonic acid (MVA) pathway to catalyze isopentenyl pyrophosphate (IPP) and dimethylallyl pyrophosphate (DMAPP) from glucose or other carbon source.

In some embodiments, the microbial host cell is engineered to express or overexpress one or more enzymes of the MEP pathway. In some embodiments, the MEP pathway is increased and balanced with downstream pathways by providing duplicate copies of certain rate-limiting enzymes. The MEP (2-C-methyl-D-erythritol 4-phosphate) pathway, also called the MEP/DOXP (2-C-methyl-D-erythritol 4-phosphate/l-deoxy-D-xylulose 5-phosphate) pathway or the non-mevalonate pathway or the mevalonic acid-independent pathway refers to the pathway that converts glyceraldehyde-3-phosphate and pyruvate to IPP and DMAPP. The pathway typically involves action of the following enzymes: 1-deoxy-D-xylulose-5-phosphate synthase (Dxs), 1-deoxy-D-xylulose-5-phosphate reductoisomerase (IspC), 4-diphosphocytidyl-2-C-methyl-D-erythritol synthase (IspD), 4-diphosphocytidyl-2-C-methyl-D-erythritol kinase (IspE), 2C-methyl-D-erythritol 2,4-cyclodiphosphate synthase (IspF), 1-hydroxy-2-methyl-2-(E)-butenyl 4-diphosphate synthase (IspG), and isopentenyl diphosphate isomerase (IspH). The MEP pathway, and the genes and enzymes that make up the MEP pathway, are described in U.S. Pat. No. 8,512,988, which is hereby incorporated by reference in its entirety. For example, genes that make up the MEP pathway include dxs, ispC, ispD, ispE, ispF, ispG, ispH, idi, and ispA. In some embodiments, the microbial host cell expresses or overexpresses of one or more of dxs, ispC, ispD, ispE, ispF, ispG, ispH, idi, ispA, or modified variants thereof, which results in the increased production of IPP and DMAPP. In some embodiments, artemisinin, DHAA, or AA is produced at least in part by metabolic flux through an MEP pathway, and wherein the microbial host cell has at least one additional gene copy of one or more of dxs, ispC, ispD, ispE, ispF, ispG, ispH, idi, ispA, or modified variants thereof.

In some embodiments, the microbial host cell is engineered to express or overexpress one or more enzymes of the MVA pathway. The MVA pathway refers to the biosynthetic pathway that converts acetyl-CoA to IPP. The mevalonate pathway typically comprises enzymes that catalyze the following steps: (a) condensing two molecules of acetyl-CoA to acetoacetyl-CoA (e.g., by action of acetoacetyl-CoA thiolase); (b) condensing acetoacetyl-CoA with acetyl-CoA to form hydroxymethylglutaryl-CoenzymeA (HMG-CoA) (e.g., by action of HMG-CoA synthase (HMGS)); (c) converting HMG-CoA to mevalonate (e.g., by action of HMG-CoA reductase (HMGR)); (d) phosphorylating mevalonate to mevalonate 5-phosphate (e.g., by action of mevalonate kinase (MK)); (e) converting mevalonate 5-phosphate to mevalonate 5-pyrophosphate (e.g., by action of phosphomevalonate kinase (PMK)); and (f) converting mevalonate 5-pyrophosphate to isopentenyl pyrophosphate (e.g., by action of mevalonate pyrophosphate decarboxylase (MPD)). The MVA pathway, and the genes and enzymes that make up the MVA pathway, are described in U.S. Pat. No. 7,667,017, which is hereby incorporated by reference in its entirety. In some embodiments, the microbial host cell expresses or overexpresses one or more of acetoacetyl-CoA thiolase, HMGS, HMGR, MK, PMK, and MPD or modified variants thereof, which results in the increased production of IPP and DMAPP. In some embodiments, artemisinin, DHAA, or AA is produced at least in part by metabolic flux through an MVA pathway, and wherein the microbial host cell has at least one additional gene copy of one or more of acetoacetyl-CoA thiolase, HMGS, HMGR, MK, PMK, MPD, or modified variants thereof.

In some embodiments, the microbial host cell is engineered to increase production of IPP and DMAPP from glucose as described in PCT Application Nos. PCT/US2018/016848 and PCT/US2018/015527, the contents of which are hereby incorporated by reference in their entireties. For example, in some embodiments the microbial host cell overexpresses MEP pathway enzymes, with balanced expression to push/pull carbon flux to IPP and DMAPP. In some embodiments, the microbial host cell is engineered to increase the availability or activity of Fe—S cluster proteins, so as to support higher activity of IspG and IspH, which are Fe—S enzymes. In some embodiments, the host cell is engineered to overexpress IspG and IspH, so as to provide increased carbon flux to 1-hydroxy-2-methyl-2-(E)-butenyl 4-diphosphate (HMBPP) intermediate, but with balanced expression to prevent accumulation of HMBPP at an amount that reduces cell growth or viability, or at an amount that inhibits MEP pathway flux.

Conversion of IPP and DMAPP precursors to farnesyl diphosphate (FPP) is typically through the action of a farnesyl diphosphate synthase (FPPS). Exemplary FPPS enzymes are disclosed in US 2018/0135081, which is hereby incorporated by reference in its entirety.

In some embodiments, the host cell is engineered to downregulate the ubiquinone biosynthesis pathway, e.g., by reducing the expression or activity of IspB, which uses IPP and FPP substrate.

In some embodiments, the microbial host cell is a bacterium selected from Escherichia spp., Bacillus spp., Corynebacterium spp., Rhodobacter spp., Zymomonas spp., Vibrio spp., and Pseudomonas spp. For example, in some embodiments, the bacterial host cell is a species selected from Escherichia coli, Bacillus subtilis, Corynebacterium glutamicum, Rhodobacter capsulatus, Rhodobacter sphaeroides, Zymomonas mobilis, Vibrio natriegens, or Pseudomonas putida. In some embodiments, the bacterial host cell is E. coli.

In some embodiments, the microbial host cell is a species of Saccharomyces, Pichia, or Yarrowia, including, but not limited to, Saccharomyces cerevisiae, Pichia pastoris, and Yarrowia lipolytica.

In some aspects, the present invention is related to the method of making AA, DHAA, or artemisinin. This method includes culturing any of the microbial cells as described herein and recovering the AA, DHAA, or artemisinin. In some embodiments, the microbial cell expresses a biosynthetic pathway comprising a heterologous enzyme having an amorphadiene synthase activity (ADS), a heterologous enzyme having an amorphadiene oxidase activity (AO), and a heterologous enzyme having a double bond reductase activity (DBR), as described herein. Cells expressing ADS, AO and DBR enzymes can produce AA, DHAA or artemisinin from a carbon source. In some embodiments, the microbial host cell may further comprise one or more peroxidases capable of converting artemisinic acid (AA) or dihydroartemisinic acid (DHAA) to artemisinin. In some embodiments, the microbial host cell may further comprise one or more alpha-ketoglutarate-dependent dioxygenases capable of converting AA or DHAA to artemisinin. Alternatively, the method described herein further include a step of converting AA or DHAA to artemisinin. In some embodiments, the step converting of DHAA to artemisinin is done photochemically. In some embodiments, the conversion is conducted enzymatically.

In some aspects and embodiments, the present invention provides enzymatic methods for converting AA or DHAA to artemisinin, including through whole cell, cell lysate, or recombinant enzyme based bioconversion of a fed substrate. In some embodiments the method comprises: culturing a microbial host cell that expresses one or more enzymes described herein for converting AA or DHAA to artemisinin, feeding the culture a substrate selected from one or more of AA and DHAA, and recovering artemisinin from the culture. In other embodiments, the method employs contacting the substrate with a cell lysate of the microbial host cells or purified recombinant enzyme under suitable reaction conditions.

In some embodiments, the methods described herein include a microbial host cell that further expresses one or more alcohol dehydrogenases disclosed herein. In some embodiments, the methods described herein include a microbial host cell that further expresses one or more aldehyde dehydrogenases disclosed herein. In some embodiments, the methods described herein include a microbial host cell that expresses one or more ADS, AO/CPR, DBR, ADH, and ALDH, enzymes disclosed herein.

In some embodiments, the invention provides a microbial host cell that further expresses a heterologous enzyme having an activity for converting AA or DHAA to artemisinin. In some embodiments, the heterologous enzyme has a peroxidase activity and has at least 70% sequence identity to one of SEQ ID NOs: 11 to 200 (as described herein). In some embodiments, the heterologous enzyme is an Artemisia annua alpha-ketoglutarate-dependent dioxygenase, or variant thereof. In some embodiments, the heterologous enzyme having an alpha-ketoglutarate-dependent dioxygenase activity comprises an amino acid sequence that has at least 70%, at least 80%, or at least 90% sequence identity to one of SEQ ID NOs: 201 to 488 (as described herein). In some embodiments, the heterologous enzyme having an alpha-ketoglutarate-dependent dioxygenase activity comprises an amino acid sequence that has at least 70%, at least 80%, or at least 90% sequence identity (as described) to one of SEQ ID NOS: 302, 323, 361, 366, and 369. In some embodiments, the heterologous enzyme having an alpha-ketoglutarate-dependent dioxygenase activity comprises an amino acid sequence that has at least 70%, at least 80%, or at least 90% sequence identity (as described) to one of SEQ ID NOS: 302, 361, and 369.

In some embodiments, the host cell is cultured to produce DHAA or artemisinin. In some embodiments, microbial cells are cultured with carbon substrates (sources) such as C1, C2, C3, C4, C5, and/or C6 carbon substrates. In exemplary embodiments, the carbon source is glucose, sucrose, fructose, xylose, and/or glycerol. Culture conditions are generally selected from aerobic, microaerobic, and anaerobic.

In various embodiments, the host cell is cultured at a temperature between 22° C. and 37° C. While commercial biosynthesis in bacteria such as E. coli can be limited by the temperature at which overexpressed and/or foreign enzymes (e.g., enzymes derived from plants) are stable, recombinant enzymes (including the terpenoid synthase) may be engineered to allow for cultures to be maintained at higher temperatures, resulting in higher yields and higher overall productivity. In some embodiments, the host cell is a bacterial host cell, and culturing is conducted at about 22° C. or greater, about 23° C. or greater, about 24° C. or greater, about 25° C. or greater, about 26° C. or greater, about 27° C. or greater, about 28° C. or greater, about 29° C. or greater, about 30° C. or greater, about 31° C. or greater, about 32° C. or greater, about 33° C. or greater, about 34° C. or greater, about 35° C. or greater, about 36° C. or greater, or about 37° C.

AA, DHAA and/or artemisinin can be extracted from media and/or whole cells, and recovered. In some embodiments, AA, DHAA or artemisinin is recovered in a process comprising aqueous extraction followed by precipitation. AA, DHAA or artemisinin can be quantified by any suitable process, including, for example, liquid chromatography. The desired product can be produced in batch or continuous bioreactor systems.

In some embodiments, the microbial host cells and methods disclosed herein are suitable for commercial production of AA, DHAA, and/or artemisinin, that is, the microbial host cells and methods are productive at commercial scale. In some embodiments, the size of the culture is at least about 100 L, at least about 200 L, at least about 500 L, at least about 1,000 L, at least about 10,000 L, at least about 100,000 L, or at least about 1,000,000 L. In some embodiment, the culturing may be conducted in batch culture, continuous culture, or semi-continuous culture.

In some aspects, the present disclosure provides methods for making a product comprising artemisinin, including a pharmaceutical product. In some embodiments, the method comprises producing artemisinin as described herein through microbial culture, recovering artemisinin, and incorporating the artemisinin into the pharmaceutical product.

As used in this specification and the appended claims, the singular forms “a”, “an” and “the” include plural referents unless the content clearly dictates otherwise. For example, reference to “a cell” includes a combination of two or more cells, and the like.

As used herein, the term “about” in reference to a number is generally taken to include numbers that fall within a range of 10% in either direction (greater than or less than) of the number.

### EXAMPLES

Artemisinin is a sesquiterpene lactone (FIG. 1) and has been used for the treatment of malarial and parasitic worm (helminth) infections. The biosynthesis of artemisinin involves cyclization of the C15 sesquiterpene precursor substrate farnesyl diphosphate (FPP) to amorphadiene (AD) by amorphadiene synthase (ADS). AD is then hydroxylated at position 12 by an amorphadiene oxidase (AO), such as CYP71AV1/CPR while CB5 and CBR assist in electron donation to make artemisinic alcohol (A-OH). A-OH is then oxidized by alcohol dehydrogenase (e.g., AaADH1) and the resulting artemisinic aldehyde intermediate (A-CHO) is further oxidized by an aldehyde dehydrogenase (e.g., AaALDH1) to form artemisinic acid (AA). Hydrogenation of AA by double bond reductase (e.g., AaDBR2) can produce DHAA, which can be converted to artemisinin either enzymatically (with peroxidase or alpha ketoglutarate-dependent dioxygenase) or photochemically. FIG. 2.

### Example 1: Engineering Enzymes to Improve Production of Artemisinin

The amorphadiene precursor farnesyl diphosphate (FPP) is a terpenoid, and can be produced by biosynthetic fermentation processes, using microbial strains that produce high levels of MEP pathway products along with heterologous expression of artemisinin biosynthesis enzymes.

For example, in bacteria such as E. coli, isopentenyl pyrophosphate (IPP) and dimethylallyl pyrophosphate (DMAPP) can be produced from glucose or other carbon source, and which can be converted to farnesyl diphosphate (FPP) by recombinant farnesyl diphosphate synthase (FPPS). FPP is converted through a cyclization reaction to amorphadiene by recombinant expression of ADS.

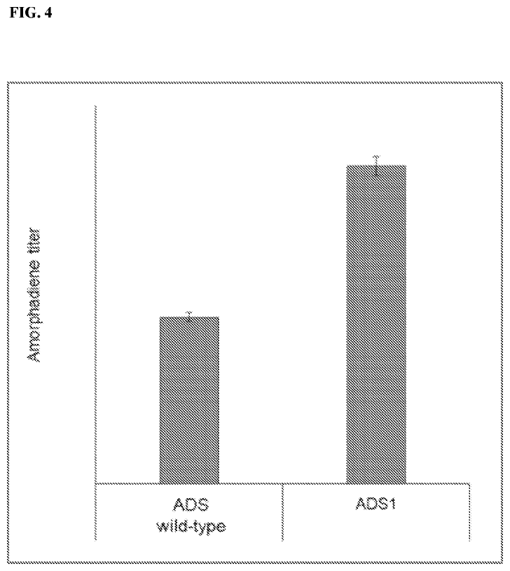

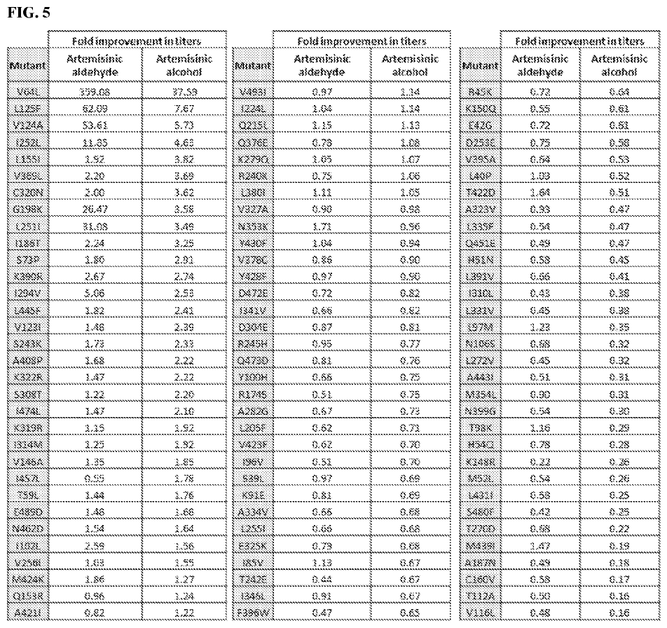

Using an E. coli background strain that produces high levels of the MEP pathway products IPP and DMAPP (see US 2018/0245103 and US 2018/0216137, which are hereby incorporated by reference), several mutants of Artemisia annua amorphadiene synthases were screened by co-expression with FPPS. Fermentation was performed in 96 well plates for 48 hours. Several mutant enzymes, as shown in FIG. 3, demonstrated improved production of amorphadiene in E. coli. FIG. 4 shows results for amorphadiene production with combined mutations from round 1 screening. A mutant combining top substitutions (T118S, D162E, I173S, S322D, G363A, V396A, and Y474E) designated ADS1 was tested alongside the wild type ADS. Fermentation was performed in a 96-well plate for 48 hours. ADS1, as shown in FIG. 4, was selected for subsequent studies based on its productivity. As shown in FIG. 4, ADS1 exhibits close to two times amorphadiene production as compared to the wild-type ADS.

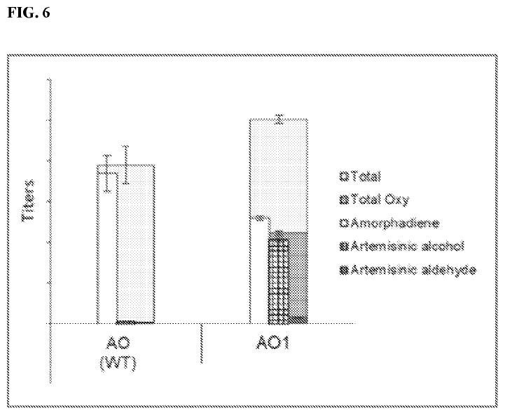

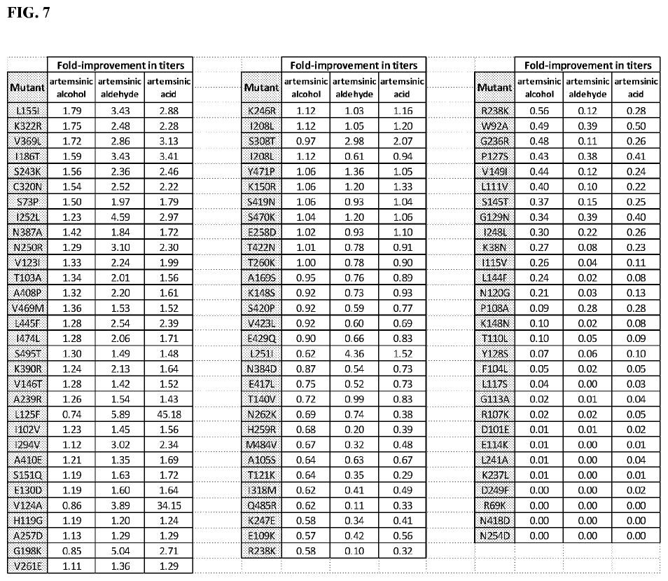

Artemisia annua AO was engineered to conduct multiple oxygenations of AD, to produce the AA. AaAO was engineered to delete a portion of the transmembrane domain, with the addition of a membrane anchor derived from E. coli yhhm. See, US 2018/0251738, which is hereby incorporated by reference. A panel of mutations to the AO were screened for their ability to catalyze multiple oxygenation events. Mutants were screened by fermentation in 96-well plates for 48 hours. FIG. 5 shows several mutants (i.e., amino acid substitutions) and the associated fold-improvement in artemisinic alcohol and artemisinic aldehyde production. FIG. 6 shows that AO mutant with V64L substitution (AO1) shows a significantly improved titer of artemisinic alcohol (the first oxygenation event).

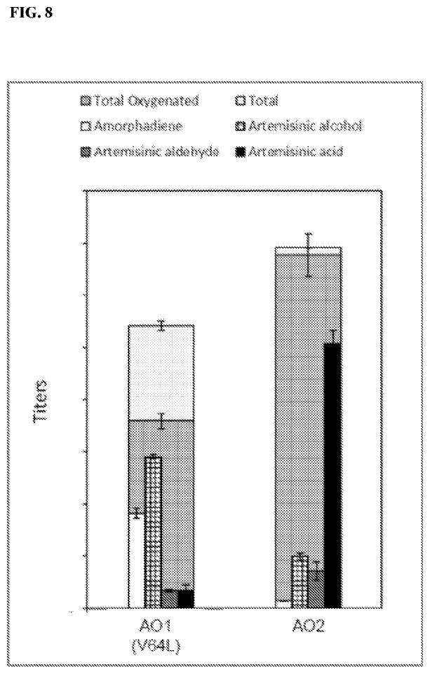

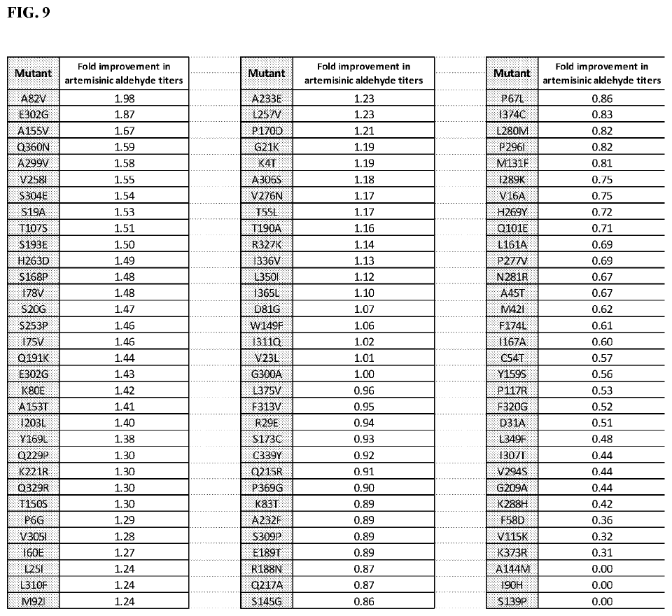

A second round of AO mutations were screened for their ability to produce artemisinic acid from AD (FIG. 7). Fermentation was performed in a 96-well plate for 48 hours. FIG. 8 shows that the production of the alcohol, aldehyde and acid with AO2, a variant having the following mutations: V64L, S73P, L155I, C320N, K322R, and V369L. AO2 produced substantial amounts of artemisinic acid as the major oxygenation product, thus conducting the three oxygenation events from AD to AA.

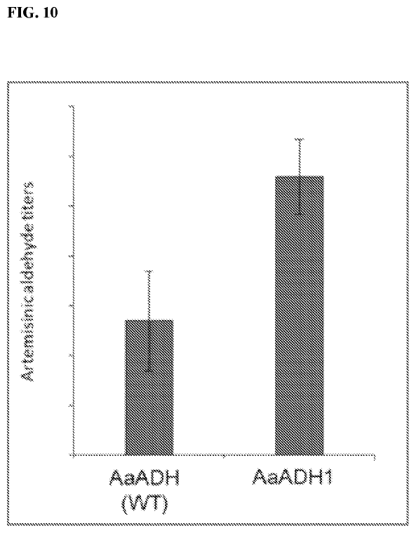

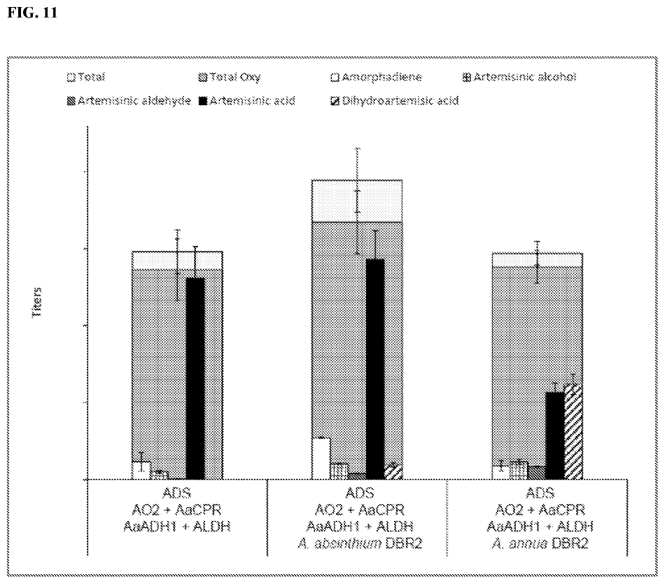

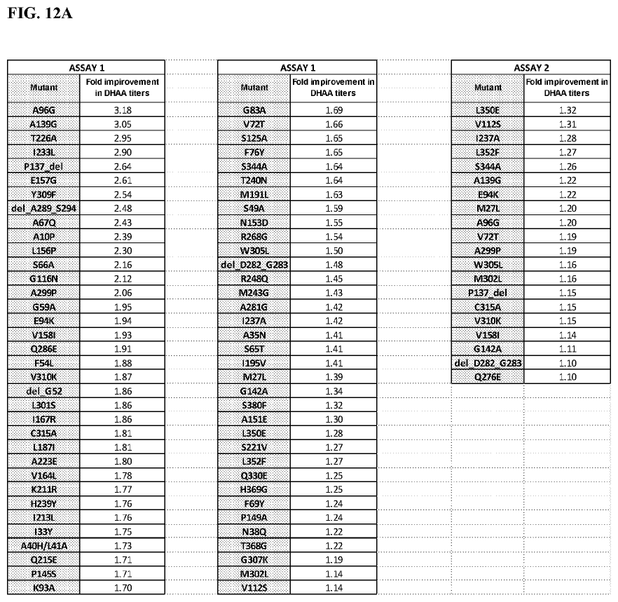

To support production of A-CHO, AaADH was engineered for activity in E. coli. A panel of mutations to AaADH were screened for their ability to improve production of artemisinic aldehyde. Mutants were screened by fermentation in 96-well plates for 48 hours. FIG. 9 shows several mutants (i.e., amino acid substitutions) and the associated fold-improvement in artemisinic aldehyde. FIG. 10 shows that ADH mutant with A82V substitution (AaADH1) shows a significantly improved titer of artemisinic aldehyde as compared to wild-type.

### Example 2: Production of DHAA

To produce dihydroartemisinic Acid (DHAA) in microbial cells, candidate double bond reductase (DBR) enzymes from Artemisia absinthium and Artemisia annua where screened by co-expression in E. coli with ADS, AO2, AaCPR, ADH, and ALDH. Fermentation was performed in 96-well plates for 48 hours.

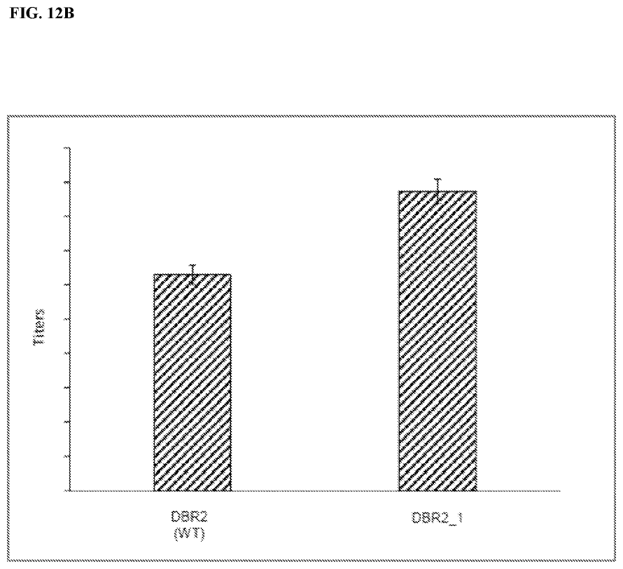

Co-expression of A. absinthium DBR2 along with ADS, AO2, AaCPR, ADH, and ALDH produces DHAA (FIG. 11). Co-expression of A. annua DBR2 along with ADS, AO2, AaCPR, ADH, and ALDH shows substantial production of DHAA (FIG. 11). Thus, microbial fermentation systems can be employed to produce DHAA.

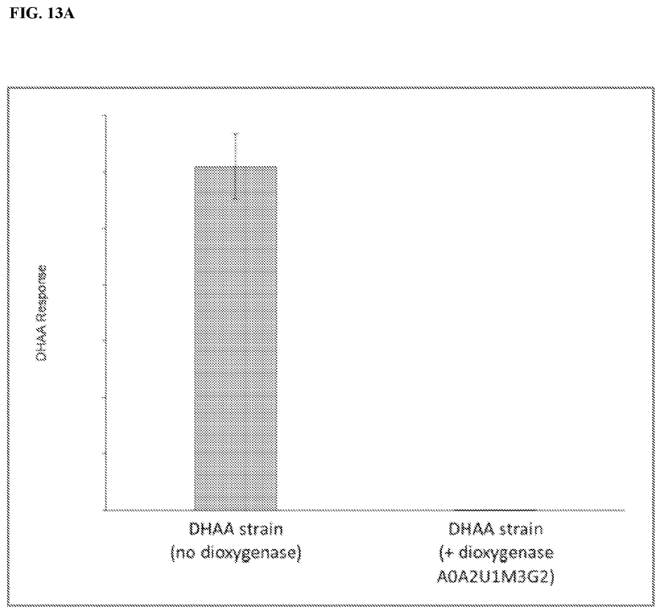

To support production of DHAA, A. annua DBR2 was engineered for activity in E. coli. A panel of mutations to DBR2 were screened for their ability to improve production of DHAA. Mutants were screened in two separate 96-well plate fermentation assays for 48 hours. Assay 1 and assay 2 differ by the organic solvent used as a culture overlay. FIG. 12A shows several mutants (i.e., amino acid substitutions) in either assay 1 or assay 2 and the associated beneficial fold-improvement in DHAA. FIG. 12B shows that DBR2 mutant with T241N substitution (DBR2_1) shows a significantly improved titer of DHAA as compared to wild-type.

### Example 3: Production of Artemisinin Using a Peroxidase and/or an α-Ketoglutarate-Dependent Dioxygenase

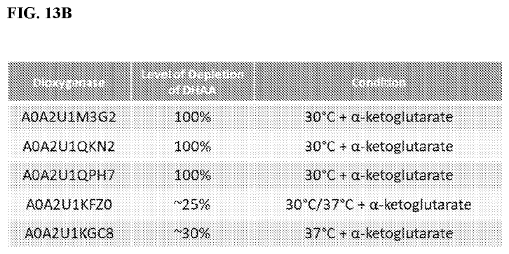

To produce artemisinin in microbial cells, candidate non-heme Fe(II) α-ketoglutarate-dependent dioxygenase enzymes from Artemisia annua were screened in an AA-producing E. coli strain (co-expressing ADS, AO2, AaCPR, AaADH1, and ALDH) or a DHAA-producing E. coli strain (co-expressing ADS, AO2, AaCPR, AaADH1, ALDH, and DBR2). Fermentation was performed in 96-well plates for 48 hours. FIG. 13A shows a plot of DHAA with and without overexpression of dioxygenase candidate A0A2U1M3G2. FIG. 13B shows a summary of dioxygenase candidates that caused DHAA depletion under specific conditions.

SEQUENCES

SEQ ID NO: 1
Artemisia annua amorphadiene synthase (ADS, wild-type)
MSLTEEKPIRPIANFPPSIWGDQFLIYEKQVEQGVEQIVNDLKKEVRQLLKEALDIPMKH
ANLLKLIDEIQRLGIPYHFEREIDHALQCIYETYGDNWNGDRSSLWFRLMRKQGYYVTCD
VENNYKDKNGAFKQSLANDVEGLLELYEATSMRVPGEIILEDALGFTRSRLSIMTKDAFS
TNPALFTEIQRALKQPLWKRLPRIEAAQYIPFYQQQDSHNKTLLKLAKLEFNLLQSLHKE
ELSHVCKWWKAFDIKKNAPCLRDRIVECYFWGLGSGYEPQYSRARVFFTKAVAVITLIDD
TYDAYGTYEELKIFTEAVERWSITCLDTLPEYMKPIYKLFMDTYTEMEEFLAKEGRTDLF
NCGKEFVKEFVRNLMVEAKWANEGHIPTTEEHDPVVIITGGANLLTTTCYLGMSDIFTKE
SVEWAVSAPPLFRYSGILGRRLNDLMTHKAEQERKHSSSSLESYMKEYNVNEEYAQTLIY
KEVEDVWKDINREYLTTKNIPRPLLMAVIYLCQFLEVQYAGKDNFTRMGDEYKHLIKSLL
VYPMSI
SEQ ID NO: 2
Artemisia annua amorphadiene synthase (ADS1, engineered
variant)
MSLTEEKPIRPIANFPPSIWGDQFLIYEKQVEQGVEQIVNDLKKEVRQLLKEALDIPMKH
ANLLKLIDEIQRLGIPYHFEREIDHALQCIYETYGDNWNGDRSSLWFRLMRKQGYYVSCD
VFNNYKDKNGAFKQSLANDVEGLLELYEATSMRVPGEIILEEALGFTRSRLSSMTKDAFS
TNPALFTEIQRALKQPLWKRLPRIEAAQYIPFYQQQDSHNKTLLKLAKLEFNLLQSLHKE
ELSHVCKWWKAFDIKKNAPCLRDRIVECYFWGLGSGYEPQYSRARVFFTKAVAVITLIDD
TYDAYGTYEELKIFTEAVERWDITCLDTLPEYMKPIYKLFMDTYTEMEEFLAKEGRTDLF
NCAKEFVKEFVRNLMVEAKWANEGHIPTTEEHDPVAIITGGANLLTTTCYLGMSDIFTKE
SVEWAVSAPPLFRYSGILGRRLNDLMTHKAEQERKHSSSSLESYMKEYNVNEEEAQTLIY
KEVEDVWKDINREYLTTKNIPRPLLMAVIYLCQFLEVQYAGKDNFTRMGDEYKHLIKSLL
VYPMSI
SEQ ID NO: 3
Artemisia annua amorphadiene oxidase (AO, wild-type)
MAKSILKAMALSLTTSIALATILLFVYKFATRSKSTKKSLPEPWRLPIIGHMHHLIGT
TP
HRGVRDLARKYGSLMHLQLGEVPTIVVSSPKWAKEILTTYDITFANRPETLTGEIVLY
HNTDVVLAPYGEYWRQLRKICTLELLSVKKVKSFQSLREEECWNLVQEIKASGSGRPV
NLSENVFKLIATILSRAAFGKGIKDQKELTEIVKEILRQTGGFDVADIFPSKKFLHHL
SGKRARLTSLRKKIDNLIDNLVAEHTVNTSSKTNETLLDVLLRLKDSAEFPLTSDNIK
AIILDMFGAGTDTSSSTIEWAISELIKCPKAMEKVQAELRKALNGKEKIHEEDIQELS
YLNMVIKETLRLHPPLPLVLPRECRQPVNLAGYNIPNKTKLIVNVFAINRDPEYWKDA
EAFIPERFENSSATVMGAEYEYLPFGAGRRMCPGAALGLANVQLPLANILYHENWKLP
NGVSYDQIDMTESSGATMQRKTELLLVPSF
SEQ ID NO: 4
Artemisia annua amorphadiene oxidase (AO2, engineered
variant with yhhM anchor shown in bold) [symbol]
VYKFATRSKSTKKSLPEPWRLPIIGHMHHLIGTTPHR
GL
RDLARKYGPLMHLQLGEVPTIVVSSPKWAKEILTTYDITFANRPETLTGEIVLYHNTD
VV
LAPYGEYWRQLRKICTLELLSVKKVKSFQSIREEECWNLVQEIKASGSGRPVNLSENV
FK
LIATILSRAAFGKGIKDQKELTEIVKEILRQTGGFDVADIFPSKKFLHHLSGKRARLT
SL
RKKIDNLIDNLVAEHTVNTSSKTNETLLDVLLRLKDSAEFPLTSDNIKAIILDMFGAG
TD
TSSSTIEWAISELIKNPRAMEKVQAELRKALNGKEKIHEEDIQELSYLNMVIKETLRL
HP
PLPLLLPRECRQPVNLAGYNIPNKTKLIVNVFAINRDPEYWKDAEAFIPERFENSSAT
VM
GAEYEYLPFGAGRRMCPGAALGLANVQLPLANILYHFNWKLPNGVSYDQIDMTESSGA
TM
QRKTELLLVPSF
SEQ ID NO: 5
Artemisia annua AaCPR
MAQSTTSVKLSPFDLMTALLNGKVSFDTSNTSDTNIPLAVFMENRELLMILTTSVAVL
IGCVVVLVWRRSSSAAKKAAESPVIVVPKKVTEDEVDDGRKKVTVFFGTQTGTAEGFA
KALVEEAKARYEKAVFKVIDLDDYAAEDDEYEEKLKKESLAFFFLATYGDGEPTDNAA
RFYKWFTEGEEKGEWLDKLQYAVFGLGNRQYEHFNKIAKVVDEKLVEQGAKRLVPVGM
GDDDQCIEDDFTAWKELVWPELDQLLRDEDDTSVATPYTAAVAEYRVVFHDKPETYDQ
DQLTNGHAVHDAQHPCRSNVAVKKELHSPLSDRSCTHLEFDISNTGLSYETGDHVGVY
VENLSEVVDEAEKLIGLPPHTYFSVHADNEDGTPLGGASLPPPFPPCTLRKALASYAD
VLSSPKKSALLALAAHATDSTEADRLKFLASPAGKDEYAQWIVASHRSLLEVMEAFPS
AKPPLGVFFASVAPRLQPRYYSISSSPRFAPNRIHVTCALVYEQTPSGRVHKGVCSTW
MKNAVPMTESQDCSWAPIYVRTSNFRLPSDPKVPVIMIGPGTGLAPFRGFLQERLAQK
EAGTELGTAILFFGCRNRKVDFIYEDELNNFVETGALSELVTAFSREGATKEYVQHKM
TQKASDIWNLLSEGAYLYVCGDAKGMAKDVHRTLHTIVQEQGSLDSSKAELYVKNLQM
AGRYLRDVW
SEQ ID NO: 6
Artemisia annua alcohol dehydrogenase (AaADH, wild-type)
MAQKAPGVITCKAAVVWESSGPVVLEEIRVDPPKASEVRIKMLCASLCHTDVLCTKGF
PI
PLFPRIPGHEGVGVIESIGKDAKGLKPGDIVMPLYLGECGQCLNCKTGKTNLCHVYPP
SF
SGLMNDGTSRMSIARTGESIYHFASCSTWTEYAVADCNYVLKINPKISYPHASFLSCG
FT
TGFGATWRETQVSKGSSVAVFGIGTVGLGVIKGAQLQGASKIIGVDVNQYKAAKGKVF
GM
TDFINPKDHPDKSVSELVKELTHGLGVDHCFECTGVPSLLNEALEASKIGIGTVVPIG
AG
GEASVAINSLILFSGRTLKFTAFGGVRTQSDLPVIIDKCLNKEIQLDELLTHEIHLDN
IQ
EAFEILKKPDCVKILIKF
SEQ ID NO: 7
Artemisia annua alcohol dehydrogenase (AaADH1, engineered
variant)
MAQKAPGVITCKAAVVWESSGPVVLEEIRVDPPKASEVRIKMLCASLCHTDVLCTKGF
PI
PLFPRIPGHEGVGVIESIGKDVKGLKPGDIVMPLYLGECGQCLNCKTGKTNLCHVYPP
SF
SGLMNDGTSRMSIARTGESIYHFASCSTWTEYAVADCNYVLKINPKISYPHASFLSCG
FT
TGFGATWRETQVSKGSSVAVFGIGTVGLGVIKGAQLQGASKIIGVDVNQYKAAKGKVF
GM
TDFINPKDHPDKSVSELVKELTHGLGVDHCFECTGVPSLLNEALEASKIGIGTVVPIG
AG
GEASVAINSLILFSGRTLKFTAFGGVRTQSDLPVIIDKCLNKEIQLDELLTHEIHLDN
IQ
EAFEILKKPDCVKILIKF
SEQ ID NO: 8
Artemisia annua aldehyde dehydrogenase (AaALDH, wild-type)
MASSGANGSSKSASHKIKFTKLFINGEFVDSISGNTFDTINPATEEVLATVAEGRKED
ID
LAVKAAREAFDNGPWPRMSGEARRKIMLKFADLIDENADELTTLEVIDGGKLFGPVRH
FE
VPVSSDTFRYFAGAADKIRGATLKMSSNIQAYTLREPIGVVGHIIPWNGPAFMFATKV
AP
ALAAGCTMVIKPAEHTPLTVLFLAHLSKLAGVPDGVINVVNGFGKTAGAAVSSHMDID
MV
TFTGSTEVGRTVMQAAALSNLKPVSLELGGKSPLIVFDDADVDKAAEFAILGNFTNKG
EM
CVAGSRVFVQEGIHDVFVKKLEGAVKAWATRDPFDLATRHGPQNNKQQYDKVLSCINH
GK
KEGATLVTGGKPFGKKGYYIEPTLFTNVTDDMTIAKEEIFGPVISVLKFKTVEEVIKR
AN
ATKYGLASGVFTKNIDVVNTVSRSIRAGAVWVNCYLALDRDAPHGGYKMSGFGREQGL
EA
LEHYLQIKTVATPIYDSPWL
SEQ ID NO: 9
Artemisia annua double-bond reductase (AaDBR2) (wild-type)
MASEKPTLFSAYKMGKFNLSHRVVLAPMTRCRAINAIPNEALVEYYRQRSTAGGFLIT
EG
TMISPSSAGFPHVPGIFTKEQVEGWKKVVDAAHKEGAVIFCQLWHVGRASHQVYQPGG
AA
PISSTSKPISKKWEILLPDATYGTYPEPRPLAANEILEVVEDYRVAAINAIEAGFDGI
EI
HGAHGYLLDQFMKDGINDRTDEYGGSLENRCKFILQVVQAVSAAIGTDRVGIRISPAI
DH
TDAMDSDPRSLGLAVIERLNKLQFKLGSRLAYLHVTQPRYTADGHGQTEAGANGSEEE
VA
TDAMDSDPRSLGLAVIERLNKLQFKLGSRLAYLHVTQPRYTADGHGQTEAGANGSEEE
VA
QLMKTWRGAYVGTFICCGGYTRELGLQAVAQGDADLVAFGRYFVSNPDLVLRLKLNAP
LN
RYDRATFYTHDPVVGYTDYPSLDKGSLL
SEQ ID NO: 10
Artemisia absinthium double-bond reductase (AbDBR2) (wild-
type)
MSEKPTLFSPYKMGNFNLSHRVVLAPMTRCRAINAIPNEALVEYYQQRSTAGGFLITE
GTMISPSSAGFPHVPGIFTKEQVEGWKKVVDAAHKEGAVIFCQLWHVGRASHQVYQPG
GAAPISSTSKPISKKWKILMPDATYGTYPEPRPLAANEILEVVEDYRIAAINAIEAGF
DGIEIHGAHGYLLDQFMKDGINDRTDEYGGSLENRCKFILQVVQAVSAAIGADRVGLR
ISPAIDHLDAMDSDPRSLGLAVIERLNKLQFKLGSRLAYLHVTQPRYTAYGQTEAGAH
GSEEEVAQLMKAWRGAYVGTFICSGGYTRELGLQAVAQGDADLVAFGRHFISNPDLVL
RLKLNAPLNRYVRATFYTHDPVVGYTDYPSLDKGNVGVERLSRL
SEQ ID NO: 11
Artemisia annua peroxidase 1 (AAO45182.1)
MGRIIVFQVLALCSLLVFPNIAFAQLKQNYYANICPNVESIVQKAVAAKVKQTFVTIP
GTLRLFFHDCFVQGCDASVMIQSSGSNTAEKDHPDNLSLAGDGFDTVIKAKAAVDANP
SCRNKVSCADILTMATRDVVKIAGGPSYSVELGRLDGLSSTAASVGGNLPKPNQNLDQ
LNALFAANGLTQADMIALSGAHTLGFSHCNQFSNRIYNFSKQNPVDPTLNPSYATQLQ
QQCPKNVDPRIAINMDPNTPRTFDNVYYKNLQNGQGLFTSDQVLFTDTRSKQTVISWA
NSPTAFNNAFITAMTKLGRVGVKTGTKGNIRKDCAAFN
SEQ ID NO: 12
Artemisia annu peroxidase 1 (PWA70787.1)
MSRFIFIQVLAFLSLLIFPNIAFAELKQNYYANICPDVETIVRKAVAAKLAETPITVQ
GTLRLLFHDCFVEGCDASIMIQSSGSNTAEKDHPENLSLAGDGFDTVMKAKAAVDAVP
SCKNKVSCADILTMAARDVVQMAGGPSYPVELGRLDGLSSTAASVNGRLPKANQNLDQ
LNAIFAANGLNQTEMIALSGAHTLGFSHCDQFLNRIYNFSKQNPVDPTLIPSYAAELQ
QRCPKNFDPDTVIDLDPSTTGIFDNMYYKNLQNGQGLLTSDQVLFTDMRSKQTVISWA
NSPKDFSDAFVKAMTKLGRVGVKTGKNGNIRRDCSAFN
SEQ ID NO: 13
PWA86724.1 peroxidase 1 [Artemisia annua]
MSRFIFIQVLAFLSLLIFPNIAFAELKQNYYANICPDVETIVRKAVAAKLAETPITVQ
GTLRLLFHDCFVEGCDASIMIQSSGSNTAEKDHPENLSLAGDGFDTVMKAKAAVDAVP
SCKNKVSCADILTMAARDVVQMAGGPSYPVELGRLDGLSSTAASVNGKLPKANQNLDQ
LNAIFAANGLNQTEMIALSGAHTLGFSHCDQFLNRIYNFSKENPVDPTLIPSYAAELQ
QQCPKKNFDPNNGQGLLASDQVLFTDTRSKQTVISWANSPKDFSNAFVKAMTKLGRVG
VKTGQNGNIRRDCSAFN
SEQ ID NO: 14
PWA42822.1 heme peroxidase [Artemisia annua]
MKKQILCLLFLSLFIAPSYAQLRQKFYQNTCPNVESIVTAAVTKKFQQTFVTIPGTLR
LFFHDCFVRGCDASVILANPNSEKDHPDDQSLAGDGFDTVIKAKAAVDSNPSCRNKVS
CADILALATRDVIRLAGGPFYSVELGRRDGRISTTASVTNNLPHPEFKLDQLNSMFAK
NGLSQTDMIALSGAHTLGFSHCSKFASRIYSRNGIDPTLNRQYATQLQQMCPQNVDPN
IAINMDPTTPRTFDNVYYKNLQQGKGLFVSDQILFTDSRSRATVNLFASNSTAFNKAF
VAAMTKLGRVGVLTGNLGEIRRDCARVN
SEQ ID NO: 15
PWA34719.1 heme peroxidase [Artemisia annua]
MKKQVLCLLFLSFFVASSYAQLRQNFYQNTCPNVESIVRSAVAKKFQQTFVTAPGTLR
LFFHDCFVRGCDASIFLSNNLNAEKDHPDDQSLAGDGFDTVIQAKAALDSNPNCRNKV
SCADILALATRDVVALTGGPSYKVELGRRDGRISTKASVQHKLPHADFNLDKLNTMFA
AHGLSQTDMIALSGAHTLGFSHCGQFSRRIYSKAGIDKTLNRNYALQLRQMCPLKVDP
RIAINMDPTTPRTFDNTYYKNLQQGMGLFSSDQILFTDQRSRPTVNLFASNNNAFNQA
FVNAMTKLGRVGVLTGNQGEIRRDCNRVN
SEQ ID NO: 16
PWA74744.1 heme peroxidase [Artemisia annua]
MKKQVLCLLFLSFFVASSYAQLRQNFYQNTCPNVESIVRSAVSKKFQQTFVTAPGTLR
LFFHDCFVRGCDASIFLSNNLNAEKDHHDDQSLAGDGFDTVIQAKAALDSNPNCRNKV
SCADILALATRDVVALTGGPSYKVELGRRDGRISTKASVQHKLPHADFNLDKLNTMFA
AHGLSQTDMIALSGAHTLGFSHCGQFSRRIYSKAGIDKTLNRNYALQLRQMCPVNVDP
RIAINMDPTTPRTFDNAYYKNLQKGMGLFSSDQVLFTDQRSRPTVNLFASNNNAFNQA
FVNAITKLGRVGVLTGNQGEIRRDCNRVN
SEQ ID N: 17
PWA70790.1 peroxidase 1 [Artemisia annua]
MVMKAKAAVDAVSSCKNKVSCADILTMAARDVVQMPENANCYKNPVDPTLIPSYAAEL
QQQCPKNFDPNTVIALGPSTTGIFDNMYYKNLQNGQGLLTSDQGCDASIMIQSSGSNT
AEKDHQDNLSLAGDGFDMVNKAKAAADAVPSCRNKVSCADILTMATRDVIKMAGGPSY
PVELGRLDGLSSTAASVNGKLPIPNWNLDQLNDMFAFNGLTQIDMIALSGAHTLGFSH
CDQFANRIYNFSKQNPLDPTLNPSYATQLQQLCPKDITPLVAIHMDPNTPTTFDMYFK
NLQQGQGLFTSDQVLYTDTRSKQTMISWANDPKAFNDAFIEAMT
SEQ ID NO: 18
PWA91189.1 peroxidase superfamily protein [Artemisia
annua]
MKVLCWWLMVLVLAGRGNGQLVENFYAATCPMAEAVVKQAVDAKVSQTFTTIPATLRL
FFHDCFVEGCDASVMIQSPNGDAEKDSSDNLSLAGDGFDTVIKAKEAVESFCPGVVSC
ADVLALAARDVVVAAGGPMENVQLGRRDGLVSKASRVAGNLPEPTFNRIQLNTMFARN
NLSELDMIALSGAHTLGFSHCNRFADRLYSSSVVDPSLDPTYAQQLMAACPQNVDPDI
AIDMDPETPHTFDNVYYQNLVAGKGLFTSDQVLFTDTSSQPIVNDFANSAGDFNAAFI
TAMRKLGTTGVKVGNQGEIRRDCTAFN
SEQ ID NO: 19
PWA42821.1 heme peroxidase [Artemisia annua]
MKKHVLCLLFLSFFVASSYAQLRQNFYQNTCPNVESIVRSAVAKKFQQTFVTAPGTLR
LFFHDCFVRGCDASIFLSNNLNAEKDHHDDQSLAGDGFDTVIQAKAALDSNPNCRNKV
SCADILALATRDVVALTGGPSYKVELGRRDGRISTKASVQHKLPHADFNLDKLNTMFA
AHGLSQTDMIALSGAHTLGFSHCGQFSRRIYSKAGIDKTLNRNYALQLRQMCPVNVDP
RIAINMDPTTPRNFDNAYYKNLQQWDGAFVNAITKLGRVGVLTGNQGEIRRDCNRVN
SEQ ID NO: 20
PWA47267.1 peroxidase superfamily protein [Artemisia
annua]
MSSKPLILILFISLLSSISSSYPQPLTTTFYQKTCPRFDQIMQDTTTNKQIASPTTAG
AALRLFFHDCLVEGCDASVLISSTHENKAERDADINLSLPGDGFDVVVRAKTALELAC
PGVVSCADILAVATRNLVTMMGGPFYQVKLGRRDGMVSRAARAETILPKPTMSMNQII
KIFSNIGLSVQEMVALTGAHTIGFSHCSEISHDIYNYSRTQMSDPSYNTRYADGLRNA
CKDFKKNPSLSVFNDIMTPHDFDNNYYKNLPKGLGVLRSDRAMMMDVRTRKYVELYAR
DQKAFFDAFGRAIEKLSLVGVKSGRNGQIRRRCDSFN
SEQ ID NO: 21
PWA63464.1 peroxidase 5 [Artemisia annua]
MSYIYAIFILVTLRCVVHSEAQLQVGFYSHSCPAAEFIVKDEVSKAFSQNNGLAAGLV
RLHFHDCFVRGCDGSVLIDSTPSNTAEKDSPANNPSLRGFNVIDNAKSRLEKACPGVV
SCADIVAFAARDGFQITGGLGYDVPAGRRDGRVSRIADTSGLPPPTSNLNQLTQLFAS
NGLTQEEMVTLSGAHTIGRSHCTSFVNRLYNFSPSAKQDPTLDKLYASKLKQQCPKGS
NNVNLVVPMNPSSPTVSDTEYYVDVLNNRGLFTSDQSLLTSKSTANQVHQNAMDPLLW
KSKFAKAMVQMGKNGVLTGRQGEIRKNCRVINKCNLKNKQENLELCSEELKQEGTAWQ
AES
SEQ ID NO: 22
PWA39629.1 heme peroxidase [Artemisia annua]
MAIMSALLISLLILSNFSLGTSLSSNYYTKTCPNVESLIRKAVRDAATSDKKVPAALL
RMHFHDCFIRGCDASVLLDSKGKNTAEKDGPPNVSLHAFYVIDNAKKAVESACPGIVS
CADILALAARDAVVLSGGPSWDVPKGRKDGRTSKASETIQLPAPTFNISQLQQNFSQR
GLSLEDLVALSGGHTLGFSHCSSFNNRIHNFNSTTDVDPSIHPSFAASLRSICPAKNN
AKNAGVPMDPSSTSFDNTYYKLIFQQKALFSSDKALLDSPKTKNLAMKFASSKDAFTK
AFIKSMIKMSSITGGQEIRKDCKMVN
SEQ ID NO: 23
PWA97466.1 heme peroxidase [Artemisia annua]
MAAMSALLTSLLILSNFSLGTSLSSNYYAKTCPNVESVIRKAVRDAATRDKKVPAALL
RMHFHDCFIRGCDASVLLDSKGKNTAEKDGPPNVSLHAFYVIDNAKKAVEAACPGIVS
CADILALAARDAVVLGSKRSMFLMFLVQSGGPSWDVPKGRKDGRTSKASETIQLPAPT
FNISQLQQSFAHRGLSLEDLVALSGGHTLGFSHCSSFKNRIHNFNSTTDVDPSIHPSF
AASLRSTCLAKNNAKNAGVPMDPSSTSFDNTYYKLIFQQKALFSSDKALLDFPKSKNL
AMKFASSKDAFTKAFIKSMIKMSSITGGQEVRKDCKVVN
SEQ ID NO: 24
PWA70538.1 peroxidase 5 [Artemisia annua]
MMSTTSLNATFFVKLMLVLMLMSPLQAQQLQVGFYSGSCFMAEFIVKDEVTKAFFRDR
GLAAGLVRLHFHDCFVRGCDASVVLDSTPSNTAEKDSPANNPSLRGFDVIDNAKARIE
SACPGVVSCADIIAFAARDSIILTGGLGYDVPAGRRDGRVSLIAETRALPPSTANLNQ
LTQMFSTHGLTQEDMVTLSGAHTIGRSHCTSFSSRLYSYNTTVNQDPTLNAFYASQLM
QQCPKGGPDSVVPINPASPTITDVGYYVDILNNRGLFTSDQTLLSTTSTANQVTRYAI
NPLLWKTKFAAAMVKMGKIGVLTGAQGEIRSNCSVINN
SEQ ID NO: 25
PWA38586.1 heme peroxidase [Artemisia annua]
MAALLSSLLILSFFSLGTSLSSNYYQKTCPNAESIIKKAVIDATKNDKTAPAALVRMH
FHDCFIRGCDGSVLINSKGKNTAEKDGPPNKSLHAFYVIDNAKKALESACPGVVSCAD
ILAVAARDATVVSGGPSWDVPKGRKDGRISRASETSQLPAPTFNISQLQQSWSQRGLS
MEDLVALSGGHTLGFSHCSSFNNRIHNENSTFDIDPSLQPSFAATLRRVCPARNNAKK
AGVTMDPSSTTFDNTYYKLILQNKVVFSSDNALLNSPKTKSLVSKFASSSDAFFKAFA
KSMIKMSSITGGQEIRKDCRVVN
SEQ ID NO: 26
PWA37746.1 heme peroxidase [Artemisia annua]
MAVKNTTIFSLLFSLAFLGLTCVHLTNALSSNYYDKTCPNVEATITSVVKKAMLNDRT
VPAALLRMHFHDCFIRGCDGSVLLNSTKKNQAEKDGPPNISLHAFYVIDNAKKALEAL
CPKTVSCADILALAARDAVTLSGGPNWNVPKGRKDGRVSKATDTRQLPGPTFNISQLQ
QSFAQRGLGTEDLVALSGGHTLGFAHCSSFQNRIHNFSPKKSVDPTLQSSFAASLKNV
CPAKNTPKNAGANLDSTPTTFDNRYYKLLLQGKSIFSSDQALATMANTKALVTKYASS
KKEFEKAFVKSMIKMSSINGGGQEVRHDCRVVN
SEQ ID NO: 27
PWA35354.1 protein kinase domain-containing protein
Artemisia annua]
MSYIYAIFILVTLRCVVHSEAQLQVGFYSHSCPAAEFIVKDEVSKAFSQNKGLAAGLV
RLHFHDCFVRGCDGSVLIDSTPSNTAEKDSPANNPSLRGFNVIDNAKSRLEKACPGVV
SCADIVAFAARDGFQITGGLGYDVPAGRRDGRVSRIADTSGLPPPTSNLNQLTQLFAS
NGLTQEEMVTLSGAHTIGRSHCTSFVNRLYNFSPSAKQDPTLDKLYASKLKQQCPKGS
NNANLVVPMNPSSPTVSDTEYYVDVLNNRGLFTSDQSLLTSKSTANQVHQNAMDPLLW
KSKFAKAMVQMGKNGVLTGRQGEIRKNCRVINKCLDVVCKQLSDFECSFPVEVACDIL
RLDKWDGDERSEFVPSNITTISFSSTPLFLDTSVPQIPYSTARVENTSSFTYTFPVSE
GPKFVRLYFYPATYSGLNANLSSFSVSSNGYSLLTNFSAFLTALFLAKTCDGPPVPHF
VKEFLIYVQDTQFLSVVFTPAPNSYAFINGIEIVSMPDNLYFNAKNPKPVGMSTGHVI
NSDTALETVYRLNVGGAQISGINDTGIYRSWDQDINYIYGPIGVTPLNENPIAYTETT
PNYTAPEAIYQTQRSMGIQSELYNLTWILPVDSGFYYMLRFHFSSIIPQYTKDLQMIF
RIFINNQTAEDDADIIYWAQGSRIPVYRDYVVLVNNPSGHRSKQDLWLQMHPNLEVSK
EYLDAYLNGLEAFKLSMNHDLSSPMLSEKNLSSPTPNLVSSRKENNNKTPPSALIIGG
AGGGLVLLSVLLLLVLYQRRRTKHHTTTNNKPSLLSDCCRRFSLKQVKVATNKFNENN
VIGNGGFGKVYKGYMGNAKTTVAIKRLNPSSSQGFHEFQTEIQMLSRLRHEHLVSLIG
YCDDNGEMILVYDYMSRGNLREHLYKTNNPPLSWKTRLNICIGAAKGEQVTLAEWGKT
CYQRGTLVEIIDQNLSDQIAPGSLRKFGEVANSCLHEEVSERPTMDAVVQGLEVALQL
QEDCEEMGGIVGEEMLESQDLHFHMQVELTKTDDDGIEDGEVVAHQGAHS
SEQ ID NO: 28
PWA70233.1 peroxidase superfamily protein [Artemisia
annua]
MAINTSHEPQAPTHKLIFMAMAFSKASCQSTLIFALFLLCNLRALEAQTPAPAPGLSY
TFYRTTCPNLETIIRQQLQTVFASDIGQAAGLLRLHFHDCFVQGCDASVLLDGSASGP
SEKDAPPNLTLRAQAFVIIENLRRLVHNACNRTVSCADITALAARDAVFLSGGPNYSI
PLGRRDGLTFATVNVTLANLPPPTANASTILSSLALKGFTPTDVVSLSGGHTIGIGHC
GSFNNRIYPSRDPSMDQTFFNNLRLTCPTNTSNNTTVLDIRSPNVFDNKYYVDLMNRQ
GLFTSDQDLYTDSRTRTIVTNFAVNQTLFYQNFINAMIKMGQLNVLIGTQGEIRANCS
RRNSNDLFILPTMENDQDEKVASY
SEQ ID NO: 29
PWA70232.1 peroxidase superfamily protein [Artemisia
annua]
MAFSKASCQSTLIFALLLLCNLRALEAQTPAPAPGLSYTFYRTTCPNLETIIRQQLQT
VFASDIGQAAGLLRLHFHDCFVQGCDASVLLDGSASGPSEKDAPPNLTLRAQAFVIIE
NLRRLVHNACNRTVSCADITALAARDAVFLSGGPNYSIPLGRRDGLTFATVNVTLANL
PPPTANASTILSSLALKGFTPTDVVSLSGGHTIGIGHCGSFNNRIYPSRDPSMDQTFF
NNLRLTCPTNTSNNTTVLDIRSPNVFDNKYYVDLMNRQGLFTSDQDLYTDSRTRTIVT
NFAVNQTLFYQNFINAMIKMGQLNVLTGTQGEIRANCSRRNSNDLFILPTMENDQDEK
VASY
SEQ ID NO: 30
PWA43433.1 peroxidase superfamily protein [Artemisia
annua]
MTNKIQPQQFPTITENLLSFDSMFHQLHTLLILLATMGNSRLFQLIFLIITSILTSID
ASAKYLHRNNHKNPPPPPLTLDYYAKTCPRFHEIVRTTVVPKQLSHPTTAAATLRLFF
HDCMVGGCDASVLIASNAYNKAERDYDINESLAGDGFDVVTRVKMALEVECPGVVSCS
DVLAITTRDLLIQVGGPHYEVKLGRKDGLESKASNVEGKLGRANMTLNEVIRVFESHK
YTHREMVALMGGGHTIGFAHCKEFESRLFGPKPDPSVHPKLAERLKAMCVNRSKDPSV
SAFLDPISAGNFDNMIFKNILNGLGVLGTDQAMASDPRTRPLVEEYARDSAKFFNDFA
RAMEKTSVYQVKTGNQGEVRHASAKYLHRNNHKNPPPPPLTLDYYAKTCPRFHEIVRT
TVVPKQLSHPTTAAATLRLFFHDCMVGGCDASVLIASNAYNKAERDYDINESLAGDGE
DVVTRVKMALEVECPGVVSCSDVLAITTRDLLIQVGGPHYEVKLGRKDGLESKASNVE
GKLGRANMTLNEVIRVFESHKYTHREMVALMGGGHTIGFAHCKEFESRLFGPKPDPSV
HPKLAERLKAMCVNRSKDPSVSAFLDPISAGNFDNMIFKNILNGLGVLGTDQAMASDP
RTRPLVEEYARDSAKFFNDFARAMEKTSVYQVKTGNQGEVRRRCDAFNNLPNGRKT
SEQ ID NO: 31
PWA66914.1 peroxidase [Artemisia annua]
MILMALETTEAQAQLKMGFYRTTCPKAEWIVQDTVNKAVKANPGIAAGIIRLYFHDCF
VRGCDASLLLKTVQGSENESEQDHPANAGTLRGLEIFDQAKARLEAACPNTVSCADIL
AFAARDSTNAVGGFSYAIPSGRRDGRISNIGDVDLPGPDSDLRNMRSKFMAKGLSTRD
LVALSGAHSIGRSGCNLVAPRLYPTDPSLDPKYAAFLKTKCPKNRLSGTTNLDPVSAN
RLDNQYYRNVKQHKVLFSSDEALLDSSETARIVATYTNNTNAWRMDFAAAMVRMGSVE
VLTGTKGEIRKKCSVIN
SEQ ID NO: 32
AOS50886.1 peroxidase [Artemisia annua]
MTMAFSKASCQTLIFALLLLCNLRALEAQTPAPAPGLSYTFYRTTCPNLETIVRQQLQ
TVFASDIGQAAGLLRLHFHDCFVQGCDASVLLDGSASGPSEKDAPPNLTLRAQAFVII
ENLRRLVHNACNRTVSCADITALAARDAVFLSGGPNYSIPLGRRDGLTFATVNVTLAN
LPPPTANASFILESLSTKGFTPTDVVSLSGGHTIGIGHCGSFSNRIFPSRDPSMDQTF
FNNLRLTCPTNTSNNTTVLDIRSPNVFDNKYYVDLMNRQGLFTSDQDLYTDSRTRTIV
TNFAVNQTLSYQNFINAMIKMGQLNVLTGTQGEIRANCSRRNSNDLFILPNMERDQDE
KVASY
SEQ ID NO: 33
PWA47104.1 heme peroxidase [Artemisia annua]
MAEKNTAILSFLFSLVFLGLTSLSLINALSSNYYDQTCPKAEAEITSAVKKAMLNDRT
VPAALLRMHFHDCFIRGCDGSVLLNSTANNQAEKDGPPNISLHAFYVIDNAKKAVEAL
CPKTVSCADILALAARDAVTLSGGPTWNVPKGRKDGRVSKATDTRQLPGPTFNISQLQ
QSFAQRGLGTDDLVALSGGHTLGFAHCSSFQNRINNFSSKQSVDPTLQSSFAASLKSV
CPAKNTPKNAGANLDSTPTTFDNRYYKLLLQGKSIFSSDQALVTMGNTKALVTKYASS
KQEFEKAFVKSMIKMSSISGGGQELAWCCNQTSYKIGLRKLVSKPEGHTKLLIIDTAT
TNLKGVESKFAPALFTLLCIGHTLRKLAAKACCSDGSTSCVVAKLWILFWNEEQ
SEQ ID NO: 34
PWA39798.1 peroxidase 1 [Artemisia annua]
MGAHTLGFSHCDQFLNRIYNFSKENPVDPTLIPSYAAELQQQCPKKNFDPNVVIPLDP
STTGIFDNMYYKNLQNGQGLLASDQVLFTDTRSKQTVISWANSPKDFSNAFVKAMTKL
GRVGVKTGQNGNIRRDCSAFN
SEQ ID NO: 35
PWA81992.1 peroxidase superfamily protein [Artemisia
annua]
MNQKNIIIAFLVSCSLLLANGQLDYYYYYSFCPNLEMIVKYGVWAAMKNDTRIAASLL
RLHFHDCFVNGCDGSILLDDTKTFKGEQNAGPNINSARGYEVIDNIKADVERACPSTV
SCVDILTLAAREAVVLSGGPNWPVALGRRDGVTANLKAANTNLPSPFEPLANITAKFA
AVGLDIRDVVVLSGAHTIGLAQCFTFKNRLFDFKGTGQTDPNLDTSLATSLKTSCPNV
DSSNSNLNSLDLVTTYKFDNAYYKNLVNNAGLLESDQALMGHPLTASMVKDYSMYPYL
FYKDFATSMVKLGNIGVITGQNGQVRKKCGEVNR
SEQ ID NO: 36
PWA36708.1 peroxidase 27 [Artemisia annua]
MAIQKQLIAVLQLLLIALVFDIANGKGLELGFYRYTCPRVEAIVRKTTANYIHRAPSL
AAGLLRMHFHDCFVRGCDGSVLLNSTANNQAEKEGIPNLTLRGFQVVDAAKTALEAAC
PGVVSCADILALVARDAIYQIKGPFWPVPLGRRDGRVSIANEALTSLPSPFANITQLK
ANFASKGLNTKDLVVLSGGHTVGVSHCPVIVNRLYNFTGKGDTDPSLDPKFIPQLKSI
CSPTDTTSVLAMDRTPRSFDEDYYTVVLRKRGLFQSDAALLNDKQTSAYVKLQSKSFG
YTFFNDFSKSMVKMGKIGVLTGSAGEIRRTCALVN
SEQ ID NO: 37
PWA37745.1 heme peroxidase [Artemisia annua]
MVSCNLSSLLLGVFVLATIHSSLSITLSPNYYDQTCPNVEAAVTSAVKKAMLNDRTVP
AALLRMQFHDCFIRGCDGSVLLNSTKNNQAEKDGPPNISLHAFYVIDNAKKALEAMCP
GTVSCADIIALAARDAVHLSGGPSWVVPKGRKDGRISKATETRQLPAPTFNISQLQQS
FSQRGLDINDLVALSGGHTLGFAHCSSFQNRIHNFATTQDVDPSLQQAFAASLRSVCP
MHNKVNSAGANLDSTPTIFDNSYYKLLLEGESIFSSDQALLTNPTTQKLVTKYAYSRE
EFEKAFVESMIKMSSITGGQEKTDVTRFSSTPTRAECLSSRNLETKTS
SEQ ID NO: 38
PWA97649.1 peroxidase superfamily protein [Artemisia
annua]
MKSLSTNGFAWFITISMLCLLANSQLTTNFYSNSCPNLFSIVRKQVLNAVKTETRMAA
SLLRLHFHDCFVNGCDASVLLDGSDGEKTALPNNNSARGFEVVDAIKTAVESSCSGIV
SCADILAIAARDSVLLSGGPSWKVLLGRRDGLVANQTGANSMLPGPFEPVDNITLKFQ
AVGLNLTDVVSLSGAHTIGRASCATFGNRLFNFSGTNAPDSTLLDANMVSGLQNLCPV
NGDSSRVTDLDWNSTDLFDNHYFQNLINGRGILESDQFLYSSDQAVSTTRSIVELYSN
NTKLFFNDFANSMIKMGNIQPLTGSAGEIRKNCRVVNS
SEQ ID NO: 39
PWA36179.1 peroxidase [Artemisia annua]
MAIQKHNTLGLRFLLVALLLNIANGNDLKLGFYKKTCPRAEAIVKRTTANFISRAPSL
AAPLLRMHFHDCFVRGCDGSVLLNSTKNNQAEKAGIPNLSLRGFQVIDAAKTAVEAAC
PGVVSCADILALVARDSIQQIKGPYWPVPLGRRDGRVSIASESFTLPAPFANITTLKS
QFKSRGLSVKDLVVLSGGHTVGVSHCSTIASRLYNFTGKGDTDPTLDPKYVPHLKSKC
KPTDTTTLLEMDPGSVKTFDEDYYTIVSKRRGLFESDAALLNDKQTSAYVKLQAKSHG
ATFFKDFSDSMVKMGKIGVLTGKNGEIRKHCALIN
SEQ ID NO: 40
PWA58751.1 heme peroxidase [Artemisia annua]
MEGSSLTKTLVMLVLLLATLTTLALGQSVRGGGGGTRVGFYSSTCRNVESIVQSAVRT
AVQANPTIAPGLLRMFFHDCFVNGCDASILINGTNTEKSARPNSLLRGFEVIDAAKSR
LETSCPGVVSCADILALAARDSVVLTGGRSWPVPLGRRDGLVSQASDTANLPAFNDPM
TVQIQKFADKGLNIQDLVTLVGGHTIGTAACALFSYRLYNFNNTNAPDPDINPAFLPT
LRALCPNGGDGSRRVALDTDSVNSFGSSYYNNLRNGRGVIESDAKLWSDQRTQRFVQG
FLGANGARFNAEFGRAMVRLGNVEVKTGTQGQIRRVCTATN
SEQ ID NO: 41
PWA71300.1 heme peroxidase [Artemisia annua]
MASSSSASLFLILSSLLIFSSLLYVSEADYSAPIVKGLSWDYHHKTCHKVEKIVRKHL
KKVFKEDIGQAAGLLRLHFHDCFVQGCDGSVLLDGSHSGPSEQTAPPNQSLRKQAFTI
IEDLRGLIHRECGRVVSCSDIVAIAARDAVHLSGGPDYDVPLGRKDGLTFATENATLA
NLPAPTSNASTILSALATKNLTPTDVVALSGGHTIGISHCTSFTPRLYPTQDPTMEKT
FAHGLKEVCPTNTTDATTVMDILSPIKFDNKYFVDLMNRQGLFTSDQDLYTHKETKPI
VESFAHDEKLFFEKFVKAMIKMGQMEVLTGGKGEIRAHCSIRNPDNKAYFSYLVEEDE
QSYLESRAELR
SEQ ID NO: 42
PWA47367.1 heme peroxidase [Artemisia annua]
MASNMALLASVLVVMLMHLQSLEAQQPPLVKGLSWKYYESSCPKLESIIRKQLEKVIK
KDVGLAAALLRMHFHDCFVQGCDGSVLLDGSASGPVDSEKTAPPNLSLRGFDVIEDLR
RRVHKECGAGSVSCADITAIVARDSIVLTGGPTYSVPLGRRDGLSFATRDATLANLPS
PFVPTDNLLRTLAAKNFDATDAVALSGAHTIGIAHCSSFTRRLYPTQDTTLDKTFATT
LKGVCPAPDTNASTFMDLRSPNVFDNKYFVDLMNKQGVLTSDQDLYTDKRTRNIVTSF
AVNQTLFFEKFVNVMIKMGQMEVLTGTQGEVRNKCSVTNSNDLLISSVVEGDNMDSVE
SF
SEQ ID NO: 43
PWA95898.1 heme peroxidase [Artemisia annua]
MAFLKASCLPTLIFVLLLCNIRASDAQTPPLAEGLSYTFHQSSCPQLESIVRSQLQTE
LAADIGQAAGLLRLHFHDCFVLGCDGSVLLNGSASGPSEQSAPPNLTLRPRAFVIIEN
IRSLVHAACNATVSCSDITALAARDAVVLSGGPNYSVPLGRRDGVSFASREDTLANLP
PFNATAANLTASLALKGFDVTDVVALSGAHTIGRAECGSFERRMFPTPDPTMDQIFYT
NLLQTCPILNTTNTTIMDIQTPNAFDNRYYVNLMNRQGLFTSDQDLFTDNTTSGIVTS
FANNQTLFFENFVIAMIKMGQLNVTTGTQGEIRANCSFVNSNNTLSLPAMENANDNQK
IASY
SEQ ID NO: 44
PWA95899.1 heme peroxidase [Artemisia annua]
MASKMTLLASVLVVMLMHLQSLEAQQPNLVKGLSWKYYESSCPKLESIIRKQLEKVIK
KDVGLAAALLRMHFHDCFVQGCDGSVLLDGSASGPVDSEKTAPPNLSLRGFDVIEDLR
RRVHKECGAGSVSCADITAIVARDSIVLTGGPTYSVPLGRRDGLSFATRDATLANLPS
PFVPTDNLLRTLAAKNFDATDAVALSGAHTIGIAHCSSFTRRLYPTQDTTLDKTFATT
LKGVCPAPNTNASTFMDLRSPNVFDNKYFVDLMNKQGLLTSDQDLYTDKRTRNIVTSF
AVNQTLFFEKFVNVMIKMGQMEVLTGTQGEVRNKCSVQNSNDLLISSVVEGDNMDSVE
SF
SEQ ID NO: 45
PWA35355.1 peroxidase 5 [Artemisia annua]
MSSIILNHVCNSIFILVLLGCVFNSKAHLQVGFYSESCSIAESIVKDEVYKAYSEDSG
LAAGVVRIHFHDCFVRGCDGSVLIDSTPSNTAEKDSPVNNPSLRGFNVIDDAKARLEE
ACPGVVSCADIVAFAARDAIHITGGYGYDVPAGRRDGVVSLLAETIGLPPPTFNLSQL
TQMFESNGLTQEEMTGGYGYDVPAGRRDGVVSLLAETIGLPPPTFNLSQLTQMFESNG
LTQEEMVTLSGTHTIGRAHCTSFADRLVNQDPTLDASYASSLKQQCPNGGNDVNLVVP
MDPSSPAVTDTAYYVDVLHNRGLFTSDQTLLTSASTAHQVYQNAIYPSLWKSKLADAM
VKMGKIGVLTGDQGEIRLNCRVINK
SEQ ID NO: 46
PWA40773.1 peroxidase superfamily protein [Artemisia
annua]
MSHNLFLLPLVFLALFPVVAPQLRQGYYSDTCPNAEMTVTARMQKAMIKEPRSGASVM
RFQFHDCFVNGCDASLLLDDTPTMLGEKLSLSNIDSLRSYEVVDEIKEALERICPMTV
SCADIIIMASRDAVALSAGPFWEVKLGRLDSLSASQKASDDIMPSPRSNASMLIDLFA
RFNLSVQDLVALSGSHSIGNARCFSIVHRIYNQSDDGKPDPSIEPGFLKKLQELCPLG
GDENVIGDLDFTPRTFDNEYFKDLVNGKGFLNSDETLFTNPQTREYVQRYSEDQSAFL
KDFVEGMIKLGDLQSGRPGEIRKNCRVVNSPPPANVMVRK
SEQ ID NO: 47
PWA75161.1 peroxidase superfamily protein [Artemisia
annua]
MKGVCLIFFVLAILGTVQGDLKMGFYSRSCPKAEKIIQDYVNQHIPRVPSLGAALIRM
HFHDCFVRGCDGSVLLNSTASSGNQTEKTAIPNRTVTGFDFIDKLKSLLESACPGIVS
CADIIALATRDTIVVTGGPTWRVPTGRRDGTVSIASEALAQIPAPSDNITLLQQSFAN
KSLDLKDLVLLSGAHTIGIARCPSFSNRLYNFSASSSTDPSLDSEYADVLRASKCRTP
NDNTTLVEMDPGSRRTFDLSYYRLLLKRRGLFESDSALTKSSQTLRYVNKILQGSLDN
FYKEFGLAMEKMGRIDVKTGSSGEIRRNCAFVNS
SEQ ID NO: 48
PWA63748.1 peroxidase superfamily protein [Artemisia
annua]
MKGVCLIFFVLAILGSVQGDLKMGFYSRSCPKAEKIIQDYVNQHIPRVPSLGAALIRM
HFHDCFVRGCDGSVLLNSTASSGNQTEKTAIPNRTVTGFDFIDRLKSLLESACPGIVS
CADIIALATRDTIVVTGGPSWRVPTGRRDGTVSIASEALAQIPAPSDNITLLQQSFAN
KSLDLKDLVLLSGAHTIGIARCPSFSNRLYNFSASSSTDPSLDSEYADVLRASKCRTP
NDNTTLVEMDPGSRRTFDLSYYRLLLKRRGLFESDSALTKSSQTLRYVNKILQGSLDN
FYKEFGLAMEKMGRIDVKTGSSGEIRRNCAFVNS
SEQ ID NO: 49
PWA49266.1 heme peroxidase [Artemisia annua]
MGYPKSIGTILVPIVVLLVSMNYMPCKAQLSTTFYDNTCPTALTTIRDSISSAVSRNT
RMAALIIRLHFHDCFVQGCDASILLEGGEKAAPANNGVLGYDAIDDAKAAVESVCPGV
VSCADILAVAARDASVAVGGPSWAVRLGRRDSPDSNPGQAATDLPRGNNNLAQLIASF
DRKGLSVRDMVALSGSHTIGQARCVTYRARIYDNTSNIDATFAANLRSDCPQTGTTTT
SAPLDLQTPNTFDNNYFRNLQASRGLLTSDQVLFNGDSTDSIVTEYVDNPSLFDSDFA
TAMINMGDIDPLTGTIGTIRTLCNTAN
SEQ ID NO: 50
PWA60167.1 peroxidase [Artemisia annua]
MGYPKSIGTILVPIVVLLVSINYMPCKAQLSTTFYDNTCPTALTTIRDSVRSAVSSNR
RMAALLIRLHFHDCFVQGCDASILLDGTGSERRSLANDGVGGYEAIDDAKAAVESVCP
GVVSCADILAVAARDASVAVNGPSWAVRLGRRDSSDSNAAEAGTDLPRGNNNLNELIA
SFARKGLSVRDMVALSGSHTIGQARCVTFRARIYDNNSNIDPTFRDNLRRNCPQAAPN
GNNNSEPLDLQTPNTFDNNYFLNLRVSRGLLTSDQELFNGGSTDSIVTEYVDNPSLFD
SDFATAMVNMGDIDPLTGTSGTIRTLCSTAN
SEQ ID NO: 51
PWA96214.1 peroxidase superfamily protein [Artemisia
annua]
MSKITISAVVAFLLILAVVPFSRAALNAHYYDQTCPQAESIIFQTIRNASIYDPKVPA
RLLRMFFHDCFIRGCDASLLLDSTPGNKAEKDGPPNVSVRSFYVIDDAKTKLEKACPR
TVSCSDVLAIAARDVVAMSRGPWWHVLKGRKDGRVSKANETINLPSPFSNTTTLIQSF
AKRGLNVKDLVTLSGGHTLGFSHCSSFSARLQNSTDTSINSEFAMSLRKKCPLHNKDR
NAGEFLDSTSAKFDNDYYKRITMGKGVFGSDQALYGDDRTKSIVDSYAKDEKRFFTEF
AASMVKLGNVGVIEDGEIRVKCSIVN
SEQ ID NO: 52
PWA57889.1 peroxidase superfamily protein [Artemisia
annua]
MSKITISAVVAFLLILAVVPFSRAALNAHYYDQTCPQAESIIFQTIRNASIYDPKVPA
RLLRMFFHDCFIRGCDASLLLDSTPGNKAEKDGPPNVSVRSFYVIDDAKTKLEKACPR
TVSCSDVLAIAARDVVAMSRGPWWHVLKGRKDGRVSKANETINLPSPFSNTTTLIQSF
AKRGLNVKDLVTLSGGHTLGFSHCSSFSARLQNSTDTSINSEFAMSLRKKCPLQNKDR
NAGEFLDSTSAKFDNDYYKRITMGKGVFGSDQALYGDDRTKSIVDSYAKDEKLFFTEF
AASMVKLGNVGVIEDGEIRVKCSIVN
SEQ ID NO: 53
PWA54319.1 heme peroxidase [Artemisia annua]
MGYHKSLRMILVPIVVLLVSVNYMPCKAQLCPKFYDDTCPSALATIRDSVSSAVSSNR
RMAAFLIRLHFHDCFVQGCDASILLEGTGSEKESPMNDGVLGYEAIDDIKAAVESVCP
GVVSCADILAVAARDASVEVGGPSWEVRLGRRDSIGTNASQAITDIPRGHNNLSELIA
SFDRKGLSVRDMVALSGSHTIGMARCVRFRDRIYDNTSNIDASFAANLRSNCPQTAPN
GDGNLEPLDLQTPNTFDNNYFRNLVTRRGLLTSDQVLFNGDSTDSIVTEYVDNPSLFD
SDFATAMVKMGDIEPLTGTSGTIRTRCNTAN
SEQ ID NO: 54
PWA49438.1 heme peroxidase [Artemisia annua]
MKGVCLIFGVLVILGSVRCELTTGFYSHSCPKAEKIIQDYVNQHILKVPSLAAALIRM
HFHDCFVRGCDASLLLNVTPTSGNQTEKGAIPNQTILGFDFIDKLKSLVEAECPGVVS
CADIITLAARDSIVVIGGPSWKVPTGRRDGLISIAFDTFTQIPAPFDNITILQQKLVN
KGLDLKDLVVLSGVHTIGVAHCSSFSNRLYNFSGRGDQDPTLDGEYANNLRSRKCTTP
NDSTTLVEMDPGSSKTFDRSYYTLLLKRRGLFQSDSALTKNATTLKYINKILKGSLKK
FYKQFALSMQKMGRIDVKTGSAGEIRRNCAFVNS
SEQ ID NO: 55
PWA46444.1 peroxidase [Artemisia annua]
MDSKKSILFVVLLATLITLALGQTRVGFYQTSCPRVETIVQTAVRSAINSNPAIAPGL
LRMHFHDCFVNGCDASILIDGASSEKTAPPNGFLRGFEVIDAAKTQLETACPGVVSCA
DILALAARDSVVQTGGASWQVPLGRRDGLVSRASDTGSLPGENEPLNALIRKFADKNL
NTQDLVTLSGAHTIGTASCLTFSYRLYNYNNTNGPDPSIDPSFLPTLRNLCPNGGDGT
RRVTLDTSSVGNFDNSYYRNLRNRRGVLESDAALWNNPATQTLVNRFLGIRGLAGLTF
NVEFGRSMVKMGNIELKTGSQGEIRRVCTAIN
SEQ ID NO: 56
PWA88515.1 heme peroxidase [Artemisia annua]
MQRLTSVVFWFLVFGPFVYGQLDYKFYDSTCPNLTKIVRFGIWSAMANDTRIAASLLR
LHFHDCISNGCEGSVLLDKTSDFSSEKDANPNRNSARGFELIDTIKANVEKACPKTVS
CTDILTLAAREAVFLSGGPFWSLAMGRRDGLTANMSAANTDTPSPFEPLENITAKFTS
KGLNLKDVVALSGAHTIGFAQCFTFKSRLFNFDGSGAPDPQLDSSLASSLQTVCPNQD
NSDTNLVPLDSVTTAKFDNIYYKNILNNSGILGSDQALMNDNITATMVINYSKYPYLF
AKDFGASMVKLSTIGIITGQNGQVRNRCNIVNQN
SEQ ID NO: 57
PWA70791.1 peroxidase 1 [Artemisia annua]
MVMKAKAAVDAVSSCKNKVSCADILTMAARDVVQMPENANCYKNPVDPTLIPSYAAEL
QQQCPKNFDPNTVIALGPSTTGIFDNMYYKNLQNGQGLLTSDQGCDASIMIQSSGSNT
AEKDHQDNLSLAGDGFDMVNKAKAAADAVPSCRNKVSCADILTMATRDVIKMAGGPSY
PVELGRLDGLSSTAASVNGKLPIPNWNLDQLNDMFAFNGLTQIDMIALSGNNPAKQYE
LR
SEQ ID NO: 58
PWA46198.1 peroxidase [Artemisia annua]
MVVLSHNKKSILCLLLLATLITLSLGQGTRLGFYRTSCPRVESIVQNAVNSAVRANPT
IAPGLLRMFFHDCFVNGCDASILIDGASSEKTARPNSLLRGFEVIDAAKRQLEIACPR
VVSCADILALAARDSVVQTGGTGWSVPLGRRDGLVSRAADTANLPAFNDPINVQIRKF
TDKGLNTQDLVALSGAHTIGTAACALFSYRLYNYNNTNGPDPTIDASFLPTLRNLCPN
GGDGTTRVTLDTGSVRNFDNSYYSNLRNRRGVLESDAALWNNPATQTLVNRFLGLRGL
LGLRFNLEFGRSMVKMGNIEVKTGTQGEIRRVCTATN
SEQ ID NO: 59
PWA89543.1 peroxidase 4 [Artemisia annua]
MGFLVKFAFVMFLVGVASAQLSTNFYSKSCPNLFPAVKSAVQAAISKETRMGASLLRL
HFHDCFVNGCDGSILLDDTSSFTGEKNAAPNFQSARGFDVVDNIKSAVEKACPGVVSC
ADILAITARDSVQILGGPTWNVKLGRRDSRTASQAAANSSIPPPTSSLSALISSFNRV
GLSAKDMVALSGSHTIGQARCTSFRPRIYNETNIDASFAASRRSNCPRTSGSGDNNLA
PLDLQTPTKFNNDYYKNLIAQKGLLHSDQQLFNGGSADSTVRQYSSNPSQFSSDFAAA
MIKMGDFRPLTGSSGEIRKNCRKPN
SEQ ID NO: 60
PWA60646.1 peroxidase [Artemisia annua]
MVVLFNNKKPILCLLLLATLITLSLGQGTRLGFYRTSCPRVESIVQNAVNSAVRANPT
IAPGLLRMFFHDCFVNGCDASILIDGASSEKTARPNSLLRGFEVIDAAKRQLEIACPR
VVSCADILALAARDSVVQTGGTGWSVPLGRRDGLVSRAADTANLPAFNDPINVQIRKF
TDKGLNTQDLVALSGAHTIGTAACALFSYRLYNYNNTNGPDPTIDASFLPTLRNLCPN
GGDGTTRVTLDTGSVRNFDNSYYSNLRNRRGVLESDAALWNNPATQTLVNRFLGLRGL
LGLRFNIEFGRSMVKMGNIEVKTGTQGEIRRVCTATN
SEQ ID NO: 61
PWA99879.1 heme peroxidase [Artemisia annua]
MAINLLIIISLSLTLIPVAKSQAGLKSGFYSSTCPKAEATVRSIVETHFKDDPTVAAA
LLRLHFHDCFVEGCDGSILIKGKSAEINALANLGLRGFEVIDDAKTQLETLCPGVVSC
ADILALAARDSVDLSDGPNWAVPTGRRDGRVSLASKASNLPSPLDSVDTQRKKFADKG
LDDRDLVTLAGAHTIGQTDCRFFRYRLYNFTTTGNSDPSLSQSALTQFQAQCPKNSDG
TERVALDKDSQMKFDVNYFKNVRDGNGVLESDQRLWGDPTTHDIVKNYAGTIRGLLGL
RFDFEFKKSMVKMSSIEVKTGDQGEIRKICSKFN
SEQ ID NO: 62
AGH28086.1 peroxidase [Artemisia annua]
MVVLFNNKKPILCLLLLATLITLSLGQGTRLGFYRTSCPRVESIVQNAVNSAVRANPT
IAPGLLRMFFHDCFVNGCDASILIDGASSEKTARPNSLLRGFEVIDAAKRQLEIACPR
VVSCADILALAARDSVVQTGGTGWSVPLGRKDGLVSRAADTANLPAFNDPINVQIRKF
TDKGLNTQDLVALSGAHTIGTAACALFSYRLYNYNNTNGPDPTIDASFLPTLRNLCPN
GGDGTTRVTLDTGSVRNFDNSYYSNLRNRRGVLESDAALWNNPATQTLVNRFLGLRGL
LGLRFNIEFGRSMVKMGNIEVKTGTQGEIRRVCTATN
SEQ ID NO: 63
PWA56365.1 peroxidase superfamily protein [Artemisia
annua]
MKGFWSLGLMVVGVLSILGSVQGDLEMGFYSSSCPKAEKIVQDFVNQHIPNAPSLAAT
LIRMHFHDCFVRGCDGSVLLNFTSSSGNQTEKVAVPNRTLRGFGFIDAIKSLVEAECP
GVVSCADIIALAARDSIVLTGGPSWKVPTGRRDGLLSNASEALAQIPAPTDNITILIQ
KFANKSLDLKDLVLLSGAHTIGIAHCPSFSNRLYNFTGVGDRDPSLDSEYADVLRSTK
CRTQNDNTTIVEMDPGSRKTFDLSYYTLLLKRRGLFESDSALTRNSNTLTYINQLLQG
SLQNFFSEFALSMEKMNQIGVKTGTTGEIRKNCAVVNS
SEQ ID NO: 64
PWA81621.1 peroxidase superfamily protein [Artemisia
annua]
MSHNLFLLPLVFLALFPVVEPRLRQQYYSETCPNAETIVTFMMQKAMIKDPRSGASVM
RFQFHDCFVNGCDASLLLDDTPTMLGEKLSLSNIDSLRSYDVVDWIKGALERTCPMTV
SCADIIIMASRDAVTLSGGPHWGVKLGRLDSLSASQKDSDDIMPSPRANASMLIDLFA
RFSLSVQDLVALSGSHSIGNARCFSIVHRIYNQSDDGKPDPSIELGFLKKLQELCPLG
GDENVIGDLDFTPKTFDNEYFKDLVNGKGFLNSDETLFTNPQTREYVQRYSEDQSAFF
KDFVQGMIKLGDLQSGRPGEIRKNCRVVNNPPPVNLMSGGPDWEVKLGRNDSLTASQK
DSHDIMPSPSANASTLIDLFAKFDLSIKDLVALSGSHSIGNGRCFSIVHRLYNQTDDG
RPNPSIEPRYRQKLDKLCPLGGDEEVIGDLDFTPRVFDNQYFKDLVNERGFLNSDETL
YTFWQTRGYVQRYSENQSTFFKDFVKGMIKMGDLQSGQGKFIGIVEWLMMRHRWK
SEQ ID NO: 65
PWA72837.1 heme peroxidase [Artemisia annua]
MGYDKSIGTILVPIVVLLVSVNYMPCKAQLCTTFYDDTCPSALATIRDSVSSAVSSNR
RMAALLIRLHFHDCFVQGCDASILLEGTGSEKASPANDGVLGYETVDDIKAAVESVCP
GVVSCADILAVAARDASVEVGGPSWEVKLGRRDSTGSNASQAVTDLPRGNNNLTELIA
SFDRKGLSVRDMVALSGSHTIGKARCVRFRDRIYANNSNIDATFAANLRRNCPQTAPN
GDGNLEPLDLQTPNKFDNNYFRNLVASKGLLTSDQVLFNGDSTDSIVTEYVDNPSLFD
CDFAKAMVNMGDIEPLTGTSGIIRTLCSTAN
SEQ ID NO: 66
PWA88165.1 heme peroxidase [Artemisia annua]
MIQKNFGFFLASILVVGCMGGGGLKMKYYKKRCDPISVEDTVREIVWSKVDKTPSLAA
KLLRLHYHDCFVRGCDGSILLDSTQNVTTEKTAGPNRSVTGYDIIDEIKTVLEASCPG
IVSCADIVALAARDAVSFQFQTEMWPVFTGRKDGSISLASEVGTNLPSANANFTTLLS
QFRTKGLDLKDLVTLSGAHTIGNSRCALVARRLYNFTGIGDADPSLDETYAQTLRKLC
PNPQNPATILEMDPNSSLSFDSNYYQALNQHKGLFVSDAALLTNLQSALIAKVFEDPT
VFFAEFARSMVRMGAIEVLTGGQGEVRKNCRAINDQ
SEQ ID NO: 67
PWA82380.1 peroxidase superfamily protein [Artemisia
annua]
MIKMRIMMLVMIIVFKFFIGFTEAQLEVGFYNKVCPAAETVVANVVKDATKSDSQTPA
AMLRLHFHDCFVEGCDGSILIDNGNNSERIANGHQGVRGYDVIENAKAQLESMCPGVV
SCADIVAMAARDAVALSHGPVYQVETGRKDGLVSRMELAGRMPDVQDSIQILKQKFIE
KGLNDRDLVILSAAHTIGTTACFFMTTRLYDFASSEGPDPSINPSFLPELTSACPKEG
NPNDRLPMDHGSGDIFDKQIFQNIRSGFAVLQSDAKLMDDPTTKGIVDSYFGLLSPLI
GPSFEGDFVKSMVRMGRIGVKTGSSGDIRRVCKAFN
SEQ ID NO: 68
PWA63444.1 peroxidase superfamily protein [Artemisia
annua]
MKGFWSLGLMVVGVLSILGSVEGDLQMGFYSSSCPKAEKIVQDFVNQHIPNAPSLAAT
LIRMNFHDCFVRGCDGSVLLNFTSSSGNQTEKVAVPNRTLRGFGFIDAIKSLIEAECP
GVVSCADIIALAARDSIVLTGGPSWKVPTGRRDGLLSNASEALAQIPAPTDNITILIQ
KFANKSLDLKDLVLLSGAHTIGIAHCPSFSNRLYNFTGVGDRDPSLDSEYADVLRSTK
CRTQNDNTTIVEMDPGSRKTFDLSYYTLLLKRRGLFESDSALTRNSNTLTYINQLLQG
SLQNFFSEFALSMEKMNQIGVKTGTTGEIRKNCAVVNS
SEQ ID NO: 69
PWA89542.1 peroxidase 4 [Artemisia annua]
MGSSAIVKFAIVMFLVGVASAQLSTNFYAKSCPNLFFAVNSAVRSAISRETRMGASLL
RLHFHDCFVNGCDGSILLDDTPSFTGEKNAAPNFRSARGFDVVDNIKSAVERACPGVV
SCADILAITARDSVHILGGPYWNVKLGRRDSRTASQAAANNSIPPPTSSLSALISSEN
RVGLSAKDMVALSGSHTIGQARCTNFRARIYNETNNLDASFATSRRSNCPRASGSGDN
NLAPLDIQTPNAFNNDYYKNLIAKKGLLHSDQQLFNGGSADSTVRKYSSNPRQFSSDF
AAAMIKMGDFRPLTGSSGEIRRNCRKPN
SEQ ID NO: 70
PWA36178.1 peroxidase [Artemisia annua]
MAIQKHNTLGLRFLLVALLLNIANGNDLKLGFYKKTCPRAEAIVKRTTANFISRAPSL
AAPLLRMHFHDCFVRGCDGSVLLNSTKNNQAEKAGIPNLSLRGFQVIDAAKTAVEAAC
PGVVSCADILALVARDSIQQIKGPYWPVPLGRRDGRVSIASESFTLPAPFANITTLKS
QFKSRGLSVKDLVVLSGGHTVGVSHCSTIASRLYNFTGKGDTDPTLDPKYVPHLKSKC
KPTDTTTLLEMDPGSVKTFDEDYYTIVSKRRGLFESDAALLNDKQTSAYVKLQAKSHG
ATFFKDFSDSMVKMGKIGVLTGKNGGHTVGVSHCSTIASRLYNFTGKGDTDPTLDPKY
VPHLKSKCKPTDTTTLLEMDPGSVKTFDEDYYTIVSKRRGLFESDAALLNDKQTSAYV
KLQAKSHGATFFKDFSDSMVKMGKIGVLTGKNGEIRKHCALIN
SEQ ID NO: 71
PWA90362.1 peroxidase N1 [Artemisia annua]
MDSRKSILLVVFLATLITLALGQTRVGFYRTSCPRVETIVQTAVRSAVNSNPTIAPGL
LRMFFHDCFVNGCDASILIDGASSEKTALPNSLLRGFEVIDAAKTQLETACPGVVSCA
DILALAARDSVVQTGGASWQVPLGRRDGLVSRASDTASLPGFNEPINALITKFADKNL
NTQDLVTLSGAHTIGTAACVLFSYRLYNYNNTNGPDPTIDPSFLPTLRNLCPNGGDGT
RRVTLDTGSVGNFDNSYYRNLRNRRGVLESDAALWNNPATQTLVNRFLGIRGLAGLRF
NVEFGRSMVKMGNIELKTGSQGEIRRGKRKNETGAIRRSNCCAEVIYTEVKPAPFKEK
IVGIYKNLDDASTTNHVNPMVRADVEEAKNLENTK
SEQ ID NO: 72
PWA56366.1 peroxidase superfamily protein [Artemisia
annua]
MKGYCSILVILAILGLVQGDLKLGFYKSSCPKAEKIVQEYVNQHIPNAPSLAAALIRM
NFHDCFVRGCDASILLNFTSASGNQTEKVAIPNQTVRGFGFIDAIKSLVEAACPGVVS
CADIIALATRDSIVATGGPYWKVPTGRRDGLISNASEALAQIPAPFDNITSLIQKFAI
KTLDLKDLVLLSGAHTIGIAHCPSFSNRLYNFTGVGDRDPSLDSEYADNLQSRKCRTP
NDNTTKVEMDPGSRKTFDLSYYSLLLKRRGLFESDSALTRNSNTLTYINQLLQGSLQN
FFSEFALSMEKMNEIEVKTGTSGEIRRNCAVVN
SEQ ID NO: 73
PWA88227.1 heme peroxidase [Artemisia annua]
MSILLFLLLATLATSALGQGTRVGFYRTSCPRAESIVQSAVQSGARSNPRAAPGILRM
FFHDCFVNGCDGSILIDGPSAEKTAVPNVPIRGFDVIDAAKTQLEATCPGVVSCADIL
ALAARDSVVLSGGRRWEVPTGRRDGLVSQASDTANLPAFNDPISVQIRKFSEKGLNTQ
DLVTLVGGHTIGTAACAVFSYRLYNFNNTNGPDPDINQAFLPQLRALCPNGGEATTRV
GLDTGSVNSFDNSYYANLRNGRGVLESDSKLWSDSTTQRFVQRFLGVRGLQGLRFDVE
FGRSMVKMGNIELKTGSQGEIRRVCTATN
SEQ ID NO: 74
PWA66053.1 peroxidase [Artemisia annua]
MDSKKSILLVLLLATLITLALGQTRVGFYQTSCPRVETIVQTAVRSAINSNPTIAPGL
LRMFFHDCFVNGCDASILIDGASSEKTAPPNGFLRGFEVIDAAKTQLETTCPGVVSCA
DILALAARDSVVQQNFLTLQTGGASWQVPLGRRDGLVSRASDTGSLPGFNEPLNALIR
KFADKNLNTQDLVTLSGAHTIGTASCLTFSYRLYNYNNTNGPDPSIDPTFLPTLRNLC
PNGGDGTRRVTLDTGSVGNFDNSYYSNLRNRRGVLESDAALWNNPATQTLVNRFLGIR
GLAGLTFNVEFGRSMVKMGNIELKTGSQGEIRRVCTAIN
SEQ ID NO: 75
PWA82975.1 heme peroxidase [Artemisia annua]
MEASSLNKILIMTVLLLATFTSFALGQGNRVFSRGGTRVGFYRSTCPRAEFIVQSAVQ
TAVRANPTIAPGLLRMFFHDCFVNGCDASILLDGASTEKTAGPNAFLRGFEVIDAAKS
RLETACPGVVSCADILALAARDSVVLTGGHSWHVPLGRRDGLVSRASDTATLPAFNDP
MTVQIQKFADKGLNIQDLVILVGGHTIGTAACALFSYRLYNFNNTNAPDPDINQAFLP
QLRALCPNGGNALTRVALDTGSVNSFGNSFYDNLRIGRGVIESDAKLWSDKRTQREVQ
GFTGGRSGFRFNAEFGRAMVKMGNIEVKTGRQGQIRRVCTATN
SEQ ID NO: 76
PWA62902.1 peroxidase 4 [Artemisia annua]
MGSSVIVKFAFVMFLVGVSSAQLSTNFYAKSCPNLFLAVNSAVRSAISRETRMGASLL
RLHFHDCFVNGCDGSILLDDTSSFTGEKNAAPNFRSARGFDVVDNIKSAVERACPGVV
SCADILAITARDSVHILGGPYWNVKLGRRDSRTASQAAANNSIPPPTSSLSALISSFS
RVGLSAKDMVALSGSHTIGQARCTTFRARIYNETNNLDASFGASRRSNCPRASGSGDN
NLAPLDVQTPNAFNNDYYKNLIAKKGLLHSDQQLFNGGSADSTVRQYSSNPRQFSSDF
AAAMIKMGDFRPLTGSSGEIRRNCRRPN
SEQ ID NO: 77
PWA92428.1 peroxidase 27 [Artemisia annua]
MAIQKQLIAVLQLLLIALVFDIANGKGLDLGFYRYTCQRVEAIVRKTTANYIHRAPSL
AAGLLRMHFHDCFVRGCEGSVLLNSTVNNQAEKEGIPNLTLRGFQVVDAAKTALEAAC
PGVVSCADILALVARDAIYQIKGPFWRVPLGRRDRRVSIANEALTSLPSPFANITELK
ANFASKGLNTKDLVVLSGRHTVGVSHCLVIVNRLYNFTGKGDTDPSLDPKFIPQLKSI
CSPIDTTSVLAMDRTPTSFDEDYYTVVLRKRGLFQSDAALLIDKQTSAYVKLQSKSFG
YTFFNDFSKSMVKMGKTGVLTSSAGEIYRTCA
SEQ ID NO: 78
PWA80553.1 heme peroxidase [Artemisia annua]
MSILLFFLLATLATSALGQGTRVGFYRTSCPRAESIVQSAVQSGARSNPRAAPGILRM
FFHDCFVNGCDGSILIDGPSAEKTAVPNVPIRGFEVIDAAKTQLEATCPGVVSCADIL
ALAARDSVVLSGGRRWEVPTGRRDGLVSQASDTANLPAFNDPISVQIRKFSEKGLNTQ
DLVTLVGGHTIGTAACAVESYRLYNFNNTNGPDPDINQAFLPQLRALCPNGGEATTRV
GLDTGSVNSFDNSYYANLRNGRGVLESDSKLWSDSTTQRFVQRFLGVRGLQGLRFDVE
FGRSMVKMGNIELKTGSQGEIRRVCTATN
SEQ ID NO: 79
PWA78924.1 heme peroxidase [Artemisia annua]
MARSIIMLMVLISVMAIANASYGSYLYPQFYDHSCPQAKNIVQSVVAKAVAKEARMAA
SLLRLHFHDCFVKGCDASILLDNSGTIISEKGSVPNRNSARGFEVIDEIKAALEKACP
QTVSCADIMALAARDSTVLAGGPSWEVPLGRRDSLGASLSGSNQNIPAPNNTFQTILT
KFKLKGLDIVDFVTLSGSHTIGNARCTSFRQRLYNNTGNGKPDLSLDQSYAAQLRQNC
PRSGGDQNLFVMDPVSPTKFDNNYYKNLIAAKGLLSSDEILFTQNQQTMQYVKQYAAN
QELFFQQFAKSMVKMGNITPLTASHETLYMITHVYTILEEHMLAVDTSHKLLIFDPSL
LKYGNKQLTLRTVDCDAPNVGFTTVREVTYSSLPYPHSFLNSLYVLSSFDGLVCLASP
LTKELALLNPLTGAFKSLPENSYSPHFYNRYSDVLGFYMDSFSDDYKLLHIVVSKGFL
GAYVYSLKMDSWKKIEYLVDSIDHRSDYSWTPATLLGQCLYFVVWESSSEGLFHES
SEQ ID NO: 80
PWA84241.1 heme peroxidase [Artemisia annua]
MASRNVSTTVFFLLFLAVTNNTLCEAQLSTTFYDRTCPNALSTIRTSIRRAISRERRM
AASILRLHFHDCFVQGCDASILLDDGPTIISEKNALPNKGSVRGYEVIEAAKTEVEKL
CTGVVSCADILTVAARDASEMVGGPSWSVKLGRRDSDTASLVLAETGALPSFKAPLDS
LISTFRDNGLSARDMVALSGAHTIGQAQCFLFRDRIYSNGSDIDAGFASTRRRRCPIN
DGNENLAALDLVTPNSFDNNYFKNLLQKKGLLETDQVLFSGGSTDSIVTEYSRSPAKF
KSDFAAAMVKMGEIRTLTGQQGVIRRICGALP
SEQ ID NO: 81
PWA34474.1 peroxidase superfamily protein [Artemisia
annua]
MRGFWSFGIIVLGFISILGSVQGGLQIGFYSSSCPKAEKIIKDYVNQHIPNAPSLAAT
LIRMHFHDCFVRGCDASILLNFTSASGNQTEKVAIPNQTVRGFDFIDRIKSLLEAECP
GIVSCADIISLAARDSIVVTGGPSWKVPTGRRDGLLSNASEALAQIPAPFDNITILTQ
KFVNKSLNLKDLVLLSGAHTIGIAHCPSFSNRLYNFTGRGDQDPSLDSEYADNLRSRK
CTTPNDNTTIVEMDPGSRKTFDLSYYSLLLKRRGLFESDSALTRNSNTLTFVNQLLQG
SLQNFFSEFALSMEKMNQIGVKTGTTGEIRRNCAVVNS
SEQ ID NO: 82
PWA41813.1 heme peroxidase [Artemisia annua]
MGASSLNKILIVTVLLLATFTSLALGQGNRWFSRGGTRVGFYKSTCPRAEFIVQSAVQ
TAVRANPTIAPGLLRMFFHDCFVNGCDASILLDGASTEKTAGPNAFLRGFEVIDAAKS
RLETACPGVVSCADILALAARDSVVLTGGHSWHVPLGRRDGLVSRASDTATLPAFNDP
MTVQIQKFADKGLNIQDLVILVGGHTIGTAACALFSYRLYNENNTNAPDPDINQAFLP
QLRALCPNGGNALRRVALDTGSVNSFGNSFYDNLRIGRGVVESDAKLWSDRRTQREVQ
GFTGRRSGFKFNAKFGRAMVKMGNIEVKTGRQGEIRRVCTATN
SEQ ID NO: 83
PWA62658 .1 heme peroxidase [Artemisia annua]
MNIISLLALFVFLIFTVATASSDSGLSTKYYDRVCPQALPTIKRVVEDAVAQERRMGA
SLLRLHFHDCFVNGCDASVLLDQTSTIDSEKNALANANSARGFEVIDRIKSEVDKICG
RPIVSCADILTLVARDSVVALGGPSWKVKLGRRDSTTASRATANANLPSPFMDLPALI
KNFKDQGLDEKDLVLLSGAHTLGFAQCFTFRDRIYNDTNIDPAFAGHLRTICPRVGGD
SNLAPLDPTPSSFDTKYFNNLIRKRGVLISDQVLLNGRKTNELVSEYNEDKTKFFKDF
SKSMIKMGKIDLLTGNRGQVRHNCRRVNSQ
SEQ ID NO: 84
PWA56112.1 peroxidase N1 [Artemisia annua]
MDSKKSILYVFLLATLITLALGQARIGFYQTSCPQVETIVQTAVQSAINSNPTIAPGL
LRMFFHDCFVNGCDASILIDGASSEKTALPNSLLRGFEVIDAAKTQLETACPGVVSCA
DILALAARDSVVQTGGASWQVPLGRRDGLVSRASDAASLPGFNEPINALITKFSDKNL
NIQDLVTLSGAHTIGTAACVLFSYRLYNYNNTNGPDPTIDPDFLPTLRNLCPNGGDGT
RRVALDTGSVGNFDTSYYSNLRNGRGVLESDAALMNNPATKALVNRFLGIRGMAGLSF
NVEFGRSMVRMGNIELKTGNQGEIRRVCTTTN
SEQ ID NO: 85
PWA45869.1 peroxidase superfamily protein [Artemisia
annua]
MRYIVTKMMMVLVTMVVMLRHCNGLNMNYYMMNGCPMADFIVKNTVNAALRADPTLAA
GLIRMHFHDCFIQGCDASILLDSTDDNTAEKDSPANLSLRGYKVIDDAKQELESRCPG
VVSCADIVAMAARDAVFFAGGPVYEIPKGRKDGTRSKIEDTRNLPPPTFNASELITMF
GQHGFTAQEMVALSGGHTLGVARCASFKNRLKSFDSTHDVDPSIDTQFVKTLAKTCNG
GDNAEQPFDQTRNMFDNNYYNGLQRQAGVLSSDQTLMTDPKTRNIVNAYAFNQAMFFF
DFQQAMVKMGLLDVKETGQVRQNCRKIN
SEQ ID NO: 86
PWA50902.1 peroxidase N1 [Artemisia annua]
MYSKNSILYVVLLATLITLALGQAQIGFYQTSCPQVETIVQTAVQSAINSNPTIAPGL
LRMFFHDCFVNGCDASILIDGASSEKTALPNSLLRGFEVIDAAKTQLETACPGVVSCA
DILALAARDSVVQTGGASWQVPLGRRDGLVSRASDAASLPGFNEPINALITKFADKNL
NIQDLVTLSGAHTIGTAACVLFSYRLYNYNNTNGPDPTIDPDFLPTLRNLCPNGGDGT
RRVALDTGSVGNFDTSYYSNLRNGRGVLESDAALMNNPATQALVNRFLGIRGMAGLSF
NVEFGRSMVRMGNIELKTGNQGEIRRVCTTTN
SEQ ID NO: 87
PWA84240.1 heme peroxidase [Artemisia annua]
MGYRNYFAIMFFLLFLSVTTNTPCKAQLSTKFYDDTCPNALRTIRNTIRTSISRERRM
AASLLRLHFHDCFVQGCDASILLDDAPMIISEKNALPNKGSVRGFEVIEAAKAKVEKL
CPGVVSCADILTVAARDASEMVGGPSWSVKLGRRDSTTASLVQAETSLPSFMAPLESL
ISSFKAQGLDARDLVALSGAHTIGQAQCVLFRDRIYNNGSHIDAGFARKRRHHCPKNE
GNGNLAPLDLVTPNSFDNNYFKNLIQKKGLLESDQVLYSGGSTDKIVLEYSRNPKKFK
SDFAAAMIKMSEIKPLTGHKGVIRRVCGALP
SEQ ID NO: 88
PWA58590.1 peroxidase superfamily protein [Artemisia
annua]
MIIFFTFLIGFTEAQLKVGFYNKVCPAAETVVANVVKDATKSDPQTPAFMLRLHFHDC
FVEGCDGSILIDNGDNSERTAPGHIGLQGFKVIENAKAQLEIMCPGVVSCADIVAMAA
RDAVALLLFFDSHGPVYQVETGRKDGLVSRMELAARMPDVQDSIQILKQKFIEKGLND
SDLVILSAAHTIGKAACFFMFKRLYNFSSSGGPDPSINPSFLPELTSACPEGGDAGHR
LPMDHGSGEIFDKQIFQNIRSGFAVLQSDAQLMDDPTTKEIVDSYFGLLSPLPGPSFE
SDFMSSMVRMGRIGVKTGSSGNIRRVCNAFN
SEQ ID NO: 89
PWA80413.1 peroxidase superfamily protein [Artemisia
annua]
MMLVMIILFKFLIVFTEAQLKVGFYNKVCPAAETVVANVVKDATKSDPQTPAFMLRLH
FHDCFVEGCDGSILIDNGVNSERAANGHQGVLGYDVIENAKAQLESMCPSVVSCADIV
AMAARDAVALSHGPVYQVETGRKDGLVSRIELAARMPDVQDSIQILKQKFIEKGLNDK
DLVILSAAHTIGTSACFFMTTRLYDFASSGGPDPSINASFLPELTSACPKGGNPNDRL
PMDHGSGDIFDKQIFQNIRSGFAVLQSDAKLMDDPTTKGIVDSYFGLLSPLTGPSFEG
DFMSSMVRMGRIGFKTGSTGNIRRVCKAFN
SEQ ID NO: 90
PWA94345.1 peroxidase [Artemisia annua]
MHFHDCFVRGCDGSVLLNSTANNQAEKEGIPNLTLRGFQVVDAAKTALEAACPGVVSC
ADILALVARDAIYQIKGPFWPVPLGRRDGRVSIANEALTSLPSPFANITELKANFASK
GLNTKDLVVLSGGHTVGVSHCPVIVNRLYNFTGKGDTDPSLDPKFIPQLKSICSPTDT
TSVLAMDRTPRSFDEDYYTVVLRKRGLFQSDAALLNDKQTSAYVKLQSKSFGYTFFND
FSKSMVKMGKIGVLTGSAGEIRRTCALVN
SEQ ID NO: 91
PWA67603.1 heme peroxidase [Artemisia annua]
MVSFKHIYATFTLLVLLSSTFCFGFPEFNFRWGHGRDGVSRPIGGGRSTGLFPGFYSD
SCPQANDIVMSVLERAISKDSRMAASLLRLHFHDCFVQGCDASVLLDDHPTFVSEKKA
VPNKNSLRGFEVIDEIKSKLEQACPKTVSCADILALSARGSTVLSGGPNWELPLGRRD
SKKASLNGANKFIPPPNSTIQSLLTFFQRQGLNEVDLVSLSGAHTIGVARCTTFKQRL
YNQNGNNQPDSTLERSYYHDLKAVCPKTGGDSNISPLDYTSPVTFDNTYFKLIMSGKG
LLTSDQVLLSGNVGKTMQLVKAFADDHTLFFNHFARSMVKMGKISPLTGYKGEVRKNC
HRIN
SEQ ID NO: 92
PWA45227.1 heme peroxidase [Artemisia annua]
MVVLAIIFSHADGQGLKIGFYEKSCPHAEKIVFNVMKDVMAVAPSLSGPLLRMHFHDC
FIRGCDGSVLLDSPTNQSEKFSIPNLSLRGYNIIDRVKTELEKYCSGVVSCADISALV
ARDVTVLTKGPFWEVETGRRDGRISLIGDPINPVSGLPPFFLNITALKQSFALRGLSV
KDLVVLSGGHTLGMSHCSSFENRLYNFTGIGDTDPTMDQNYIARLKLKCKPNDQTTIA
ELDPGSFKTFDDSYFRLVNKRRGLLQSDAALLDDPETKAYMIQATSYGSTFFKDFGVS
MVKMGRIGVLTGSQGEIRKVCSKVN
SEQ ID NO: 93
PWA39487.1 heme peroxidase [Artemisia annua]
MGYRNYFAIMFFLLFLSVTTNTPCKAQLSTKFYDDTCPNALRTIRNSIRASISRERRM
AASLLRLHFHDCFVQGCDASVLLDDAPKIISEKNALPNKGSARGFEVIEAAKAKVEKL
CPGVVSCADILTVAARDASEMVGGPSWSVKLGRRDSTTASLVEAETSLPSFLAPLESL
ISSFKVQGLDARDLVALSGAHTIGQAQCVLFRDRIYNNGSHIDAGFARKRRHHCPKNE
GNGNLAPLDLVTPNSFDNNYFKNLIQKKGLLESDQVLYSGGSTDKIVWEYSRNPKKFK
SDFAAAMIKMSEIKPLTGHKGVIRRAKNRKNIYKAFFTTGANYISESSRVLA
SEQ ID NO: 94
PWA39488.1 heme peroxidase [Artemisia annua]
MASYNVFAPIFFLLLILITNNTPSEAQLSNTFYDSTCPNALRTIRTSIRTAISRERRM
AASILRLHFHDCFVQGCDASILLDDGPTIVSEKNALPNKGSVRGYEVIEAAKSEVEKL
CPGVVSCADILTVAARDASEMVGGPSWAVKLGRRDSTTASLVLANTGLPSFKAPLDSL
ISTFNDNGLSPRDMVALSGAHTIGQAQCFLFRDRIYANGSDIDAGFASTRRRRCPINE
GNENLAALDLVTPNSFDNNYFKNLIQKKGLLESDQVLFSGGSTDSIVREYSNNPSKFK
SDFAAAMVKMSEFRTITGQEGVIRRICGSLP
SEQ ID NO: 95
PWA66812.1 heme peroxidase [Artemisia annua]
MAHSLTMTITIMCIALMALTPVFSKITNGGYLYPQFYDQSCPQATNIVRSIVAKNVAK
DPRMAASLLRLHFHDCFVKGCDASVLLDNGGPRNVVSEKGSVPNNNSARGFEVIDEIK
AALETACPQTVSCADILALAARDSTVLAGGPSWEVPVGRRDSLGASLRGSNNNIPAPN
NTFQTILTKFKLRGLDIVDLVALSGSHTIGNARCTSFKQRLYGNKGNDQINQLFAQKL
RAMCPQSGGDQNLSFLDNMTPMKFDNAYYKNLMESKGVLSSDQILFKDNQETMQLVKE
YAANEEMFFQQFAKSMVKMGNISPLTGFRGEIRKTCRSING
SEQ ID NO: 96
PWA87473.1 peroxidase [Artemisia annua]
MTSSSSSYLSSLPTCILCLCVLARMASGQLTANFYASSCPNFSTTISTAVNSAVSNEA
RMGASLLRLHFHDCFVNGCDASVLLDDTANFTGEKTAAPNNNSLRGFDVIDTIKTQLE
SSCPGVVSCADILATAARDSVVALGGPSWNLVFGRRDSTTASLSAANNNIPAPTLNLS
GLISSFSNQNFTTNEMVALSGSHTIGQARCTVFRARIYNENNINSSFATSVRANCPSS
GGDNNLSPLDVVSPTSFDNSYFSNLLNQNGLLHSDQELFNGGTTDAQVRTYSSNSATF
STDFANAMVKMSNLNPLTGSSGEVRTNCRRTN
SEQ ID NO: 97
PWA36813.1 heme peroxidase [Artemisia annua]
MNIISFLSFFVFLMLSIATASSDDYGLSTKYYDRVCPQALPTIKRVVEDAVAQERRMG
ASLLRLHFHDCFVNGCDASILLDQTSTIDTEKNAIANTNSARGFEVIDKIKSEVDKIC
GRPIVSCADILTVVARDSVVALGGPTWDVKLGRRDSTTASQVTANVTIPSPFMDLPAL
IKSFEDQGLDEEDLVVLSGAHTLGFAQCRTFRRHIYNDSNIDPAFAGQLRTNCPQVGG
DSNLVPLDPTPSSFDNRYFNNLVSKRGVLGSDQALFNGGETDELVSKYNENYEEFFKD
FAKSMIKMGNINLLTGNRGQIRDNCRRINSQ
SEQ ID NO: 98
PWA79839.1 peroxidase superfamily protein [Artemisia
annua]
MAFLIYNTCMLMVLLSSSVTSFAFPGFSFGWGGGGGAGDGSFGGGDGSFGGVLGYSGL
FPEFYSFSCPQANDIVMSVLEDVIAKDPRMAASLLRLHFHDCFVQGCDASVLLDDSAM
FTSEKNAGPNANSIRGFEVIDEIKSKLEEACPETVSCADILALAARGSTVLSGGPNWE
LPLGRRDATQANFKESNNNLPPPNSTIQNLITLFRRQGLDEVDLVSLSGAHTIGMARC
TSFKQRLYNQDGNNQPDSTLERTYSNGLKEICPKTGGDSNISPLDFSSPARFDNTYFK
LIMWGKGLLTSDQVLLNGNVEETMFLVKAFAEDDSLFYDQFARSMVKMGNINPLTGFN
GEIRKNCRMVNQL
SEQ ID NO: 99
PWA93003.1 heme peroxidase [Artemisia annua]
MFYTVYEDYNIDQVLHVVRLRVSFHYAEIMIISTYNKKMNIIRFLSFFVFLMLSIATA
SSDDYGLSTKYYDRVCPQALPTIKRVVEDAVAQERRMGASLLRLHFHDCFVNGCDASI
LLDQTSTIDTEKNAVANVDSARGFDVIDKIKSEVDKICGHPVVSCADILTVAARDSVV
ALGGPTWDVKLGRRDSTTASQATANVTIPSPFMDLPALIKSFEDQGLDEEDLVVLSGA
HTLGFAQCRTFRPHIYNDNNIDPAFASQLRNNCPQVGGDSNLAPLDPTPSSFDTRYFN
NLVSKRGVLRSDQALFNGGETDELVSKYNENYEKFFKDFAKSMIKMGNINLLTGNRGQ
IRDNCRRINSQ
SEQ ID NO: 100
PWA43335.1 peroxidase superfamily protein [Artemisia
annua]
MEIKSTIFIFSLITLCCLTFSHAADPPLTLDYYKSTCPNAEAIVRKEMECAVTSDLRN
AAAILRLHFHDCFVQGCDGSVLLDDTYMLKGEKNAPTNLNALKGFDIIDRIKNKLESE
CPGTVSCADVLTYAARDATVLVGGPYWHVPAGRKDSKTASASLVESNIPGANNGLLTM
ISKFMYQSLSVTDMVALSGAHTIGMARCTNYRARIYGDYQRTATMSSIADSNLKTLKS
TCPAAGGGDNNESAMDYISPNLFDNSYYHILLRGEGLLASDQELYSSMLGVQTSNLVK
KYAENQLAFFEQFSESMVKLGNITNPETYVDGEVRKNCRFVNT
SEQ ID NO: 101
PWA67189.1 heme peroxidase [Artemisia annua]
MACFKNLFGILMLQMVVLAIISSHADGQGLKIGFYEKSCPQAEKIVENVMKDVMAVAP
SLSGPLLRMHFHDCFIRGCDGSVLLDSPNNQSEKFSIPNLSLRGYNIIDRVKTELEKY
CPGVVSCADISALVARDVTVLTKGPFWDVETGRRDGKISLIGDPINPVSGLPPFFLNI
TALKQSFALRGLSVKDLVVLSGGHTLGMSHCSSFENRLYNFTGIGDTDPTMDRNYIAR
LKLKCKPNDQTTIAELDPGSFKTFDDSYFRLVNKRRGLLQSDAALLDDPETKAYMIQA
TSYGSTFFKDFGVSMIKMGRIGVLTGSQGEIRKVCSKVNESPN
SEQ ID NO: 102
PWA37630.1 Plant peroxidase [Artemisia annua]
MEIYSHIRTPILYVVLLAAMSTLALSCTQVGFYKTTCPPAEGIVKSVVQSAIRSNLTI
APGLLRMFFHDCFVNGCDASILIDGLSAEKIVVQNLALRGFEVIQAAKSKLETVCPGV
VSCADILALAARDSVVQSGGMNWEVPTGRRDGLVSRDTDAKKLPGSTDNVTSQIKKFA
DKGLSIQDLVTLVGGHTIGTAACGRFDYRLYNYNNTNKPDPDIDQEFLAKLKTHCPCK
SNNTNRVPLDTNSETNFDNSYYGNVRNGQGVLESDSKLWSDGRTHKFVQQFLGSSNRL
KFKKQFGRAMVKLSNVEVKTGNQGEIRRSGGMTWEVPTGRRDGLVSRDTDAKKLPGST
DNVTSQIKKFADKGLSIQDLVTLVGGHTIGTAACGRFDYRLYNYNNTNKPDPDIDQEF
LAKLKTHCPCKSNNTNRVPLDTNSETNFDNSYYGNVRNGRGVLESDSKLWSDGRTHKF
VQQFLGSSNRLKFKKQFGRAMVKLSNVEVKTGNQGEIRRGYNISKQKF
SEQ ID NO: 103
PWA72564.1 heme peroxidase [Artemisia annua]
MKITLLSLVSILFFLSVSASSNPSLLHSLKVPSLTDTTHFEDELSYSLYRHSCSHAEG
ITYRKVRDWVKKDPTIGPSLIRLHFHDCAVRGCDASILLDHSGSEKSANVSKSLRGFE
LINEIKAELEKKCPKTVSCADILTTAARDATVLAGGPFWMIPFGRKDGRVSLAKEATA
VPMGRESITQLIEFFQSKGLNVLDLVVLSGAHTIGRTTCESVQHRLYNYKGTKKPDPS
IDPQYLNYLRRKCRWASENVYLDATTPKTFDVQYYQNLKKKMGLLSTDQLLYSDSRTK
PIADALSFESSLFSNQFSVSMVKLANILDYKSQDYGEIRTRCNRVNK
SEQ ID NO: 104
PWA88167.1 heme peroxidase [Artemisia annua]
METTMINIFVLLVASILVAGGMASHGNGIARGRGHGGGGGLKMRYYAKRCGSVSVENT
VRGIVWNKVAADPSLAGKLLRLHNHDCFVRGCDGSILLDPTQNMTTEKTAGPNRSVSG
FELIDEIKTKLEANCPGIVSCADIVALAARDAVSFQFRRDAWPVFTGRKDGRVSLASE
VGDNLPSANADFTTLLSQFESKGLNLNDLVTLSGAHTIGMSRCALVARRLYNFTGVGD
ADPSLDATYAQKLRNLCPKPITPSTPLEMDPKSSLSFDSNYYRILNQHKGLFVSDAAL
LTNRTSAKITKVLENPKVFFDQFAQSMVHMGAIEVLTNGQGEVRRNCRVVNGH
SEQ ID NO: 105
PWA70555.1 heme peroxidase [Artemisia annua]
MRYYEKRCGSVSVENSVREIVWNKVAADPSLAAKLLRLHYHDCFVRGCDGSILLDPTQ
NTTTEKTAGPNRSISGYEVIDEIKTKLEADCPGIVSCADIVALAARDAVSFQFRRDAW
PVFTGRKDGRVSRDSEVGGNLPSANANFTTLLSQFGSKGLNLIDLVTLSGAHTIGMSR
CALVARRLYNFTGVGDADPSLDATYAQTLRNLCPNPVNPSTPLEMDPKSSFTFDSNYY
RILNQHKGLFVSDAALLTNRQSAKITKVLENPKVFFDQFAQSMVHMGAIEVLINGQGE
VRRNCRVVNGH
SEQ ID NO: 106
PWA89755.1 heme peroxidase [Artemisia annua]
MTLKVPSLADLSSLEDDLSYSLYRESCYDLEGITYRKVKEWVTKDPTLAPSLLRLQYH
DCVVRGCDASILLDHEGSEKTSNMSKSLRGFEVIDDIKAEIEKKCPKTVSCADILTTV
ARDATVLAGGPYWMIPYGRKDGRVSLAKEASIVPKGSESVTNLIEFFQSKGLNVLDLV
ILSGAHTIGKTTCESVQHRLYDYKGTKKPDPSLDPKYLNYLRRKCRWASENVNLDGET
PNTFDTQYYHNLKKNMGLLSTDQMLYSDSRTKPIADGLSFQSSLFKNQFGVAMVKLSS
ILDVVSQDEGEIRTDCKYVNH
SEQ ID NO: 107
PWA80441.1 heme peroxidase [Artemisia annua]
MAGSKNFFGILMLQMVVLAIIFSHADGQGLKIGFYEKSCPHAEKIVFNVMKDVMAVAP
SLSGPLLRMHFHDCFIRGCDGSVLLDSPTNQSEKFSIPNLSLRGYNIIDRVKTELEKY
CPGVVSCADISALVARDVTVLTKGPFWEVETGRRDGRISLIGDPINPVSGLPPFFLNI
TALKQSFALRGLSVKDLVVLSGGHTLGMSHCSSFENRLYNFTGIGDTDPTMDQNYITR
LKLKCKPNDQTTIAELDPGSFKTFDDSYFRLVNKRRGLLQSDAALLDDPETKAYMIQA
TSYGSTFFKDFGVSMVKMGRIGVLTGSQGEIRKVCSKEVVSDLKSVRCLMKCLKGIFS
STIISNSSWLCAKDTSFDCQFNETNQSVATVNQSSQLLEEDTSFDCRREMVHHAIVQG
KRENEAETVQEMWSRAKGFKRIFHASLMIAFELKGREIPIVHVHRLIKIFTETLLEEE
IGGLKELFKMIDTDNSGTITFEELKESLRRLHNKNIPQNSTPQSLLQNTTPAPFFTMF
QGTPPNSTSYFSDLKLQRMVFYLRLQETYTWDVPVTTKIKSHVTTPYNKKDGRVFNKV
ISHHTTIGISVSNRIGQVIESGKVNNFDKNVHPWVYGDSEWMLDSKLLLLNEKESVLN
YDFKGIERD
SEQ ID NO: 108
PWA81503.1 heme peroxidase [Artemisia annua]
MSVLAYESNDYSSMTLKVPSLADLSSLEDDLSYSLYRESCYDLEGIIYRKVKEWVTKD
PTLAPSLLRLQYHDCVVRGCDASILLDHEGSEKTSNMSKSLRGFEVIDDIKAEIEKKC
PKTVSCADILTTVARDATVLAGGPYWMIPYGRKDGRVSLAKEASIVPKGSESVTNLIE
FFQSKGLNVLDLVILSGAHTIGKTTCESVQHRLYDYKGTKKPDPSLDPKYLNYLRRKC
RWASENVNLDGETPNTFDTQYYHNLKKNMGLLSTDQMLYSDSRTKPIADGLSFQSSLF
KNQFGVAMVKLSSILDVVSQDEGEIRTDCKYVNH
SEQ ID NO: 109
PWA71470.1 heme peroxidase [Artemisia annua]
MQKMNIISFLFIFVFLMFSIVTASTDDYGLSTKYYDRVCAQALPTIKRVVEDAVAQER
RMGASLLRLHFHDCFVNGCDASILLDQTSTIDTEKNAAPNVNSARGFEVIDRIKSEVD
KICGRPVVSCADILTVAARDSVVALGGPTWDVKLGRRDSTTASQVTANVTIPSPFMDL
PALIKSFEDQGLDEEDLVVLSGAHTLGFAQCRTFRRHIYNDSNIDPAFAGQLRTNCPQ
VGGDSNLVPLDPTPSSFDTRYFNNLVSKRGVLGSDQALFNGGETDELVSKYNENYEEF
FKDFAKSMIKMGNINLLTGNRGQIRDNCRRINSQ
SEQ ID NO: 110
PWA98359.1 heme peroxidase [Artemisia annua]
MKVSSFNKMGILVIILLATMTNIAQGQGTRVGFYRTTCPRVESIVQSAVRQAVRSNPT
MAPGLLRIFFHDCFVNGCDASLLLNGSSSEKTARPNLPLRGLQVIDAAKAQLERACPG
VVSCADIVALAARDSTGGTSWQVPLGRRDGLVSRASDTANLPAFNDPISVQIRKFSDK
GLNTQDLVALVGHTIGTAACSVFSYRLYNENNTNRPDPSINQAFLPQLRAICPNGGNG
TTRVGLDRGSVNKFDNSFFKNLRNGRGVLESDAKLLSNPTTQRFVQRFSGVRGTPKLR
FNVEFGRSMVKMGNIGLKTGRQGEICRRWKRRTCDTDYDRNELITRALKLGACLLEAG
NRSASPCNCSGGGAFNRCLYVMVVNKRRWTSGSSLCYGQRKKEKMHVLGGYQVSEKMH
MLGGYQGLHIGEKKKRIQFIKGFANYMNNFY
SEQ ID NO: 111
PWA57816.1 heme peroxidase [Artemisia annua]
MQKMNIISFLSIFVFLMFSIATASSDDYGLSTEYYDRVCPQALPTIKRVVEDAMAQER
RMGASLLRLHFHDYFVNGCDASILLDQTSTIDTEKNAAPNVNSARGFEVIDRIKSEVD
KICGRPVVSCADILTVAARDSVVALGGPTWDIKLGRRDSTTASQAAANFKIPSPFMDL
PALIKSFEDQGLDEEDLVVLSGAHTLGFAQCRTFRSHIYNDTNIDPDFAGQLRTNCPQ
VGGDSNLAPLDPTPSSFDTRYFNNLVSKRGVLGSDQALFNGGETDELVSKYNENYEEF
FEDFAKSMIKMGNIDLLTGNRGQIRENCRRIN
SEQ ID NO: 112
PWA89188.1 peroxidase superfamily protein [Artemisia
annua]
MSSYNTFAPILFLLLILITNNTQSEAQLSTTFYDGTCPNALHTIRTSIRTAISRERRM
AASILRLHFHDCFVQGCDASILLDDSPTIVSEKNAFPNKGSLRGYEVIEAAKSEVEKL
CPGVVSCADILTAAARDASVMVGGPSWSVKLGRRDSTTASLDLANTGLPSFKAPLDSL
ISTFNDNGLSPRDMVALSGAHTIGQAQCFSFRDRIYANGSDIDAGFASTHRHRCPINE
GNENLAALDLVTPNSFDYNYFKNLIQKKGLLESDQVLFSGGSTDSIVREYSKNTSNFK
SDFAAAMVKMSEFRTITGQEGVIRSICGALP
SEQ ID NO: 113
PWA60647.1 heme peroxidase [Artemisia annua]
MEIYSHIRTPILYVVLLAAMSTLALSCTQVGFYKTTCPPAEGIVKSVVQSAIRSNLTI
APGLLRMFFHDCFVNGCDASILIDGLSAEKIVVQNLALRGFEVIQAAKSKLETVCPGV
VSCADILALAARDSVVQSGGMNWEVPTGRRDGLVSRDTDAKKLPGSTDNVTSQIKKFA
DKGLSIQDLVTLVGGHTIGTAACGRFDYRLYNYNNTNKPDPDIDQEFLAKLKTHCPCK
SNNTNRVPLDTNSETNFDNSYYGNVRNGQGVLESDSKLWSDGRTHKFVQQFLGSSNRL
KFKKQFGRAMVKLSNVEVKTGNQGEIPEKIVVQNLALRGFEVIQAAKSKLETVCPGVV
SCADILALAARDSVVQSGGMNWEVPTGRRDGLVSRDTDAKKLPGSTDNVTSQIKKFAD
KGLSIQDLVTLVGGHTIGTAACGRFDYRLYNYNNTNKPDPDIDQEFLAKLKTHCPCKS
NNTNRVPLDTNSETNFDNSYYGNVRNGQGVLESDSKLWSDGRTHKFVQQFLGSSNRLK
FKKQFGRAMVKLSNVEVKTGNQGEIRRELKF
SEQ ID NO: 114
PWA81752.1 hypothetical protein CTI12 AA183790 [Artemisia
annua]
MASSKIISFAQVASVLCLMSITSSGQLTHDFYSESCPSVFEAVGSVVQDAVSREKRMG
ASLLRLHFHDCFVNGCDGSVLLDDTPSFTGEKTAGPNANSARGFDVIDDVKSKVEEVC
PGVVSCADVLAIAALESVVALGGPAWEIKLGRRDSRTASLSAADGGVIPPPSSTLSNL
INQFQAQGLSVKDLVALSGAHTIGQARCTTFRARVHNDTNIDTSFARSLQSNCPLPTG
LGDNNLAPLDVRSPTHFDNSYFRNLITEKGLLTSDQQLYSGGATDFFVEQYSQNPESF
HEDFAAAMIKMGDISPLTGSNGEIRTNCRVVNS
SEQ ID NO: 115
PWA82379.1 peroxidase superfamily protein [Artemisia
annua]
MLRLHFHDCFVEGCDGSILIDNGNNSERIANGHQGVRGYDVIENAKAQLESMCPGVVS
CADIVAMAARDAVALSHGPVYQVETGRKDGLVSRMELAGRMPDVQDSIQILKQKFIEK
GLNDRDLVILSAAHTIGTTACFFMTTRLYDFASSEGPDPSINPSFLPELTSACPKEGN
PNDRLPMDHGSGDIFDKQIFQNIRSGFAVLQSDAKLMDDPTTKGIVDSYFGLLSPLIG
PSFEGDFVKSMVRMGRIGVKTGSSGDIRRVCKAFN
SEQ ID NO: 116
PWA89541.1 peroxidase 4 [Artemisia annua]
MGSSVIVKFAFVMFLVGVSSAQLSTNFYAKSCPNLFLAVNSAVRSAISRETRMGASLL
RLHFHDCFVNGCDGSILLDDTSSFTGEKNAAPNFRSARGFDVVDNIKSAVERACPGVV
SCADILAITARDSVHILGGPYWNVKLGRRDSRTASQAAANNSIPPPTSSLSALISSFS
RVGLSAKDMVALRDSRTASQAAANNSIPPPTSSLSALISSFSRVGLSAKDMVALSGSH
TIGQARCTTFRARIYNETNNLDASFGASRRSNCPRASGSGDNNLAPLDVQTPNAFNND
YYKNLIAKKGLLHSDQQLFNGGSADSTVRQYSSNPRQFSSDFAAAMIKMGDFRPLTGS
SGEIRRNCRRPN
SEQ ID NO: 117
PWA92532.1 peroxidase superfamily protein [Artemisia
annua]
MGSSKIMSFSFLACTLFLMSGTSLAQLTPDFYDLTCPKVIEVVGSVVKAAVLREKRMG
ASLLRLHFHDCFVNGCDGSILLDDTPSFRGEKTANPNVNSARGFEVIDDIKSHVERLC
PGVVSCADILAIAALESVLALGGPYWRVNLGRRDSISASFDAANGNAIPPPTSSLDNL
VRTFQAVGLTADDMVALAGAHTIGQARCTSFRPHVYNDTNVDPLFASLRRTNCPIPNG
LGDNNLAPLDARTSNHFDNSYYNNLLINQGLLHSDMQLHSGGFTDRLVEEYGRDPIKF
NVDFSAAMIKMGDISPLMGNDGEIRRNCRVPN
SEQ ID NO: 118
PWA43764.1 peroxidase superfamily protein [Artemisia
annua]
MGSSKIMSFSFLACTLFLMSGTSLAQLTPDFYDLTCPKVIEVVGSVVKAAVLREKRMG
ASLLRLHFHDCFVNGCDGSILLDDTPSFRGEKTANPNVNSARGFEVIDDIKSHVERLC
PGVVSCADILAIAALESVLALGGPYWRVNLGRRDSISASFDAANGNAIPPPTSSLDNL
VRTFQAVGLTADDMVALAGAHTIGQARCTSFRPHVYNDTNVDPLFASLRRTNCPIPNG
VGDNNLAPLDARTSNHFDNSYYNNLLINQGLLHSDMQLHSGGFTDRLVEEYGRDPIKF
NVDFSAAMIKMGDISPLMGNDGEIRRNCRVPN
SEQ ID NO: 119
PWA43766.1 hypothetical protein CTI12_AA531910 [Artemisia
annua]
MASSKIISFALVASVLCLMSITSSGHELTHDFYSRSCPRVFEVVGSVVRSAVSREKRM
GASLLRLHFHDCFVNGCDGSVLLDDTPSFTGEKTAGPNANSARGFNVIDAVKSKVEEA
CPGVVSCADVLAITALESVVALGGPAWRIKLGRRDSRTASLSAANSGVIPSPSSTLSN
LINRFQAQGLSVKDLVALSGAHTIGQARCTTFRARVHNDSNIDTSFARSRQSNCPLPT
GLGDNNLAPLDVKSPAYFDNSYFRNLINEKGLLRSDQQLHSGGATDFFVEQYSRNPER
FHEDFAAAMIKMGDISPLTGRNGEIRKNCRVVNS
SEQ ID NO: 120
PWA66878.1 heme peroxidase [Artemisia annua]
MAALLIRLHFHDCFVQGCDASILLNDPATTERTAQSNIGVGGYEVIDAAKSALETICP
GIVSCADILTVAARDASVMVGGPSWSVRLGRRDSTTANLAQANNLPRADQDLQSLISD
FRDKGLSERDMVALSGSHTIGQSRCVAFRARIYSNGTDIDANFASTRRRGCPSNRGNG
DENLAPLDLVTPNSFDNNYFRNLVQRRGLLQSDQVLFSGGSSDSIVLEYSNNRARFAS
DFAAAMVRMSEIDLLTGNNGIIRTICSAAN
SEQ ID NO: 121
PWA96373.1 heme peroxidase [Artemisia annua]
MANGKAVALAVGFIFLNLAGHCYGYYGGLQYGFYKGKCRTSDVEDIVSKTVYSKFLRD
RTIAPALIRMQFHDCFVNGCDASILLDGPNSEKTAPPNLSVRGFDVIDAAKAAVEKVC
PGVVSCADIIIMATRDVVSLSGGGRYKVQTGRRDGLVSLAQNTISLPPPTASVATAIQ
LFALKGLTATDMIYLFGGHSIGIAHCSLFKDRLYNFKNTGKPDPTMDWALLTSLRKTC
AQNATIDRTANLDQNPYSSAVVDKSFYSQIIKRRGVLKFDQDLASDRLSKSTVARIAR
SSNFNTKFGQAMVKLGPVQASDPDILFDYIVPPNVTKVDGDFFTYTKIRGFFDGSSNS
ADSKSMLASMTEFPALNGQSVSLSLLRLAPGGVSAPHTRPHATGLFFVLEGTFEVGFV
DTTNKLYTQTLQTGDMFIFPKGLVHYQYNSDMKNPAVAVAAFGSASASTVLIPTTLFD
IDIEDVILAKSFKTNVATIRKLKAGVGSKS
SEQ ID NO: 122
PWA68872.1 peroxidase superfamily protein [Artemisia
annua]
MKRQVCLFLSIVVILAASSVDTVAADGLPPEAAPLIRHFYKVHNTCDNVEPFIRHQMK
LMFDKDKTITPKLVKLLYADCMINGCDASILLDGPNTEKSSPKTRGLAAFVIIDRLKT
VVEARCPGAVSCADILNIATRDAIFYSGGPSYPVFLGRRDGKKSDASWVDLPPPSISW
EASLAYFTSKGLDVQDMVTLLGGHMMGRTRCSNVLDRLYNFNNTGKPDPTMEPVTLTY
LQKQCPKKVKLGQANPLINLNPQNPTHKFTNSYYSRILANKAVLGVDQQMLYGDDTHE
LTDEFAKGLEDFRGAFAYSMSRMGGLKVLTGTKGEIRRNCHVVNK
SEQ ID NO: 123
PWA98192.1 peroxidase superfamily protein [Artemisia
annua]
MKFRQIFLSMFIHFLLLTLLHASDPPLSLDYYKSTCPNAEAIVRKEMECAVLSDPRNA
AYILRLHFHDCFVQGCDGSVLLEDTVTFQGEKNAPTNLDALKGFDIIDRIKNKLESEC
PGTVSCADVLTIAARDATILVGGPYWHVPVGRKDSRTGSFAQVETNIPGANDGLLSMI
SKFMYQGLSVTDMVALSGAHTIGMARCTNYRDRIYGDYQITSIANPIAKSNLKTLQST
CPAAGGGENNEAAMDYVSPNLFDNSYYLLLLNGEGLLNSDQELYSSVLGIETKKLVKK
YALEPIAFFEQFSESMVKLGNITNPETYVNGEVRKNCRFMNT
SEQ ID NO: 124
PWA86723.1 peroxidase 1 [Artemisia annua]
MGAHTLGFSHCDQFSNRIYNFSKQNPVDPSLNPSYATQLQQQCPKNVDPRIAINMDPN
TPRTFDNVYYKNLQNGQGLYTSDQVLLTLDQNRL
SEQ ID NO: 125
PWA52963.1 heme peroxidase [Artemisia annua]
MKVSSFNKMGILVIILLATMTNIAQGQGTRVGFYRTTCPRVESIVQSAVRQAVRSNPT
MAPGLLRIFFHDCFVNGCDASLLLNGSSSEKTARPNLPLRGFQVIDAAKAQLERACPG
VVSCADIVALAARDSVVLTGGTSWQVPLGRRDGFVSRASDTTNLPAFNDPITVQIRKF
SDKGLNTQDLVALVGGHTIGTAACSVFSYRLYNENNTNRPDPSINQAFLPQLRALCPN
GGVLESDAKLLSNPTTQRFVQRFSGVRGTPNLRFNVEFGRSMVKMGNIELKTGRQGEI
RRVCTTTN
SEQ ID NO: 126
PWA62775.1 peroxidase superfamily protein [Artemisia
annua]
MTKQQRSVCVAILALIALGMVAGVAHAAIGLPPEAAPLIRHFYKVHNTCANVEPFVRH
QVKLVYDNDRTVAPKLIKLLYADCMINGCDASILLDGPNTEKESPKTRGLGAFVLIDK
IKQVLEARCPGAVSCADILNLATRDAVFFSGGPSYPVFLGRRDGRDSNAAWVDLPSPS
ISWESSLAYFESKGLDVQDMVTLLGAHTMGRVRCSNILDRLYNFNNTGKSDPSMESLT
LRYLRKQCPPKLKLGQPNPLINLNPSNPTHRFINSYYTRVLTNKSVLGVDQQLRYGGD
TYDLTDEFASSIEDFKGGFAYSMSRMGGLKVLTGTKGEIRLNCRMVNK
SEQ ID NO: 127
PWA 65612.1 heme peroxidase [Artemisia annua]
MHIFISLFLESYTRSSQKLSHTNEKKHTRSVGRMNNTLIVMCTMIISCCKILAISETS
LRVGFYDETCPDAESIVNGFVKDAARFDNQMPAILLRLHFHDCFVQVLSLIFFFKSTQ
LSWFHLVLINNAWFEFDQGCDGSILIDDDPISEKLAEGHQGVKGFDVIENAKSRLEFV
CPGVVSCADIVAIAARDAVAFTFGPFYEVETGRRDGFVSNVSLADNMPDFRDSIQLLK
QKFFDKGLNEKDLVVLSGAHTIGTTACFFLMDRLYNFVSGGGPDPSIDPDLLPELMET
CPPNGDTDFRLPIDHNSRDTFDDLILDNIRSGFAVLQSDAKLMDDRVTKQILDSYFGS
SNQSVQPSFETDFVNSMIKMGRIGVKTSSKTGEIRRVCNTFND
SEQ ID NO: 128
PWA46240.1 peroxidase 70 [Artemisia annua]
MDEFSEKTIEKIRGVRDSEIAPSRESQLTANFYARTCPNFRSVITRAVNSAVSSEARM
GASLLRLHFHDCFGCDASVLLDTTANFTGEKGAGPNAGSIRGENVIDTIKTQLERQCA
GVVSCADILAAAARDSVVALGGPGWNLVFGRRDSTTASANTANSNLPAPTLSLSGLIS
SFSNQNLNANELVALSGAHTIGQARCTTFRSRLANDNNINASFATSTRANCPTSGGDN
NLSPLDATPTRFDNRYFSDLISQRGLLHSDQELFNGGSTDALVRTYASNQATFFRDFA
SAMVKMSNLNPLTGSNGQIRTNCRRTN
SEQ ID NO: 129
PWA50921.1 peroxidase superfamily protein [Artemisia
annua]
MSHNLFLLPLVFLALFPVVEPRLRQRYYSETCPNAETIVTFMMQKAMIKDPRSGASVM
RFQFHDCFVNGCDASLLLDDTPTMLGEKLSLSNIDSLRSYDVIDWIKGALERTCPMTV
SCADIIIMASRDAVVLVDLLKILFLIFSLSVQDLVALSGSHSIGNARCFSIVHRIYNQ
SDDGKPDPSIEPGFLKKLQELCPLGGDENVIGDLDFTPKTFDNEYFKDLVNGKGFLNS
DETLFTNPQTREYVQRYSEDQSSFFKDFVEGMKKLGDLQSGRPGEIRKNCRVVNNPPP
VNLMVRK
SEQ ID NO: 130
PWA94561.1 peroxidase superfamily protein [Artemisia
annua]
MQGCDGSVLLDSSGTINSEKRSVPNRDSVRGFEVIDEIKSVLEKACPQTVSCADILAL
AARDSTVLTGGPSWDVPLGRRDSLGASLSGSNQNIPAPNNTFQTILTKFKLKGLDIVD
FVALSGSHTIGNARCTSFRQRLYNNTGNGQPDFALDQTYAAQLRANCPRTGGDQNLFV
MDPGSPTKFDNGYYKNLIALKGMLSSDEILYTQNQQTFDLVKQYAENQEEFFKQFAKS
MIKMGNITPLIGNHGEIRKICRRINH
SEQ ID NO: 131
PWA76218.1 heme peroxidase [Artemisia annua]
MEGSSLTKTVVMLVLLLATLTTLALGQSIRGGGGGTRVGFYRSTCRNVESIVQSAVRT
AVQANPTIAPGLLRMFFHDCFVNGCDASILINGAATEKSAPPNGFLRGFEVIDSAKSQ
LETTCPGVVSCADIVALAARDSVVLAGGRSWPVPLGRRDGLVSRASDTANLPAFNDPM
TVQIQKFADKGLNIQDLVILVGGHTIGTAACALFSYRLYNFSNTNAPDPDINQAFLPQ
LRALCPNGGNGLRRVALDTGSVNSFGNSFYEELEEWAGSN
SEQ ID NO: 132
PWA45107.1 peroxidase superfamily protein [Artemisia
annua]
MDVVDEIKEALERICPMTVSCADIIIMASRDAVALSAGPFWEVKLGRLDSLSASQKAS
DDIMPSPRSNASMLIDLFARFNLSVQDLVALSGSHSIGNARCFSIVHRIYNQSDDGKP
DPSIEPGFLKKLQELCPLGGDENVIGDLDFTPRTFDNEYFKDLVNGKGFLNSDETLFT
NPQTREYVQRYSEDQSAFLKDFVEGMIKLGDLQSGLPGEIRKNCRVVNSPPPANVMVR
K
SEQ ID NO: 133
PWA82528.1 heme peroxidase [Artemisia annua]
MEAFKGDILIFTTINLETLLTSQYLSHLSFFVYLLESNMAIQKQLIVVLQLLLIALVL
KIANGKGLELGFYKNTCPRVEAIVRKTTANYIHRAPSLAAGLLRMHFHDCFVRGCDGS
VLLNSTTNNQAEKEGIPNLTLRGFQVIDAAKTALEAACPGVVSCADILALAARDAIYQ
GCDGSVLLNSTANNQAEKEGIPNLTLRGFQVIDAAKTALEAACPGVVSCADILALAAR
DAIYQIKGPFWPVPLGRRDGHTVGVSHCPVIVNRLYNFTGKGDTDPSLDPKFIPQLKS
ICSPTDTTTVLAMDRTPRSFDEDYYTVVLRKRGLFQSDAALLNDKQTSAYVKLQSKSF
GYTFFNDFSKSMVKMGKIGVLTGFAGEIRRTCALVN
SEQ ID NO: 134
PWA70020.1 heme peroxidase [Artemisia annua]
MQKMNIICFLSLFVLFMFQIATPSSDDYGLSTEYYNRVCPQALPTIKRVVEDAVAQER
RMGASLLRLHFHDCFVNGCDASILLDQTSTIDSEKNAGANLNSARGFELGGPSWEVKL
GRRDSTTASRTTANANLPSPFMDLPALIKNFKDQGLDEKDLVLLSGAHTIGFAQCFTF
RDHIYNDTNIDPAFAGHLRSICPRVGDDSNLAPLDPTPSSFDTKYFNNLMKKRGLLKT
DQVLFSKGETGELVSEYNEDQTKFFEDFAKSMIKMGNINLLTGNRGEVRDNCRRVNSQ
SEQ ID NO: 135
PWA68044.1 heme peroxidase [Artemisia annua]
MATHKQISNICLAFILFFHFYTGTCEVQLNYYSKSCPNAEEIVKQQVIALYHKHGNTA
VSWIRTLFHDCMVKSCDASILLESINGIISEKTSKQNMGMRNFKYINTIKDALEEACP
MTVSCADIVALSARDGVVMLGGPHMEMKTGRKDSKESHLAEIDALLPNHNDSMTFVLS
RFQSVGIDAEGTVALLGAHSVGRVHCVNLVDRLYPDVDPTLDPDYAEYLKRRCPHPEP
NPRDVEYSRTDLITPMTLDNMYYKNLINNRGLLIVDQQLLSNPITSPYVKKMAADNDY
FHEQFSKALLILSENNPINEDVGEVRKDCRFVNK
SEQ ID NO: 136
PWA96027.1 heme peroxidase [Artemisia annua]
MKVSSENKMGILLIILLAAMTNIAQGQGTQVGFYRTTCPRVESIVQSAVRQAVRSNPT
MAPGLLRIFFHDCFVNGCDASLLLNGSSSEKTARPNLPLRGFQVIDAAKAQLERACPG
VVSCADIVALAARDSVVLTGGTSWQVPLGRRDGFVSRASDTTNLPAFNDPITVQIRKF
GGTTTTNRRRVWVHHRMGGLTIPCSEDKFTNLACSLGTL
SEQ ID NO: 137
PWA60752 .1 peroxidase superfamily protein [Artemisia
annua]
MNNSVLFNHNYNGFHFCVDDLMLNSQGCDGSILIDNGEDSERKANGHQGVQGYNVIEN
AKAQLEIMCPSVVSCADIVAMAARDAVDLSHGPVYQVETGRKDGLVSRMELAARMPDV
QDSIHILKQKFIEKGLNDRDLVILSAAHTIGTSACFFMTRRLYDFASSGGPDPSINPS
FLPELMSSCPKRGNPNDRLPMDHGSGDIFDKQIFQNIRSGFAVLQSDAKLMDDPTIKG
IVDSYFGLLSPLTGPSFEGDFMSSMVRMGRIGVKTGSSGDIRRVCNAFN
SEQ ID NO: 138
PWA39486.1 heme peroxidase [Artemisia annua]
MASRNVSTTVFFLLFLAVINNTLCEAQLSMTFYDRTCPNALSTIRTSIRTAISRERRM
VASILRLHFHDCFVQGCDASILLDDGPTIISEKNALPNKGSVRDYEVIEAAKTEVEKL
CPGVVSCADILTVAARDASEMVGGPSWSVKLGRRDSDTASLVLAETGALPSFKAPLDS
LISTFRDNGLSARDMVALSAALELVTPNSFDNNYFKNLLQKKGLLETDQVLFSGGSTD
SIVTEYSKSPSKFKSDFAAAMVKMGEIRTLTGQQGVIRRICGALP
SEQ ID NO: 139
PWA38387.1 heme peroxidase [Artemisia annua]
MENVRVFLPPRRVHGWFKSCKSEVQLNYYSKSCPNAEEIVKQQVIALYHKHGNTAVSW
IRTLFHDCMVKSCDASILLESINGIISEKTSKQNMGMRNFKYINTIKDALEEACPMTV
SCADIVALSARDGVVMLGGPHMEMKTGRKDSKESHLAEIDALLPNHNDSMTFVLSRFQ
SVGIDAEGTVALLGAHSVGRVHCVNLVDRLYPDVDPTLDPDYAEYLKRRCPHPEPNPR
DVEYSRTDLITPMTLDNMYYKNLINNRGLLIVDQQLLSNPITSPYVKKMAADNDYFHE
QFSKALLILSENNPINEDVGEVRKDCRFVNKSLNHKAFCFLKSRDVMCYSGATRRSLN
HKAFCFLKSRDVMCYSGATRRSLNHKAFCFLKSRDVMCYSGATRRSLNHKAFCFLKSR
DVMCYSGATRRSLNHKAFCFLKSRDVMCYSGATRRSLNHKAFCFLKSRDVMCYSGATR
RSLNHKAFCFLKSRDVMCYSGATRR
SEQ ID NO: 140
AOS50885.1 peroxidase [Artemisia annua]
MALKHLIFFTLLSILAISSFADTESSTTTDSGLVLNFYKDSCPQAEDIIREQVGLLYK
RHKNTAFSWLRNIFHDCAVERCDASLLLDSTRRQLSEKETDRSFGLRNFRYLEEIKEA
VERECPGVVSCADILVLSGRDGIVAVGGPYIPLKTGRRDGRKSRADILEQYLPDHNES
MTVVLERFKSIGIDTPGVVALLGSHSVGRTHCVKLVHRLYPEVDPAPNPGHVEHMLHK
CPDAIPDPKAVQYVRNDRGTPMKLDNNYYRNILDNKGLLLVDYQLAVDKRTKPYVKKM
AKNQDYFFKQFARAITILTENNPLTGNEGEIRQQCNVANKHH
SEQ ID NO: 141
PWA86603.1 peroxidase 20 [Artemisia annua]
MLGWVVFRLVESCNKGFYKGLSLADSGLNVSLLQFADDALFFGGWSRLNAKNLIHILK
CFELASGLKVNITKSRLMGVGVSNVDIAVVASSLGCTHDSLPFTYPGLPVGRKMRGCK
SWTEVINRFRERLSSWKAKSLLAGCRLTLIKSILGSLPMYYLSLFKAPAKDSNRGISW
VKWKSILLDKDIGGLGVECLQAKNLGLLGKWKWRFLTEDKALWRIVIKEFYGEDGGFN
SSLNRIASGALVVLSQTRPSLCYNEPLVLGYYEETCQLLEKIVRRQVAIAVYKEPRMA
ASLLRLHFHDCFVMGCDASVLLDDFEGVQSEKNAGPNLNSLRGFEVIDEIKYLVEEAC
PCTVSCADLLAIVARDAVALRGGPKWNVYLGRKDSMKASLDGANQLIPAPNSSLETLI
ANFRYQGSHTIGKAQCKSFRQRIYDYDDSEKNSYHHHRNDNEFQRVLKSICPKSGKDN
ALAPLDIATPLRFDNHYFQNIKQGLGLLISDNVLISEDIEGEIRDLVWEFASDEKKFF
GMFADSLIKMGNIRVLTGQQGEIRKNCRFINT
SEQ ID NO: 142
PWA86746.1 heme peroxidase [Artemisia annua]
MLVLLLATLTTLALGQSIRGGGGGTRVGFYRSTCRNVESIVQSAVRTAVQANPTIAPG
LLRMFFHDCFVNGCDASILINGAATEKSAPPNGFLRGFEVIDAAKSQLETTCPGVVSC
ADIVALAARDSVVLAGGRSWPVPLGRRDGLVSRASDTANLPAFNDPMTVQIQKFADKG
LNIQDLVILVGKRVALDTGSVNSFGNSFYDNLRNGRGVIESDAKLWSDRRTQRFVQGF
LGGRSGSRFNAEFGRAMIKMGNIEVKTGTQGEIRRVCTATN
SEQ ID NO: 143
PWA74551.1 peroxidase 20 [Artemisia annua]
MEDNTNEIINRSEYGGSIPVDNVQALASSKDLKDIPSHYIRPEIELDVVLTDESLQIP
VIDISKLAVAGQPGYDDELENLHVACRDWGFFQIYILYLFSDSITMNNIVTLLVALLV
ISQTKTSLCDNEPLVLGYYEETCPLLEEIVQRQVEIAVHKEPRMAASLLRLNFHDCFV
MGCNASVLLDDFEGVQSEKNAGPNLNSLRGFEVIDEIKYLVEEACPCTVSCADLLAIV
ARDAVALRGGPKWNVYLGRKDSMKASLDGANQLIPAPNSSLETLIANFRYQGLNIQDL
VALSGSHTIGKAQCKSFRQRIYDYDDSEKNSYHHHHNDNEFQRVLKSICPKSGKDNAL
APLDIATPLRFDNHYFQNIKQGLGLLISDNVLISEDIEGEIRDLVWEFASDEKKMIPG
TNLHQFDVHNDGFFAHLPLRYDDGVILNMASVRMPYEKFAEFLEEKVGNYFQGLYYKV
PNVELEKGLVKVSDDKQIAHMFDVAELYGRLDLYLDHLDMDLSEYLEKADTDDMDECV
YRAKGPPKPRYCNKFSVDEMVNWAEMEVEYEASSSNHPRTSAEGDVPRASENVEVPMT
SSEVDVATTRVDGYNVRKELIELRKRIKASRLKEPESVTEMNEPNDQNQIHTENTDSH
RSETMIEHDLFMNTLMRRLQSSDENGMHQDPFVCVEKHVDRYPVYDESTHWRLRHPKK
NMLTGTQLEIAKQIQGQGSTPQTALDLDDA
SEQ ID NO: 144
PWA89952.1 peroxidase superfamily protein [Artemisia
annua]
MALKHLIFFTLLSILAISSFADTESSTTTDSGLALNFYKDSCPQAEDIIREQVGLLYK
RHKNTAFSWLRNIFHDCAVERCDASLLLDSTRRQLSEKETDRSFGLRNFRYLEEIKEA
VERECPGVVSCADILVLSGRDGIVAVGGPYIPLKTGRRDGRKSRADILEQYLPDHNES
MTVVLERFKSIGIDTPGVVALLGMYIITIMHTVHVRHSLDNPLIXPALNPGHVEHMLH
KCPDAIPDPKAVQYVRNDRGTPMKLDNNYYRNILDNKGLLLVDHQLAIDKRTKPYVKK
MAKNQDYFFKQFARAITILTENNPLTGNEGEIRQQCNVANKHH
SEQ ID NO: 145
PWA42692.1 peroxidase superfamily protein
[Artemisia annua]
MEMKSTIFIFSLITLCCLTFSHAADPPLTLDYYKSTCPNAEAIVRKEMECAVTSDLRN
AASILRLHFHDCFVQGCDGSVLLDDTYMLKGEKNAPTNLNALKGFDIIDRIKNKLESE
CPGTVSCADVLTYAARDATVLVGGPYWHVPAGRKDSKTASASLVESNIPGANDGLLTM
ISKFMYQSLSVTDMVALSENQLAFFEQFSESMVKLGNITNPETYVDGEVRKNCRFVNT
SEQ ID NO: 146
PWA23402.1 peroxidase [Artemisia annua]
MGYPKSIGTILVPIVVLLVSMNYMPCKAQLTTTFYDDTCPTALTTINDSISSAVSRNG
RMAAFIIRLHFHDCFVQGCDASILLEGGEKAAPANDGVEGYEAIEAAKAAVESVCQGV
VSCADILAVAARDASVAVGGPSWAVRLGRKDSLDSNPEQAATDLPRGDNNLDQLIASF
ARKRLSVRDMVALSG
SEQ ID NO: 147
PWA87179.1 heme peroxidase [Artemisia annua]
MACFKNLFGILMLQMVVLAIISSHADGQGLKIGFYEKSCPQAEKIVFNVMKDVMAVAP
SLSGPLLRMHFHDCFIRTKGPFWEVETGRRDGKISLIGDPINPVSGLPPFFLNITALK
QSFALRGLSVKDLVALSGGHTQGMSHCSSFENRLYNFTGIGDTDPTMDQNYIARLKLK
SKPNDQTTIAELDPGSFKTFDDSYFRLVNKRRGLLQSDAALLDDPETKAYMIQATSYS
STFFKDFGVSMVKMGRIGVLRGSQGEIRKVCSKVN
SEQ ID NO: 148
PWA73018.1 peroxidase superfamily protein [Artemisia
annua]
MMLVMIILFKFLIVFTEAQLKVGFYNKVCPAAETVVANVVKDATKSDPQTPAFMLRLH
FHDCFVESHGPVYQVETGRKDGLVSRIELAARMPDVQDSIQILKQKFIEKGLNDKDLV
ILSAAHTIGTSACFFMTTRLYDFASSGGPDPSINASFLPELTSACPKGGNPNDRLPMD
HGSGDIFDKQIFQNIRSGFAVLQSDAKLMDDPTTKGIVDSYFGLLSPLTGPSFEDDFM
SSMVRMGRIGFKTGSTGNIRRVCKAFN
SEQ ID NO: 149
PWA50920.1 peroxidase superfamily protein [Artemisia
annua]
MPSPRANASTLIDLFAKFDLSIKDLVALSGSHSIGNGRCFSIVHRLYNQTDDGRPDPS
IEPRYRQKLDKLCPLGGDEEVIGDLDFTPRVFDNQYFKDLVNGRGFLNSDETLYTFWQ
TRGYVQRYSENQSVFFKDEVEGMIKMGDLQSGRPGEIRKNCRVVNDAPSVEIVMRGSK
DRKILSF
SEQ ID NO: 150
PWA72921.1 peroxidase superfamily protein
[Artemisia annua]
MASSKIISFALVASVLCLMSITSSGGPAWRIKLGRRDSRTASLSAANIGVIPSPSSTL
SNLINRFHAQGLSVKDLVALSGAHTIGQARCTTFRARVHNDSNIDTSFTRSRQSNCPL
PTGLGDNNLAPLDVKSPAYFDNSYFRNLISEKGLLRSDQQLRSGGATDFFVEQYSRNP
ERFYEDFTAAMIKMGDISPLTGRNGEIRKNCRVVNS
SEQ ID NO: 151
PWA67845.1 heme peroxidase [Artemisia annua]
MKLEPLSTTINLSFSCSSLALDPRKMFTYEIHVYCVAFINTWLLVTVKAKVITEVDSN
IPLCENLTLLKLNCIQGCDASVLLDTTANFTGEKGAGPNAGSIRGENVIDTIKTQLES
QCAGVVSCADILAAAARDSVVALGGPGWNLVFGRRDSTTASASTANSNLPAPTLSLSG
LISSFSNQNLNANELVALSGAHTIDQARCTTFRSRLTDDNNINASFATSTRANCPTSG
GDNNLSPLDATPTIFDNSYYKDLINQRGIKLWDGLGPEPRAQYFRRAPSMIVHRYMVL
VHEAFSQAQQ
SEQ ID NO: 152
PWA77776.1 Peroxidase 48 [Artemisia annua]
MRTSSKVEVFGRAISTEGFSIQDKVVDIVHDNAWRWPSTWLVRFPTLSIVQVPILHNN
QEDVLYLKSHDGLFKDFIVRDIWHAIHDRETEVSWHSFVWSTYGFPRHSIHVWLLMRK
RLKTQDRLRQWDVGSDVDLNQLRCTLYLGAIAGCDASVLLDQTEVMGTEKTTLPNESL
KGFEHIDLIKSELENVCPGVVSCADLLVMAARESVVLVGGPFYPLHTGRKDSVQSYPV
DSFQLPSPLDTHHQSIARFTARNFTEKETVTLLGAHSTGKIHCKFFEKRLYNFDGTNQ
PDPSMDPEFVELLRTILLQGRGILFVDQQMTAGENTSNWVRQYASDVSMFHKDFAQVM
MKLSNYQVLTGSQGDVRQNCREVTTSLWQVVP
SEQ ID NO: 153
PWA70792.1 peroxidase 1 [Artemisia annua]
MKVGNICTYFAPQVDVDCNEVCTLYMWTPSKVSTAQMHAMGHHEDSWAGGPSYSVELG
RFDWLSSTAASVGGKMLKPNQNLDQLNALFTADGLTQADMIALSGFYFVSFVALVKTG
HDVALINRFHVFNIYKIENELDLNQHVPYLVVKDASSEGMQHPAVASHTKKNVYLRED
GFPVIEGSNINTSVIISRCRS
SEQ ID NO: 154
PWA70022.1 heme peroxidase [Artemisia annua]
MDLPALIKSFEDQGLDEEDLVVLSGAHTLGFAQCRTFRPHIYNDNNIDPAFASQLRNN
CPQVGGDSNIAPLDPTPSSFDTRYFNNLMSKRGVLRSDQALFNGGETDELVSKYNENY
EKFLKDFAKSMIKMGNINLLTGNRGQIRDNCRRINSQ
SEQ ID NO: 155
PWA49093.1 peroxidase superfamily protein [Artemisia
annua]
MRGFWSFGITVLGFISILGSVQGDLQTGFYSSSCPKAEKIIKDYVNQHIPNAPSLAAT
LIRMHFHDCFVRGCDASILLNFTSASGNQTEKVATPNQTVRGFDFIDRIKSLLEAECP
SIVSSADIISLAARDSIVVTGGPFWKVPTGRRDGLFSNASEALAQIPAPFDNITILTQ
KFANKSLNLKDLVLLSGGFFVKSVRNLVDGSLISSDMLLTRWVKVVPIKINLFAWKVQ
NDKLLTRLNLSWLGIDIHSIMYPICEEEAESLAHLLFSCSIARKVLSKARRWLGVSSQ
DFQSYEEWLLWFSDLRMRMQVKNMQEGVFM
SEQ ID NO: 156
PWA76335.1 heme peroxidase [Artemisia annua]
MDSRKSILFVVFLATLITLALGQTRVGFYRTSCPRVETIVQTAVRSAVNSNPTIAPGL
LRMFFHDCFVNGCDASILIDGASSEKTALPNSLLRGFEVIDAAKTQLETACPGVVSCA
DILALAARDSVVQDRVISVNVPSVVVKELKERFLPCGCISHQTHFGIMGQST
SEQ ID NO: 157
PWA89447.1 peroxidase protein [Artemisia annua]
MASGQLTANFYASSCPNFSATISTAVNSAVSNEARMGASLLRLHFHDCFVNGCDASVL
LDDTANFTGEKTAAPNNNSLRGFDVIDTIKSQLESSCPGVVSCADILATAARDSVVAV
RNINQFC
SEQ ID NO: 158
PWA95897.1 peroxidase superfamily protein [Artemisia
annua]
MAINTSHEPQAPTHKLIFMAMAFSKASCQSTLIFALFLLCNLRALEAQTPAPAPGLSY
TFYRTTCPNLETIIRQQLQTVFASDIGQAAGLLRLHFHDCFVQGHDAHDNQQXRLTCP
TNTSNNTTVLDIRSPNVFDNKYYVDLMNRQGLFTSDQDLYTDSRTRTIVTNFAVNQTL
FYQNFINAMIKMGQLNVLTGTQGEIRANCSRRNSNDLFILPTMENDQDEKVASY
SEQ ID NO: 159
PWA42010.1 heme peroxidase [Artemisia annua]
MGYPKSIGTILVPIVVLLVSMNYMPCKAQLSATFYDDTCPTALTTINDSISSAVSRNR
RMAAFIIRLHFHDCFVQGCDASILLEGGEKAAPANDGVEGYPAIDAAKAAVESVCPGV
VSCADILAVAARDASVGVMTKP
SEQ ID NO: 160
PWA35984.1 heme peroxidase [Artemisia annua]
MDLPALIKNFEDQGLDEEDLVVLSGVHTLGFAQCFTFRDHIYNNTNIDPAFAGHLKII
CPRVGGDSNLTPLDPTPSPFDTKYFNNLMRKRGVLKSDQVLFSKGETSELVSEYNEGQ
TKFFKDLQSL
SEQ ID NO: 161
PWA70021.1 uvrD-like Helicase, ATP-binding domain, P-loop
containing nucleoside triphosphate hydrolase [Artemisia
annua]
MDLPALIKNFEDQGLDEEDLVVLSGVHTLGFAQCFTFRDHIYNNTNIDPAFAGHLKII
CPRVGGDSNLTPLDPTPSPFDTKYFNNLMRKRGVLKSDQVLFSKGETSELVSEYNEGR
FDSLNSLTHLRILSLKNNRLTGPVPDLSNLTSLKLLFLSNNFISGAFPPSLPSLFGLY
RLDLSFNNFSGEIPATLNTMIDIVFPLDWRKSVDEDLISLKETDLSVNLLEDIILQAV
DIKALERVIIICLCSRVSVAVYENLINKLKDKPQFKSFVEKFRGGGLKDVLVAPTLQR
ALEDNFRVASGFLSPHSFGVLVERIQDILCFSENTMAPNYSPKLLYFLSGYDNIAYFL
PKKFLCSLLRKRVNQKLNLDPEVVAEAFLSIDDPLLIVRSENVGPKVNAPCAIFLDLE
KSKEEIMSILFPSKNTHNVHTPSNNVNAGTIPEILSSDTLQDTNIMNRVELQINWKVL
EEIYEAINGKKGVALNKLSAATMIKNELDINRGTLLTTFAAQTSCSNKDARAIRDANE
DLKLLSFAFYTSQLELKHSVILKGVSRAVKGLQSHRPKLDNFLNRSSMNQAIKLSLRG
NHDHFNVQQDDYGLSTKYYDRVCPQALPTIKRVVEDAVAQERRMGASLLRLHFHDCFV
NGCDASILLDQTSTIDTEKNAVANVDSARGFDVIDKIKSEVDKICGRPIVSCADILTV
AARDSVVAVS
SEQ ID NO: 162
PWA61783.1 hypothetical protein CTI12 AA368000 [Artemisia
annua]
MPKRYRFPVHGTVPRVRNKVIKRVPFKMVWELFEKSRFGTRIQKRGLKAVNVSGGKLG
TAVTTKCFASDPAQLKSAREDIKELLKTTFCHPILMGSFLNEMLANNWTCVRRVAVRL
GWHDAGTYNKNIEEWPQRGGANGSLVNALKLLQPIKDKYTGVTYADLFQLASATAVEE
AGGPKLPMKYGRVDVSGPEQCPEEGRLPDFTAVFLHFPLVSVMHVFSCAFIIAYELRS
QHVRLGWHDAGTYNKNIEEWPQRGGANGSLVNALKLLQPIKDKYTGVTYADLFQLASA
TAVEEAGGPKLPMKYGRVDVSGPEQCPEEGRLPDAGPPSPADHLRDVFYRMGLSDKDI
VALSGAHTLGRSRPERSGWGKPETKYTKEGPGAPGGQSWTVKWLRFDNSYFKDIKEKR
DEDLLVLPTDAALFDDPSFKVFAEKYAEDEKAFFTDYAEAHAKLSNLGAKFDPAEGFS
IDDEPAAPVGEKFVAAKYSSGKRELSDAMKQKIRSEYVGFGGSPDKPLQSNYFLNIII
VIAVLAILTSLVGN
SEQ ID NO: 163
PWA83814.1 ascorbate peroxidase 3 [Artemisia annua]
MAGKGLVVDAEYLKEIEKARRDLRALISNKNCAPIMLRLAWHDAGTYDAKTKTGGPNG
SIRNEQEYRHAANNGLKIAIDLCEEVKAKHPRITYADLYQLAGVVAVEVTGGPTIDFV
PGRKDSNESPNEGRLPDAKQGSSHLRDVFYRMGLSDKDIVALSGGHTLGKAHPERSGF
EEKPWTKDPLKFDNSYFVELLKGDDAGLLKLPTDKALVEDPKFRYYVELYAKDEEAFF
KDYAESHKKLSELGFKPPLSCLKLSSKNAALLAQSAVGVAVAATVVILSYFYEVNRKF
K
SEQ ID NO: 164
PWA61870.1 L-ascorbate peroxidase 4 [Artemisia annua]
MAMPIVDTEYLKQIEKARRELRAFIANKNCAPIMLRLAWHDAGTYDVNTKTGGPNGSI
RSEEEYTHGSNNGLKIAIDFCEEIKSKHPRITYADLYQLAGVVAVEVTGGPTVDFVPG
RKDSTISPKEGRLPNATKGAPHLRDIFYRMGLSDKDIVALSGGHTLGRAHADRSGFEG
PWTREPLKFDNSYFVELLKGESEGLLKLPTDIALLDDPAFRHYVDLYAKDEDAFFNDY
ALSHKKLSELGFTPGSSKKVKDSVVLAQSAVGVIVTAAVVILSYVYEARKRSK
SEQ ID NO: 165
PWA37152.1 heme peroxidase [Artemisia annua]
MQKMNIISFLSLFVFFMFQIATASSDDYGLSTEYYNRVCPQALPTIKRVVEDAVAQER
RMGASLLRLHFHDCFVNGCDASILLDQTSTIDSEKNAGANVNSARGFELGVLGESLIR
GGVEAITAVIRRQWAEENPVITFSFTLVPAAYKSRSGDGMRRRLVDNEA
SEQ ID NO: 166
PWA52449.1 heme peroxidase [Artemisia annua]
MFFHDCFVNGCDGSILIDGPSAEKTVVPNVPIRGFEVIDAAKTQLEATCPGVVSCADI
LALAARDSVVLLCISCRVVDVDGKCQRDGEMDWFHKHLILQIYQPLTIQLASKSESFL
KKVLTLKILLLLLELTNLLGQQTKRLAGFEGVQEVRDISGSPHSHLIDDIAIGLEIRV
YQIELRVLIWGQTGIAMVELSGV
SEQ ID NO: 167
PWA35009.1 hypothetical protein CTI12_AA613540 [Artemisia
annua]
MGSHDFYSRTCPNVFEAVGSVVRSAVSREKRMGASLLRLHFHDYFVNGCDGSVLLDDT
PSFTGEKTAGPNANSAEVESCRSLSWCSPY
SEQ ID NO: 168
PWA38558.1 hypothetical protein CTI12_AA580250 [Artemisia
annua]
MDGDITLIDSKEVTEEVVELHVETDVEVAGGKIAETLFTAADLGSNEVDMVSPTLTLL
QLLPDTTIKALLLRVSSHDFYSRTCPNVFEAVGSVVRSAVSREKRMGASLLRLHFHDY
FVNGCDGSVLLDDTPSFTGEKTAGPNANSARGCDGSVLLDDTPSFTGEKTAGPNANSA
RVLT
SEQ ID NO: 169
PWA81822.1 peroxidase 4 [Artemisia annua]
MDGDITLIDSKEVTEEVVELHVETVVEVAGGKIAETLFTAADLGSNEVDMVSPTLTLL
QLLPATTIKVGSVIRSAVSREKRMGASLLRLHFHDCFVNGCDGSVLLDDTPSFTGEKT
AGPNANSARSKVVEVCPGVVSCADVLAITARQSVVAGGFLLNGILLTREYVQHQTMSS
GMAIKHDKMLLPSIAAPMTEMCGSGQGKHIIQVLVVGRIYDFGQQRILKISTIKYAIQ
TLVRKSSACETMGSLTVICTDKTGTLTLNQMKGIMAACGYSHACGLNCNWQTCFILLQ
LSYKSRVTWTEEEHRSFLIGLEKLGKGDWRGISKNYVPSRTPTQSAVTPVTTVRPPVA
PIFRSDSIENFNRLSYAASFLIYQMYDLSHCLSSFLYYHRLQDANSNGTEILQSFILT
QQYVDSVLGYEREKMKCCEIKYKSPN
SEQ ID NO: 170
PWA98016.1 heme peroxidase [Artemisia annua]
MDPGSRKTFDLSYNSLLLKQRVLFESDSALTRNSITLTYINQLLQGSLQNFFSEFALS
MEKMNEIEVKTGTSGEIRRNCAVVNN
SEQ ID NO: 171
PWA47880.1 peroxidase [Artemisia annua]
MGDVDLSYNRRVFDNVYFQNLPKGLGVLKSDRGLVMNKRTRGYVEWYATDQKAFFRHL
GRRWRS
SEQ ID NO: 172
PWA97467.1 heme peroxidase [Artemisia annua]
MAIMSALLTSLLILSFLSRGTSLSSKYYEKTCPNAESLIRKAVRDVVTKDKKVPAELL
RMHFHDRFIRVSRIPHCSKTESTTSTPQWMLTRQSGLHFLKFKEHLPSQEQCKECRCS
NGSFFNKF
SEQ ID NO: 173
PWA57815.1 heme peroxidase [Artemisia annua]
MTFLYLFVFLMFSTATACLDSVLSTRYYDHVCPEALPTIKRVVEDAVAQERRMGASLL
RLQFHDCFVNGAHTLGLADECRFLRPHIYNDTNIDPAFQPSPSVTICPRVEGDSNLTP
LDSTSGSFDEKYFPNLVSKRGVLRSYQVLFNDSETDKIVSRYYEDQKKFFKDFAKSMI
RMGDINPLTGNRG
SEQ ID NO: 174
PWA94167.1 heme peroxidase [Artemisia annua]
MLVPDPEGKLPEETLDAFGLKQSFQRKGFSTQELVALSGAHTLGSKGFGNSVVFDNAY
YKVLLEKPWQSSAGMTAMVGLPSDRALIEDDECLRWISKYAEDQELFFEDFKNAYIKL
VDSGAKMEKGLIVPTFDIITL
SEQ ID NO: 175
PWA86800.1 hypothetical protein CTI12_AA077770 [Artemisia
annua]
MGKTYPVITEEYRLALEAAKPKLKAFLTESGSAPLMLRLAFHTAGTFDVKTRTGGPFG
TIRQKEEQAHGANVGLDVAINMLDPLRQEFSILSWGDFLMLVGALAVEVTGGPIVPFH
PGRPGSGHKDKSGFEGSWTSNPLVFDNSYYKELLAGEKAGLLKLPSDKALLADPIFRP
LVEKFAADQNAFFQAYSESYMKLSELGWADIP
SEQ ID NO: 176
PWA58067.1 hypothetical protein CTI12_AA372170 [Artemisia
annua]
MALTMFNLNKSLGFVGALMERVFGTNLFDTNLALSIYTVHRFVLIAHTNREVAQVGLS
VPPRLSTISTEACNEYVQIGKKLPSGLWDEILEGLQYVQQELGSSLGDLTKPLLISVR
SGVVLLYITFGIMVLLKNSKKETPELKSESKCINDCNLITSFDCANILKNGFRNDDLM
VVADVSSGDCRKFNVAILTCFLGNGQLGWVLSQAGTLGGPSWKVKLGRRDSETASRAN
ANANLPSPFMDLPSDVKSVTIAGELTHCFIWPEHHMTDIIVPVLEEVLHETKVEDTWK
ALTNVIAIGIGESFLGPLFVHTALPTVETVQLTDNSPRRSRFCVSVGHSLLEWLIHSL
AYKIVCL
SEQ ID NO: 177
PWA54376.1 UDP-Glycosyltransferase superfamily protein
[Artemisia annua]
MTAHHRILIVVFPGQGHINPSLQFAKRILSMGVEEVTFCTNMSVVRRIEKQTTHHGLT
EAPISENDPKPNETLEQFCSDFATNGAPAVSDIIKSAAASGNPFDHLVYTTIVPWAAG
VAKAHGIKSTLLWCQSATILDLYYYYFNGFGDLISCSNKNPSFAITLPGLPPLNTCDL
PTFFLPSRPQEHDFAIPLFKDHIDVLGENPRVLISTFNELEVEPIRALKNLELLPVGP
LIPSAFLDGQDPSDNSFGGDLFEKSVEDYIKWLNTKPKSSVVYVSFGSMSTLSFDQAE
EMANGLLESGRPFLWVIRDGGETLEVSKIEEMKKQGMIVGWCSQVEVLSHQAIGCFVS
HCGWNSTVEALAAGVPTVAFPQWTDQGTNAKMIEDVWKTGVRVRKREGHEVVDGKEIE
RCVKMVMGNEEMRRNAEKWKDLARKAVNNGGSSTINLQAFLDDA
SEQ ID NO: 178
PWA87358.1 protein kinase-like domain-containing protein
[Artemisia annua]
MNSPQAIIHLSSSSLISFLAITCLTSATVCASYGGLEGSLSPHVGNLSFLRNLSLWNN
SFQGTIPHELGRLSRLRHLFLAENNFNGVIPTNLSGCSNLEQLWLAENKLTGSIPKEM
SLLSKLVYLGIHDNKLTGGIPPFFGNITSMEVFSARRNPLGGSIPDTLGHWKSLTHFY
SGGCNLYGSIPHSIFNLSLLVNLSLSTNHLTGSLPLEIGNQLPNLEFLQLRNNKLTGV
LPPSISNCSKLGFLEMSSNNFSRKLTIDFSKLRDIYKINLHDNNFHGSGEADDMRFIN
SLKNCTKLVTLQLYNCNLRGVLPISIGNLSHQLSLLGLGGNQLFGSLPSSIGGHIPSS
IGNCKQLNGLYLANNRLSGNIPKQLFQLPSLNNFLILSQNSLSGSIPSEITNLKTLSY
LDLSYNNLSGNITSNLGECVSLTTLNLRGNRFQGVIPSSFSSLGGLGVLDISENNLSG
RIPQFLGKWKSLEYLNLSFNDFEGEVPVEGVFANASLFSVLGNDKLCGGLKRVTALRL
VSQGLEGSLSPHVGNLSFLREISLWNNSFQGTIPHELGRLSRLRGLYLSINKFSGVIP
TNLSGCSNLEHLWLYKNELTGSIPKEMSLLSKLVYLGIDDNKLTGGIPPFLGNITSME
WFYASGNLLGGNIPDTLGLWKNLTEFVCGGCNLYGSIPHSIFNLSLLVNFSLPINHLT
GSLPSEIGNQLPNLELLQLWGNELTGVLPPSLSNCSKLGYFEMSDNNFSGKLTIDFSK
LRDIYIIGLYDNNFHGRGEADDLRFVDSLKNCTRLAVLELSSCNLRGVLPISIGNLSK
QLEFLHLGGNQLFGNLPSSIGSLVGLTTLSLENNRFKGNIPTSVGKLQKLQALGLDRN
HFSGPIPDAIRNLSLLNKLYLNFNKLEGHIPSSLGNCKELIGLNLADNRFTGKIPKQL
LQLPSLTYFFNLSHNKMSGSIPSEIKDLKTLSYLDLSYNNLSGNITSNLGECMSLTTL
KLRGNRFQGVIPSSFSSLGGLGVLDISENNLSGRIPQFFGKWKSLEFLNLSFNDFEGE
VPVEGVFANASLFSVLGNDKLCGGLVTLGLPKCKETGSKKKRFPFFIFVMVSAPTLLI
VLCCVYLLCKKKQNSQQSQSSRSERFMKVSYGQLLKATDGFSGANLIGQGGFSSVYRG
ILNSDDDKSVAIKVLHLQNRGAQKSFVAECEAWPNIRHRNLLKIITSCSSTDFQGNDF
KALVYEFMPNGSVHDWLHSSANTSKLNLLQRINILRDVATALDYLHNRCQTTIVHGDL
KPSNILLDADMVAHVGDFGLAHLIGTDLNQYSSTGVKGTIGYAPPEYGLGSEMTSIGD
VYSFGILLLEVMTGKRPTDDMFSDGLSLHKFAYMALTDHVIDVVDGDAILLQSMEANA
KKVEECLVATIKIGVSCSVDSPPQRMKIEIVVNELQRILDVLQNI
SEQ ID NO: 179
Aa547
MVVLFNNKKPILCLLLLATLITLSLGQGTRLGFYRTSCPRVESIVQNAVNSAVRANPT
IAPGLLRMFFHDCFVNGCDASILIDGASSEKTARPNSLLRGFEVIDAAKRQLEIACPR
VVSCADILALAARDSVVQTGGTGWSVPGLRKDGLVSRAADTANLPAFNDPINVQIRKF
TDKGLNTQDLVALSGAHTIGTAACALFSYRLYNYNNTN-
GPDPTIDASFLPTLRNLCPNGGDGTTRVTLDTGSVRNFDNSYYSNLRNRRGVLESDAA
LWNNPATQTLVNRFLGLRGLLGLRFNIEFGRSMVKMGNIEVKTGTQGEIRRVCTATN
SEQ ID NO: 180
Aa540
MTMAFSKASCQTLIFALLLLCNLRALEAQTPAPAPGLSYTFYRTTCPNLETIIRQQLQ
TVFASDIGQAAGLLRLHFHDCFVQGCDASVLLDGSASGPSEKDAPPNLTLRAQAFVII
ENLRRLVHNACNRTVSCADITALAARDAVFLSGGPNYSIPLGRRDGLTFATVNVTLAN
LPPPTANASTILSSLALKGFTPTDVVSLSGGHTIGIGHCGSFNNRIYPSRDPSMDQTF
FNNLRLTCPTNTSNNTTVLDIRSPNVFDNKYYVDLMNRQGLFTSDQDLYTDSRTRTIV
TNFAVNQTLFYQNFINAMIKMGQLNVLTGTQGEIRANCSRRNSNDLFILPTMENDQDE
KVASY
SEQ ID NO: 181
Aa528
MALKHLIFFTLLSILAISSFADTESSTTTDSGLALNFYKDSCPQAEDIIREQVGLLYK
RHKNTAFSWLRNIFHDCAVERCDASLLLDSTRRQLSEKETDRSFGLRNFRYLEEIKEA
VERECPGVVSCADILVLSGRDGIVAVGGPYIPLKTGRRDGRKSRADIWSKLPDHNERM
TVVLERFKSIGIDTPGVVALLGSHSVGRTHCVKLVHRLYPEVDPALNPGHVEHMLHKC
PDAIPDPKAVQYVRNDRGTPMKLDNNYYRNILDNKGLLLVDHQLAVDKRTKPYVKKMA
KNQDYFFKQFARAITILTENNPLTGNEGEIRQQCNVANKHH
SEQ ID NO: 182
AAL35364
MEYYYNYNSINKMVSIIFILVLAIDLTMVLGQGTRVGFYSSTCPRAESIVQSTVRSHF
QSDPTVAPGLLTMHFHDCFVQGCDASILISGSGTERTAPPNSLLRGYEVIDDAKQQIE
AICPGVVSCADILALAARDSVLVTKGLTWSVPTGRRDGLVSRASDTSDLPGFTESVDS
QKQKFSAKGLNTQDLVTLVGGHTIGTSACQFFSYRLYNFNSTGGPDPSIDASFLPTLR
GLCPQNGDGSKRVALDTGSVNNFDTSYFSNLRNGRGILESDQKLWTDDSTKVFIQRYL
GLRGFLGLRFGVEFGRSMVKMSNIEVKTGTNGEIRKVCSAIN
SEQ ID NO: 183
CAG25463
MAYYNYNLINKMVTIIFILVLVIVDVTMVFGQGTRVGFYSSTCPRAESIVQSTVRSHF
QSDPTVAPGLLRMHFHDCFVQGCDGSILISGTGTERTAPPNSNLRGFEVIDDAKQQIE
AVCPGVVSCADILALAARDSVLVTKGLTWSVPTGRTDGRVSSASDTSNLPGFTESVAA
QKQKFAAKGLNTQDLVTLVGGHTIGTSACQFFSYRLYNFNSTGGPDPSIDATFLSQLQ
ALCPQNGDGSKRVALDTGSVNNFDTSYFSNLRNGRGILESDQILWTDASTKVFVQRYL
GLRGFLGLRFGLEFGKSMVKMSNIEVLTGTNGEIRKVCSAFN
SEQ ID NO: 184
NP_001312671
MEYYHHSINKMAMFMVILVLAIDVTMVLGQGTRVGFYSSTCPRAESIVQSTVRAHFQS
DPTVAPGILRMHFHDCFVLGCDGSILIEGSDAERTAIPNRNLRGFDVIEDAKKQIEAI
CPGVVSCADILALAARDSVVATRGLTWSVPTGRRDGRVSRAADAGNLPAFFDSVDVQK
QKFTAKGLNTQDLVALTGAHTIGTAGCAVIRGRLFNFNSTGGPDPSIDATFLPQLQAL
CPQNGDAARRVALDTGSANNFDTSYFSNLRNGRGVLESDQKLWTDASTKVFVQRFLGI
RGLLGLTFGVEFGRSMVKMSNIEVKTGTNGEIRKVCSAIN
SEQ ID NO: 185
AAL93154
MDVSCFSQNVLLVTLLLAIAVSLVESQGTRVGFYSTSCPRVESIVRSTVQSHFGSDPT
IAPGLLRMHFHDCFVHGCDASILIDGPGTEKTAPPNLLLRGYEVIDDAKTQLEAACPG
VVSCADILALAARDSVVLSSGASWAVPTGRRDGTVSQASDAANLPGFRDSVDVQKQKF
AAKGLNTQDLVTLVGGHTIGTTACQFFRYRLYNFTTTGNGADPSITAAFVSQLQALCP
QNGDGSRRIGLDTGSVNRFDNSFFANLRDGKGILESDQRLWTDASTKTFVQRFLGIRG
LLGLTFNIEFGRSMVKMSNIEVKTGTVGEIRKVCSKVN
SEQ ID NO: 186
ADP37430
MAMASLKASSVATLLLFAVLISLRGLEAQTPPVAPGLSYTFYQTTCPQLQTIIRQQLQ
TVFNSDIGQAAGLLRLHFHDCFVQGCDGSVLLDGSASGPSEKSAIPNLTLRAQAFVII
ENLRRLVHNACNRTVSCADITALAARDAVVLSGGPNYNIPLGRRDGLNFATTNVTLAN
LPPPFANTTTLLNSLALKGFNPTDVVALSGGHTIGIAHCTSFESRLFPSRDPTMDQTF
FNNLRTTCPALNTTNTTFMDIRSPNVFDNRYYVDLMNRQGLFTSDQDLYTDSRTRGIV
TNFAINQTLFFQNFVNAMIKMSQLSVLTGTQGEIRANCSRRNGNDLFILPGMEKNDDE
KVASY
SEQ ID NO: 187
BAE20169
MASTLTSLSSLFFFFSSLSLLLYHLHVSEAQTPPIVKGLSLAFFDSTCPKLESIVRKQ
LEKEFKADIGQAAGLLRLHFHDCFVQGCDASVLLDGSASGPSEKNAPPNLSLRAKAFT
IIEDLRRQVHKQCGKIVSCADITALAARDAVVLSGGPNYQVPYGRRDGLQFATRQATL
ANLPPPFANTTTILNSLVTKNFDPTDVVALSGAHTIGLSHCSSFIRRLFPTQDSTMAQ
SFAKDLRITCPTNTTDNTTNLDFRTPNVFDNKYYVDLVNRQGLFTSDQDLFTDNRTRG
IVTSFANNQTLFFEKFVNAMLKMGQLSVLTGTQGEIRGNCSVKNSNNLFLSTVVEEGM
ENLLGF
SEQ ID NO: 188
ADN96694
MSLSTISLRSLLLLSLLSLHVSLNAIQAQTYPPLVNGLSWTFHDFDCPNLESIITNRL
RQVFQNDIGQAAGLLRLHFHDCFVQGCDASVLLVGSASGPSEQAAPPNLSLRQQAFRI
IEDLRNRVHRQCGRVVSCADITALAARDAIVLSGGPAYEVPLGRRDGTTFATRNETLR
NLPAPTSNTSVLLTSLATNQNLNATDLVALSGGHTIGIGHCTSFEGRLFPARDPTMDQ
TFFNNLRGTCPALNTTNTTVLDIRSPNVFDNRYYVDLMNRQGLFTSDQDMYTDSRTRG
IITSFAVNQTLFFEKFVYSMTKMGQLNVLTGTRGEIRANCSVRNSGNDFLSLVVESQG
GDLSGF
SEQ ID NO: 189
CAP72490
MVFMSSFSSSSSSTSLLLFLISSLLISTHFNVHIEAQTTPPIVSGLSFTFYDSSCPDL
ESIIRNRLRRVFRNDIGQAAGLLRLHFHDCFVQGCDGSVLLVGSASGPGEQDAPPNLS
LRQEAFRIINDLRRRVHSRCGRIVSCSDILALAARDSVVLSGGPEYDVPLGRRDGLNF
ATQNETLNNLPPPSSNTSELLTSLATKNFNATDVVALSGGHTIGVGHCVSFEERLYPT
QDPTMDQTFARNLRLTCPALNTTNTTVLDIRSPNRFDNRYYVDLMNRQGLFTSDQDLY
TDRRTRGIVTDFAVNQTLFFEKFVYAMIKMGQLNVLTGNQGEIRANCSVRNAASGRSS
SLVSVVEDAAEVGLSMF
SEQ ID NO: 190
AAK52084
MASITLSSFAASLLLILSVNFYQTEAQGTQPIVKGLSWTFYDSICPNAESIIRSRLQQ
VFRQDIGQAAGLLRLHFHDCFVQGCDGSVLLDGSASGPSEKDAPPNLTLRQQAFRIIE
DLRRRVHRDCGRVVSCADITAIAARDSVFLSGGPDYDLPLGRRDGLNFATRNETLANL
PPPSFNASAILTSLATKNFTPTDVVALSGGHTIGIGHCTSFTERLYPNQDPSMDKTFA
NNLKNTCPTSNSTNTTVLDIRSPNKFDNKYYVDLMNRQGLFTSDQDLYTDRRTRGIVT
SFAINESLFFEEFVNSMIKMGQLNVLTGTQGEIRANCSVRNSANYNLLLSTSVAEEQQ
RTWSEI
SEQ ID NO: 191
AEX97056
MAAASSRTRVCSWLLLFSAILVVSHLLVTQAAASDVPIVNGLSFSFYDKSCPKLESIV
RTELKKIFKKDIGQTAGLLRLHFHDCFVQGCDGSVLLDGSASGPSEKDAPPNLSLRAQ
AFKIIDDLRARVHKRCGRIVSCADITALAARDSVFLSGGPEYDIPLGRRDGLTFATRN
VTLANLPAPSSTAAXILDSLATKNLNPTDVVALSGGHTIGIGHCSSFTNRLFPQDPVM
DKTFAKNLKLTCPTNTTDNTTVLDIRSPNKFDNKYYVDLMNRQGLFTSDQDLYTDKKT
RGIVTSFAVNQSLFFEKFVDAMLKMGQLSVLTGSQGEIRANCSVRNTDNKSILTSIVV
DVVEEILEM
SEQ ID NO: 192
ADD54644
MGARIIFFFALSSFSALSLRPAFGDNEEGDTGLIMNFYKDTCPQAEDIIKEQVKLLYK
RHKNTAFSWLRNIFHDCAVQSCDASLLLDSTRRVLSEKETDRSFGMRNFRYVETIKEA
VERECPGVVSCADILVLSARDGIVALGGPFIPLKTGRRDGRKSRADVLEEYLPDHNES
ISVVLDRFASMGIDTPGVVALLGAHSVGRTHCVKLVHRLYPEVDPVLNPDHVEHMLHK
CPDALPDPKAVQYVRNDRGTPMILDNNYYRNILDNKGLLIVDHQLATDKRTKPYVKKM
AKSQGYFFKEFSRAITILSENNPLTGNKGEIRKQCNVANKLH
SEQ ID NO: 193
AAA99868
MRKIMGAKVLFFFALLSFSAVSAFAQDEEDQGLVMNFYKDSCPQAEDIIKEQVKLLYK
RHKNTAFSWLRNIFHDCAVQSCDASLLLDSTRRSLSEKETDRSFGLRNFRYIETIKEA
VERECPGVVSCADILVLSAREGIVSLGGPYIPLKTGRRDGRRSRADVVEEYLPDHNET
ISGVLDRFAAMGIDTPGVVALLGAHSVGRTHCVKLVHRLYPEVDPALSPDHVPHMLHK
CPDQIPDPKAVQYVRNDRGTPMVLDNNYYRNILDNKGLLIVDHQLAYDKRTRPYVKKM
AKSQDYFFKEFSRAITLLSENNPLTGSKGEIRKQCNLANKLH
SEQ ID NO: 194
ABN46984
MDSKALFFFALVCFCAVALRPVFAENEEDPGLVMTFYKDSCPQAEDIIKEQVKLLYKR
HKNTAFSWLRNIFHDCAVQSCDASLLLDSTRRDLSEKETDRSFGLRNFRYLDTIKEAV
ERECPGVVSCADILVLSARDGIVALGGPYIPLKTGRRDGRRSRADVIEQFLPDHNESI
SVVLDRFAAMGIDTPGVVALLGAHSVGRTHCVKLVHRLYPEVDPALNPDHVEHMFKKC
PDPIPDPKAVQYVRNDRGTPMKLDNNYYRNIMDNKGLLIVDHQLANDKRTKPYVKKMA
KSQDYFFKEFARAITILSENNPLTGTKGEIRKQCSVANKHH
SEQ ID NO: 195
ACT21094
MGSKVLFFFAILSLSVLFSLNLNLAFAENEEIEEQVGLVMNFYKDTCPQAEEVIREQV
KLLYKRHKNTAFSWLRNIFHDCAVTSCDASLLLDSTRRSLSEKETDRSFGLRNFRYLD
TIKEAVERECPGVVSCSDILVLSARDGIVALGGPYIPLKTGRRDGRKSRAEVLEQYLP
DHNESMSVVLERFASIGIDTPGVVALLGAHSVGRTHCVKLVHRLYPEVDPVLNPDHVE
HMLHKCPDPIPDPKAVQYVRNDRGTPMKLDNNYYRNILDNKGLLIVDHQLATDKRTKP
FVKKMAKSQDYFFKEFARAITILSENNPLTGTKGEIRKQCNVANKLH
SEQ ID NO: 196
AAY26520
MASKTLFFLVILSFSALSTFAENEAEADPGLVMNYYKDSCPQAEDIIREQVKLLYKRH
KNTAFSWLRNIFHDCFVESCDASLLLDSTRRVLSEKETDRSFGMRNFRYLEDIKEALE
RECPGVVSCADILVLSARDGIVSLGGPFIPLKTGRRDGRRSRAEILEQHLPDHNESLT
VVLERFGSIGINTPGLVALLGAHSVGRTHCVKLVHRLYPEVDPAFPESHVQHMLKKCP
DPIPDPKAVQYVRNDRGTPMKLDNNYYRNILDNKGLLLVDHQLATDKRTKPFVKKMAK
SQDYFFKEFARAITILSENNPLTGTKGEIRKQCNVANKLH
SEQ ID NO: 197
AAD33072
MGFKVFFFFAILFFSAVSAFAEDNSGLVMDYYKDSCPQAEDIIREQVKLLYKRHKNTA
FSWLRNIFHDCFVESCDASLLLDSTRRMLSEKETDRSFGMRNFRYIETIKEAVERECP
GVVSCADILVLSGRDGIVALGGPYVPLKTGRRDGRKSRADILEQHLPDHNESMSVVLE
RFANVGINAPGVVALLGAHSVGRTHCVKLVHRLYPEVDPQLNPDHVPHMLKKCPDPIP
DPKAVQYVRNDRGTPMKLDNNYYRNILENKGLMLVDHQLATDKRTKPYVKKMAKSQDY
FFKEFARAITILTENNPLTGTKGEIRKQCNLANKLH
SEQ ID NO: 198
B9W4V6
MKYFPLFPTLVFAARVVAFPAYASLAGLSQQELDAIIPTLEAREPGLPPGPLENSSAK
LVNDEAHPWKPLRPGDIRGPCPGLNTLASHGYLPRNGVATPVQIINAVQEGLNFDNQA
AVFATYAAHLVDGNLITDLLSIGRKTRLTGPDPPPPASVGGLNEHGTFEGDASMTRGD
AFFGNNHDFNETLFEQLVDYSNRFGGGKYNLTVAGELRFKRIQDSIATNPNFSFVDFR
FFTAYGETTFPANLFVDGRRDDGQLDMDAARSFFQFSRMPDDFFRAPSPRSGTGVEVV
IQAHPMQPGRNVGKINSYTVDPTSSDFSTPCLMYEKFVNITVKSLYPNPTVQLRKALN
TNLDFFFQGVAAGCTQVFPYGRD
SEQ ID NO: 199
AaAPO1_Nterm_recode477
MAEPGLPPGPLENSSAKLVNDEAHPWKPLRPGDIRGPCPGLNTLASHGYLPRNGVATP
VQIINAVQEGLNFDNQAAVFATYAAHLVDGNLITDLLSIGRKTRLTGPDPPPPASVGG
LNEHGTFEGDASMTRGDAFFGNNHDFNETLFEQLVDYSNRFGGGKYNLTVAGELRFKR
IQDSIATNPNFSFVDFRFFTAYGETTFPANLFVDGRRDDGQLDMDAARSFFQFSRMPD
DFFRAPSPRSGTGVEVVIQAHPMQPGRNVGKINSYTVDPTSSDFSTPCLMYEKFVNIT
VKSLYPNPTVQLRKALNTNLDFFFQGVAAGCTQVFPYGRD
SEQ ID NO: 200
PaDa_I
MAEPGLPPGPLENSSAKLVNDEAHPWKPLRPGDIRGPCPGLNTLASHGYLPRNGVATP
AQIINAVQEGFNFDNQAAIFATYAAHLVDGNLITDLLSIGRKTRLTGPDPPPPASVGG
LNEHGTFEGDASMTRGDAFFGNNHDFNETLFEQLVDYSNRFGGGKYNLTVAGELRFKR
IQDSIATNPNFSFVDFRFFTAYGETTFPANLFVDGRRDDGQLDMDAARSFFQFSRMPD
DFFRAPSPRSGTGVEVVVQAHPMQPGRNVGKINSYTVDPTSSDFSTPCLMYEKFVNIT
VKSLYPNPTVQLRKALNTNLDFLFQGVAAGCTQVFPYGRD
SEQ ID NO: 201
PWA66067.1
MNKKKKTTIFLVVVLLFTSFPCFSTATQSRKELRVKEVVNAIQLSRVVPPNTIDPSRV
THLSWRPRVFLYNRFLSDEECQHLISLANDDNKSSGNSSAKVMEGATNLDISLEIKDE
ITSKIEERISAWTFLPKGNGKPFHIHHHVGPEEAKENHNYLGNTSTQQQNGESVMATV
VMYLSNVTQGGQIIFPKAESGSSRPKDKVWSDCTMNSPITKPIKGNALLFFNLHPNTS
LDPMSLHARCPITEGDMWWTTKIFMVKPINQPKSVALKTSSDDLCTDEDDNCQQWAAF
GECERNPIFMVGTADYYGTCRRSCNVC
SEQ ID NO: 202
PWA76411.1
MPHQIKESQPKANKDWSECAKKGYAVKPKKGDGLLFFSLHPNATTDVLSLHGSCPVIE
GEKWSVINEPNGFISGTLTNQITQVTNAMMKMLTARHGLPVVNA
SEQ ID NO: 203
PWA37855.1
MMVAIAYFNYIKYHVVVLDLPGHRDFVQNLISGATQADASILVIDASIGAFETDIEIG
GGQTREHAQLIRSFMLYYYHLVCNSWKGYDISFLITSYLLVCNSWNYVGSFLINSYIT
NYYQRGEHFLYEKKDFLMLELSRVVPPNTIDPSRVPQLSWKPRSGENEFVVFTECNGA
YSVFSECYSDSDDTPEYLSASTCKGGFGIVFKWSLTDGMVVAGKRLKDDNSFGGDIQF
QTEVETISLVVHRNLLVRFSFKLHSTFYLVKCASICVPGTELSDFTPVILQSTQSQAV
ISNGF
SEQ ID NO: 204
PWA55244.1
MPFFDFFDSNRGGVMKAFPNQYRFFVQVGLGWVWPVGNKSIQVFCTDNGASDLPGQVW
GVYFGWVEGHSQERLQIVVSNLASDGTIVVALSGETFTCWLNQGILYQANFFDRCSNT
QTKIKSVAEACLNLPAYSYHQLSYLNLSNAYFSGVIPPHIGNLSNLKVLDLRSSTYSY
NRLVSDDMSWMSGLSSLEYLDLSKIDLSKAQNRDMVFYLMPSLVKLSLSGCSLTNADL
GTLSYSSTTLANIKHLDLGYNHFKGQFPGLFQNMTSLEFLDLSYFNFSLSLNSTNFLS
TIHYFPKMRLSSCGLHNMHLSPNHLNFTTISIIQHLDLSQNLLEGGFPSALTNMSSLL
YLDLASNMLNSSVPIMPNLLRFDLSRNNFEHIEDVGIWRQCHLKELTVSDNYFKCSQY
AFKRLHLDGSLKGAFPADTLGRMVNLRVLRLSDNELTGQIPESLGRLRLLEELDLSHN
QLRSHSDIHWKLVSLQMFSVDLNSLNGTVPVSIGQLAKLKYLDISENSLEGVITEAHF
ANLSMLKYLFTSFNSVSLEYTTTFQYVINMDLSSNNLVGEIPVELMSLSALLGLNLSN
NHLNGSISYSIGNMKALTSLDFSRNQLTSPIPPSIATFNF
SEQ ID NO: 205
PWA69361.1
MKFTRRPSAPATSSQDAHLLVRETLRISANLASSSAAPPSEDVIRSSEIKRLEDQFVD
ASLRLICYEEVDGRRFKYLRNASSSNSNSNSIRAVSLQSRQAPLDELVSFIRSYVVPE
GFPDSVSPSYVPYMTWRALKHFFGGAMGVFTTQALLHSVGVSKSQAMPGAVAINWIIK
DGSGRIGKMLFSRQGKKFDYDLKQLRLSGDLLMELGAGVELATAAAPHLFLPLACAAN
VAKNVAAVTSTSTRTPIYKAFAKGENIGDVTAKGECVGNVADLLGTGLSIIIAKRNPS
LFTTFALLSCGYLFSSYREVKSVVLHTLNRARFTVAVETFLKTG
SEQ ID NO: 206
PWA66043.1
MTTLSKVYRNVPTTSYQITPLDFDSLDGVPESHIWSQPDEPQQKIQTQEPQELSIPVI
DLTDPNALDLIGQACKTWGIFQVINHGVPLALIKKLESESRRLFALPTDEKHKVLRSA
NGVTGYGTASISKFFDKCMWHEGFGIMGSCVEDAKILWPYDYKNFWYV
SEQ ID NO: 207
PWA68690.1
MTTLSEVYRNAPMTSYQITPLDFDSLDEIQTQEPQELSIPIIDPNALDLIGQASKTWG
IFQVINHGVPLALIKKLESESRRLFALPTDEKHKVLRSANAVTGYGTARISPFFDKCM
WHEGFAIMGSCVEDAKALWPHDYKNFW
SEQ ID NO: 208
PWA63537.1
MEPKGTGLGSSLLVPSVQELAREPITKVPARYVRLDQDPPIISRPPSSLPKVPVIDMA
RLSSENSADHELEKLHIACKDRGFFQIINHGVSISLMDKVKEETQEFFKLPMEEKKKF
WQTTDDVEGFGQAFVISEEQKLDWADIFYLITLPHGIRKPYLFPNLPLPFR
SEQ ID NO: 209
PWA60234.1
MSYLSESFRAPPPSPVASGRRSFVANDDFLTEFLHNSLRVPDLVLPEHVSPRQNTKIN
NLPKLDYKSLNLLHDHKASDTFDVVAKTGCFELVNHGINDLLIRFVAEFGGKIFELSY
EEKKAVSRSSDRMYGFVESNDDEKETSEEFVWCRDDELRAKMEAIMPLQYSNFSESLD
ILSSEIENICGTILKFLHKNTSPKSESDDDEREVKQIVGSVCYLYRHHKDPNTNPNDS
EHTSSLRYDMIRMLIRGSEHPHTLCLHVCDGLSEFHVYSKKGRVSFCPEKDALVITIG
DQLQKWSDGKYKHVMGRPIYKGGDDHISLAFMYNPPTLTHMKEQKTITIDIEYILANT
TSCSESLDILSSEIENICGTILNFLLKNTSRKSGLDDDEREEKQIGGSVCYLYRHHKD
PNTNPNDSEHTSSWRYDMIRMLIRGSEHPHTLCLHVCDGLSEFHVYSKKGRVSFCPEK
DALVITIGDQLQKWSDGKYKHVMGRPIYNGGDDHISLAFMYNPPSTLTHMKEHKTITI
GHQAYFFFILIVRFYVVSC
SEQ ID NO: 210
PWA65832.1
MELKGIGLGSSLLVPSVQELAKEPITKVPPRYVRLDQDPPIISRPPSSSPDVPVIDMA
RLSSENSADQELEKLHLACKDYGFLQIINHGVSISLMDKVKKETQEFFKLSMEEKKKL
WQTTDDNEGFGQAFVFSEEQKLDWADIFYLTTLPHGIRKPHLFPNLPVPFR
SEQ ID NO: 211
PWA56995.1
MAPSTPIINSNSLNILDFVVNKGHGVKGLSELGLKTLPHQYIQPPQERFDSSNEEPNK
DSIPVIDMSSPNDPHVAKAICDAAQKWGFFQIVNHGVPIHVLEDVKNATHKFFALAPQ
EKQKYSKAQSVTNNVRFGTSFTPEAEKALEWKDYLSLFFVSNDEAASLWPPICG
SEQ ID NO: 212
PWA80821.1
MAPFLTDMHELKTFVVNEGNGVKGLSELKIKTVPELFIQPLEKRLDMSKVVLKESIPV
IDMSNYEDPEVMDSICDAAEKWGFFQVVNHDVPLSVLEDVKKATRRFFELPADEKKKY
LFNNSPTKNVRLLSSFVPEVDKAMEWKDYLSCFYVSDDEAMAFWPSVCR
SEQ ID NO: 213
PWA38931.1
MAPSFSDENSLTNFVVKEGNGVKGLVDSGLTEVPSRYIQPPHNRISKQHTTASIDQSM
TIDLSELDGPNHDQVVKSIAHAAETLGFFQVVNHGVPLELLDSLKVAAHQFFNQPVEK
KAVYLKGVSPSPMVKYGTSFNPDKEKALEWKDYVSMIYTNDDDAHMFWPNECKEPPRN
SRLELFSLRTAATKHFRSGLEGDILGKLFGLISVSDKYGLVTDGRSQLFEPDFAHVPL
FNHDWDSPINMIDWGIVYLGNSTARHSVPFASSLEFYMELYVTKEKNTCYQVCNHKEE
IDLSAFWNKESDSASGSLIVQGEDGPTHMFYIFMKDAVDISLEVKFRTETLGRKVRGY
VIAHYGDDFLYECQCDPRGRDLYSALLFLSDGVLEPGTVKLIKSVMAVPTKGNLIIKA
YLEDVGSGEVIMKNSCKLKPQLESCRRESTISGIEGGDCDLHLIVD
SEQ ID NO: 214
PWA91852.1
MTRSISITNTKSLNILDLVVSGAELRGWKGHGVKGLAELGLKALPHQYIQPHQERFAS
TTNEPNKYFIPVIGISNWDYLNVAKAICEAASKWGFFQIVNHGVPIHVLEDVKFATNK
FFALPLPPPEKVIYSKEQSVTNNVRFVTRFTPDAEKALEWKDYLF
SEQ ID NO: 215
PWA34370.1
MVIAILDKPKKGQSIDKKKEKMLPVIDLSSSSEDRISTAHSIRQACTDYGFFYLINHG
VDEQNVFEQSRLFFSLPLEDKMKSSRNIGGDFGFSPLYAENLDSSSSSKTKGGDSKET
FHIGPLQNQLNQWPSQDLLPSWRSVMETYYASLL
SEQ ID NO: 216
PWA81334.1
MHSILKKNPDTSPILDIQRQRYQDPEVEKTMNMIQLPDLVEVNPQNYEVTSRELTTAI
TVLQLFLSSSKPVLRFAVVQTLSKDELLKQPIASNDSDSSDVNEIPTAVIEVSQDELM
KNRPRKDQVNQLKDANAKPKYRIGSSLKHRIASEMCKNPKLQLLMYKSCIKLIVCVHP
AEFQNYNEELNQDSILVIDMSNFDDPNVANAICAAASKWGFFQIVNHGVPIQVLDDVK
DATRKFFTLPLKKFEILKRKFAEKALEWNDYLSFFFVLDVEAATLWPPVCRIRSTTVM
LPLFNLDLTALMVPSSNPDGGNCDVIFSCDGDMLVASTCKKDFICTIFFVKDRSSKSS
QKDDQSVESPGSPRNQKLIQITTRWDPSDACKPDVEDALIFYPTAEEFEDTLGYL
SEQ ID NO: 217
PWA74551.1
MEDNTNEIINRSEYGGSIPVDNVQALASSKDLKDIPSHYIRPEIELDVVLTDESLQIP
VIDISKLAVAGQPGYDDELENLHVACRDWGFFQIYILYLFSDSITMNNIVTLLVALLV
ISQTKTSLCDNEPLVLGYYEETCPLLEEIVQRQVEIAVHKEPRMAASLLRLNFHDCFV
MGCNASVLLDDFEGVQSEKNAGPNLNSLRGFEVIDEIKYLVEEACPCTVSCADLLAIV
ARDAVALRGGPKWNVYLGRKDSMKASLDGANQLIPAPNSSLETLIANFRYQGLNIQDL
VALSGSHTIGKAQCKSFRQRIYDYDDSEKNSYHHHHNDNEFQRVLKSICPKSGKDNAL
APLDIATPLRFDNHYFQNIKQGLGLLISDNVLISEDIEGEIRDLVWEFASDEKKMIPG
TNLHQFDVHNDGFFAHLPLRYDDGVILNMASVRMPYEKFAEFLEEKVGNYFQGLYYKV
PNVELEKGLVKVSDDKQIAHMFDVAELYGRLDLYLDHLDMDLSEYLEKADTDDMDECV
YRAKGPPKPRYCNKFSVDEMVNWAEMEVEYEASSSNHPRTSAEGDVPRASENVEVPMT
SSEVDVATTRVDGYNVRKELIELRKRIKASRLKEPESVTEMNEPNDQNQIHTENTDSH
RSETMIEHDLEMNTLMRRLQSSDENGMHQDPFVCVEKHVDRYPVYDESTHWRLRHPKK
NMLTGTQLEIAKQIQGQGSTPQTALDLDDA
SEQ ID NO: 218
PWA42889.1
MEACANWFSLAIKDDDKQQCSSSHKLDDSRLSNDSRKESEQCWHLTTPRTSYKIDHKH
PEADIAGETAATFAASSIAFKYSDPDYPNILLKHTKQTVHPTEFQNYNEESNQDSIPV
INMSNFDDPNVANAICAAASKWGFFQIVNHGVPIHVLDDFKDAMNLSCCTKFLERSHV
HPKVVMCDVDASIEAWQPGPLAKNKAITEAEVIWADRYSAVACKHI
SEQ ID NO: 219
PWA83612.1
MSINEEYRGSHFHEATFINQNGELQTSKIPVVQELAREGLTNHFPKRFISLPQRVDTI
IFETPPVIDIAKLKHNDTRKMELQKLAEAAKEWGVFLIKNHGVDDTVLDDVKDVVKGF
FALSFEEKKVNVGMYRSVDNMGYGKTHVMSEDQPLDWVDRLTIKAAPVDESTNDDLRV
WPRKPTNFRDYFNTFIEIEIISLFLVYFDLKIRKLGQHAWHRVRTQLDAERISLAYFY
NPPPQCEIGPMVNSGSMEEIYKKVVVEDYVSHFNKISPTTSKEAIMYAKVK
SEQ ID NO: 220
PWA38449.1
MLTPIKFAGCDGALANSSCTEVENWRKANNGYIGYSFLFDCVIASCCSYSQQLFKPTI
LHRYGQQIQTTAGISKSKSTEDQLSRRCMKGSSHDVDGGKALDANFHVMVIVSVTSVT
SADIAQGYTNQTVEEEFVSPRISSRRNRSAARDPLLIYLRIRASTSMDKENRAGDAKN
EQPEKVIKKKLDKDQKILDNDGFKLSNNHLTGTISGPMIARMMNLQSVHPAEFQNYNE
EPNQDSIPVIDMSNFDDPNVANAICATASKWGFFQIVNH
SEQ ID NO: 221
PWA77944.1
MVLDNTSLSQPNYDRKAELKAFDETKTGVKGLVDAGITQVPRIFHVPSPQNLNSKQSC
PKLILPTIDLEGINEDSVRRKEVIQKVNDALKTWGFFQIVNHGISNSTLEEVKKGVRE
FFEQDDEVKKEWYTRDMSGKTRVVYNSNFDLYAAPVTNWRDTFFCMMAPYHPEPHELP
QPCRF
SEQ ID NO: 222
PWA39354.1
MSYLSESFRAPPPSPVASGRRSFVANDDFLTEFLHNSLRVPDLVLPEHVSPRQNTKIN
NLPKLDYKSLNLLHDHKASDTFDVVAKTGCFELVNHGINDLLIREVAEFGGKIFELSY
EEKKAVSRSSDRMYGFVESNDDEKETSEEFVWCRDDELRAKMEAIMPLQYSNFSESLD
ILSSEIENICGTILKFLHKNTSPKSESDDDEREVKQIVGSVCYLYRHHKDPNTNPNDS
EHTSSLRYDMIRMLIRGSEHPHTLCLHVCDGLSEFHVYSKKGRVSFXNKKCKVMCRMG
SGDRWNWYRAPESSCELEELDFLVNVVELVELSGRKDKWNWIGDKSGKFSVRSVRSLL
YSVSDFSNIYVLKWCKWIPAKCNIFMWQAEMSRIPSADSLIRRKMVGIEGMCSICGEG
PESVEHLFTSCWVAMMVWNHICNWTRVQRFFAFSFRDLIEVHEHVGLKGKAKLVFQGI
VIISCWAIWRARNKRRFEGIDVKIVEVISEIKSLGFLWASSRAKLSRFSWSDWCKYEI
V
SEQ ID NO: 223
PWA73871.1
MVLDNMSLSKPSYDRKAELKAFDETKTGVKGLVDAGITQVPRIFHVPSPQNLKSNQPC
PKPSLPTIDLEGIHEDSLRRKEVIQKVKDALETWGFFQIVNHGISNSMLEEVKKGVRG
FFEQDDEVKKEWYTRDFSGNRKLITNDKFVSAQHRVLANKIGPRVSAVSFFTTSWIPT
LKVFEPIKELLSKDNPPKYRGTTVKEYVEYINKKGLDETSAFLHLKL
SEQ ID NO: 224
PWA70612.1
MVLDNMSLSKPSYDRKAELKAFDETKTGVKGLVDAGITQVPRIFHVPSPQNLKSNQPC
PKPSLPTIDLEGIHEDSLRRKEVIQKVKDALETWGFFQIVNHGISNSMLEEVKKGVRG
FFEQDDEVKKEWYTRDFSGNRKVLYNSNFDLFVAPVTNWRDTFFCMMAPYPPESHELP
QPCRFQL
SEQ ID NO: 225
PWA68274.1
MVLDNTSLSKPNYDRKAELKAFDQTKTCVKGLVDTGITQVPRIFHVPPPQNLNSNQPC
PKPSFPTIDLEGINEDQIKRKKVIKEVNDALESWGFFQIVNHGISNSTLEEVKKGVRG
FFEQDDEVKKEWYTRDMSGKTRVVYNSNFDLYAAPVTNWRDTFFCSMAPNPPEPHKLP
QPCRLVF
SEQ ID NO: 226
PWA39072.1
MASSTQNHQQHHFPNTTTSAPPPTPSTTHPNPATSSAATDLLSRLLNRLPPTLSATPL
RRKSSLTPTTISPPIIPYTDLNSTISTTLHSISELGYFQLTDHPIPPHLAHTAQKESL
SIFKLSRQEKKHHFSKNWPIGFTNNNEDEDHEDEDDDSTESLFLDSDSLSDSSDEFSL
SSLHGFMHAMEKVGLSVVEGLTCAMGLVKDGTDGVCSLLWLGDNEGGDQMLGSGSGKF
YPYVVGLHYMFSSGRCGLLSDSGLVSVKTEVDSILVTVGDIAQVWSNGKLKKVRGKPT
ISMEEGHTSSSLSMSLLVTLPLESTVSPLLPRAVVISNHDDTDDNDNQNQDDQNESIE
ESMFSSFSFEDYAWRVYHERVHLKDPLDRYRIQA
SEQ ID NO: 227
PWA80742.1
MASLTTEHKYDRLKEVKQFDESKIGVKGLLDTGITTIPSFFHQPPENLPSPQPKNQPR
LTVPVIDLSQDRSTVVDEIRGASSTLGFFQIVNHSISVTSIESVIKDMKNFYEQTTEY
KMKFYNREVEKGVTYSTNMDLYKSKAASWRDTIQDISSRTSAIKSLQSTVDRLRAQI
SEQ ID NO: 228
PWA95395.1
MDGSDIYKAANSIRLGSLRAGSSSRASSLRNGSTSVWRNSGMDAFSKSVREEDDEEAL
KWASLEKLPTFDRLKKGLLFGSTGPSNEVDVDNLGANDRRHILDRLVKTADEDNEGFL
LKLRNRLDRVGIELPTIEVKYEHVTVEADVNTGSRALPSFINFHIELVEVLLGLFHLL
PNSKRHITILDDVSGVVKPKRMTLLLGPPSSGKTSLLLAMAGTLAKELKCSGKVTYNG
HELHEFVPERTSAYISQNDVHIGEMTVRETLAFSARCQGVGSRYDMLAELSRRERDAN
IKPDPDLDIFMKAAATEGQEANVVTDYTLKLLGLDICADTMVGDQMIRGISGGQRKRV
TTGEMIVGPSKVLLMDEISTGLDSSTTFQIVKSLKQFIHILEGTAVISLLQPAPETYD
LFDDIILLTDGKIVYQGPREHVLEFFESMGFKCPERKGVADFLQEVTSKKDQQQYWKR
RDMPYRFVTAKEFAEAYQSFHVGRKLATDLATPYDKSKSHPAALTTEKYGLNKKELLK
ACTDREILLMKRNSFVYFFKLFQLTVMAFISMTVFFRTEMHKRTLTDGGFYTAAAFFG
VIMLMENGMSEISMTIAKLPVFYKQRNFLFYPSWAYAIPSWIMKIPVSFVEAAVWTIL
TYYVMGFDPNVYRFFRYFFILMLVNQMSSGLFRFIGALGRNMIVANTFGSFALLLVFA
LGGFILSRDDIKGWWIWGYWASPLMYAMNGIVVNEFRGHSWRTPLNGSTLGIQTITGR
GFFAEAYWYWIAAAALFGFIIVLNICYALSLSLLDAFGKKKADISSLDESDAAVELSS
MNKEDEGSQNKKKGMILPFEPHSITFDDVKYSVDMPQEMKEQGVSEDRLLLLKGVSGA
FRPGVLTALMGVSGAGKTTLMDVLAGRKTGGYIEGDIKVSGYPKKQETFARISGYCEQ
NDIHSPHVTVYESLIYSAWLRLASDVDENQRKAFVDQVIDLVELNSLKDSLVGLPGVN
GLSTEQRKRLTIAVELVANPAIIFMDEPTSGLDARAAAIVMRTVRNTVDTGRTVVCTI
HQPSIDIFEAFDELFLMKRGGQELYAGPIGRNSCELIEYFEAINGVSKITDGYNPATW
MLEVSTSAQEAALGVDFTSIYRNSELYKRNKALIAELSVPRPGTKDLFFPTQYSQSFL
VQCIACLWKQRWSYWRNPPYTAVRFVFTTFIGIIFGTMFWDIGGKRNTQQNLTNSIGS
MYGAVLFLGIQNASAVQPVVDIERTVFYRERAAGMYSALPYAFAQVLVEIPYVFAQAV
VYSLIVYAMIGFEWTAAKFFWYLFFQFCCFLYMTYYGMMTVAITPNASIAAIIAASFY
GIFNLESGYIIPRPSIPVWWRWYYWGNPLAWTIYGMIVSQFGDYTNEFPNGQTVKEYL
DDYLAIEATSALEEYGCFIAEFDKVTPELNDAVFLEDLFDIPIETKVLNMSTKPLYGY
VANVGQIPFIPLYESMGFDYSNTLDGVRSFTEVMWPNGNEAFR
SEQ ID NO: 229
PWA36976.1
MRRLPLTYFIYQKQNIFSLLFGYFVLPHIDFNFFIALDWMKGSSPTLLIGVLTSVLPT
DAFVTGVGHQYRVYDLRLLLACVCSCFDSHLEFCYNLGCTYSFCVQYPQGVDVSKGPK
WEPVPEMCSTPDITLGITSHTDPNVLTVLIQNEIGGQLQAKCGEDWVVVERFLVHREE
MNSGVWRWDWENGYYGSLISYSYINENTLFKMDSANDMEVTSQDDDTSAYNSPYKSTI
SAIFLSCATTATILAQKLFTFLATSAATVSLMILKGLDQPIVLKTSPAWVLFLKVAGG
RVSYQTASIRTTLDNMVQLPNDC
SEQ ID NO: 230
PWA36725.1
MAEAIVSSTVGKVADLLFGKVIKEINHIRNCTENVEKFKTEVEMLKVMRGKVQQQIEI
ANSKGDNLITGVEDWVEKADTEISKAEAFLNEEANAKKTCFKIGLCGNWHTLYHYGKT
ATKMSPYLLQHQEVGKGYETCVSVYTPAPGPLEVYQNKNLDDIATQNSALGDIITAIE
DEDKSKQIIGIYGIGGVGKTTLAMEVSARVKHVFAAVAFTTVSQTVSVEKIKNDIGDA
TKRIMKGEKILIILDDVWEKVELEKLCIPCGINHMNCKILLTSRSEYVCEKMNAHKIC
VNALPEKEAWILFKRVVGEEVERETDLKQVAMKVAEECGGLPLFLNAVGNALKNKSIE
DWDKALTRLQEHAPTRVDPEIGKAFTRLKLSYDFLDNNEARLCFLLCSMFEEDKEIWL
EDLVQYAVGLAKFHGLKSMDDARQRVEDAVKILTSSGLLLKLDDKEYTKMHDVVRDVA
LLIASEGDGKSKFLVEAGKGFTEWLPRKNELESYTGISLMKNNISKLPNYELHLPHLD
IFLISVNDKLPMFSDELIRGMKEVRVLDMSWCEVQPLPQSFKFLTKLRMLDLQGNKSL
HDISILGEMQDLEILILNFTGIKEIPQEIGLLVNLRRLEAIGCENLYHVAPGVISKLW
RLEELLIGFMWMLEGICERIVEIMNLSNLTYLALHVPRFGMIPEGFNPQKLKGFVIQI
GGYFEYYSAANLKRRHLVIQTDYAESSFLKWNKQLIEACDTTDLDSIQNLNNILPQLY
HEGFNELEHINLYKCRYVSCLIDTANWDQLHTFNLPKHIVEGKIKEMFLGKLKHLRLV
RLQNLVVLWKCPDEYISLSNLVTLEIIECDELVKVFPVSVAQGLVNLQNLTVAMCGSL
EEVIWDGDEETSKSETEHSEYTVFRSLAKINLYELERLERFYSGHSTIKYPSLVDADI
EYCPSMKMWGPGIHETPKLKFVYPYVQLDGPDATINYAVNKLLETEKHRWELRFAKRV
LRQHRRKQKMKIQ
SEQ ID NO: 231
PWA71686.1
MGDVAAKELFGRVFNSLVGFAIKEINYMRKCSENVEKNRDELRNLKAMRGKVQQQIDI
AIRKGDNLIIGVEEWVKKVDVEISKAEEFLEQEANAKKTCFKIGLCGNWHTLYHYGKM
ATKMSPYLLRHQEGGKGYETCVSMATKMSPYLLRHQEGGKGYETCVSVYTPAPGPLEV
YQNKNLDDIATQNSALGDIITAIEDEDVSKQIIGIYGLGGVGKTTLAMEVSARVKHLF
AAVAFTTVSQTVSIEKIQNDIGDATKRIMKGEKILIILDDVWEKVDLENLCIPCGINH
PNCKILLTSRSESVCEKMNAHKICVNALPKKEAWILFKRVVGVRVETDADLKRVAPKV
VEECGGLPLFLKTVGNALKHKSIEDWEKALTRLQEHAPTRVDPEIGKAFTRLKLSYDF
LDNEARLCFLLCSMFEEDREIWLEDLVNYAVGLAKFHGLKSIEDARQRVEDAVNILTS
SGLLLNLDDKRRTKMHDVVRDVALLIASEGDGKNNFLVEAGKGLTEWLPRKNELESYT
GISLCKNKISKLPNHELYLPHLDIFLIQRNDELPKFSDELIRGIKEVRVLDMSWCKIQ
PLPSQFKFLTKLRMLDLQGNKSLHDISILVEMQDLEILILDKTGIKEIPQEIGQLVNL
RRLRALNCSNLSHVAPGVISQLWRLEELLIGFMWELEGIYERIVEIMNLSNLTYLALH
VPRFDVIPEGFNLRKLKGFFIQIGGSLEFFYPLANLKSQHLVIKTDYVEIPFLKSLKQ
LIEASHSTDLYCIENLNNILPQLYHEGFNELEHIKLNNCPNVSCLVDTTNWDQFHSSK
HLGEGKIKDKFFGKLKHLHLAYLPSLEVLWNCPDQYISLSNLVTLSIFQCDKLVKVFP
VSVAQGLVNLQNLHISLCNSLEKVIWDGDEETSKSETEHSEYIVFRSLAKIDFFILER
LERFYSGHSTIKYPALVEVNIYSCPSMKMWGPGIHETPKLKFVKNVQLDGPDATINYA
VNKIYEAEKKLWNELMMMEPRIRCE
SEQ ID NO: 232
PWA89508.1
MALSFNDENSLFNFVVKDGNGVKGLVDSGLTQVPGQYIQPPIHRINKQNTTVSLENMI
IDLSELDGPNHDQVVKSIAHAAETPGFFQVVNHGVPIELLESLKVSAHEFFNQPSEKK
AVYLKGVSPSPMVKYGTSFVPEKEKALEWKDYISMIYTNDADALEFWPNECKEVALEY
IKTSTGMSKTRTPSPSLSDENLSDSVASVGREGNLGQSGRVVDDVQDDQLEQILYPGT
SQQPAEPSSATPLDENVNSEGNAIP
SEQ ID NO: 233
PWA75762.1
MSNSLDFRAPPPSPVGPGRRSTVANDDILTDFLHQTLIVPDLVLPDRVFPRQNPNIQS
LPKLDFKKFSEDCKVDEVIEVIAQTGCFELVNHGISSKLLRDVKECGKGVFGLEDEKK
RVVLRSNERLYGFVDVNGDDKDVSEEFVWCRDESLRSEMEGVWTNYTDFSEKMESLMS
KIENISESLLKLFLDPSTPKSRLPDDSNGDKETMGSMCYLYKHSKNMDNLSNDDEYMD
SLSYDVIRMLIRGSEHKHTLCFHVCDGSSEFHVYSKKGWVSFSPDKNALVGTIGDQLQ
TWSEGKYKHVIGRPIFKGELADCISMAFLYSPPISRGQEDNTISLDHRGFIGPMTPPA
TDKGKNKMYQYTDKDLISDELLCEIENNDISLLNEQYGCSYFAPSSLSPIRGQTNEAF
NIYPTIRQDNARVSSVAPTLAEGQGSSSSMPEWPFPSTPSVAPRSSSRAGAGMPSQVR
QGLMLSSLLTQCLKLTGDTVQKQDPIEQGVSGKVLQQHTCIWTSAIKYAVTATLFSGS
MNA
SEQ ID NO: 234
PWA79321.1
MFLKRRAMEQNAAAAADNELVSAVEFWKALLEQEDEPFYPYRYRYRDRPFLSVRSHRD
LQVAHAKFDNILTKKMQQLGCSRPDAIAFIESTLPKPKSYKPIYPADSFNFFPPNRYR
DFDVADEMIRSKHELALESIYDLFDDLTTNVEILYKADIEKEARKLRSLRLRRFYYTF
IPDEDYLENAFRREQIGASVRVNFYPKCPQPHLKLRISPCYPGGITILVDDVESCQSD
LPQGYGDIVASDVGCHGRDHVHPEYN
SEQ ID NO: 235
PWA62974.1
MSNSLDFRAPPPSPVGPGRRSTVANDDILTDFLHQTLIVPDLVLPDRVFPRQNPNIQS
LPKLDFEKLNYSEDFKLEDVIEVIAQTGCFELVNHGISSNLLWDVKECGKGVFRLEDE
KKQVVLRSNERLYGFVEVNGDDKDVSEEFVWCRDESLRSEMEGFWTNYTNFSEKVENL
MSKIENISESLLKLFLDPSTPKSRFQDDSTADKETMGSMCYLYKHSKNTDNLSNDDEY
VYEFHVYSKKGWVSFSPDRHALVVTIADQLQVNSQHLCYLL
SEQ ID NO: 236
PWA46527.1
MEPINEFHISSIFISSSSIDYDIYDDPTLENEEQAKVKNESQEGKRKEKLLNELHAIT
WSKQCQCSVEVVKYLRNLVGTETVELEAKNMKIWRNTIAHGCEEWLYFRLLLSHGAST
EAKANNGMTPLHLSVWHSLRAKDSSIVKTLLEHNADCVDDSSLYKWKVGFDNNIVSPN
LVSYNYSNNTNELCESTGFSNDAFRKKVHIPFQCLKDVIVRHRMLLYFIKEFPLLERV
AIIDSLRLNCVEVPVSVSTCYVDVLDLGRSGCVMRDVTFVVMEMSEDIGSVTGDYESG
V
SEQ ID NO: 237
PWA74914.1
MSLVKRRREQGICGGNKWRMVETSTKQRNRWHADCPKTEINQESSNPTSNLTAWMTQS
KIDYKELLVNHGVDKLLVERMKKEMLDFYNLPVEEKMIYKLKAGEYEGYGQTIIHDQD
QKVDWPDRFYMITNPLHRRKPHLLPKLPPSLRSLLCSTYRIIMVVYVAIRCFNMNLQS
RVNLIDFAKLNMSSGNGDEFVVCYMQSMCLFSTRKCVQGQGVYLIEVAVGEIGMTMKI
TPLQEITPDKGKANIKVKIVSLWNQYYNNNPSKVAGMDMILMDEQGTKIHATINSSVV
CDFDSLLKENNYHIISNFNVKRNVDSTKLSKHEFKIHFYRKTNVRNCSEFICTDDVME
FISFKDFLDARIDQSYSFGLILQMLHLPKSVKIKIHIRVQDETGSATFCLFQQEVAKL
LGKSVGYLISLIDKDEENISYPSDLESIVSKKFVFKLQVSAYNVNNNYHIFTVNKLTD
DKGVMNLIGTKDTEEEENKTAEVGTKRKNIEFQNSEMEKKPKVEHQDTEDDTVINEID
EKDVMKSSLSKQTEEQQCKTTAGETYGDNMEG
SEQ ID NO: 238
A0A2U1L160
MMMMIKRIQGTVKPLARYASVNASFRNSRNDNSHHDKYKPNHKTPANYDLNQTKPFSL
ILPRSTDSFSGIKMPENLERSCQVLGPGMVLLKNYIDISDQVEIVNTCQEFGMGPRGF
YQPGYNGGAKLNLHMMCFGRNWDPQTKYDPYYRSDGSQAPPIPDQLISLVECSIQDSQ
AHDDEIPSIYPDICIVNFYTTTGRLGLHQDRDESQNSLNRGLPVVSISIGDSAQFMYG
NNRDVEKANGVLLESGDVLIFGGKSRHIYHGVKTVVPNSAPLPLLIQSMLRPGRLNLT
FRQY
SEQ ID NO: 239
A0A2U1LWH1
MSGPDSTQPDDISERYSEPSTVTNKGSSIKRESNKYPEQAHSRGWTRYSEKFSPTSVD
SKHITSSTQSKSGRLTEVELVSDDPKERLGLANVVRKTNFTHFEKVNGKNVNVVQGLE
LHTQVFDADEQKKIVEAVYEFQRMGQKGRLRERTYTEPSKWMRGKGRVTIQFGCCYNY
AVDKNGNPPGIIRDEEVDPLPPIFVQMIKRMVRWHILPPTCVPNSCIVNIYDEGDCIP
PHIDHHDFVRPFCTISFLTQCNILFGSNLKIVGPGEFSGPVSIPLPVGSVLVLKGNGA
DVAKHCVPAVPAKRISITFRKMHESKIPYGFSPDPELRGVRPLAMTPLESPSQQKHIE
NKEASSHTPASAPPQVVSKPNNASLFNKEDEFPPLGAMNASRRRR
SEQ ID NO: 240
A0A2U1LWJ5
MSVKEGLVSILKGLSRDEIEELLLNQELCGHCELSLHNHVQLIHKSKHDNKSSMSGPD
STQPDDISERYSEPSTVTNKGSSIKRESNKYPEQAHSRGWTRYSEKFSPTSVDSKHIT
SSTQSKSGRLTEVELVSDDPKERLGLANVVRKTNFTHFEKVNGKNVNVVQGLELHTQV
FDADEQKKIVEAVYEFQRMGQKGRLRERTYTEPSKWMRGKGRVTIQFGCCYNYAVDKN
GNPPGIIRDEEVDPLPPIFVQMIKRMVRWHILPPTCVPNSCIVNIYDEGDCIPPHIDH
HDFVRPFCTISFLTQCNILFGSNLKIVGPGEFSGPVSIPLPVGSVLVLKGNGADVAKH
CVPAVPAKRISITFRKMHESKIPYGFSPDPELRGVRPLAMTPLESPSQQKHIENKEAS
SHTPASAPPQVVSKPNNASLFNKEDEFPPLGAMNASRRRR
SEQ ID NO: 241
A0A2U1M9G2
MVSAKSPELESTDKRLTKRKMGDREKEGKKSKTNWPVIKAKSNLRVTRLKDFDLFTVK
NCLTPAESNAFVKAAETIGFVHQGSLGPTMGEAYRDNDRIAVDDPVLADTLWESGLNK
LFTDLTIRGRVAVGLNPNIRLYRYKVGQRFGRHIDESVDIGEGKRTHYTLLIYLNGGE
SKAKSDVNGSHDSSEPLVGGETVFYGSRNSLVAEVSPAQGMALFHLHGAKCMLHESRN
VAKGVKYILRSDVVFA
SEQ ID NO: 242
A0A2U1N603
MINGAPVEEEDDPFLSGYNDADLRTVSEFLSNWLPFLSKGLCHHCSDSLVHRVHSLNR
FEYASEQSHQEVEVHLSTPNHTDLKDETYIVDTEDTNSLGSWKDGAYGVSGHIEETSG
SERVQSPEYFKTPTPRRSWADMAQEELEADEEEETREHFGNYNGRLQGKGEVRTAQKL
ELSREQRERIRFTNVTRKKDFICLERVNGKLVNILEGLELHYGVFSSPEQKRIVNFVY
ELQEKGKNGKLKERTFTAPQKWMRGKGRVTIQFGCCYNYATDRNGNPPGILRNELVDP
IPHLFKVIIKRLVAWHVLPPTCVPDSCIVNIYDEGDCIPPHIDNHDFLRPFCTVSFLS
ECNILFGSKLEIQGPGEFSGSYKIPLPVGSVLVLNGNGADVAKHCVPAVPTKRISITF
RKMDESKWPAGFHPEPDLQGLEPLVYESDTPKGGSSISKPQSQVSNRQAFRRDGFVAE
TDLRGLEPVAYESDRPKSSSSISKPHSHPSNRQGFRRDGFAPEPDLRGLERVSGSSIS
KPQSQFSNRQASRRDGFVAEPDLRRLEPVAYESDRPKSVSSISKPHIHPSNREPYRRD
ETRGLLGSQPRFYGQSQTRQQGPSPGYRRNGRLEY
SEQ ID NO: 243
A0A2U1N614
MINNGAPVEEEDDPFLSGYNDADLRTVSEFLSNWLPFLSKGLCHHCSDSLVHRVHSLN
RFEYASEQSHQEVEVHLSTPNHTDLKDETYIVDTEDTNSLGSWKDGAYGVSGHIEETS
GSERVQSPEYFKTPTPRRSWADMAQEELEADEEEETREHFGNYNGRLQGKGEVRTAQK
LELSREQRERIRFTNVTRKKDFICLERVNGKLVNILEGLELHYGVFSSPEQKRIVNFV
YELQEKGKNGKLKERTFTAPQKWMRGKGRVTIQFGCCYNYATDRNGNPPGILRNELVD
PIPHLFKVIIKRLVAWHVLPPTCVPDSCIVNIYDEGDCIPPHIDNHDFLRPFCTVSFL
SECNILFGSKLEIQGPGEFSGSYKIPLPVGSVLVLNGNGADVAKHCVPAVPTKRISIT
FRKMDESKWPAGFHPEPDLQGLEPLVYESDTPKGGSSISKPQSQVSNRQAFRRDGFVA
ETDLRGLEPVAYESDRPKSSSSISKPHSHPSNRQGFRRDGFAPEPDLRGLERVSGSSI
SKPQSQFSNRQASRRDGFVAEPDLRRLEPVAYESDRPKSVSSISKPHIHPSNREPYRR
DETRGLLGSQPRFYGQSQTRQQGPSPGYRRNGRLEY
SEQ ID NO: 244
A0A2U1QHH9
MGSEAPFQLPVVDFSALNKQNPDIVIWESAKTKAIEALQEYGCFEATFDEISPDLQKS
VFSELENLFNLPLETKQRNANDREFHGYIGQIPFMPLYESMGIDTPYVQEKVDEFTNV
MWPQGNPKFSKSIQAYSKKLWELDEMVKKMVFEGLNLRKYLEEHLEATSYHLKVMKYR
VADPSESTMGLDSHADTSILTILHQNGVQGLELRTKDETWVTVNVSPNSFVVMAGESF
NVLSNGRLHAPFHRVVMNANKTRLSVGLFSMPKVGSIVKPPKEMVDEEHPLLFKPFDY
GEFMDYFCMAGVKKDTYGLKTYCGLSI
SEQ ID NO: 245
A0A2U1LH70
MEIKKDKDSRWFDVELVPESYIFSPTDRPQNLDVAICDSIPVIDLAKHIQPIESILKA
SQEFGFFQVINHGVPEKIMSDAMSVLKEFFHMPSKEATGYVPHNKGWIYTNNDCTKDG
VHIWRENLKHLCHPLDKCTKLWPNKPTRYQEVIAAYLLEINKLSFRILEMICEGLGLE
PGYLSDTSEVQILSSNFYPSCPDPSLTLGILAHQDTSLITLVYQGDSTGLQFLKDGQW
INVGSIPNSFVVNIGNQLEIVSNGKLRSIDHRVVTSKDKTRISIATFVNPSYDCIIEP
AKALVNKNEPSRYKASRYKEYVDRNKAFGDYTVALRDVVISES
SEQ ID NO: 246
A0A2U1KKP1
MAQEDLEADEEVEARKRFGNYNGRLQGKGEVRTAQKPVLSREQRERIRFTNVTRKKDF
ICVERVNGKLVNILEGLELHCGVFSLEEQGWIVNFVYELQEKGKNGKLKESSAPQKWM
RGKGRVTIQFGCCYNYATDRNGNPPGILHNELVDPIPHPFKSIIKRLVAWHVFPPTCV
PDSCIVNIYDEGDCIPPHIDNHDFLRPFCTVSFLSECNILFGSNLKIDGPGEFSGSYK
IPLPVGSVLVLNGNGADVAKHCVPAVPTKRISITFRKMDESKWPAGFHPEPDLQGLEP
LVYESDTPKSGSSISKPQSQFSNRQAFRRDRFVAETDLRGLEPVAYESDRPKSGSNIS
KPHSHPSNRQVFGRDGFAPEPDLRGLERVSGSSISKPHSQFLNRKASRRDGFVAEPDL
RRLEPVAYESDRPKSVSSISKPHIHPSNREPYRRDDARGLLGLEPRFYGQSQTRQQGP
SPGYRRKASKANAIHMLILVSQSHRNVIHGEWQTRYIGSVSLLVKKFDLHQAVSKKLS
C
SEQ ID NO: 247
A0A2U1MKU7
MGQVDPTFIQELEHRPKPVVIEAQGIPQIDLSPSVPIQELVDQIHDACKNWGFFQVIN
HGVPIESREKVQKAAKRFFDQTTEEKRKVKRDEANPLGYYDTEHTKNVRDWKEVFDFV
VEVPTLMSASYEADDDEITEYRNQWPHHPSELREACEEYVKDVQKLSNKLLELISLSL
NLPAKRLEPFFKDQTTFVRLNHYPPCLAPDLALGVGRHKDAGALTILAQDDVGGLEVK
RKTDGEWIFVKPTPNAFIINVGDIIQVWSNDTYESVEHRVRVNSTRERFSIPFFANPA
HYTMVEPLPELTNAQNPPKYKAYNWGKFFATRKRSNFKKLDVENIQIYHFKKSDERKI
DDVVSRIENVVI
SEQ ID NO: 248
A0A2U1Q5F9
MTRTTEPFSLIIPTNQVCKTLGVTPAENENKVKSSYKVLRPGMILLKNYLSLSDQVEI
VHICEELGIGPGGFGQPGYEDGRKLQLQMLCLGRYWDPQTRYSKGYGFKVPQIPDKFI
SLVETALQDSQTLMNSEDELPWMSPNIGIVNFYTATGRLGIHQDRDESWDSISKGSPV
VSISIGDSAEFLYGDKRDVDKLDKVVLESGDILIFGGKSRNVYHGVKAIIPNSAPEVL
LKESMLRPGRLNLTFRQY
SEQ ID NO: 249
A0A2U1NM47
MYGSENLNEDSERTAFRKTEKKYKLYYDSNKKKKKLPRAVDLSDVIDFKAISESFVRN
EELPVGVSKAQSRFDNPVFCLDACPGFYFIPAALSIEEQHKWIKESLVSFPQPPNRTN
HNAVYGPIHSLFRAANDKQILVEEANDVEGDVCSHRWASSEEIDSSLNGNSCKSISAS
VLLRKLRWSTLGLQFDWSKRGYNVSLPHNKIPDALSQLAKKMAAPAMPAGEDFQPEAA
IVNYFGSGDMLGGHLDDMEVDWSKPIVSMSLGCKAIFLLGGKSRNDEPLAMFLRSGDI
VLMSGEARERFHGVPRIFTDAEHAEIGSLEKLLSDEDDICYLEYIKTSRININIRQVF
SEQ ID NO: 250
A0A2U1LBT3
MYGSENLNDDSERTAFRKTEKKYKLYYDSNKKKKKLPRAVDLSDVIDFKAISESFDRN
EELPVGVSKAQSRFDNPVFCLDACPGFYFIPAALSVEEQHKWIKESLVSFPQPPNRTN
HNVVYGPIHSLFRAANDKQILVEEANDVEGDVCSHRWAPSEESDSSLNGNSCKSISAS
VLLRKLRWSTLGLQFDWSKRGYNVSLPHNKIPDALSQLAKKMAAPAMPVGEDFQPEAA
IVNYFGPGDMLGGHLDDMEVDWSKPIVSMSLGCKAIFLLGGKSRNDEPLAMFLRSGDI
VLMSGEARERFHGVPRIFTDAEHAEIGSLEKLLSDEDDICYLEYIKTSRININIRQVF
SEQ ID NO: 251
A0A2U1PMD5
MTRTEQPFSLILPSDPVCKTLYAEKENKVQSTCEVLRSGMILLRNYLSLSEQVEIVNI
CEELGIGPGGFGQPGYEDGRKLQLQMLCLGRYWDPQTRYSKGYGFKVPQIPDKFISLV
ETALEDSQSFIKSEEDQLPWMSPNIGIVNFYTATGRLGIHQDRDESWDSINKGSPVVS
ISNGDSAEFLYGDERNKLDEVVLKSGDILIFGGKKSRNIYHGVKAIIPNSAPEALLKA
SMLRPGRLNLTFRQY
SEQ ID NO: 252
A0A2U1NFU8
MEASDPLKRYKVGSVPTVYYIPDFISDTDQTQLLNKIYTAPVSKWKSLKNRRLQDWVP
SWLTKITEKISEESSLFPSAINHVLINEYLPNQGIMPHQDGPAYYPVVAILSLGSPVV
MDFTPHAKLVGTASNIEDTGSIEGTDDEMLPNHHPFSIALMPRSLLIFKDLAYSEYLH
GIKDCEIQPYDTAVNATEVSRQQNVIEEHDGDLKAIHRSITRVSLTCRVVSKVHKNLF
RF
SEQ ID NO: 253
A0A2U1LRF6
MYEPNNTIPAIDLIPTEPFSLIVPNGSENSLCGTKMKKNNTLKTSYAVLRPGMVHLKN
YISVTDQVEIINTCQEFGMGPGGFYQPGFNGGSKLNLHMMCFGRNWDPQTKYVARYRC
DGSQAPPIPDKLVALAKASIEDSQRHDNVPSMHPDICIVNFYTNTGRLGLHQDRDESS
NSLRKGLPVVSISVGDSAQFLYGHSRDMRKANEVLLKSGDVLIFGGKSRDIYHGVKAI
IPNSAPLSLLKQSKLRPGRLNLTLRQY
SEQ ID NO: 254
A0A2U1P280
MIPPLCPLRWAPLQTSGQKALPSGENNRYSKKAINKPQETGKSAGFGVHFPLVPKPVN
RGMGKKEILLPLWGVAEAPLVTERERVYVTTFEQPEITLITHKDEFCVLVQEVDDADH
AMYEPNHKTLANDLNQTNSFESTDFLSGIKLKKKDLKTLGPGMVLLKNFISISGQVEI
VKTCQEFGKGPGGFYQPALLNLRLMCFGRNWDPQTKYNARYRGDGSEAPPIPDKLVSL
VQTSIKDSQAYDSSIPSMNPDICIVNFYATSGRLGLHKDIDESPDSLNKGLPVVSISV
EIVKTCQEFGKGPGGFYQPALLNLRLMCFGRNWDPQTKYNARYRGDGSKAPPIPDKLV
SLVQTSIKDSQAYDSSIPSMNPDICIVNFYATSGRLGLHKDIDESPDSLNKGLPVVSI
SVGDSAKFLYGRYRDERKANEVLLKSGDVLIFGGRSRRIYHGVKTIIPNSAPLPLQEI
MLGPGRFNLTFRQF
SEQ ID NO: 255
A0A2U1PMG1
MYKPNNMIPAIDSIPTKPFSLILPEKSTNSLSEIEMKKDYNLKSSCVVLKPGMVLLKN
YISITDQVEIVKICQEFGMGPGGFYQPGLNSGAKLNLHMMCFGRNWDPQTKYAARYRY
DGSQAPPVHDKLVSLAKTSIKDSQAHDDKIPSMHPDICIVNFYTATGRLGMHQDRDES
SDSLRKGLPVVSISVGDSAQFLYGHNRDVRKANEVLLESGDVVVFGGKSRNVYHGVKT
IIPNSAPLPLLQQSKLRPGRLNLTFRQF
SEQ ID NO: 256
A0A2U1LRF7
MMKRIQEMMKPHSLMLVRYASYKPNHKTPVNDLNQTKTFSLTNLRSTTSLCGIDMPED
LESSCAILGPGMVLLKNYININDQAEIVNTCQEFGMGPQGFYQPSYKGGAKLNLHMMC
FGRNWDPQTKYDPYYRSDGFQAPPVPDQLISLVESSIQDSQAHDDEIPSMYPDICIVN
FYTTTGRLGLHQDRDESQSSRNRGLPVVSISIGDSAQFVYGDSRDVEMANEVLLKSGD
VLVFGGKSRHVYHAVKKIIPNSAPLPLLQQSMLRPGRLNLTFRQY
SEQ ID NO: 257
A0A2U1Q1L4
MAKGSLPQKQEKKKEVGSQLVSPQMFQRVNLPAAMHLFEWKYESQINNEKKNDNMEKF
MRVHQALPGSHFNSLLLNRYKTGNHYVGWHADNETLYGPTPEISSVSFGCECHFLLKK
RNTNNEPPTKRPKNNEGTYCTHKCCFTLKHGSLLVMRGNTQRDWLHSVPKRAKANSAR
INLTFRRVIM
SEQ ID NO: 258
A0A2U1KHL4
MSSCCIGKVLNEYSFKIESLNVFVLKALARSLNLEEDCFMNQYGKTSKINARFNYYPP
CPLPEKVLGLKPHGDGSAITFLLQDKEVEGLQLLKDDEWVGVPVVSNSLTINIGDQME
IMSNCIFKSPLHRVFVNSKNERMTLAMFCMRQTEKNIGPVDGLITDETPRLYKNITFT
LDEMHKNYQQGRRAINACKI
SEQ ID NO: 259
A0A2U1KHY6
MMCFGRNWDPKTKYDKQYRSDGSEAPPAPPIPGKLVSLVQRSLQDCQAYENSRDEFPS
INPDICIAKFFTTTSRLVMFQEDDHHSEDSVRRRLPLVSLFIGDSAEFLHSRYYGAAN
PDKVLLESGDVLIYNGYNRYLYYGVKRIIPDSAPLPLLNESMLRPGCLNLNLKQV
SEQ ID NO: 260
A0A2U1N7G6
MEVRMYSPVRTKGFGLIFGHHYLEVEHFKKHKFVYKLINILTENKDMVGIILNIFLLY
KPIVHYGLLFSLTINCYETALILGCTTCKQPIRLHKTLVVGLLTWTNVNLCQKLSFNV
SAFEECISNGEKLGFHKMLLGRRWHYEKHSFTITVPDKFISLVKSSIQDCQSHLNTKD
EKISVCSNNCVVKFYTNTGHLTQHEVGEDDFWYSYRTKGRPVVSMFIGDSAEFFCSDS
RNVNEADKVWLESGDVLIFSNKSRDISLGLHRIIPNSAPLPFLEESMLKPGLLNLSFR
RF
SEQ ID NO: 261
A0A2U1Q4M7
MEASDPLKRYKVGSVPTVYYIPDFISDTDQTQLLNKIYTAPVSKWKSLKNRRLQDWGG
IVHGKGLLPQDLPSWLTKITEKISEESSLYPSAINHVLINEYLPNQGIMPHQDGPAYF
PVVAILSLGSPVVMDFTPHYLHGIKDCEIQPYDTAVNATEVLKQQSAVETHDEDLKAI
HRSNTRVSLTCRVVSKVHKNLFRF
SEQ ID NO: 262
A0A2U1LU01
MERQLIKDSRGKCGAYLSLHRDNKDKTSSRSLESGVVLLKNYVSLSDQVEIVNICQEL
CLGRRGFYEPTHSHGGKLQLHMMCFGRSWDPKTKYGKQYRSDGSEAPPIPEKLVSLVE
FSLQNSQAYVDPGDEIGSMHPDICIANFCTTTGPLGIYMAITTSIGLEPIEQVGPVPN
Y
SEQ ID NO: 263
A0A2U1KKN1
MVALRWSMGHGYRYDGSHRPLLLDHNSNMYKRKLSYLSKSLRKGKASKKFQQYININK
IPALDLHKEKPFSLILPRSTASVLKANNKDKTSSGSILDGVVLLKNYISLSDQGYWGC
LVTRNIECMSRTMLRSRGFYEPTHSHGGKLRLHMMCFGRNWDPKTKYDKQYRSDGSEA
PPAPPIPGKLVSLVERSLQDCQAYENSRDEFPSINPDICIAKFFTTTSRLGMFQEDDH
HSEDSVRRRLPLVSLFIGDSAEFLHSRYYGAANPDKVLLESGDVLIYNGYNRYLYYGV
KRIIPDSAPLPLLNETMLRPGCLNLNLKQV
SEQ ID NO: 264
A0A2U1NE74
MFIFKTMVKSLELEENYFSKHFTEEKDVLQGRFILYPPCPTPDKVFGLKAHSDRSEIT
LLLQDPGVEGLRVLNDGKRCMVPVIAEALFINLGKR
SEQ ID NO: 265
A0A2U1PEW9
MKYRVADPSESTMGLDSHADTSILTILHQNGVKGLELRTKDETWVTVLSNGRLHAPFH
RVVMNANKTRLSVGLFSMPKVGSIVKPPKE
SEQ ID NO: 266
A0A2U1QDY6
MNEVYKATKSIIRRKIQLMKMGDRKKEGKKSKTNWPVVKPKSNLRVNRLKDFDLFTVK
NCLTPAESNAFVKAAETIGFVHQGSLGPTMGEAYRDNDRIAVDDPVLADMLWESGLNK
LFTDLTIRGRVAVGLNPNIRLYRYKVGQRFGRHIDESVDIGEGKRTHYTLLIYLNGGE
SKAKSDVNGSHDSSEPLVGGETVFYGSRNSLVAEVSPAQGMALFHLHGAKCMLHESRN
VAKGVKYILRSDVVFA
SEQ ID NO: 267
A0A2U1MY15
MAVTSDRSPYGASCSEKDKSPLNEKSAVEDMSSGNLDFKETPNLLDDDSISEKLKNIS
HEPQENKRYDICYKSASKAVKLKPSLAVINREKRNQLNRSKQGQNIIMLRPGMVLMKG
YISLNDQVSIVKKCRELGLSDGGFYQPAYREGTKLHLKMMCLGKNWDPQSKLYSDTRP
IDNAKPPGIPDTFSDMVKKAIRDSNAYLEEKNPNAYAGQLIPSMSPDICIVNFYTESG
KLGLHQDKDESPKTLKQGLPVVSFSIGDSAEFLYGDDRDIDTADKVILESGDVLIFGG
KSRLVFHGVTSILPKTAPISLQEATDMIPGRLNLTFRXSDGSPYGASCSEKDKSPLNE
KSAVEDMSSGNLDFKETPNLLDDDSISEKLKNISHEPQENKRYDICYKSASKAVKLKP
SLAVINREKRNQLNRSKQGQNIIMLRPGMVLMKGYISLNDQVSIVKKCRELGLSDGGF
YQPAYREGTKLHLKMMCLGKNWDPQTKLYSDTRPSDNAKPPAIPDTFSDMVKKAIRDS
NAYLEEKNPNAYAGQLIPSLSPDICIVNFYTESGKLGLHQDKDESPKTLKQGLPVVSF
SIGDSAEFLYGDDRDIDTADKVILESGDVLIFGGKSRLVFHGVTSILPKTAPISLQEA
TDMIPGRLNLTFRRVDGRTLKKIIGYCNKHVTDKDNKEALEAFDYEFVEYFDDLTLTH
VSYVAKYLDIKSLQELCDVGLRFRLRRSYNLRYAN
SEQ ID NO: 268
A0A2U1NQD9
MGLPKFTRPKGGHEELSSNLYVANCGPAVGLTLDSIESAFSLYGTVKGVYPADESGCR
VIVSYDDESSAQTALKALDRKTCSDLGGRSLHIRYSVQRPLSQVVPTDSVQVSLEASE
MNIPGVYLIHDFISAQEEEQLLAAVDARPWHNLAKRRVQHYGYEFCYNIRNVDTQQRL
GELPSFVACVLDKVPVFQKLDEAADVSLDQLTVNEYPSGVGLSPHIDTHSAFEGSIYS
LSLSGPCIMEFRRYSNTNACFGDRDAESCDNSSNCIRKAIYLPPRSMLLLSGEGRYAW
QHYIPHHKIDMVKDTIIRRGSRRVSFTFRKVRKGPCQCDYPQFCDSPR
SEQ ID NO: 269
A0A2U1KY10
MSGNNRQSNRKSDNNNNDNNKIKQTYTCFRAEAVPLPKIASDGSPYGASCSEKDKSPL
NEKSAVEDMSSGNLDFKETPNLLDDDSISEKLKNISHEPQENKRYDICYKSASKAVKL
KPSLAVINREKRNQLNRSKQGQNIIMLRPGMVLMKGYISLNDQVSIVKKCRELGLSDG
GFYQPAYREGTKLHLKMMCLGKNWDPQTKLYSDTRPSDNAKPPAIPDTFSDMVKKAIR
DSNAYLEEKNPNAYAGQLIPSLSPDICIVNFYTESGKLGLHQDKDESPKTLKQGLPVV
SFSIGDSAEFLYGDDRDIDTADKVILESGDVLIFGGKSRLVFHGVTSILPKTAPISLQ
EATDMIPGRLNLTFRRY
SEQ ID NO: 270
A0A2U1LLH4
MTEENETIELYNLNYSDLIALSSSAPQASKNTEYLQSVMNTIIKNLGPSGPGLLAVTG
FPDASDLRRTLLPMARKLALLNNDDRKCVLKDHGLGSDVPLKNFDRTVSPFAMQLKYN
RDLDVNSICNVNYLGISGCGSDDKSLSEFKNLGNVFKELGNCMIDVGLRLARVCDKVI
GGHELEESLLESCSAKGRLIHYHSVLDNLILQALNGTPSKSKLAKKTSSNQSDLWQQW
HYDYGVFTILTDPMFMFASESNGVSLSYDEKECPSPSGHTYLKILDPNTNSVVKVKAS
SGSFIVQVGESADILSKGKLRATLHSVCRPKNLENLSRETFVVFLQPAWSKTLSLYDF
SMKSSSLNDEYSRKRDEENQSQNPSSDIGKLVPPLCDRLKDGMTFAEFSRETTKQYYG
SSGLQTKR
SEQ ID NO: 271
A0A2U1KY58
MSSGNLDFKETPNLLDDDSISEKLKNISHEPQENKRYDICYKSASKAVKLKPSLAVIN
REKRNQLNRSKQGQNIIMLRPGMVLMKGYISLNDQVSIVKKCRELGLSDGGFYQPAYR
EGTKLHLKMMCLGKNWDPQTKLYSDTRPSDNAKPPAIPDTFSDMVKKAIRDSNAYLEE
KNPNAYAGQLIPSLSPDICIVNFYTESGKLGLHQDKDESPKTLKQGLPVVSFSIGDSA
EFLYGDDRDIDTADKVILESGDVLIFGGKSRLVFHGVTSILPKTAPISLQEATDMIPG
RLNLTFRRY
SEQ ID NO: 272
A0A2U1PVD6
MCQQGVNKGFLQGARGLVCETLGLEKMDEKVNDLQLHKDGEWVDVPSMRHSIIINLGD
QIEVMMDSEFDDEGNMHSFVAISFGAM
SEQ ID NO: 273
A0A2U1LW75
MTEENETIELYNLNYSDLIALSSPDHKSQHSSKNTEYLQSVMNTIMKNLGPSGPGLLA
VTGVPDASDLRQTLLPMARKLALLNNDDRKRVLKDHGLGSDVPLKNVDRTVSPFAMQL
KYNRDLDVSSICNVNYLGINGCGSDDGSLSEFKNLGNVFKELGNCMIDVGLRLARVCD
KVIGGHELEESLLESCSAKGRLIHYHSVLDNLILQALNATASKSKSAKKTSSNQSDLW
QQWHYDYGVFTILTDPMFMFASESNGVSLSCDGKECPSPSGHTYLQILDPNKNSVVKV
KASSGSFIVQVGESADILSKGKLRATLHSVCRPKNLENLSRETFVVFLQPAWSKTLSL
YDFSMQSLSLNDEYSRNHDEENQCQNPSSDIGKLVPPLCDRLKDGMTFAEFSRETTKQ
YYGSSGLQTKR
SEQ ID NO: 274
A0A2U1Q9W5
MEIPVIDLTPYVDAVSGKFTCDEHLHPELKSVCLEVSRILKETGALLVRDPRCSTEDN
DRFIDMMEKYFEQPEEVKRVQERPHQHYQVGSTPGGVEIPRSLVDLEMQKRLRALPKD
RQPKIPTGPDLKSRYMWRVGPRPSKTRFQELNSEPVIPEGFPEWKKIMDSWGFKMIST
VEAVAEMAAIGFGLPKDSFTNLMKLGPHLLAPTGSDLGSHGEEGTVFAGYHYDINFLT
IHYKSKFPGLFIWLRNGEKIEVKVPDGCLLIQAGKQLEWMTAGDCKAGMHEVVVTDST
LRAIEAAKKENRSLWRVSSTLFSNVASDAVLKPLGHFAKSPLAENYPPMLAGDYFEKE
LSVINLKGKDGA
SEQ ID NO: 275
PWA66490.1|A0A2U1MYZ7
MGFRVLLMFNLLLILFLSNLSIKASSLRKIRESVIRLPNGDSYGHPFDPTRVTQISWH
PRAFLYRNFLTDQECDHFIQLAKDKLEKSMVADNESGKSIESEVRTSSGMFLNKAQDE
VVAGVEARISAWTFLPVENGESMQILHYENGQKYEPHWDYFHDKANQALGGHRIATVL
MYLSNVQKGGETVFPESEIKESQPKANEDWSECAKKGYAVKPKKGDALLFFSLHPNAT
TDVLSLHGSCPVIEGEKWSATKWIHVRNFDKPDDTSDECKDENVNCATWAASGECIKN
PVYMVGSAEGLGYCRKSCKVC
SEQ ID NO: 276
PWA91738.1|A0A2U1Q140
MNNLCLLVLISLLFIVPRSSCSSIINPSKVKQVSWKPRAFVYEGFLTEEECDHLVSLA
KSELKRSAVADNVSGKSQLSEVRTSSGMFIHKNKDPIVAGIEEKIATWTFLPKENGED
IQVLKYESGQKYDPHFDYFSDPVNVARGGHRIATVLMYLSDVEKGGETVFPSAEENSR
HKTPKPDDLSECAKKGIAVLISLLFIVPRSSCSSIINPSKVKQVSWKPRAFVYEGFLT
EEECDHLVSLAKTELKRSAVADNVSGKSQLSEVRTSSGMFINKNKDPIVAGIEEKIAT
WTFLPKENGEDIQVLKYESGQKYDPHFDYFSDPVNVARGGHRIATVLMYLSDVEKGGE
TVFPSAEENSRHKTPKPDDLSECAKKGIAVKPKKGDALLFFSLYPTAVPDTMSLHGGC
PVLEGEKWSATKWIHVDSFDKIIDNGGSCKDENENCERWAALGECTKNKEYMVGTPEL
PGYCRRSCKLC
SEQ ID NO: 277
PWA56204.1|A0A2U1M4Q5
MRLFLMLIVYAYTSSFFRDQYDRVGINLGFYITSLRTSVIRLPNGDSYGHPFDPSRVI
RISWHPRFFTHIVVVYKIYVDMAKDKLEKSMVADVKTGKSIESTVRTSKGMFIRKAQN
RNEIIIVSLKDEVVGRIESRISVWTFLPVENGEAIQVLHYENGQKYLPHWDYFQDKTN
LALGGHRIVTVLMYLSNVEKGGETIFPESEVCSVDLLLYAISYVSNHVDWFLDDLQMK
ESQPKTDEDSECASQGYAVKPKKGDALLFFSLHPNATVDPLSLHGSCPVIEGEKWLAT
KWIHVRSFERKRVSASKNEGCRDEHITCPEWAAHGECVKNGPYMVGSANSPGKCRKSC
KVYHFPQLKFDTFLSDTMVIYTAIEYLS
SEQ ID NO: 278
PWA50233.1|A0A2U1LMM0
MQQKGKPYTRGAPSRKPSSSSSSTLIFSILIICLFFILILLALGILSIPNTSSSKDLT
SILQRLTNPAPTRSDGRLDRWVEIISWEPRAVIYHNFLSQEECEHLINIAKPHMEKST
VVDSVTGKSKDSRVRTSSGTFLDRGQDETVRAIEKRIADFTFLPVEHGEGLQVLHYEV
GQKYEPHYDYFLDDYNTKSGGQRMATVLMYLSDVEEGGETVFPSAKGNFSAVPWWNEL
SECGKEGLSVKPKMGDALLFWSMKPDATTDPSSLHGGCPVIKGNKWSSTKWIRVNEYD
A
SEQ ID NO: 279
PWA61999.1|A0A2U1ML70
MQQKGKPYTRGAPSRKPSSSSSTLIFSILIICLFFILILLALGILSIPNTSSSKDLTS
ILQRLTNPAPTMSDGRLDRWVEIISWEPRAVIYHNFLSQEECEHLINIAKPHMEKSTV
VDSVTGKSKDSRVRTSSGTFLDRGQDETVRAIEKRIADFTFLPVEHGEGLQVLHYEVG
QKYEPHYDYFLDDYNTKSGGQRMATVLMYLSDVEEGGETVFPSAKGNFSAVPWWNELS
ECGKEGLSVKPKMGDALLFWSMKPDATTDPSSLHGGCPVIKGNKWSSTKWIRVNEYDA
SEQ ID NO: 280
PWA81678.1|A0A2U1P7D6
MSGAMVWSSYTTGDALTIICSFFLLALGILSILYKSSNSPKANDPSSTVHRTVDRSDV
EKGGETVFPSAKPNISVVPGWNELSKSGKKGLFVKPTMGDPLLFWSMKPDGSVDPLSL
HASLTSSANSTLIPSILIIGSSVLLTLGILLIPKSLSNLLKAHYRTTVDREDQSVEVL
SSEPRAVIFHNFLSPEECDYLISLAKPHMSRSTVNDRVTGKSIYSSTRTSSSMYLARE
RGKMIQSIEKRIADITSLPVENGEGLSITHYEVGQKIENHHDYFTNDFMIKNGGQRLA
TVLMYLSDVEEGGETVFPSAKGNINRKWGKKGLSVKPKKGDALLFYSMKPDASLDPLS
LHGGRPVIKGDKWCAVKWIRVNKYKV
SEQ ID NO: 281
PWA72969.1|A0A2U1NHH0
MTDKMDGGDGARACGANRGVGLELLEGAIGWGELNLIGLGCSSDGASDGMGKRGDQWT
EILSWEPRAFVYHNFLSKEECEYLINLAKPHMAKSTVVDSTTGKSKDSRVRTSSGTFL
RRGRDKIIRNIEQRIADFTFIPVEHGEGLQVLHYEEGQKYEPHFDYFLDEFNTKNGGQ
RIATVLMYLSDVEEGGETVFPDAHGNYSSDPDWNKLSECAKRGIHVKPKMGDALLFWS
MRPDATLDPSSLHAYIPINLDLDELNYDQWSKLYNTTAKILVFMGTSLVPQKPRVQPM
KNERSETSLSSFESWNDMANLLANIEALLSDKSLVTYIVNRLGNKFAHIASIIRHHFP
TFDTTRSMLLVEESIVNRETSSSSF
SEQ ID NO: 282
PWA46890.1|A0A2U1LD09
MNFRNSFILFRLCLNILSPDFTLLSSGIPDIRTSVIRLPNGDSYGHPFDPSRVIRISW
HPRAFLYKKFLTDEECDHLIKVAKDKLEKSMVADVKTGKSIESTVRTSKGMIIRKAQD
EVVGRIESRISVWTFLPVENGEAIQVLHYENGQKYLPHWDYFQDKPNQALGGHRIVTV
LMYLSNVEKGGETIFPESEEDQGHWLNGVAFEDSLANKHIAYLKDEQKDEIILSAGMA
DNPMSTLFIPSPIAVNPSGVQIVGITGSGSYIETMGGFIAEKVNGPLSMGELKIKNRN
PYDNP
SEQ ID NO: 283
PWA74967.1|A0A2U1NN71
MTETEQSAILDALPQTSSVKHKFLILIALGTKISLKIPTVHIYYFMLFLIDGTLQSIF
PSLTLILAVNFIPIRNHMSVFKSQDECDHLINLAKPHMEKSTVVDNDTGKSKDSRVRT
SSGTFLARGVDETVAAIEKRIADFTFLPVENGEGLQVLHYEVGQKYEPHYDYFLDQFN
TVNGGQRMATVLMYLSDVEEGGETVFPSAKGNISAVPWWNELSECGKEGLSVKPKMGD
ALLFWSMRPDASLDSSSLHGGCPVIRGNKWSSTKWIHVEEYKVFA
SEQ ID NO: 284
PWA90731.1|A0A2U1PY84
MDSNLLKLLIPVTACHALGRVTSKVSFAAVAVSFTHTIKDLEPFFNAAASQFILGQLI
PITLWLSLAPVVLGPRETLSYNTVGEGNKWYRSYGQEIYSPLLLAFTGQSQEECEYLI
NLAKPHMVKSTVVDSDTGKSKDSRVRTSSGTFLARGRDKTIRTIEKRIADFTFLPVEH
GEGLQILHYEVGQKYEPHYDYFQDDFNTKNGGQRMATILMYLSDVEEGGETVFPSAKG
NISAVPWWNELSECGKEGLSVKPKMGDALLFWSMKPDASLDPSSLHGGCPVIKGNKWS
STKWIRVNEYKA
SEQ ID NO: 285
PWA36747.1|A0A2U1KJ01
MLRVLEDGKGGGHLVFLIGGRISRNSIDRKRNCCQEKTGTEKQHGVNIMALEFKSGGF
GCGQGRGRQSLKSQDVNERFQRDAMLDVTTVENLIRVMSDVEKGGETVFPSAKPNISV
VPGWNELSKSGKKGLFVKPTMGDPLLFWSMKPDGSVDPLSLHASLTSSANSTLIPFIL
IIGSSVLLTLGILLIPKSLSNWLKAHYRTNVDREDQSVEVLSSEPRAVIFHNFLSPEE
CDYLISLANPHMSRSMVIDSVTGKSIYSSTRTSSSMYLARGRDKMIQSIEKRIANITS
LPVEHRGGLQIILYKVGQMIENHHDYLTKDFMIKNGGQRLATVLMYLSDVEEGGETVE
PSAKGNVNRKYGKNGLSIKPKKGDALLFYSMKPDASLDPLSLHGK
SEQ ID NO: 286
PWA40801.1|A0A2U1KVM6
MKSRGKTGAGIITGWSSMGLPAVFLLCIFFFFLGFFGSSLFSQQVENESASLHITRPR
PRMLNQNEYDPMPQGDSGDDYFTTIPFQILSWKPRALYFPGFATEQQCNGIIKMAKVK
LAPSTLALRKGETAENTKDIRTSSGMFVSSYEDKTGILDQIEKKIERATMIPRRHGEA
FNVLRYDLGQKYNSHYDAFNPTEYGPQKSQRIASFLLYLSDVEEGGETMFPFENGENA
DSNYDERKCIGLKIKPRKGDGLLFYSLYPNGTIDPTSLHGSCPVIKGQKWVATKWLRN
EEDFD
SEQ ID NO: 287
PWA62006.1|A0A2U1MLA9
MASAMKIVIFLLTLVTIGMIFGSLLQLAFIRNLEDSYGSTFPSFRRSLGSQNGNQLPP
VISGWGNDKEAAILRIGYVKPEIISWSPRIIVFHNFLSAEECDYLRAVAKPRLKVSTV
VDAKTGKGIKSNVRTSSGMFLNHEERKYPMVHAIEKRIATYSQVPIENGELIQVLRYE
PNQFYRPHHDYFSDTFNLKRGGQRVATMLMYLTDNVEGGETFFPLAGSEECSCGGKMV
KGLCVKPNKGDAVLFWSMGLDGQSDPSSIHGGCEVLSGEKWSATKWMRQKATS
SEQ ID NO: 288
PWA51043.1|A0A2U1LPW2
MHFSWQSQEECEYLINLAKPHMVKSTVVDSDTGKSKDSRVRTSSGTFLARGRDKTIRT
IEKRIADFTFLPVEHGEGLQILHYEVGQKYEPHYDYFQDDFNTKNGGQRMATILMYLS
DVEEGGETVFPSAKGNISAVPWWNELSECGKEGLSVKPKMGDALLFWSMKPDASLDPS
SLHGQYIYPSMSDVEEGGETVFPSAKGNISAVPWWNELSECGKEGLSVKPKMGDALLF
WSMKPDASLDPSSLHGGCPVIKGNKWSSTKWIRVNEYKA
SEQ ID NO: 289
PWA66066.1|A0A2U1MXX2
MLTSKTSLQLPIFDVSKPLSPSCLSSLSLACKEWGFFHIINHGISKELYNKLHFLSNQ
LFDLPYEQKLKVGPSSDIKTYTPHFIASPFFESIRVSGPDFCASAQSSAEVLLNQSKT
EFSEVLKEYGSIMRTLSNKIIEMLLMCLGEDFEKKFESEFRNSEGYLRINNYSPPESI
EKDVEGLGMHTDMSCITIVYQDKSGGLQVRSKEGKWMDIDPCAETLVVNIGDLMQAWS
NGKLRSSEHRVVLKERKNRFSLAFFWCFEDNKVVFAPDEVVGENSMRAYKPFLCDDYM
KFRQNSERGKFEKVGFTVKDFAGTGLV
SEQ ID NO: 290
PWA61654.1|A0A2U1MKA4
MLTSKTSLQLPIFDISKPLSPSCLSSLSLACKEWGFFHIINHGISKELYNKLHFLSNQ
LFDLPYEQKLKVGPSSDIKTYTPHFIASPFFESIRVSGPDFYASAQSSAEVLLNQSKT
EFSEVLKEYGSIMRTLSNKIIEMLLMCLGEDFEKKFESEFKNSEGYLRINNYSPPESI
EKDVEGLGMHTDMSCITIVYQDKSGGLQVRSKEGKWMDIDPCAETLVVNIGDLMQAWS
NGKLRSSEHRVVLKERKNRFSLAFFWCFEDNKVVFAPDEVVGENSMRAYKPFLCDDYM
KFRQNSERGKFEKVGFTVKDFAGTGLV
SEQ ID NO: 291
PWA84981.1|A0A2U1PGU3
MPASNTSIQLPIVDISKSISPSSISSLSLACREWGFFHIINHGISKDLYSKLRLSSNQ
VFNLPYEAKIKAGPTSNVKTYTPHFIASPFFESLRVSGPDFLASAKNSAEAIMNQPDT
EFSEVLKEYGDKMRNLSKKVTDILLMCLGKDFATKFASEFSNCEGYLRINNYSLPEKR
VMEKEVEGLGMHTDMSCLTIVYQDDVGGLQVRSKDGKWLDINPCEETLVVNIGDLLHA
WSNGKLRSSEHRVVLKECKTRFSLAFFWCFEDNKVISAPNEVVGDEFRHYEPFMCGDY
LKFRENSVRGKFEKVGFTVKDFAGI
SEQ ID NO: 292
PWA51762.1|A0A2U1LRY2
MPASNTSIQLPILDISKSISPSSISSLSLACREWGFFHIINHGISKDLYIKLRLSSNQ
VFNLPYEDKIKAGPTSNVKTYTPHFIASPFFESLRVSGPDFHASAKNSAEVIMKQPDI
EFSEVLKEYGDKMRNLSKKVTEILLMCLGKDFVTKFASEFSNCEGYLRINNYSLPEKR
VMEKEVEGLGKHTDMSCLTIVYQDDVGGLQVRSKDGKWLDINPCEETLVVNIGDLLHA
WSNGKLRSSEHRVVLKECKTRFSLAFFWCFEDNKVISAPNEVVGDEFRHYEPFMCGDY
LKFRENSVRGKFEKVGFTVKDFAGI
SEQ ID NO: 293
PWA38898.1|A0A2U1KQ86
MVSNSCFETTLNLPSIDLSKPLNPSSISSLSEACHQWGFFNIVNHGISKSLYEDIRSF
SNHVFDLSSETKLKLGPSSSVKTYTPHFIASPYFESLRVSGPDFHSSAQDSIDVIFHE
DRHEFSEILEEYGNKMTELSKKIMKIVLMILGDGFDTRLYDSDENNCHGYLRINRYSP
PTDLEEKETTEGLGMHTDMSCITIVYQDETGGLQVKSKDGTWMDIVPSEGTLVVNIGD
LLQAWSNDKFISSEHRVVLKKPVNRLSIAFFWCFEDEKVILAPNEVVGNENTRLYEPF
VCLDYLKFRESNEEGKFEKVGFTVKDFVAHNTKNCLPMPVS
SEQ ID NO: 294
PWA65809.1|A0A2U1MX46
MVSNSCFETTLNLPSIDLSKPLNPSSISSLSEACHQWGFFNIVNHGISKSLYEDIRSF
SNHVFDLSSETKLKLGPSSSIKTYTPHFIASPYFESLRVSGPNFHSSAQDSIDVIFHE
DRHEFSEILEEYGNKMTELSKKIMKIVLMILGDGFDTRLYDSDFNNCHGYLRINRYSP
PTDLEEKETTEGLGMHTDMSCITIVYQDETGGLQVKSKDGQWMDIVPSEGTLVVNIGD
LLQAWSNDKFISSEHRVVLRKPVNRLSIAFFWCFEDEKVILAPNEVVGNENTRLYEPF
VCLDYLKFRESNEEGKFEKVGFTVKDFVAHNTKNCLPMPVS
SEQ ID NO: 295
PWA53651.1|A0A2U1LXD5
MPAREYVSSVGDMGSETPTKLPCIHFSNLDKHTNKLEWDSTKSQVHQALQEFGCFEAT
FHNIPPELQKSMFESLQLLFDLPLQTKLKNRSSKPFHGYVGQYPMVPLYESMGIEDAP
VPEKTESFTKMLWPEGNPKFCETIQEFSEKLSKFDQMVRMMVLESLGLEKYMDEHMDS
TNYLLRVMKYKGPETNESKLGLNSHTDKNIVTILHQNQVNGLEVQTKSGDWIKIQPSP
DSFIVMIGDSLYAWTNGRLHSPYHRVMMSGEKARYSFGLFSIPKAGYVIRAPIEVVDE
QHPLLFKPFDHVEFLQFYYTEDGQRAQSALKTYCGV
SEQ ID NO: 296
PWA87958.1|A0A2U1PQC3
MIKGHSSLLSLHKEHMDCQKTPLVFDSLILQHETNIPQQFIWPDHEKPNLQKAKELVV
PLVDLGGFLSGRPSSAKEASVVVGDACKKHGFFLVTNHGVDASLIADAHRYMDLFFEL
PLSDKQRAQRKLGESCGYASSFTGRFSSKLPWKETLSFQFSGEKSSANGVKDYFENTL
GKEFTRLGKVYQEYCNAMSRLSLGIMELLGMSLGVHRAHFKEFFEEHDSIMRLNYYPR
CQKPDLTLGTGPHCDPTSLTILHQDSVGGLEVFVDNEWRSIAPNTNAFVINIGDTFMA
LSNGRYKSCLHRAVVNNRIHRKSLAFFLCPKKDKVVSPPDELVDENNPRIYPDFTWST
FLEFTQKHYRADMNTLQAFSNWIQQRNS
SEQ ID NO: 297
PWA83096.1|A0A2U1PBG3
MEESDPPFFEFYKNLLEKEDTSKKNVIGVFDNVEEWDLPVIDLSRLCGEGWESAKCKK
EIAEASKKWGFFQVINHGVSNDILEKMRYEQMKVFKNTFHVKAKNKCESNFPEGSYRW
GTPSATRLRQVAWSEAFHVPLTDISNMGGLTSLSNTMEQFATMVSDLAENIAEILAEQ
LGHKSDFFRKNCLPSTCYLRMNRYPPCPISSQVYGLMPHTDSDFLTILYQDQIGGLQL
VKDGKWIAVKPNKDALIINIGDLFQAWSNDVYKSVKHRVLANKQSQRFSTAYFFCPSY
DTMIQSGAENSIYKRFSFREFRQQVQDDVKRLGYKIGLPRFIL
SEQ ID NO: 298
PWA73824.1|A0A2U1NJY1
MDPFDPPFRETYKTLFDNHFNNELSRKGNKIEDLQEANECEFELPLVDLSRLNQGGIE
SEKCKQEIAKASQEWGFFQVINHGVSHEVLENMRSEQVKTFKKPFHDKVNGQCDQLDF
LAGSYRWGTPSATCLRQLAWSEAFHVPLSEISAVDGVTGLSTTMKEYTNIVSILAEKL
AEILAEKIGQKPKFFEENCVPSMCYIRMSRYPSCPISPHVFGLMPHTDSDFLTILHQD
HIGGLQLLKNGKWIAVNPKQETLIVIIGDLFQAWSNGTYKSVEHRVVANKLFERFSTA
YFLCPSYDTIIESCDETPVYRRFSFKEFRQQVQEDVKNYGHKIGLSRFLL
SEQ ID NO: 299
PWA67016.1|A0A2U1N0M0
MPPTILTSPMIVSPPKETNPFVFDASILKNETNIPSQFVWPDHEKPQLEPSPPLNVPT
IDLQAFFSGEPHAISNAVRLVNDACKDHGFFQVSNHGVDSNLIDKAIKIMDVFFEMPF
LEKQKAQRKCGENYGYASSFTNRFTAKLPWKETLSFRYVPDPKCSNIVQDYFLNVLGE
DFNEFGKVCQEYSEAMNKLSLDIMELLAMSLGIKQFFLKDFYEDNDSIVRLNYYPTCQ
QPDLTLGTGPHCDPTSLTILHQDDVGGLEVFFKEKWHPVAPCPGTFVVNIGDTLMALS
NGLYKSCLHRAVVNNETPRKSIAYFLCPRMDKVVKPPKALVENNNNQRMYPDFTWSAF
LEFTQKHYRSDMNTIDAFSRWLQESKEIKTT
SEQ ID NO: 300
PWA53134.1|A0A2U1LVY5
MPPFLLTSPSVSPTIDTKSRQNPLIFDASFLQNETSIPQQFVWPEDEKPYHGPPPPQL
YVPPIDLKGFLSYEPIAMSNASRLVDLACRKHGFFQVINHGIDSRLINEAHKIMEYFF
RMPLLEKQRAQRRAGEYCGYASSFTNRFSSKLPWKETLSFRYSADPQCLNLVPDYFLN
VMGEDFSHFGRVCQEYCEAMNKLSLVIMELLGLSLGISYSCFTDFYKDNDSIMRFNYY
PPCQKPDQALGTGPHCDPTSLTILHQDNVGGLEVFVEDKWHSVAPCSNAFVINIGDTF
MALSNGLYKSCLHRAVVNSHTPRKSLAFFLSPRMDKVICPPKELVADDDQRIYPDFTW
STFLEFTQKHYRADMNTLEAFSNWLRNETKC
SEQ ID NO: 301
PWA93023.1|A0A2U1Q4X2
MDPFDPPFRETYKTLFDNHFKNELSTKGNKIEDLQEANECEFELPLVDLSRLSQGGIE
SEKCKQEIAKASQEWGFFQVINHGVSHEVLENMRSEQVKTFKKPFHDKVNGQCDQLDF
LAGSYRWGTPSATCLRQLAWSEAFHVPLSEISAIDGVTGLSTTMKEYTNIVSILAEKL
AEILAEKIGQKPKFFEENCVPSMCYIRMSRYPSCPVSPHVFGLMPHTDSDFLTILHQD
HIGGLQLLKNGKWIAVNPKQETLIVIIGDLFQAWSNGTYKSVEHRVVANKLFERFSTA
YFLCPSYDTIIESCDETPVYRRFSFKEFRQQVQEDVKNYGHKIGLSRFLL
SEQ ID NO: 302
PWA99916.1|A0A2U1QPH7
MPPTILTSPMIVSPPKETNPFVFDASILKNETNIPSQFVWPDHEKPQLEPSPPLNVPT
IDLQAFFSGEPHAISNAVRLVNDACKDHGFFQVSNHGVDSNLIDKAIKIMDVFFEMPF
LEKQKAQRKCGENYGYASSFTNRFTAKLPWKETLSFRYVPDPKCSNIVQDYFLNVLGE
DFNEFGKVCQEYSEAMNKLSLDIMELLAMSLGIKQFFLKDFYEDNDSIVRLNYYPTCQ
QPDLTLGTGPHCDPTSLTILHQDDVGGLEVFFNEKWHPVAPCPGTFVVNIGDTLMALS
NGLYKSCLHRAVVNNETPRKSIAYFLCPRMDKVVKPPKALVENNNNQRMYPDFTWSAF
LEFTQKHYRSDMNTIDAFSRWLQESKEIKTT
SEQ ID NO: 303
PWA34832.1|A0A2U1KDH9
MKELPDSYTWASVDDGPLLSCSSEPVPVINLEDPNVMQHVGHACKTWGVFHVTNHGIP
TAVLDDMEASVRKLFYLPIQQKLKAARAPDGVSGYGAVRISSFFHKHMWSEGFTIIGS
PYEHAKKLWPQDYKNFCEVVDEYNKEMKKLTNRLMWLVLGSLGITEGDVKWARQKQDI
KEACPALQLNSYPACPDPDRAMGLAAHTDSSLLTILHQNNTSGLQVQRERMGWVTVPP
IKGALVVHVGDLLHILSNGLYSSVLHRAIVNRTQHRLSIAYLYGPPSNVRISPLSKLT
SIVRPPLYQPVTWSEYLAMKAKYFNKTLSVLRLSAPVNGFVDLKDHKSVIVG
SEQ ID NO: 304
PWA41221.1|A0A2U1KWT2
MSLKIQKAEKHMDLSSMKQLPDSYTWASVDDGYLLSCSSEPVPVINLEDPNVMKHVGH
ACKTWGVFHVTNHGIPKAVLDDMEASVRKLFYLPIQQKLKAARAPDGVSGYGAVRISS
FFHKHMWSEGFTIIGSPYEHAKKLWPQDYKNFCEVVDKYNKEMKKLTNRLMWLVLGSL
GITEGDVKWARQTQDIKEACPALQLNSYPACPDPDRAMGLAAHTDSSILTILHQNNTS
GLQVQREGMGWVTVPPIKGALVVHVGDLLHILSNGLYSSVLHRAIVNRTQHRLSIAYL
YGPPSNVRISPLSKLTSIVHPPLYQPVTWSEYLAMKAKYFNKTLSVLRLSAPVNGFID
LKDHKSVIVG
SEQ ID NO: 305
PWA75824.1|A0A2U1NQP0
MGELDPNFIQEPDHRPKLAIIEAEGIPQIDLSPLINSSPADYNAAAVQDLIGEVRDAC
KNWGFFQVFNHGVPFESRENLFSASKRFYDQPVEEKRKVRRDEANPQGYYDTEHTKNV
RDWKEVFDITVENPTKMFASHEADDERITQYINRWPEHPPELREACEAYVKDVQQLSY
KLLELISLSLGLPANQFEPFFSKDQTSYIRLNHYPPCPVPDLALGVGRHKDAFALTIL
AQDDVGGLEVKRKTDGEWIFVKPTPNTFIINVGDMIQVWSNDRYESVEHRVMVNSTKE
RFSIPFFLSPSYYTVVEPLAELIDEQNPPKYKGYSWGKFFATRKRSNFQKLKVENIQI
YHFKIPNEQKSDDVVSCVENLAI
SEQ ID NO: 306
PWA49201.1|A0A2U1LJQ3
MEDNTNEIINRSEYGGSIPVDNVQALASSKDLKDIPSRYIRPEIELDVVLTDESLQIP
VIDISKLAVAEQPGYDDELENLHVACRDWGFFQLINHGISESIDAMKKVTHEFFNLPL
EEKMKCAQMPNNIEGYGQAFVVSQEQKLDWGDMLFILPLPIPQRNMMFWPQNPDSFKT
TLDKYSDTLYGVSMKLLKLISINLGIKPEAITKMFENCNQGIRMNHYPPCPEANKVLG
LTPHSDATGLTLLVQVNDVQGLQIKKNFKWVPIKPLPGSIIVNIGDVMEIMSNGEYKS
IEHRAVVNFEDERLSIAAFHSPDIEAMIGPLTELVKDNTPKYKTIGSEDYLKLIINSK
LDGKSLIDQMKI
SEQ ID NO: 307
PWA81949.1|A0A2U1P866
MIESNQNPPLLRDYTQLLHESRDKVDVKRLVNHEPGMEECELPLVDLSGLWGENEEDR
VSCASEICKASSEWGFFQIVNHGINLELLRKMRKEQVELFKASFERKTADGLLDNSYR
WGNRTATCPKQLSWCEAFHVPLTKISDDTCYGEFDSLREVMQEYASAMQELAKSISKV
LVTNMGVQQGVWEDNCDASTCFIRLNRYPPCPISPEVFGLVPHTDSDFLTILHQDEHV
GGLQLMKDSKWVAVKPNTNALVVNIGDLFQAWSNDVYKSVEHKVIVNQEAERHSIAYF
LCPSYESFIGCCEEERSTYRRFTFGEYRSQIQQDVKTFGHKVGLPRFLAST
SEQ ID NO: 308
PWA72778.1|A0A2U1NH25
MAIDCMIKTTSNMPSLKEDQRKSFVFDASVLKHESNIPQQFIWPDHEKPNSQKSKELE
VPLVDLGGFLSGRSSSAKEASSLVGEACQKHGFFLVVNHGVDENLISDAQRYMDLFFE
LPLSVKQRAQRKVGESCGYASSFTGRFSSKLPWKETLSFRFSAEENASNIVKDYFENT
MGEEFIRLGKVYQEYCNAMSRLSLGIMELLGMSLGVSRAYYKEFFEEHNSIMRLNYYP
PCQKPDLTLGTGPHCDPTSLTILHQDNVGGLEVEVDNEWRLIAPSSNAFVVNIGDTFM
ALSNGRYKSCLHRAVVNSKTTRKSLAFFLCPKKDKVVIPPKELVDENNPRIYPDFTWS
TFLEFTQKHYRADKNTLQAFSTWGVNNAQPELQASSSKNASEGKDVITTSGETQIKPS
VPTKVNESEHEDKFLDLLQIMLVLDKRTLLDQLNAFETEEQIVQTEQAIVPVNKASGG
NNQNGFKSRNKIFNDALKFLQHDDGKTQVIALCGMGGVGKTTLMNQLKEAAKNEKMFQ
WIMVLIIGKSSNLFAHQNAIAVHTGEPLTKTDEKLRATYFSKRFREFSEHKEKSLVIL
DDVWEKIKLKDIGLAGALPNGEISNKLIDIELKMDSTLATEDERVERIQLLRDLNDID
RLESLDLLQKSRLKWAAEGDENSKFFHGLLKQKRSRQSIQGIMLDGEWISNPISHRRE
Y
SEQ ID NO: 309
PWA59012.1|A0A2U1MCV6
MSLHVQPADHLQQQNNMYQKGVKHLCDNGLTQVPNKYILPEPERPQSLVVEGCIDLPV
IDFAQLQGADKPQVLASLSKACQEFGFFQLVNHGIGNEAIGKMIDVSKRFFELPFEER
QRYMSSDLYAPVRYGTSFNQNNDGIFCWRDFLKLSCHPMQDFASLWPSSPVDLRETLG
EYSTKTQHLYKMVMEAILESIGVTNEKAISEEGNQLMMVNWYPACPQPQLTLGLPPHS
DYGLLTLLLQDQVEGLQIQHNGKWVTVKPIPNSFVVNIGDQFEIFSNGRFKSVVHRVA
VNSTRSRLSVASLHSLQANRNVTPSPKLIDESNPRRYRDTSYADFLDYLSSSDFKCKR
FLESRELH
SEQ ID NO: 310
PWA75999.1|A0A2U1NR61
MAAVSKRLLVSDLVTVDKINQVPSNYIRPLSERPNLRHVSSHETIPIIDLHDLNSPKR
CHVINQIRDACREYGFFQVKNHSVPESTITNMMQIAKDFFNLPEEERLKSYSDDPNKT
TRLSTSFNIRTEKVANWRDFLRLHCYPLEDYVHEWPTNPSSFRAHVKEYCTSVRGLAL
ELVEAISESLGLERDYIGKQLGKHGQHMALNYYPPCPQPDLTYGLPGHTDLNLITILL
QDDVPGLQVLKDGKWVAVDPVPNTFIINIGDQIQVLSNDKYKSILHRAVVNCDKERIS
IPTFYCPSRDAVISPAPEFVTEDQPAVYRPFTYGEYYDKFWNRGLATENCLDMFMATK
ST
SEQ ID NO: 311
PWA86604.1|A0A2U1PLG3
MEDNTNEIINRSEYGGSIPVDNVQALASSKDLKDIPSRYIRPEIELDVVLTDESLQIP
VIDISKLAVAGQPGYDDELENLHVACRDWGFFQLINHGISESIDAMKKVTHEFFNLPL
EEKMKCAQMPNNIEGYGQAFVVSQEQKLDWGDMLFILPLPIPQRNMMFWPENPDSFKT
TLDKYSDTLYGVSMKLLKLISINLGIKPEAITKMFENCNQGIRMNYYPPCPEANKVLG
LTPHSDATGLTLLVQVNDVQGLQIKKNSKWVPIKPLPGSVIVNIGDVMEIMSNGEYKS
IEHRAVVNFEDERLSIAAFHSPGIEAMIGPLTELIKDNTPKYKTIGSEDYLKLIINSK
LDGKSLIDQMRIE
SEQ ID NO: 312
PWA69194.1|A0A2U1N6V3
MGSQALSRLPTIDFSNVNKQNRDSVWDSTKTQVFEALQEFGCFEASFNGISHDLRNSV
FSSLKELFDLPLETKVRNFTEKLYNGYIGLAKQVPIFESMGIEDPQSFANLMWPNGND
EFSKCIKIYSDKLTELDEIVRTMVLESLNLEKYMDEHMELTSYITRVMKYRVPQKDEP
NAGLLSHADKNIVTILHEFGVEGLEVQTKDGEWFKVKLSANSFVVMVGEAFKVLTNGR
LRPAVHRVVMSGDEDRFSIGVFSVPKDEKTIKAPEEMVDEDHPLLFKPFVYEEFFKFF
RLEENVNDPLALEKYCGVSTENL
SEQ ID NO: 313
PWA97232.1|A0A2U1QGX7
MAAVSKRLLVSDLVTFDKINQVPSNYIRPLSERPNLQHVSSHETIPIIDLHDLNSAKR
RHVINQIRDACREYGFFQVKNHSVPESTITNMMQIAKDFFSLPEEERLKSYSDDPSKT
TRLSTSFNIRTEKVANWRDFLRLHCYPLEDYVHEWPTNPSSFRAHVKEYCTSVRGLAL
ELVEAISESLGLERDYIGKQLGKHGQHMALNYYPPCPQPDLTYGLPGHTDLNLITILL
QDDVPGLQVLKDGKWVAVDPVPNTFIINIGDQIQVLSNDKYKSILHRAVVNCDKERIS
IPTFYCPSRDAVISPAPELVTEDQPAVYRPFTYGEYYDKFWNRGLATENCLDMFMATK
ST
SEQ ID NO: 314
PWA81688.1|A0A2U1P7E5
MPTIDSKSLNILDFVVNQGHGVKGLAELGLETLPYQYIQPPQERFNTSHEEDNKDSIP
VIDMSNSNDPHVAKAICDAAQKWGFFQIVNHGVPTHVLEDVKDATHKFFALPPQEKLK
YSKEQSVTHNVRFGTSFTPEAEKALEWKDYLSLFFVSDTEAASLWPPTCRDQAMEYIK
SSELLVKKLLNILMNGLNVKEIDETKESILMGSKRINLNYYPKCPSPELTVGVGRHSD
VSTLTILLQDDIGGLFVRNMETMKWIHVPPVSGSLVINVGDALQIMSNGKYKSVEHRV
TANGSNNRISVPIFVNPKPSDVIGPLEEVIASGEEPLYKNVLYSDYVKHFFRKAHDGK
ATVEFAKI
SEQ ID NO: 315
PWA40905.1|A0A2U1KVY7
MGSLSQPKLPVINLSAKNLEYNSISWVKTCGEVISALEKYGCFVAKYDGVSQELHDAI
FHASHDLFDLPTEVKVLNTSDTPSHGYVGQIPVIPLYEGLGVENATTADGVERFTKLM
WPSGNGVFCETVLMFSKAVAELDQIVMKMIAKSYGIEEHYGPLLGSTTYLLKLIKYLS
PQGDERNLGIVPHTDKTFMSILHQDEVKGLEIKTKDGEWIKYDPSPSSFVVMAGDGCM
AWTNGKIEASCHRVMMQGSKERFSLGLFTFIRNIKIETPQELVDENNPPKFKAFDHYK
YLHYHATDEGMRSKFPLKSYCGI
SEQ ID NO: 316
PWA96852.1|A0A2U1QFS5
MANFPIINMETLDGDERCATMAKIQDACENWGFFEVLNHGISHELLDKVETMTKEHYK
KCMEQRFKDMVAEKALEGVKAEVTDIDWESTFFLRHLPTSNISEVPDLEDEYRELMKD
FAAKLEKLAEELLDILCENLGLEKGYLKKAFYGSKGPNFGTKVSNYPPCPTPDLIKGL
RAHTDAGGIILLFQDDKVSGLQLLKDGEWVDVPPMRHSIVINLGDQIEVITNGKYKSV
MHRVIAQTDGTRMSLASFYNPGNDAVIYPAPALLEKEPKENEQSYPKFVFDDYMKLYA
GLKFQAKEPRFEAMKAVEANVAA
SEQ ID NO: 317
PWA73281.1|A0A2U1NIH9
MGTLPQAKHLPVIDLRLEHLNSTSSTWVTTCGEVMRALEEYGCFIAMYDGVSQGLHDA
IFHASQQLFDLPTEVKALDTAYPPSHGYIGARPGAPFYEGLGIQNATTKQGIERFEKL
MWPSGNPSFSEGVLEYSKAVAELEHLVMRIIAKSYGIEENYESLLGSKTYLVRLMKYI
IPQVKENESIIGIYAHTDKTFTSLLDQYQVKGLEIKAKDGEWIEVNPLPSSFVFMAGD
VCTAWTNGRIESPVHRVMMHGDKERYSVGIYAFIRDRKIEVPQQLVDEDHPLQYKPFY
NCKLIDYFNTDEGKKSKYPLKSFCGI
SEQ ID NO: 318
PWA42077.1|A0A2U1KZ79
MAAKVISSGFRYTSLPESYVRPVTDRPNLSQVSECDDVPVIDIGCGDRELISKQIGDA
CRHYGFFQVINHGVPDEIVKKMQQVGRDFFELPLEEKMKLYSEDPTKTMRLSTSFNVQ
KEKVHNWRDYLRLHCHPLDKYSPEWPSNPAYFKEYVGNYVTAVRDLGMRVLESISESL
GLEKDRIIKILGDQGQHMAINHYPVCPEPELTFGLPGHTDPNALTILLQDTLVSGLQV
LKDGKWVAVKPHPNAFVINIGDQLEAVSNGQYKSVWHRAVVNSDKPRMSIASFLCPCN
DSILSAPKELIKDGSKPVFKDFTYAEYYKKFWSRNLDQEQHCLEFFKN
SEQ ID NO: 319
PWA39233.1|A0A2U1KRA6
MGSESHIRLPVIDFSPLTHISNDASIWESIRADVFKTIQHYGCFEVSSFVSIDLQESV
NAGLKQLFDLPLETKAKNTSEIAFHGYVRSPKVPLYESMGIGNPFITENVDSFTNLMW
PQDNPKFRAMLFVDNFLRRYLKFVTSTSIVPHNTTSMKMGSESHIRLPVIDFSPLTHI
SNDASIWESIRADVFKAIQDNGCFEVSSFVSIDLQESVNAGLKQLFDLPLETKAKNTS
EIAFHGYVRSPKVPLYESMGIGNPFITENVDNFTNLMWPQDNPKFRESIMTYSRKLRE
LDEIVKRMVFESLDLEKYLGEQMKSTSYLLKLMKYRAPEPNESDIGLHTHTDTNIMTI
LHQDEIGGLEIQTKNDEWIRVKVSPNSFVVVAGETFNFRAMLFVDNFLRRYLKFVTST
SIVPHNTTSMKMGSESHIRLPVIDFSPLTHISNDASIWESIRADVFKAIQDNGCFEVS
SFVSIDLQESVNAGLKQLFDLPLETKAKNTSEIAFHGYVRSPKVPLYESMGIGNPFIT
ENVDNFTNLMWPQDNPKFRESIMTYSRKLRELDEIVKRMVFESLDLEKYLGEQMKSTS
YLLKLMKYRAPEPNESDIGLHTHTDTNIMTILHQDEIGGLEIQTKNDEWIRVKVSPNS
FVVVAGETFNVWLNGRLHVPFHRVVMTGNMARYSLGFFSVQKSSSFVKSFDEMVDEEH
PLLYKPFQYGEFLKFFYKEGGIKTNKKLDVEVLEKSVIIKLICDKVRPRLMSLKTKKS
NAVKATEKAWEMLRFQDIFTIPWNAKMEFKGTKAAYDTWGVGMGNSVINTSADCKGSI
CNIVTCNHLFLGKGAIPRYHTSIKAAPYETLYGRKCRSPICWAEVGESQLTGPDLVHE
TTEKIVQIKNRMQAARDRQKSYADKRRKPLEFQVGDKVMLKVSPWKGVIRFGKHEKLN
PRYIGPFEILARVGPVADRLALPSELSAVHNVFHVCNLKKCLSDDDSAIPLEEIQVDE
RLNFVEEPIEIMDREVKRLKQSRIPIVKVR
SEQ ID NO: 320
PWA65801.1|A0A2U1MX13
MAPVLEFPLNVGQIDDVQELKKSQKTKIPNRFVRDLSERPMLPITALPFSSSIPVIDL
AKLMKGNKEEFHYEILKLSASCEEWGFFQVINHEINLDLLDKIEKVATEFFMLPVEEK
QKYPMAPGTVQGYGQAFVFSEDQKLDWCNMFALGLVPHSIRNPKLWPTNPPMFSDTVE
TYSTEIRKLCKHLLQFIALSLGLNGNVFEEMFGESVQAIRMNYYPACPRPDLVLGLSP
HSDGSALTVLQQGKGNAVGLQILKNNTWVPIQPVPNALVINIGDTVEVLTNGRYKSSE
HRAVTHKQKDRLSIVTFYAPSYDIEIGPMPEMVDERSPSKYRRYNHGEYSRHYVTNKL
QGKKTLDFAKIAPETV
SEQ ID NO: 321
PWA77570.1|A0A2U1NVS8
MGSLPKAKQLPVIDLCLNNLDSSSSSWVRKCDEVTRALEEYGCFIAVYDGVSQELYDA
TVVASQEVFDLPIQKKVLNSRDAAGHGYLGQLPTMPLFERLSIENATTPQGAETFTKL
MWPSGNPSFCESALAFAKVLAELEVIVMRMVAKSLGIEQDYEKLQESTTYIFKFNKYL
SPPGGEKTVGIVSHTDKGFMTIIQQQEDGKGLEIKTKDGEWIEVEFKPSSFIVMAGDV
CTAWSNGRIEAPSHRVMMEGHKNRISLVTSSFIRDQVEVPQGLVSEHHPLKFKAFDHY
KYIEYHNSTTDANRNRLEDAIRFYCGI
SEQ ID NO: 322
PWA42076.1|A0A2U1KZ75
MAAKVISSGFRYTSLPESYVRPVTDRPNLSQVSECDDVPVIDIGCGDRELISKQIGDA
CRHYGFFQVINHGVPDEIVKKMQQVGRDFFELPLEEKMKLYSEDPTKTMRLSTSFNVQ
KEKVHNWRDYLRLHCHPLDKYSPEWPSNPAYFKEYVGNYVTAVRDLGMRVLESISESL
GLQKDRIIKILGDQGQHMAINHYPVCPEPELTFGLPGHTDPNALTILLQDTLVSGLQV
LKDGKWVAVKPHPNAFVINIGDQLEAVSNGQYKSVWHRAVVNSDKPRMSIASFLCPCN
DSILSAPKELIKDGSKPVFKDFTYAEYYKKFWSRNLDQEQHCLEFFKN
SEQ ID NO: 323
PWA35669.1|A0A2U1KFZ0
MRMPREGVPVIDMKKLDTLSMEIVKASEEWGCFRIVNHGVSIDLMAEMKAVVASLFDQ
PEEIKMRTVHTEVGKGYVKRNLAGPCFEGFSIDDISLPGEFCDRINASVHQRKIINKY
IKAIRDLARLLGLKLMEGCGLNGDLFDGWCCQLRMNKYHFCPKSIGSTGVPIHTDPTF
LTILQDDENVNGLQLIDKNSGQFILVDPVPGTLAVNIGDIGKAWSNGRYWTAKHRVRC
FEPKTRYSIALFVLGPNDKMIEAPSQFVDSEHPRYYVPIDPKKYRDVRVTHRIRTGVA
LELFRTSVT
SEQ ID NO: 324
PWA93555.1|A0A2U1Q6D2
MYVEMEQTNNHNIEAKIIDPSMNTVQQTHNYFPKQFIWPKDHVSRASVEVLNEHVIDL
QGFFKHDEKATLHEANLVNESCLKHGFFQVINHGIDPKLLALAYEHGWAFFQLPITEK
SKCKKKEGSMNDFSNGHAHRFAEKLPWKECLTFEYHENDSNEVAVEFFNSTYGSQYKE
TGLIYQRFCHAMRKLSLDIMELLEISLGVTSRKYYRQLFEDCVSIVRNNYYPKCDYPE
LTYGAGPHRDPTTLTVLYQDQFGGLEVFVDNKWKAVQPRPNALVINIGDTFTALSNGK
YMSCLHRVTVNTETQGLALEFFLCPKSDKKMKPPQELIDKDGKQEYPDFTWEEFLRFT
QKHHRADENNLDSFNNWLKSSRSSNA
SEQ ID NO: 325
PWA76605.1|A0A2U1NSV4
MSMDKHNEEAVNFGRSLIVPSVQELAKQSITKIPPRYAHQHDDQVQLNVSCDGELTSS
VPVIDLQCLFANANESSVYSSELNKLHTASKEWGFFQVINHGISESLLDDFKKEVLNF
FDLPMEEKQKLWQQEDNHEGFGQLFVVSEEQKLDWSDMFYITTLPHDLRKSQLFQKLP
LNLRQKLEAYSAEIKKLAMAILGQMAKALGMDDTEITELFQDGVQSMRMNYYPPCPEP
EMAVGFSPHSDADALTILYQLNTTEGLQIRKDGKWVTIKPLPNALVVNIGDIMEIVSN
GVYKSIEHRAVVNSDYERLSVATFYSSTMGTELGPAQSLVAQHTVANFRRVPLEEYFK
GFFARKLDGKSYLDFMKVGELKENITY
SEQ ID NO: 326
PWA68827.1|A0A2U1N5S2
MAPSVSVIHSNPQSLLDFVVNKGHGVKGLSELGLETLPHQYIQPPQERLDASNEESNK
DSIPVIDMSNWDDPEVAQAICDAARKWGFFQIVNHRVPIHVLEDVKDATLKFFALPAE
EKLKYSKERSVTNNIRFGTSFTPETEKALEWKDYLSLFFVSVDEAASLWPPICRNQAL
EYIEASELIVKKLLKILMNGLNVKEIDESTESILMGSKRINLNYYPKCPNPELTVGVG
RHSDVSTLTVLLQDDIGGLFVRNTETMKWIHVPPVSGSLVINVGDALQIISNGHYKSV
EHRVSANGNSNRISVPIFVCPRPSDIIGPLPEVVQNGEKPIYKHILYSDYVKHFFRKA
HDGKATIDFAKE
SEQ ID NO: 327
PWA85013.1|A0A2U1PGY0
MEKLNGEDRQATMDKIHDACENWGFFELVNHGISPELLDRVERMTKEHYKNCMEDRFK
EMVAAKALEGVTTEVNNLDWESTFFLRHLPTSNISEITDLEDEYRDLMKDFAGKLEKL
AEELLDLLCENLGLEKGYLKRAFYGSKSNGPNFGTKVSNYPPCPTPDLIKGLRAHTDA
GGIILLFQDDKVSGLQLLKDGQWIDVPPMRHSIVINLGDQIEVITNGKYKSVLHRVIA
QTDGTRMSIASFYNPGDDAVIYPAKILMKEQTKNEHSYPKFVFDDYMKLYAGLKFQAK
EPRFEAMKAAEANVIAGPVATA
SEQ ID NO: 328
PWA89796.1|A0A2U1PVN2
MADKCTIPVIDLHNLPNQLPELISACENWGCFRLINHHEILSTKLMLEMKKVVRSLLD
LPVEIKRRSSDAIAGSGYMAPSAKNPLYETLALYDMARSGDVERFCQELDATHDQREI
IMRYAEAVHELFMRIAKKLAEGLGVKRGDIGFENWPCEFRFNKYNFIPESVGSPGVQL
HTDSAFLTILQDDESVGGLEVMDKTGKFITINPWPDTLLVNLGDMATVWSNGRFCNVK
HRVQCKEAKIRVSIASFFLGPRGVVKPLSELVDDDHPLVYMPTTYEDYRKMRLSTNLK
AGEALEHLYTSSFKK
SEQ ID NO: 329
PWA91851.1|A0A2U1Q1F3
MAPSISITNTKSLNIMDFVVNKGHGVKGLAELGLKTLPHQYIQPPQERFDSSNEEPNQ
DSIPVIDMSNWDDLNVAKAICDAASKWGFFQIVNHGVPIHVLDDVKDATRKFFALPAE
EKLKFSKEKSVTNSIRFGTSFTPEAEKALEWKDYLSLFFVSDDEAETLWPSVCRNQAL
EYIKSSELVVKKLLKILMNGLNVKEVDEAKESKLMGSKRINLNYYPKCPNPELTVGVG
RHSDVSTLTILLQDEIGGLYVRNMATMKWIHVPPVSGSLVINVGDALQIISNGKYKSV
EHRVSANGSSNRISVPIFVNPRPSDVIGPLQELVDSGEKPLYKNVVYSDYVKHFFRKA
HDGKATIDFAKV
SEQ ID NO: 330
PWA66898.1|A0A2U1N069
MAPFLTDMHELKTFVVNEGNGVKGLSELKIKTVPELFIQPLEKRLDMSKVVLKESIPV
IDMSNYEDPEVMDSICDAAEKWGFFQVVNHDVPLSVLEDVKKATRRFFELPADEKKKY
LFNNSPSKNVRLLSSFVPEVDKAMEWKDYLSCFYVSDDEAMAFWPSVCRDQVLEYMSK
SESLIKRLLEILIKRLGITKLDETNQPLLMGSRRVNLNYYPICPNPELTVGVGGHSDV
STLTVLLQDETGGLYVRKLDSDNWIHVPPVKGSLTINIGDALQIMSNGRYKSIEHHVV
ANMHENRVSVPIFVNPRPNDVIGPLPEVIKGGEKALYKQVLYSDYVKHFYKKSHNGKD
TIDFAKLSLIDFTSGSWAACHLVNRSPSTAIDIKTPQEVLSEKPSNYSDLRIFWCPAY
AHVNDSKVESRGYGVHIYRVWEWKRRDRWCHREGSGCSCLKVEFATSTPNRVMIDDKQ
QPPKEPTSKALETSGLFTGLEPEETEPHDTEHMTEEEGDSEPIALTKKKRMRSHRLYI
QVSVVKLMHCKWVFKKKEGIPGVGPERFKARLVAKGFFTKLECFKLATNLVYHERTKH
IDMRLNIIRDVLKEDIFCIQKIATEENPSDILTKPLPPKSSSFAWTWLKNPVNILLQK
PFKYKGYG
SEQ ID NO: 331
PWA76167.1|A0A2U1NRR2
MAPSVSVIHSNPLSLLDFVVNKGHGVKGLSELGLETLPHQYIQPPQERLDASNEESNK
DSIPVIDMSNWDDPEVAQAICDAARKWGFFQIVNHRVPIHVLEDVKDATLKFFALPAE
EKLKYSKERSVTNNIRFGTSFTPETEKALEWKDYLSLFFVSVDEAASLWPPICRNQAL
DYIEASELIVKKLLKILMNGLNVKEIDESTESILMGSKRINLNYYPKCPNPELTVGVG
RHSDVSTLTVLLQDDIGGLFVRNTETMKWIHVPPVSGSLVINVGDALQIISNGHYKSV
EHRVSANGNGNRISVPIFVCPRPSDIIGPLPEVVQNGEKPLYKHILYSDYVKHFFRKA
HDGKATIDFAKGDS
SEQ ID NO: 332
PWA53653.1|A0A2U1LXB2
MGSLILPKLLVVDFTNDNLKPGTSVWSSTCNDIRVALENHGCFIALYDGVSSKLQDSV
FRAAEELFDLPTETKIKNIVEKPYHGYVGQMPIVPLHEGLGIDYATDLEGAQSFTDIM
WPDGNQSFCETSMSFSRAVAKLDQTVVRMLFESYGVEKQSASHIESTTYLLRYLKYRA
PETNETTMAFPSHTDKSFLTILHQNQVSGLEIRSRDEEWISVQFPASSFVVMAGDACK
AWSNNRVLSPNHKVTMDKQEKETRYTIALFSFLSKKVQIPDEFVDADHPLQFKPFDHI
DLLNFYVTENGRKSQNILKDFCGV
SEQ ID NO: 333
PWA87730.1|A0A2U1PPS2
MEQTNNLEAKIIDQSMNTIQQTHNHFPKQFIWPKDHVSRASVEVLNEHVIDLQGFFKH
DEKATLHEANLVNESCLKHGFFQVINHGIDPKLLALAYEHGWAFFQLPVTEKSKCKKK
EGSMNDFSNGHAHRFTEKLPWKECLTFEYHENDSDEVASEFFNSTYGSQYKETGLSYQ
KFCHDMRKLSLDIMELLEISLGVTSRNYYRQLFEDCVSIVRSNYYPNCDNPELTYGVG
PHCDPTTLTVLYQDQLGGLEVFVDNKWKAVQPRPNALVINIGDTFKALSNGKYMSCLH
RVTVNTKTPRLTLAFFLCPKSDKKMKPPQELIDKDGKQEYPDFTWEEFLRFTQKHHRA
DENTLDLFNNWLKSSKSSNA
SEQ ID NO: 334
PWA52746.1|A0A2U1LUR0
MAPSTPIINSNSLNILDFVVNKGHGVKGLSELGLKTLPHQYIQPPQERFDSSNEEPNK
DSIPVIDMSNPNDPHVAKAICDAAQKWGFFQIVNHGVPIHVLEDVKNATHKFFALAPQ
EKQKYSKSQSVTNNVRFGTSFTPEAEKALEWKDYLSLFFVSNDEAASLWPPICGKQAL
EYMKSSESVVKKLLEILMHGLNVKEIDETKQSILMGSKRINLNYYPKCPNPELTVGVG
RHSDVSTLTILLQDDIGGLYVRNTETMKWIHVPPVSGSLVINVGDALQIMSNGKYKSV
EHRVSANGSNNRISVPIFVNPRPSDIIGPMAEVLQSGETPLYKHVVYSDYVKHFFRKA
HDGKATVEFAKYTWKQALEYMKSSESVVKKLLEILMHGLNVKEIDETKQSILMGSKRI
NLNYYPKCPNPELTVGVGRHSDVPTLTILLQDDIGGLYVRNTETMKWIHVPPVSGSLV
INVGDALQIMSNDKYKSVEHRVSANGSNNRISVPIFVNPRPSDVIGPIAEVLQSGETP
LYKHVVYSDYVKHFFRKAHDGKV
SEQ ID NO: 335
PWA53060.1|A0A2U1LVN6
MSTENNKEEAINLGRSLIVPSVQELAKRSITHIPPEYAHQLDQDQLLLSCDDVLTPSV
PVIDLQSLFANTIESSVEYLFELNKLHTASKEWGFFQVINHGISESLLADFKRDVLNF
FNLPMEEKKKLWQEEDKMKVSVSCLLQTLEAYCVEIKKLATVILGQMGKALEMDEDQI
NELFHDGFQSMRMNHYPPRPQPNMAMGLSPHSDAGALTIVNQLNTTDGLEIRKDGKWV
TIKPLPNALLVNIGDIMEIVSNGVYKSIEHRAIVNAHSERLSVATFYCSNIATLLGPA
KSLVAKHKVANFKQIPLEEYFKGFFARKLDGKSYLEIMKLES
SEQ ID NO: 336
PWA55571.1|A0A2U1M2W2
MSVDKHNEEAVNFGRSLIVPSVQELAKQSITQIPPRYAHQHDDQVQLNVSCDDHAVNS
SVPVIDLQCLFANANESSVYSSELNKLHTASKEWGFFQVINHGISESLLDDFKKEVLN
FFNLPMEEKQKLWQQEDNHEGFGQLFVVSEEQKLDWSDMFYITTLPHDLRKSQLFQKL
PLNLRQKLEAYSGEIKKLAMAILGQMAKALGMDDMEITELFQDGVQSMRMNYYPPCPE
PEMAVGFSPHSDADALTILYQLNTTEGLQIRKDGRWVTIKPLPNALVVNIGDLMEIVS
NGVYKSIEHRAVVNSDYERLSVATFYSSTMGTELGPAQSLVAQHKVAKFRRVPLEEYF
KGFFARKLDGKSYLDFMKVEELKENIAY
SEQ ID NO: 337
PWA68517.1|A0A2U1N4V8
MEPKDISSAVSLVVPSVQELVKEPLTRIPPRYIRLDQDPPVISSSLSSNPELPIIDMK
RLLSLDSVDSELEKLHLACRDWGFFQLINHEVSSSLIEKVKEETQEFFKLPLDEKKKY
AQKPGDFQGFGQLFVVSEEQKLDWADVFSMITLPSHLRKPHLLPKLPQPFRDTIDEYS
KEIEKVALRTLMFISKALKMEVEEIKSLFDDGLQIIRINYYPPCPQPEQVIGLTPHSD
GPGITLLLQVNQVDGLEIKKDGNWIPVMPHPNAFIVNIGDIMEMVTNGEYRSIQHRAV
VNANKERLSIATFVSPKVDANIGPATSLITPEKPPRFRSVPLSDYLKNRLSKKLDVKG
NIEQYYI
SEQ ID NO: 338
PWA80846.1|A0A2U1P504
MVTMGAPVIDMQNVDGLAEELVKACVEFGCFRVVNHGVPMELMAEMKVVVAELFNLPE
EIKRQTATNNPEHGKGYIGCNPITPFLEGFSIDEIRSPNEFCDCLGASLHQREIISKY
IKSICDLNKFLGQKLMEGCGLTGDLFDGWCCQLRLNKYHFCPESIGLNGTPTHTDPSF
LTILQDDEDVNGLQVVDKVSGEFVPFDHVPGTLAINIGDIGKAWSNGRFCSVKHRVLC
FEPKTRYSIALFVMGPNDKIVQAPSELVDSEHPRLYVPIDVEKFRHVRNTTGLRAGDA
LDLFSTT
SEQ ID NO: 339
PWA38593.1|A0A2U1KPI0
MAGKCTIPVIDLHNLPNQLHKLISACENWGCFRLINHHEILSTKLMFDMKTVVRSLLD
LPVEIKRRNSDVIARSGYMAPSAKNPLYEALGLYDMAKSGDVERFCEELDATRDQRYA
EAVHELFMRIVKKLAEGLGVKRRDIGFENWPCQFRINKYNFIPESVGSPGVQLHTDSG
FLTILQDDEIVGGLEVMDKTGKFITVNPWPDTLLVNLGDMAMVWSNGRFCNVKHRVQC
KEAKIRVSIASFLLGPRGIVEPLSELVDDDHPLVYMPTTYEDYRKLRFSTTLQAGEAL
EHLYTPRFKK
SEQ ID NO: 340
PWA88642.1|A0A2U1PSE9
MTTLSEAYRNAPVASHQITPLDFDSLDRVPESHIWSQSDEPQQNIQSQEPQELSIPVI
DLTDPNALDLIGQACKTWGIFQVINHGVPLALIKKVESESRRLFGLPTDEKHKVLRSA
NGATGYGTARISPFFDKCMWHEGFTIMGSCVEDANVLWPHDYKNFCETMDAYQNEMKL
LTHKLLHLMLQTLNLTQEEMNWAISAQDSQAALQLNSYPSCPNPSNAIGLAPHTDSLL
LTVLHQGGANGLEIFVEGLGWSPVNPVDGAFVVNIGDLLHILSNATFQAVNHRAMVNQ
AKQRISVAYFHGPPVESVVAPSSSFENPCFRSMLVKEFISLKAKNFHKALSMIRK
SEQ ID NO: 341
PWA76686.1|A0A2U1NT42
MVVLTKPTLEQLIMKTTKPVNPLTLSQIPVVDLSSHDAKHQIVNACQEFGFFKVVNHG
ISMNLINKLESQAMQFFLSPQDIKEKAGPADPFGYGNKKIGNNGDVGWVEYLLLNAKP
ETDIEKLVSAFEEHPEVFQHALNDYLTSVTKMGCGILELMAEGLELEAKNVFSKLLMD
DKSDSVFRLNYYPPCPDVQEDEINGGNLIGFGEHTDPQIISVLRSNDTSGLEIALKDG
TWVSVPADSEAFFINVGDSLQVMTNGRFKSIKHRVVANSKKSRMSMIYFGGPPLNEKI
APLPSLIEREEDSLYKEFTWFEYKNTAFTSSLSHNRLGLYEKLPSKI
SEQ ID NO: 342
PWA64213.1|A0A2U1MSG8
MASLILPKLLVVDFTNENLKPGTSVWSSTCNDIRVALENHGCFIALYDGVSSKLQDSV
FRAAEELFDLPTETKIKNIGEKPYHGYLGQKPIIPLHEALGIDYATDLEGAQSFTDIM
WPDGNQSFCEASLSFSRAVAKLDQTVVRMLFESYGVEKQSASHIESTTYLLRYLKYRA
PETNETTIAMPSHTDKTFLSILHQNQVSGLEIRSRDEEWISVQFPASSFVVMAGDVCK
AWSNNRVLSPNHKVTMDKQGKETRYTIALFSYLSKKVQIPDELVDADHPLQFKPFDHI
DLLNFYVTENGRKSQNLLKDFCGV
SEQ ID NO: 343
PWA88877.1|A0A2U1PT34
MGSLAQPKQLPVIDLCLKYLKSSSSSWVAKCDEITRALEEYGCFIAVYDGVSQELHDA
TVVASEELFDLPVEVKVLNTKDAAGHGYMGNLPKIPLFERLSIENATTPQGAETFTKL
MWPSGNPSFCESVLAFTKVLAELEVMVMRMVAKSLGIEQDYVKLLEFTTYILKFNKYL
SPPGGERKVGIYPHTDKCFMTTIQQQEDGKGLEVKTKDGEWIEVDLKPAFFIVMAGDV
CTAWSNGRIEAPPHKVMMEGNKDRISLVVSSFIQDKVEVPQGLVDEDHPLKFKAFNHY
KYIEYHNSTTDANGKRLKDAIRSYCGI
SEQ ID NO: 344
PWA96717.1|A0A2U1QFG6
MAGCAELPLIDLSSSDRIATAISIRQACMEYGFFYLINHGVEEELLQNVVDQSNKFFS
LSIEEKMKLGRKDEDFGYAPLYSENHDHSTSSKGDSKETFHIGPLDGEESLQNQWPSK
ELLPSWRFVMEKYYKMLLSTGKRLSSLIALALNLEEDFFEKIGAVDRPSAFLRLLHYP
GDMGVSDEVVYGASAHSDYGMMTLLATDAVPGLQVCREKNKHPRTWENVTHVKGAFIV
NLGDMMERWTNCLFRSTLHRVMPTGKERYSVAFFMDPNPNCIVECLKSCCSESSPPRF
PPILSGDYLRERIHDAYSK
SEQ ID NO: 345
PWA62628.1|A0A2U1MN13
MGSLDEGWPEPVVCVQSLSDSGKLSIPDRYIKPQPERPSLSVNDMNIPIIDFNGLVTS
PEATMKQISKACREWGFFQLVNHGVRTDLVDGVRETWREFFHEPMEVKQKYANSPKTY
EGFGSRLGTEKDAILDWNDYYYLHYRPPCNTKWPDQPPSLREVMEEYTSEILRLGAAL
LKVFSVNLGLKEDSFQRAFGGNDFGACLRANFYPKCPQPDLTLGLSSHSDPGGMAILL
PDEQVCGLQVRKDDQWVTIKPEPHAIIVNIGDQIQVLSNAIYKSVEHRVVVNPNKERV
SLAYFYNPKSDMLIQPISELVTSNKPALYLPMTFEEYRGFIRTKGPQGKFQVESLKSP
R
SEQ ID NO: 346
PWA43750.1|A0A2U1L429
MADKCTIPVIDLHNLPNQLPELISACENWGCFRLINHHKILSTKLMLEMKTVVRSLLD
LPVEIKRRSSDVIAGSGYMSPSAKNRLYEALGLYDMAKSGDVERFCQELDATRDQREI
IMRYAEAVHELCMRLVKKLAEGLGVKREDIGFENWPCEFRFNKYNFIPESVGSPGVQL
HTDSAFLTILQDDESVGGLEVLDKTGKFITVNPWPDTLLVNLGDMAMVWSNGRFCNVK
HRVQCKEAKIRVSIASFLLGPRGIVEPLSELVDDDHPLVYMPTTYEDYRKLREIIMRY
AEAVHELCMRIMKKLAEGLGVKREDIGFENWPCEFRFNKYNFIPESVGSPGVRLHTDS
AFLTILQDDESVGGLEVMDKTGKFITVNPWPDTLLVNLGDMATVWSNGRFCNVKHRVQ
CKEAKIRVSIASFLLGPRGIVEPLSKLVDDGHPLVYMPTTYEDYRKLRFSTKLQAGEA
LEHLYTPRFKK
SEQ ID NO: 347
PWA86203.1|A0A2U1PKB6
MENPSHSQTTPLSYPPCFRPNTPQLSHQTNTTRDQKLELEHDPLPVIDFQHIDLMKLE
EACMDWGIFRLVNHGIPLTLMSKVHEHVSNVFDIGFETKQKMFETIPASVLSYFWGTP
ALTPSGVALYKDGDKQSDQGTISYNWVEGLNYPLSQGSLDFCLDQYPMIRDMRTLLEE
YGVHQERIAKSIFGAMSQNLSICEENEGYLSPSTGILRVYRYPRSFFDKSTKVWGMEA
HTDSSVVTILNQYEVEGLQVLSPKDEWIDAKPIPNTLVVHLGDMMQAISDDKYKSVKH
RVMVNRERERISMGYFVFPDNDCVIRSSNYKPFTYLDFRSQVQDDIKNLGVKVGLSRF
KLNKYFYI
SEQ ID NO: 348
PWA80844.1|A0A2U1P500
MVAISEPQVELFFPKPCKQNSKNNPLFMPTIPIIDLSKPDLKHLLVKACQEFGFFKVI
NHGVPIKCINKLESEAIKFFNSPLSAKQKAGPPDPFGYGNKSIGQNGDMGWVEYLLLN
PKLESHYKNPTISILEDKPEKFKYTVDDYVTSVKKMTCAILELLADELNLPQNVFSNM
LRDEQSDSVFRVNYYPPCPLFQEHEMKGKKLIGFGEHTDPQIISVLRSNNTSGLEILL
KDGNWMSVPPDTNSFFVNVGDSLQVMTNGRFKSVKHRVVANSLKSRLSMIYFGGPPLC
QKIAPLHLLMKRDEASLYKEFTWYEYKKSAFNSRLSDNRLCRFEKNRNVLIKVPSLKV
ELETSN
SEQ ID NO: 349
PWA67947.1|A0A2U1N397
MAPSFNDENSLFDFVVNEGNGVKGMVDSGLMEVPSRYIQPPHNRISKQHATVSIDQSM
TIDLSELDGPNHDQVVKSIAHAAETLGFFQVVNHGVPLELLDSLKVAAHQFFNQPVEK
KAVYLKGVSPSPMVKYGTSFNPDKEKALEWKDYVSMIYTNDDDAHMFWPKECKEVALE
FIKTTNQMVQKLLQALITSLGVKLDDSRLNAFIGFRMVNMNYYPTCPNPDLTVGVGKH
SDMGTLTVLLQDGIGGLYVKKGDDSSPGNEEWVGIAPAHGALVINVGDTLQILSNGRY
KSAEHRVRTTSVESRVSIPIFTAPLPMTKIGPLAELVARDGVARYRELLFEEYMNNFF
ANSHDGKKSLDFASTT
SEQ ID NO: 350
PWA69193.1|A0A2U1N6T0
MGLEGYIQLPVIDFSAINNQNPDTHDYDSVKTKVLEAFQEYGCFQASIVGISPELQNS
VYDVMKQLFDLPLETKSKNTSTQMFHGYFGNSPLLPLYESMGIDDAYNPEHVENFTNL
MWPQGNPQACKHIQTYAKKLWKFDAMVKKMLFQSLNLEKYLNEQAEKTNYVFKLMKYR
VPEPNDSNLGLHAHADAGVMTILHQKDVEGLEIQTKYDEWLKVKLSPNMFIIMAGETL
NVWLNGRLHVPIHRVMMRATKTRFSLAFFQLPLAGNIVKPMEEMVDDDHPLLFKPFDY
GEFIQFYITGGTGIQNYAVKAYCGV
SEQ ID NO: 351
PWA35350.1|A0A2U1KF07
MVVLTKPTLEQLIMKTTKPVNPLTLSQIPIVDLSSHDAKHQIVNACQEFGFFKVVNHG
ISMNLIKKLESQAMQFFLSPQDIKEKAGPADPFGYGNKKIGNNGDVGWVEYLLLNVKP
ETDIEKLVSAFEEHPEVFQHALNDYLTSVTKMGCGILELMAEGLELESKNAFSKLLMD
DKSDSVFRLNYYPPCPDVQEDEINGGNLIGFGEHTDPQIISVLRSNDTSGLEIALKDG
TWVSVPADSEAFFINVGDSLQVMTNGRFKSIKHRVVANSKKSRMSMIYFGGPPLNEKI
APLPSLIEREEDSLYKEFTWFEYKNTAFTSSLSHNRLGLYEKLPSKI
SEQ ID NO: 352
PWA73282.1|A0A2U1NID7
MGSEAPFQLPVVDFSALNKQNPDIVIWETAKTKAIEALQEYGCFEATFDEISPDLQNS
VFSELENLFNLPLETKQKNANDREFHGYIGQIPFMPLYESMGIDTPYVQEKVDEFTNV
MWPQGNSKFSKSIQAYSKKLWELDEMVKKMVFEGLNLRKYLEEHLEATSYHLKVMKYR
VADPSESTMGLDSHADTSILTILHQNGVQGLELRTKDETWVTVNVSPNSFVVMAGESF
NVLSNGRLHAPFHRVVMNANKTRLSVGLFSMPKVGSIVKPPKEMVDEEHPLLFKPFDY
GEFMDYFCMAGVKKDTYGLKTYCGLSI
SEQ ID NO: 353
PWA76662.1|A0A2U1NT29
MPTPAIPTVNLSPFFTAGDDSGRKKAKDIIAEACADYGFFQIVNHGVPLELMNQAMDL
SKTFFSYSDEVKHKSSSGPDAPLPAGYNKQPDHSPDKSEYLLMFPPESPFNILPDNPP
EFRKTLEEMFTYFVKTGQILEDIISECLDLPLNFLKEYNNDRSWDFMVTLRYKPATET
ENNGITEHEDGNLITMVLQEDVGGLEVCKNGEWIPVIPSKGTLVVNIGDVIQVLSNKK
FKSATHRVVRPKERSRHSYVFFYSLQGDKWVEPLPQFSQEIGAKPKYKGFYYKDYQAL
RLRNKTHPPSNPEDIARLTMYEI
SEQ ID NO: 354
PWA73280.1|A0A2U1NIJ4
MHKLPSIDETNKKNLKPGSTSWLATALEATRALEEYGCFIAEFDKVTPELNDAVFHGL
EDLFDIPIETKVLNKSTKPLYGYVGQIPFIPLYESMGFDYSNTLDGVRSFTEVMWPNG
NEAFSLRTKTCGDVSALEKYGCFMATYDGVSQELHDAIFDASHDLFDLPTEVKVLNTS
DTPSHGYVGQIPVIPLYEGLALKMQPLQMELQDSQNSCGPQEMKFSGMAIAELDQIVM
KMTAKSYGVKEHYEPLLGTTTYLLKLIKYLSTQGDERNLGIVPHTDKTFMSILHQDEV
KGLEIKTKDGEWIEYDPSPSSFVVMAGDGCMAWTNGKIEASCHRVMMQGNKERFSLGL
FTFIRNIKIEMPQELVDENHPLKFKAFDPLQISPLSCSR
SEQ ID NO: 355
PWA94887.1|A0A2U1QA64
MGSLDDYWPEPVVCVQSLSDSGNLSIPDRYIKPQPERPSLSVNDMNIPVIDFNGLVTS
PEATMKQISKACREWGFFQLVNHGVRTDLVDGVRETWREFFHEPMEVKQKYANSPKTY
EGFGSRLGTEKGAILDWNDYYYLHYRPPCNTKWPDQPPSLREVMEEYTSEILRLGAAL
LKVFAVNLGLKEDSFQSAFGGNDFGACLRANFYPKCPQPDLTLGLSSHSDPGGMAILL
PDEQVCGLQVRKDDQWVTIKPEPHAIIVNIGDQIQVLSNAIYKSVEHRVVVNPNKERV
SLAYFYNPKSDMLIQPISELVTSNKPALYLPMTFEEYRGFIRTKGPQDVKRYKHQITF
ERPSDKNTLKIPSETLEPNDVKWNPIHNLIKRLREDIANVMTI
SEQ ID NO: 356
PWA62597.1|A0A2U1MMY3
MNNKQVQEIAADCEQLPERYIRKQDEEYGANVTKSDVTSAVIPVIDLSLLISSPLELD
KLKSAVITWGCFQAINHGIDGSFLEKVREISKLFFKLPADEKKKYLREENDVEGYGND
MVLSENQTLDWTDRLYLTALPQDQQRLQFWPQNPTHFREILDEYSSKIQLVNEVVLKA
LARSLNLEEDCFLNQYGTTAKMQARFNYYPPCPWPEKVLGVKPHADSSAITVLLQDKE
VEGLQLLKDDQWVRVPIVPDALTINVGDQIEIMSNGIFKSPVHRVFVNSKNERMTLAM
FCMPQTEKDIGPVDGLITDERPRLYKNVTFTLDFFFKNYQQGRRAIDACRI
SEQ ID NO: 357
PWA36455.1|A0A2U1KI73
MASTLQSWPEPVVRVQSLSDSGINAIPDCYVKAIYDRPSSLDNISSIEANDIPIINLA
NLYANDPSIRKTTMDLISDACREWGMFQVINHGVSHHLMAATKRVWREFFHLPLDMKQ
TYANSPATYEGYGSRVGVEKGAKLDWSDYFFLQFLPASMRDESKWPTQPSSCRELVAE
YNEEVLKLCSRLMKIFSLNLGLDEDYLENAFGGEEIGATLRVNFYPKCPQPDLTLGIS
PHSDPGGITILLPDDHVSGLQVRRDDAWVTVKPIPNAFIVNLADQLQVISNAKYKSIE
HRVIVNSNEERVSLAFFYNPRGDIPIKPAEQLIADTESALYEPMTFNEYRTFIRLNGL
HGKSQVESLKSPR
SEQ ID NO: 358
PWA66083.1|A0A2U1MXT9
MENPSHSQTTPLSYPPCFRPNSPQLSHQTNTPHDQTLELENDPLPVIDFQHIDLMKLE
EACMDWGIFRLVNHDIPLTLMSKVHEHVYNVFDIGFETKQKMFESIPASVLSYFWGTP
ALTPSGVALYKDGDKQSDQGTISYNWVEGLNYPLSQGSLDFCLDQYPMVRDMRTLLEE
YGVHQERIAKSIFGAMSQKLSICEENEGYLSPSTGILRVYRYPISFFDKSTKVWGMEA
HTDSSVVTILNQYEVAGLQVLSPKDEWIDAKPIPNTLVVHLGDMMQAISDDKYKSVKH
RVMVNRERERISMGYFVFPDNDCVIRSSNYKPFTYLDFRSQVQDDIKNLGVKVGLSRF
KLNKDFYI
SEQ ID NO: 359
PWA54904.1|A0A2U1M121
MAPSFGDENSLTNFVVKEGNGVKGLVDSGLMEVPSRYIQPPHNRISKQHTTASIDQSM
TIDLSELDGPNDDQVVKSIAHAAETLGFFQVVNHGVPLELLDSLKVAAHQFFNQPVEK
KAVYLKGVSPSPMVKYGTSFNPDKEKALEWKDYVSMIYTNDDDAHMFWPNECKEVALE
FIKTTNQMVQRLFQALITSLGVKLDDSRFDELIGFRMVNMNYYPACPNPDLTVGVGKH
SDMGTLTVLLQDGIGGLYVKKGDDSSPGSEEWIGIPPAHGALVINVGDTLQILSNGRY
KSAEHRVRTTSVESRVSIPIFTAPLPKTKIGPLAELVARDGIARSRELLFEEYMNNFF
ANTHDGKKSLDFASTT
SEQ ID NO: 360
PWA52177.1|A0A2U1LT61
MKEDTDSKKWFNVEYVPKDYIFPKEERPENLDTPVCDSIPVIDLSKSKSVETILMASR
ELGFFQVINHGIPEKIVTDAMSVLKEFFDMPSKDRTGIVPHKKGYIYTNSTDYAKDGV
HLWRENIKHPCHPLEECIHLWPEKPTRYQEVIGAYLKEIQKLSSRILEMICEGLGLEP
GYLEDTSEVSLLSSNLYPPCPDPSLTLGILPHQDPSLITLLYQGNSTGLQVMKDSQWI
NVGDFPNAFVVNIGNQLEIISNGKLRSIKHRVVTSTHETRISIATFVNPSPDCIIEPA
KVLVNELEPSRYTASQYKEYVKSSKAYGDYTVAIQNALHS
SEQ ID NO: 361
PWA98517.1|A0A2U1QKN2
MGVPQHFLYICTILISPLLFTYNHISSNNHTPKTLENSCGVMVSTLQSWPEPVVRVQS
LSDSGINAIPDCYVKAIYDRPSSLDNISSIEANDIPIINLANLYANDPNLRKTTMDLI
SDACREWGMFQVINHGVSHHLMAATKRVWREFFHLPLDMKQTYANSPATYEGYGSRVG
VEKGAKLDWSDYFFLQFLPASMRDESKWPTQPSSCRELVAEYNEEVLKLCSRLMKIFS
LNLGLDEDYLENAFGGEEIGATLRVNFYPKCPQPDLTLGISPHSDPGGITILLPDDHV
SGLQVRRDDAWVTVKPIPNAFIVNLADQLQVISNAKYKSIEHRVIVNSNEERVSLAFF
YNPRGDIPIKPAEQLITDTESALYQPMTFNEYRTFIRLNGLHGKSQVESLKSPR
SEQ ID NO: 362
PWA90690.1|A0A2U1PY43
MEPKGTDLGSSLLVPSVQELVKEPITKVPSRYVRLDQDPPIISQPPSSLPEVPVIDMA
RLSSENSADHELEKLHLACKDWGFFQIINHGVGVSLLDKVKEETQEFFNLPMEEKKKF
WQTTDDIEGFGQAFVVSEEQKLDWADIFYLITLPHGIRKPHLFPNLPMPFRDTLEAYS
RELKNAAIKTLLYIAKALKMESKDMIVLFEEGMQSMRMNYYPPCPQPEQVIGLTPHSD
AVGITFLLQLNEVPGLQIRKDEIWIPIKPLPNAFIVNIGEILEIVINGQYKSVEHRAI
VNSEKERLSIATFLNPKLDGDLGPAPSLITSKTPPKFKRVAVADFFKNLFSRELNRKT
NLEQYYV
SEQ ID NO: 363
PWA88791.1|A0A2U1PSQ7
MPTPAIPTVDLSPFFTAGDDSGRKKAKDIIAEACTDYGFFQIVNHGVPLEVMNQAMEL
SKTFFSYSDEVKRESSSGPDAPLPAGYNKQPDHSPDKNEYLLMFPPESPFNILPDNPP
EFRKTLEEMFKYFVKTGQILEDIISECLDLPPNFLKEYNNDRSWDFMVTMRYKPATET
ENNGITEHEDGNLITMVLQEDIGGLEVSKNGEWIPVIPSKGTLVVNISDVIQVLSNKK
FKSATHRVVRPKERSRHSYVFFYSLQGDKWVEPLPQFTQEIGAKPKYKGFYYKDYQAL
RMRNKTHPPSKPEDIARLTMYEI
SEQ ID NO: 364
PWA69815.1|A0A2U1N8H4
MGFLAQPKIPVINMVDLKQNTTFGVSACDEVRHALEEYGCFLVDCNGITEGFSSEVLD
ALQNLFDLPIETKVKNISDKAYHGYVGQIPFLPLLESMGIENATSVHGVNSFTTTMWP
SGNDNFCNMIHSYARFVSEIEETVRKMVFRSFGVEKYHQSYNDSVTYLLRVMKYRPPS
ANETKLGSISHTDKSFISILTQNQVKGLEVKTKDNKWIPVEYPPCSFLVMAGDAFKAW
SNDRVHSPYHRVVMEGGEYRYSIGVFTYHKGIVTVPEELVDNDHLRKFKSFDHFGYLD
FYDKDSLFEQEFSHIKSYCGV
SEQ ID NO: 365
PWA92497.1|A0A2U1Q3D7
MSTDFQSIPLIDIGPLVAKCDDLNAMEDKDVAEVVRLLDQACREAGFFYVKGHGIPES
LIKEVRDMTHEFFNLPYEEKLKIKLSDATGYRGYQRIGVNITKGVPDMHEAIDCYKEL
SKGMYGDLSDPLVGNNRWPMDPPKFKKLMEEYLTLCTELSRKIMRGIALALGGSVDEF
EGKIAGDPFWVLRIIGYPGALTTSEEGLPKNDVGCGAHTDYGLLTLVNQDDDKTALQV
RNLSGEWISAVPIPGTFVCNIGDMLKILTNGLYESTLHRVINNSPKYRVCVAYFYETN
FNATIRPLDMCIKKSGGNKKFEGAVYGEHLVGKVMTNFTF
SEQ ID NO: 366
PWA35844.1|A0A2U1KGC8
MALDGKSLGGSLSVPCVQELAKESLVEAPPRYVRPDQDPIFASVLSATQTEVPVIDMQ
RLLSEESANSELERLHIACKDWGFFQMINHGVSCSLLEKVKEEIQEFFNLPMAEKNKF
LQEAGDLEGFGQAYVFSEEQKLDWADMFYVVTLPYHLRKPHLLPKLPLPLRDTIEAYS
RELKDISMKTLFLMAKALNMEVEDMNVLFDEGMQSMRMNYYPPCPEPEKVIGLSPHSD
PLGITFLLQINDVEGLQIRKDGIWMPVKPLPNAFIVNIGDVLEIVTNGQYKSIEHRAV
VNSEKARLSIGTFLTPKLDGDFGPAPSLISPETPPRFARVTVVDFLRNLFSKELDRKT
NVDQYYI
SEQ ID NO: 367
PWA53059.1|A0A2U1LVL8
MSTENHKEEAINLGRSLIAPSVQELAKQSIKHIPPEYPHQHDQDQDQMLLSCDDVSVP
VIDLRSLFANTSESYSSEFNKLHTACKEWGFFQVINHGINESLLADFKRDALNFFNLP
MEEKKKLWQKEDNNEGFGQLFVVSKEQKLDWCDMFYITILPHDLWKSQLFQKLPLILR
QTLEAYCVEMKKLAMAILSQMGKALEMDEDEITGLFHDGYQSMRMNHYPPCPQPNMAM
GISPHSDANALTILYQLNTTDGLEVRKDGKWVTIKPLPNALVVNIGDIMEIVSNGEYK
SIKHRGIVKAHSERLPAKSLVAKHKVANFKQIPLEEFFKEYFARKLDGKSHLEIMKLE
G
SEQ ID NO: 368
PWA83561.1|A0A2U1PCS2
MTCPSDWPEPIVRVQTLSETESQLIPTRYVKPLLERPSFNKGDQGNSNIPLIDLFDLT
NGDARAKKAIMDKISIACREWGFFQVVNHGMSSHLVDEAREVWREFFHQPMELKQEYA
NTPKTYEGYGSRLGIQKGAILDWSDYYFLNYLPSTLKDHNKWPSQPPLLRNVTEEYSN
EIVRLGKVLLKVFSINLGLQEDYLQDAFGGDDIGACLRVNFYPKCPQPDLTLGLSSHS
DPGGMTFLLPDEHVSGLQVRKGGEWITVKPARHAIIVNIGDQIQVLTNAIYKSVEHRV
VVNPDKERVSLAYFINPKSDILIKPATELVTSNAPALYPPMTFDEYRLFIRTKGPQGK
SQVESLKSSR
SEQ ID NO: 369
PWA55783.1|A0A2U1M3G2
MVCPCDWPEPVVRVQSLSESGKPVIPERYIKPPTDRPSFNSSPPDINIPIIDLAGLTN
DDITVREATLREISEACREWGFFQVVNHGLSGELVDGVREIWREFFHESMEVKQKYAN
SPKTYEGYGSRLGLQKGAILDWSDYYFLHYLPSNLKDHNKWPATPSSLRETVEEYSKE
IVRLGSVLLEVFSINLGLQKDYLQKAFGGEDVGACLRVNFYPKCPQPDLTLGLSSHSD
PGGITFLLPDENVSGLQVRRGEQWITVKPARHAIIANIGDQIQVLSNAIYKSVEHRVI
VNPDKERVSLAYFFNPKSDLLIHPAPELVTPETPPLYPSMTFDEYRLFIRTRGPQGKS
QVESLKSPR
SEQ ID NO: 370
PWA72667.1|A0A2U1NGM2
MVCPCDWPEPVVRVQSLSESGKPFIPERYIKPPTDRPSFNSSPPDINIPIIDLAGLTN
DDITVREATLREISEACREWGFFQVVNHGLSGELVDGVREIWREFFHESMEVKQKYAN
SPKTYEGYGSRLGLQKGAILDWSDYYFLHYLPSNLKDHNKWPATPSSLRETVEEYSKE
IVRLGSVLLEVFSINLGLQKDYLQKAFGGEDVGACLRVNFYPKCPQPDLTLGLSSHSD
PGGITFLLPDENVSGLQVRRGEQWITVKPARHAIIANIGDQIQVLSNAIYKSVEHRVI
VNPDKERVSLAYFFNPKSDLLIHPAPELVTPETPPLYPSMTFDEYRLFIRTRGPQGKS
QVESLKSPR
SEQ ID NO: 371
PWA37288.1|A0A2U1KKM1
MEPKGTDLGSSLLVPSVQELVKEPITKVPLRYVRLDQDPPIISQPPYSLPEVPVIDMA
RLSSENSADHELEKLHLACKDWGFFQIINHEVGVSLLDKVKEETQEFFNLPMEEKKKF
WQTTDDIEGFGQAFVVSEEQKLDWADMFYLVTLPHGIRKPHLLPNLPMPFRDTLEAYS
RELKNTAIKTLLYIAKALKMESKDMIVLFEEGMQGMRMNYYPPCPQPEQVIGLTPHSD
AVGITFLLQLNEVPGLQIRKDEIWIPIKPLPNAFIVNIGEILIEVTNGQYKSVEHRAI
VNSEKERLSIATFLSPKLDGDLGPAPSLITPKTPPKFTRVAVADFFKNLFSRELNRKT
NLEQYYV
SEQ ID NO: 372
PWA58960.1|A0A2U1MCH7
MAPSFNDENSLFKFVVKDGNGVKGLVDSGLTEVPGQYIQPPIHRIKKQNATVSLENMT
FDLSELDGPNHDQVVKSIAHAAETLGFFQVVNHGVPIELLESLKVSAHEFFNQPSEKK
AVYLKGVSPSPMVKYGTSFVPEKEKALEWKDYISMIYTNDANALEFWPNECKEVALEY
IKTSTEMVKRLLHALMGNLGIKLDDSRLDALMGLRMVNMNFYPTCPSPDLTVGVGRHS
DMGTLTVLLQDGIGGLYVKKGEDPSSGKEDWIEIPPVHGALVINIGDALQIISNGRYK
SAEHRVRTTSVESRVSVPIFNAPLPMVKIGPLPEVVARDGVARYREVIFEEYMNNFFG
KSHDGKKSLDFVAI
SEQ ID NO: 373
PWA69465.1|A0A2U1N7H0
MTIPVVDLASILGHSDEGERKKAVNEFGEACIQFGILRIINHGLADEHVNHSFEVVQQ
FFDRPIVEKLDSKPVSTILPAGYGKIDSHFGSSEWLMVCQPCLDINMFPSDQPEVSET
LEKMYQNFQKLGAIVENMMNEYLDLPLNFLNQFNDDRSNDILMCWRYPPIAEDKPNTI
GREEHQDTNCFTFLLQDDTGGLEYEKDGSWMPVNPMKGSLVVNVGSTIQALTNKKLMA
ARHRVWKPRGRNRHSFAFFYNIGGEKWIEPLPKFAEEIGEAPKYRGFFYKELLQARLK
KEKNPLTLLDEVIDLDHFATRN
SEQ ID NO: 374
PWA97792.1|A0A2U1QIJ9
MAPSFNDENSLFDFVVKEGNGVKGLVDSGLTEVPSRYIQPPHNRISKQHATTSIDQSM
TIDLSELDGPNHDQVVKSIAHAAETLGFFQVVNHGVPLKLLDSLKDAAHQFFNQLVEK
KAVYLKGVSPSPMVKYGTSFNPDKEKALEWKDYVSMIYTNDDDAHMFWPNECKEVALE
FIKTTNQMVQRLFQALITSLGVKLDDSRFDELIGFRMVNMNYYPACPNPDLTVGVGKH
SDMGTLTVLLQDGIGGLYVKKGDDSSPGSEEWIEIPPAHGALVINVGDTLQILSNGRY
KSAEHRVRTTSVESRVSIPIFTAPLPMTKIGPLAELVARDGVARYRELLFEEYMNNFF
ANSHDGKKSLDFASTT
SEQ ID NO: 375
PWA97961.1|A0A2U1QIW0
MDCPSDWPEPIVRVQMLSETENKIIPTRYVKPLLERPSFNKGDQGNSNIPLIDLFDLT
NGDARAKKAIMDKISIACREWGFFQVVNHGMSSHLVDEAREVWREFFHQPMELKQEYA
NTPKTYEGYGSRLGIQKGAILDWSDYYFLNYLPSTLKDHNKWPSQPPLLRNVTEEYSN
EIVRLGKLLLKLFSINLGLQEDYLQDAFGGDDIGACLRVNFYPKCPQPDLTLGLSSHS
DPGGMTFLLPDEHVSGLQVRKGGEWITVKPARHAIIVNIGDQIQVLTNAIYKSVEHRV
IVNPDKERVSLAYFVNPKSDILIKPATELLTSNTPALYSPMTFDEYRLFIRTKGPQGK
SQVESLKSSR
SEQ ID NO: 376
PWA92042.1|A0A2U1Q258
MPIPVIDFSKLDGEGRAKTMAQIANGCEEWGFFQLVNHGIPLELLARVKRVSSDFYKL
EREENFIKNSPSVKQLKELVDKKNEDKLENNDWEDVILLSDDNEWPTKTPGFKETMMA
YREELKKLAEKLMEVMDENLGIPKGYIKKAFKGGEGDKAFFGTKVSHYPPCPHPEMVN
GLRAHTDAGGIILLFQDDEVEGLEILKDGKWIDVQPIPNSIVINTGDQIEVLSNGKYK
SVWHRVQAMPDRTRRSIASFYNPSYNATIEPAAQLVEKENSDVNQSGYPKFVFSDYMS
IYTEQKFLPKEPRFQAVRTVY
SEQ ID NO: 377
PWA96755.1|A0A2U1QFJ0
MAPSFNDENSLFNFVVKDGNGVKGLVDSGLTEVPGQYIQPPIHRIKKQNASVSLENMT
FDLSELDGPNHDQVVKSIAHAAETLGFFQVVNHGVPIELLESLKVSAHEFFNQPSEKK
AVYLKGVSPSPMVKYGTSFVPEKEKALEEVVLKYIKTSREMVKRLLQALMENLGVKLD
DSKLDALMSFRLVNMNFYPTCPNPDLTVGVGRHSDISTLTVLLQDGIGGLYVKKDADL
PTGNEDWIEIPPVHGALVINIGDALQIISNGRYKSAEHRVRTTIVQSRVSAPIFNGPL
PVAKIGPLAELVARDGVARYREVIFEDYMKNFYAKSHDGKKSLDFVTI
SEQ ID NO: 378
PWA80845.1|A0A2U1P502
MVMSGTPVIDMQMVDGLADEIVKACVEFGCFRVVNHGVSAELMTEMKAVVSSLFDLPE
EIKRRTANPNEHGKGYIGRNHIDQLHFSRVLVLMKFRILINFLIVLVPPFTKVYLGVT
VTFTYCHKLTNHSFVRPTDIINRYIKAIRDLNGLLGRKLIEGNGLNGDLFDGWCCQLR
MNKYHFSAESIGLTCAPIHSDPTFLTILPDDECVNGLQLVDKISGEFVPFDPVPGTLA
VNIGDIGKAWSNGRYYNVNHRVLCFEPNTRYSIALFVLGPNNKIVEAPSKLVDSEHPR
LYVPIDVEKFRHVRNTMGLRTGDALDLFSTTST
SEQ ID NO: 379
PWA39568.1|A0A2U1KS39
MENMVTKWCTTVKSVPQDYVFPEGIRPGDQVVPSASQDSGFFQVINHGINEDLIHEAM
EVIKELFNMPDEDITKIYSLDHHKSCRLYTSSFDYVSESTHLWRDILKHPCHPLEDWV
HLWPEKPSKYRDVLGKFSVEVRNLSFRILEMIREGLGLKPGYFGDELTGIQMFSVNHY
PPCPEPSLALGISKHRDPNILTILYQGNTRGLQVLKDGQWFGVEPYDNAFVVNIGQQL
KVITNGKFESSEHRVVTNSKESRYTIASFINPHSDTVIGPEKSIVDKDNGRPLYQTFR
YKDFHRAYQAHKGEGEATIGVFKIDG
SEQ ID NO: 380
PWA74214.1|A0A2U1NL26
MVIPTNTRVETLATSGIHQIPKEYIRTQDELTTITNIFDEEKKELGPQVPTIDLNNIN
SKDPKTREKCCNELVKAATEWGVMHIVNHGISSDLINRVKDAGERFFDQPVEEKEKYS
NDIATGKIQGYGSKLANNACGQLEWEDYFFHLVFPEEKRDLTVWPSTPSEYIPATAEY
ARQLRALTTKILAVLSLGLGLEEGRLEDEVGGLEELILQFKINYYPKCPQPELALGVE
AHTDVSALTFILHNMVPGLQLFYDGQWVTAKCVPDSIIMHIGDTLEILSNGKYKSILH
RGLVNKEKVRISWAVFCEPPKDKIILKPLPETISKENPPLFPPRTFHQHMEHKLFKKK
DHSVEPK
SEQ ID NO: 381
PWA97954.1|A0A2U1QIX6
MPIPVIDFSKLNGEERAKTMAEIANGCEEWGFFQLVNHGIPVELLERVKKVTSECYKF
EREEEFFKNSTPVKLLKELVDKKSEEKLENIDWEDVFLLADDNEWPPKTPGFKETMME
YRAELKKLSEKLMEIMDENLGIPKGYIKKAFNGGEGDQAFFGTKVSHYPPCSHPEMVN
GLRAHTDAGGVILLFQDDEVGGLEVLKDGKWTDVQPIPNAICINTGDQIEVLSNGKYK
SAWHRVQAMPNGTRRSIASFYNPSYDATIEPATHLVKKENKEFNQSSYPKFVFGDYMS
VYTEQKFLAKEPRFQAVKAV
SEQ ID NO: 382
PWA82235.1|A0A2U1P8Z9
MVVPSNEINPIRNEKIREVEVPVIDLSGKRSKVAKLIVKACEEYGFFKVINHGVPHHI
IKNMEDESFEFFDKPLVEKEQVGSNNPFGYGCKNIGLGGDTGELEYLLLQANQTSIHH
TSEFSCAVSNYVEGVKELTCEILELIAKGLEVPHSSFSQMVRSLDSDSLLRLNHYPPV
SDTSHTFPRGSNSTSIGFGEHSDPQILTVLRSNGVAGLQISLGNDVWVPVSPNPLAFC
VNVGDVLQAMTNGRFVSVRHRAMANTSLNESRLSMVFFGAPPPEAIITCPPELLKENE
PLYKVFTWAEYKSHTYAHRLAVTRLDHFNIA
SEQ ID NO: 383
PWA61868.1|A0A2U1MKU3
MSTFTTIPCIKFITIQSHFINHLTMGQVDPTFIQELEHRPKPVVIEAQGIPQIDLSPS
VPIQELVDQIHDACKNWGFFQVINHGVPIESREKVQKAAKRFFDQTTEEKRKVKRDEA
NPLGYYDTEHTKNVRDWKEVFDFVVEVPTLMSASYEADDDEITEYRNQWPHHPSELRE
ACEEYVKDVQKLSNKLLELISLSLNLPAKRLEPFFKDQTTFVRLNHYPPCLAPDLALG
VGRHKDAGALTILAQDDVGGLEVKRKTDGEWIFVKPTPNAFIINVGDIIQVYHRDNIG
L
SEQ ID NO: 384
PWA93518.1|A0A2U1Q694
MDKLLSSWSSSAKSLPESYVFPVDSRPGDTNVPFCNTFPVIDLEAALANDRHDAIQQI
LKACQDFGFFQVINHGVNNDLVSDTMNVVKEFFNMPNEDKASVYSEDPSKKCRLYTST
YDYDNEKVHLWRDNLRHHCHPIEDFINLWPQKPARYRDVVREYSLQVDNLSSRILELI
SEGLGLEPGYFGDGLTGSQLFSVNHYPPCPNPNLALGLPKHCDACLITFLLQDEMYGL
QVYRNGEWFGVQPIPNAFVVILGHQAQVISNGKLISPEHRAITNSKNHRTSIVYSINP
KPDTIIEPAKALINKSNYPLYRAFQFKEFLKAYEVKKDYNEGALEDFKIKEELKEESS
HKTRDQGCENQLGDLHLTK
SEQ ID NO: 385
PWA55631.1|A0A2U1M326
MEVERVQEIATLSNLNGTIPSEFIRLDNEQPASTTLHGVVLEVPVIDLSQTDNESLVD
LISKASKDWGLFQVVNHGIPSELISKLQKVGKEFFELPQEEKEVIAKPDGYKGVEGYG
TKLQKEVQGKKGWVDHLFHIVWPPSSINYQFWPKNPPSYRDTNEEYAQSLIGVANKLL
GLLSKGLGLEEDEVKQALGGEDLVYMLKINYYPPCPCPELALGVAPHTDMSSITILVP
NEVQGLQVFKDGDWYDVAYIPNALIIHIGDQIEILSNGKYKSVYHRTTVNKEKTRMSW
PVFLEPPLEFEVGPIPKLVNKDDPPKYKTKKYKDYVYCKLNKLPQ
SEQ ID NO: 386
PWA93349.1|A0A2U1Q5R3
MEPDSSFTSVMALTLERQKNIPEQYVLPPLQRPNMSLTDHIPTSLPLIDLSMINHPAH
RSQVIDEVHAACKQLGFFQVINHGIPTSVLKDALDSAEEFFNLPSEEKMCFASADVHE
PVRYGTSMNHGKDKVFYWRDFLKLYANPISEWIHLWPSNPPSYREKMGSYADAMHTLQ
KRLMEVVLESLGLNANYLHEDIDEGSQVLAVNCYPSCPEPDLALGMPPHTDYGTLTIL
NQSHQGLEIMDHDKKWHSVPFIEGALIVQLGDQSEVMSNGRYKGTPHRAILNTQKKRL
SIASLHSLAIEKKIGPAPELVDEQHPVAYKEGSFAEFLDCIALNSVSDVKYIDTLRIH
SEQ ID NO: 387
PWA72476.1|A0A2U1NG93
MATITPLITSSYDRASEIKAFDETKTGVQGLVDAAIQQIPRIFINPPEPIPKSTTSFD
IPLIDLASTNRALIVESIRKASENLGFFQVVNHGIPICIMNETLRGVRRFYEQDVEVK
KQFYTRDASKAFVYNSNFNLYSSPAANWRDTFFSFMAPSPPKPEDLPVVCRDIQIDYS
SRVLKLGESLFGFISEALGLETHHLGDMECDKGLTFVAHIYPACPQPELTMGATKHTD
IEFLTVLLQDDIGGLEVLHENQWIDVPPTPGALVINIGDLLQMISNDKLRSVEHRVVA
KEKGPRVSVACFFSTSLAPSTKVYGPIKELLSDDNPPRYRETTVQDYVEYNLSKGLDG
DPRLLHLKI
SEQ ID NO: 388
PWA39580.1|A0A2U1KS53
MNHGIESSLLEEVREIGIQFFKLPVEEKKKCLREKDDLEGYGNDMVLSNNQTLDCNDR
IYLAVVPQHQQKLQFWPQNPTHFQEVLNAYSSKIDLISKVVQKSLARSLNMEEDSFLS
QYGTTASISARYNYYPPCPWAEKVLGVKAHSDSSAITVLLQDKEVEGLQLLRDNQWVG
VPILRDALTINVGDQIEIMSNGIFKSPVHRLLVNSKKERMTLAMFCVPQTEMVIGPVD
GLITDETPRLYKNGTYTLDFFFKDFQQGRRAIDACKI
SEQ ID NO: 389
PWA75825.1|A0A2U1NQP1
MTGAKFEIEKFDGTGDFGLWRVKMRALLIQHGCEAALEVLPANMDATAKAELNKKAHS
AMILCLGNKVLREVTGETTAAGVWTKVETLYMTKSLANKLYLKKKLYTFYMPAGRRHT
TQGVIDYVHSDLWGPSQVESLGGKRYFLSIVDDYYRRVWVYILRFKHEAFGKFKEWKQ
LVENQTGRKSGLPKTFWAEATCTAAYLINRSPSTAIEKKTPMEMWSGHPSDYGMLRIF
GCVAYSHVKQGKLEPRAVKCVLLGYPEGVKGYKLYRLDNESPKIVTSRNVVFNESVMY
KDTLKDSGADQTHDQEDSDDEDAGDQDHDQTPDLTDYQLVRDREPRTRTKTLRFRDES
NMATYAFAAAEEEDTHEPLTYQEAVACEDNPKWKAAMDEEMDSLKKNETYELVDHPAG
QKLMSYKWLFKIKEGIEGVHKPRYKARDYELEQLDVKMTFLHGNLEEVIYMRQPPGYE
QGNKSPRQWYRRFDEYMLSNGFKRRSYDSCVYYRSYAPGEYIYLLLYVDDMLIACKSK
AEIGSTKSLLKKEIDMKELGEAKKILGMEIIRDRSRKILRVSQSGIDNGKSVQMPLGG
HFKLSLKDCPVRDCDVERMSKVPYSNAVGSLMYLMVCTRPDIAYAVSVVSRYLANPGK
NHWEAVKWILKYLRGTANVGLVFGTNRGNHVDVTGFVDSDYAKDPDKGRSITGYAFLV
QGCVVSWKATLQHVVALSTTEAEYMALTEAVKEAIWLRGLLEELGVELNTVAVNCDNQ
GAIHLSRNHVFHERTKHINVRYHFIRDVLEAKTVEVLNSSRSRFGWLVSQAQPVSSFQ
PTYRVGVLPPHLYKRRCEQLMSVAKKFFDQQVEEKRKVMRNEANPLGYCDTEVTKTVR
EWKEVFDFTAGVPMVMPTEGDQTVEYANQWPDHPSELREACEEYAKAVQKLSSKLLKL
ISMSLNLSPNRLEPFFKDQTAFVRLNHYPTCLAPNLSLGAGRHKDAGALTILAQDDVG
GLEVKRKTYGEWIFVKPVRNSFIINLGDVMQVWSNDIYESIEHRVRVNSTRERFSIPF
FMNPALYNVVEPLPELISEENPAKYKGYHWGKFLATQKQ
SEQ ID NO: 390
PWA59552.1|A0A2U1MEE2
MATITSSINSSYDRAAEVKAFDETKTGVQGLVNAGIQEIPRIFIHPPQNIPKTTISFD
IPVIDLACTDHASTLDKILKASENLGFFQVVNHGIPTSIMNEMLQGVRRFHEQDVEVK
KKFYTRDPSKAVIYNSNFNLYSSHVADWRDSFFSYMAPSPPLAEELPEVCRDIQIEYS
KQVLKLGGLLFRLISEALGLEPTHLGDMDCDKGMLFLGHYYPACPQPELTMGATKHTD
DGLLTVILQDETGGLQILYQNEWIDVPSTPGALVINIGDLLQLISNDKLRSVEHRVVA
NQIGPRVSAACFFSTNLAPSTKVYGPIKELITDDNPARYKETTVHDYTNLSFSKGLGV
PQLLYLKI
SEQ ID NO: 391
PWA75127.1|A0A2U1NNN4
MDISNSNDDRTKQLKAFDDTKSGVKGLLDAAAGGVVEIPQIFIRPPEEVAEDLELGRT
SLRVPAIDLTGVGDKGSCTREKIVEQVKHASEKWGFFQVVNHGVPMKVLDAMLNGARE
FNEQDVEVKKEYYSRDPERMVKFNTNYDFYMSKSASWRDTLIVDMLNSNHLDPQDLPA
PCRNSTVDYLNQVMELADTLFELLSEALGLEPNYLKQLECEKSQTLTCHYYPACPKPD
QTLGVNKHTDASFISILLQDEVGGLQVLHQNQWADVEPIPGALVVNIGDLLQIVSNDK
FKSVIHRAVGNVTQARTSVAFFLHGVVTPPKMYGPIEKIITKESPRIYREFTVKDYMM
KFYSRGLDEKTGLNYVRI
SEQ ID NO: 392
PWA44820.1|A0A2U1L730
MSGAAANSDDIGGGAVETVINISSSSDLKLIKKDKDSRWFDVELVPESYIFSPTDRPQ
NLDVAICDSIPVIDLAKHIQPIESILKASQEFGFFQVINHGVPEKIMSDAMSVLKEFF
HMPSKEATGEVIAAYLLEINKLSFRILEMICEGLGLEPGYLSDTSEVQILSSNFYPSC
PDPSLTLGILAHQDTSLITLVYQGDSTGLQFLKDGQWINVGSIPNSFVVNIGNQLEIV
SNGKLRSIDHRVVTSKDKTRISIATFVNPSYDCIIEPAKALVNKNEPSRYKASRYKEY
VDRNKAFGDYTVALRDVLNSHVIRLEVREPIGGTHVKTRRIGKVELNGGFWISAVVVC
FIILFEVYKKAIDPKADQPENDGVKTRKNGKNKTKKIGVEVDDEKPIDK
SEQ ID NO: 393
PWA82404.1|A0A2U1P9I1
MWLLLASFILRNKQLVFNPIITLPSTFPFHLQNHIHNTMVLSSPSPLRTKKTRAIGIP
IIDLSLDTATLSQHIVTACQEYGFFKVVNHGVPFEIISKMEEVAQHFFSKPTSEKVKV
QANPPSPFGYGCRNIGYNGDVGELEYLLLQANSIALGSDHPTNFRGAVEDYIQAVKQL
TCGLLEILALGLSLPDTNAFSRFIQDVDNDSCFRVNHYPGVKSESNQPKPAQRIGFGE
HSDPQIFTILRSNDVPGLQISTFDGLWIPVTNEPNDFCVFVGDALEVLTNGRFQSVRH
RVMANMSNKSRMSMMYFAAPALNERLSPIPQMIGNLRLYKSFTWNEYKKAAYSLRLGD
QRIDLFKCHST
SEQ ID NO: 394
PWA43549.1|A0A2U1L3G2
MEIKKDKDSRWFDVELVPESYIFSPTDRPQNLDVASCDSIPVINHRVPKKIMSDAMSV
LKEFFDMPSKEATGYVPQNKGWIYTNNDCTKDGVHIWRENLKHLCHPLDKCTKLWPNK
PTRYQEVIAAYLLEINKLSLRILEMICEGLGLEPGYLSDTSEVQILSSNFYPSCPDPS
LILGILAHQDTSLITLVYQGDSPGLQFLKDGQWINVGSIPNSFVVNIGNQLEIVSNGK
LRSIDHRVVTSKDKTRISIATFVNPSYACIIEPAKALVNKNEPSRYKASRYKEYVDRN
KAFGDYTVALRDVVISES
SEQ ID NO: 395
PWA44819.1|A0A2U1L744
MSGAAANSDDIGGGAVETVINISSSSDLKLIKKDKDSRWFDVELVPESYIFSPTDRPQ
NLDVAICDSIPVIDLAKHIQPIESILKASQEFGFFQVINHGVPEKIMSDAMSVLKEFF
HMPSKEATGEVIAAYLLEINKLSFRILEMICEGLGLEPGYLSDTSEVQILSSNFYPSC
PDPSLTLGILAHQDTSLITLVYQGDSTGLQFLKDGQWINVGSIPNSFVVNIGNQLEIV
SNGKLRSIDHRVVTSKDKTRISIATFVNPSYDCIIEPAKALVNKNEPSRYKASRYKEY
VDRNKAFGDYTVALRDVLNSHVIRLEVREPIGGTHVKTRRIGKVELNGGFWISAVVVC
FIILFEVYKKAIDPKADQPENDGVKTRKNGKNKTKKIGVEVDDEKPIDKVVGESHEVQ
ACAEVNDYSLEDYVGLSPLHSMTR
SEQ ID NO: 396
PWA34910.1|A0A2U1KDR6
MALLVTSWSNGVQSVPKDYIMPPERRAGDFVTISKDIPVINLQNDRVINHGVPDEMMA
DMRVLYDEFFNMPVEDKLGVYAEKFGKGCTLYTSGMNYAKEDVHYWKDTLKHPCHPLE
EHTPSWPEKPTRYREEVGKYSIEVRKMGFKILELIAEGLGLNKGHFEEVGREQSMAIN
HYPPCPDPSLAMGIGGHTDPNLITFLQQDHYGLQIQKDGQWMGIEPIPNAFVVNLGYQ
LQIISNGKLKSAEHRGVLNSTASRTSIVTFFAPNPTLPLVVEPAKELVTSTTPQVFKS
YLYNEFLAEYLAFIRKPGPRNGTPLDPYRI
SEQ ID NO: 397
PWA70384.1|A0A2U1NA39
MGANSINGDTSIPVIDLQDFPNQSSKLLAACEEWGCFRLLNFNEILPKTLMLEMQAVA
RSLCDLPEEIKRRNIDHEVPEDGYAAPSPKYPLYEAIGMQDLTSTRGVDLFCSQLDAA
PQQRETIKKYAEAIDGIAREIVKKMGESLGVKSENLKFENWKCKSRINKYQFTPQSFG
SLGAPVHTDSGILTLVHDDEEGVGGLEAMSPSGDFVPFDPWPGTLSILIADMATVWSN
GRFRNVKHKIVSKDGKSRVSVTSFVTGLAEILEPLPELVDDHHPRLFLPIDTEGYRKL
RYSKNLHDGEALALLQPDHIQT
SEQ ID NO: 398
PWA68441.1|A0A2U1N4L4
MNFVKALAESPDLNSVPSIYNYSTNSNEPLASDPQESIPTINFSLLNSTDADLRSKEI
QELDKACKEWGFFQVINHGVPEALMKMVIEKSYEFFNMTDEEKKDFEEKDVLDPIRYG
TSFNFKKDIVLYWRDFLKVIVHPEFNCPNKPLGFSEVLFEYSQRTREVARGLLNGISL
SLGLDQSYAEKALEFESGLQIFVVNLYPPCPQPELAIGMPPHSDHGLFTLLINNGVAG
LQIKHKGKWVNVNKTLPNSFLVNTADQLEIFSNGKYRSVEHRAVVNNAVTRISVVVAN
GPALDTVVRPAYKLVDEETSPVAYVPMKYKEYLEMQQGNQIVGKKCLQRIQV
SEQ ID NO: 399
PWA72477.1|A0A2U1NG38
MATITPLITSSYDRASEIKAFDETKTGVQGLVDAAIQQIPRIFINPPEPIPKSTTSFD
IPLIDLASTNRALIVESIRKASENLGFFQVVNHGIPICIMNETLRGVRRFYEQDVEVK
KQFYTRDASKAFVYNSNFNLYSSPAANWRDTFFSFMAPSPPKPEDLPVVCRDIQIDYS
SRVLKLGESLFGFISEALGLETHHLGDMECDKGLTFVAHIYPACPQPELTMGATKHTD
IEFLTVLLQDDIGGLEVLHENQWIDVPPTPGALVINIGDLLQMISNDKLRSVEHRVVA
NEKGPRVSVACFFSTSLAPSTKVYGPIKELLSDDNPPGYRETTVQDFVEYSFSKGPDG
VPRLHYLKI
SEQ ID NO: 400
PWA83917.1|A0A2U1PDX6
MGNLKNSGGIVVDATVSQRGRRDWPEKQTEKMEPDTSFKSVMALALECQKNIPEQYVL
PPLQRPNMSLTDHIPTSLPLIDLSMINHPAHRSQVIDEVRAACKQLGFFQVTNHGIPT
SVMKDALDTAEEFFNLPSEEKMCFASTDVHEPVRYGTSMNHEKDKVLYWRDFLKLYAY
PISEWIHLWPSNPPSYRERMGSYAEAVHKLQNQLMEVVLESLGLNANYLHEDIEEGSQ
VLAVNCYPSCPEPDLALGMPPHTDYGTLTILNQSHQGLEIMDHAKKWHSVPFIEGALI
VQLGDQSEVMSNGRYKGTPHRAILNVQKKRLSISSLHSFAIEKKIRPAPELVDEQHPV
AYKEGSFAEFLDCIALNSVSDVKYIDTLRIH
SEQ ID NO: 401
PWA94615.1|A0A2U1Q9D3
MADVKQVSKLNCIDLSSPDTPTSVSLLKQACMDSGFFYVTNHGISPEFMEHVFTQSKN
FFDLPLDQKMKLLRNEKHRGYTPVLDELLDPVNQLHGDHKEGFYIGIELPEDDPEAQR
KFYGPNLWPESDILPGWRQTMEKYHQMAMEVVRKIARLIALALDLEANFFDRPEFLGR
PIAVLRLLHYEGQVSDPTKGMYGAGAHSDYGLVTLLATDCVSGLQICKDKDAKPQVWE
DVKPLKGAFVVNLGDMLERWSNRIFRYKYSGLSHCFAVILVFIWSSLNICLNRSTLHR
VLGNGQERYSIPFFVEPSHDCVVECLPTCQSKLNPPKFPPIKCEEYLLQRYQDTHADL
RTYN
SEQ ID NO: 402
PWA58898.1|A0A2U1MCC8
MKLPVIDLSSSDRISTAHSIRQACTDYGFFYLINHGVDEQTVFEQSRLFFSLPLEDKM
KFSRDVAADFGFSPLYAENLDSSSSSSKAKGDSKESFRIGPLQNQRNQWPSQDLLPSW
RSVMETYYASLLSIGKKLSSLIALALNLDDHFFHSVGALDNPHTILRLLHYPGEMGDS
DEDGVVYGASAHSDYGMITLLATDGVPGLQVCREKLKHPRTWENVNHVKGAFIVNLGD
LMERWTNCLFRSTLHRVMPTGKERYSMAFFLDPNPDCGRMLEKLLQ
SEQ ID NO: 403
PWA98228.1|A0A2U1QJS9
MAFAQPLMSHESVQELAKEHNITIPDHYVQEHPEPTFVPNDCLPMPSIPIIDMNDLIM
GRLNMDLHLKHLRSVCHEWGIFQLVNHGVDKSLVEKMKNEVIEFFTLPIEEKLRYKLK
GGEYEGYGQTILHAQGQKIDWADRFYMITNPLHRRKSNLLPEFPPLLRDTLENYLLEL
QKLGRTLFGIIGQAVDIDKEEMLEIFEDGMQSVRMTYYPPCPQPDLVVGLTPHSDAAG
ITFLLQVNNVEGLQVKKDGAWIPVNFLPDAFVVNVGDILEIMSNGVYTSIEHRAVVNA
TKERISLAMFFNPKLEADVGPSKSLIKSTRNPPLYKTLMMEQDKKLAPRLLAVSVTYM
IISR
SEQ ID NO: 404
PWA40908.1|A0A2U1KVX1
MHKLPSIDFTNKKNLKPSSTSWLATALEATRALEEYGCFIAEFDKVTPELNDAVFHGL
EDLFDIPIETKVLNKSTKPFENVLAYNKLVAELEEMVTRMVFETYGVEKYLDAHRKMV
TYLCRGMKYRPPVENETNMGFVPHTDKDFITVLHQNGVNGLEVKARDGEWFSVELVPS
SYIVMSGDAAMAWSNERLYSPFHRVTMNGKESRYSIAQFSFLEGIIETPKEFVDEEHP
LLYKPFDHLKYLDFYNKEENRRLECAIRTYCGV
SEQ ID NO: 405
PWA43827.1|A0A2U1L4A6
MASMSHDFHRAKTEFDQTKAGVKGLLDSGVVKIPKIFVHPTENLVKYPSLGTCSLSLQ
VPVIDFESGKRAKIVGEIKEASESWGIFQVINHGISVSVLEEMIKGVQRFHEQRKEVK
MEWYSREHDQRVKFYSNGDLYVSKAVNWRDSISCYYADGVLDPNALPQVCRGAIENYM
ERIIKLKDTLAELLSEALGLDKDYLTNLDCMKTPTLVCHYYPPCPEPNLTLGAAKHSD
PSFLTILLQDSIGGLQVLQRNQWVDVKPIKGALIVNIGDLMQLITNDKFKSVEHRVLA
APIGPRISAACFFYPSTANNTKPYGPIKELLSEECEPIYRQTNYKEYMTHYKAKGLDG
ASSLTLFKA
SEQ ID NO: 406
PWA40907.1|A0A2U1KVY6
MHKLPSIDFTNKKNLKPSSTSWLATALEATRALEEYGCFIAEFDKVTPELNDAVFHGL
EDLFDIPIETKVLNKSTKPFENVLAYNKLVAELEEMVTRMVFETYGVEKYLDAHRKMV
TYLCRGMKYRPPVENETNMGFVPHTDKDFITVLHQNGVNGLEVKARDGEWFSVELVPS
SYIVMSGDAAMAWSNERLYSPFHRVTMNGKESRYSIAQFSFLEGIIETPKEFVDEEHP
LLYKPFDHLKYLDFYNKEENRRLECAIRTYCGVNV
SEQ ID NO: 407
PWA83611.1|A0A2U1PCX2
MTTSTTRRERERESRACQNLGSSIPVPSVKQLTSQPLETVPHRYIQDTSNDYPTATPT
PCHSLPIIDFSKLIHPQYQSSELLNLHLACKHWDQFKVVNYEGITMYQRPFVRGKLYI
RFTVEFPESFPKQCKAQQGVLPPRPSMQMTDMELDECEETTLHDVNIEEEMRRKQQEA
LKMKNDDMHGEDTMWTAISVGYELIGHRVSEESIKCMDRKTQEFFDLSSEEKKRYAQK
PGSLEGYGQAFVISEDQKLEWCDMIFLKAIPTHTRKLEFWPEEQPHNFREALDTYSHD
IKKTAVSIIEFIAMALGLDGKRFSEAFEGGNYDVRMNCYPPCPEPERVIGISSHADIS
GITLLTDCGDIPGLQVLKDEKWVFVEPITNGIVVNIGIIMEVVSNGVYKAPYHRAAVN
KQKDRFSTVTFCYPGKKFDIKPAEELINSGSLALYKSFSYDGYFESFYERTKLSGDGV
PFIDTLKI
SEQ ID NO: 408
PWA42312.1|A0A2U1KZW0
MIYPYIMFRSTMARRDSLPVKTAQQMAMDGDQPPSKYIYSNNTSYAKFGPLETSPPFA
PVPVIDIGCLLSSCKQDEQENELAKLRSALTTWGCFQAVNHGLSDSYLDNIQQVIKQF
FELSLEDKREYFRETGSVEGYGNDMTYSETQVQDWCDRLFLRILPEDQRKLRFWPENP
SNFRETIDDYTKKIMSISLVIFKAMAKSLELEENCFSKHFTEESDVLQGRFILYPPCP
TPDKVFGLKAHSDRSGITLLLQDPGVEGLQVLNDGKWYMVPVIPEALFINLGDQMQIL
SNGIFKSPVHRVVTNSDKGRISVAMFTEPEPNKEIGPVDALVDEKRPRVYKTVKDYAV
FNHECFQKGVVALDAVKL
SEQ ID NO: 409
PWA52836.1|A0A2U1LV00
MATITSSINSSYDRASEVKAFDETKTGVQGLVNAGIQEIPRIFIHPPQTIPKATVSLD
IPVIDLACTDHASTVDKILKASENLGFFQVVNHGIPISIMNEMLQGVRRFHEQDVDVK
KKFYTRDPSKAVIHNSNFDLYSTQAANWRDNFFSYMAPSPPSAEELPEVCRDIQIEYS
KQVLKLGGLLFRLISEALGLEPNHLGDMDCDKGILSLGHYYPACPQPELTMGATKHTD
DGFLTVLLQDEIGGLQILYQNKWIDVPSTPGAIVINIGDLLQASLLISNGKLRSVEHR
VVANQIGPRVYAACFFSTNLAPSIKVYGPIKELVSDDNPARYRETTVHDYTKLTFSKG
LGVPRLLYLKI
SEQ ID NO: 410
PWA72914.1|A0A2U1NHC1
MNFVKALAESPDLNSVPSIYNYSTNSNEPLASDPQESIPTIDFSLLNSTDADLRSQEI
QELDKACKEWGFFQVINHGVPEALMKMVIEKSYEFFNMTDEEKKDFEEKDVLDPIRYG
TSFNFKKDIVLYWRDFLKVIVHPEFNCPNKPLGFSEVLFEYSQRTREVARGLLNGISL
SLGLDQSYAEKALEFESGLQIFVVNLYPPCPQPELAIGMPPHSDHGLFTLLINNGVAG
LQIKHKGKWVNVNETLPNSFLVNTADQLEIFSNGKYRSVEHRAVVNNAVTRISVVVAN
GPALETVVRPAYKLVDEEMCPVAYVPMKYKEYLEMQQGNQIVGKTCLQRIQV
SEQ ID NO: 411
PWA36547.1|A0A2U1KIF2
MADCKKVSKLNCIDLSSQDIPTSVSLLKQACMDSGFFYVTNHGISEEFMEHVFTQSKN
FFHLSLDEKMKLLRNEKHRGYTPVLDETLDPANQLHGDHKEGFYIGIELPEDNPDAQR
TFYGPNLWPDSDILPGWRQTMEKYHQQALEVGRKIARLIALALDLDVNFFDRPEILGN
PIAILRLLHYGGQVSDPAKGIYGAGAHSDFGFLTLLATDNVSGLQICKNKDAKPQVWE
DVEPLKGAFVVNLGDMLERWSNCIFRSTLHRVLGNGQQRYSIPYFVEPSHDCVVECLP
TCQSVQNPPKFPPVKYEEYLLQRYRETHADLGTYSKH
SEQ ID NO: 412
PWA75998.1|A0A2U1NRA0
MIQIAKNYFKLPEEEHLKSYSDDPNKTIRLSTSFNIRTEKVKNWRDYLRLQCYPLEDY
VHEWPENPESFKEHVSEYCASVSGLALELIEAISESLGLGRDYISAQLGKHAQHIALN
YYPPCPQPDLTYGLPGHSDLNLITILLQADEVSGLQVSKDGKWISVDPVPNTFIINVG
DQIQLQKKDYFKLPEEEHLKSYSDDPNKTIRLSTSFNIRTEKVKNWRDYLRLQCYPLE
DYVHEWPENPESFKEHVSEYCASVSGLARELIEAISESLGLGRDYISEQLGKHAQHIA
LNYYPPCPQPDLTYGLPGHSDLNLITILLQADDVSGLQVSKDGKWISVDPVPNTFIIN
VGDQIQVLSNDKYKSVLHRAVVNCDKERISIPLFYCPSKDAIIEPAPMLVTDDHPAVY
RQFKYGEYHETFWDRGLATENCLDMFMAGLKSD
SEQ ID NO: 413
PWA58899.1|A0A2U1MCA1
MLPVIDLSSSSEDRISTAHSIRQACTDYGFFYLINHGVDEQLLQNVFEQSRLFFSLPL
EDKMKCSRNIGGDFGFSPLYAENLDSSSSSKTKGDSKETFHIGPLQNQLNQWPSQDLL
PSWRSVMETYYTSLLSIGKKLSSLIALALNLDDHFFHSIGALDKPYAFLRLLHYPGEM
GDSDEDGVVYGASAHSDYGMITLLATDGVPGLQVCREKLKHPRTWENVNHVKGAFIVN
LGDLMERWTNCLFRSTLHRVMPTGKERYSMAFFLDPNPDCGRMLEKLLQ
SEQ ID NO: 414
PWA58959.1|A0A2U1MCG7
MAPSFNDENSLFNFVVKDGNGVKGLVDSGLTRVPGQYIQPPNHRINKQNATMSLENMT
IDLSELDGPNHDQVVKSIAHAAETLGFFQVVNHGVPLELLESLKMSAHQFFNQPSEKK
AVYLKGVSPSPMVKYGTSFVPEKEKALEWKDYISMIYTNDADALEFWPNECKLLHALM
GNLGVKLDDSSLDALIGLRMVNMNFYPTCPNPDLTVGVGRHSDMGMLTVLLQDGIGGL
YVKKGKDSSSGKEDWIEIPPIHGALVINIGDTLQIISNGRYKSAEHRVRTTSVESRVS
VPIFNAPLPMVKIGPLPEVVARDGVARYREVIFEEEVVVEYINISKELVKRLLKIGIL
GVKLDDSKLNTLIGLRMINMNFYPTCPNQDLTVGVGRHPDIGMVTILPRHGIGGFYVK
KPENWSLGLDRDSTPEALTHP
SEQ ID NO: 415
PWA79830.1|A0A2U1P2C6
MVIPTNTRVETLATSGIHQIPKEYIRTQDELTTITNIFDEEKKELGPQVPTIDLNDIN
SKDPKTRDKCCNELVKAATEWGVMHIVNHGISSDLINRVKDAGERFFDQPVEEKEKYS
NDIASGKMQGYGSKLANNACGQLEWEDYFFHLVFPEEKRDLTVWPSTPSDYLPATAEY
ARQLRALTTKILVVLSLGLGLEEGRLENEVGGLEELILQFKINYYPKCPQPELALGVE
AHTDVSALTFILHNMVPGLQLFYDGQWVTAKCVPDSIIMHIGDTLEILSNGKYKSILH
RGLVNKEKVRISWAVFCEPPKDKIILKPLPETISKENPPLFPPRTFHQHMEHKLFKKK
DDSVEPK
SEQ ID NO: 416
PWA76914.1|A0A2U1NTU8
MVLDNTSLSQPNYDRKAELKAFDETKTGVKGLVDAGITQVPRIFHVPSPQNLNSKQSC
PKLILPTIDLEGINEDSVRRKEVIQKVNDALKTWGFFQIVNHGISNSTLEEVKKGVRE
FFEQDDEVKKEWYTRDMSGKTRVVYNSNFDLYAAPVTNWRDTFFCMMAPYHPEPHELP
QPCSDILSKYSSQVLKLGGCVFELISEALGLNPNHLLDMGCAEGLAVLGHYYPSCPQP
ELAIGTTNHADNSFITILLQDHVGGLQVFYQEHWIDIPPIPGALVVNAGDLLQLITND
KFVSAQHRVLANKIGPRVSVASFFTTSSIPTLKVFEPIKELLTEDNPPKYRGTTVKEY
VDYFNKKGLDETSALLHFKL
SEQ ID NO: 417
PWA13620.1|A0A2U1K8G5
MSLNLSANRLEPFFKDQTAFVRLNHYPTCLAPNLALGAGRHKDAGALTILAQDDVGGL
EVKRKTDGEWIFVKPVRNSFIINLGDVIQVWSNDIYESIEHRVRVNSTRERFSIPFFM
NPALYIVVEPLPELISEENPAKYKGYHWGKFLATQKQ
SEQ ID NO: 418
PWA61707.1|A0A2U1MKD9
MASLTTEHKYDRLKEVKQFDESQIGVKGLLDTGITTIPPFFHQPPENLPSPQPKNQPR
LTVPVIDLSQDRSTVVDEIRGASSILGFFQIVNHSISVTSIESVLKDMKNFYEQTTEY
KMKFYNREVEKGVTYSTNMDLYKSKAASWRDTIQVWLSPMEPAWKMVPEMCRKALADW
DKAITGLVEELMSILCEGLGIKRDKLKEWSCLEGRLSISHYYPQCPQPELTVGLTAHT
DPTVLTVLVQNEIGGLLQVKCGEQWVEVEPVPGAIVVNIGDLLQMMSNDIYRSVEHRV
LANNVEGARVSVAHLFNPSNREKLFGPFPELISAEKPAVYHEFLHEDEMRRVLANNIE
TARVSISAFFSIQATKRGDIYGPFLELKECCKESPVTSHDRCPAFGHLVASSVSLFST
TNILCSLQNLENCICNIYKESTLVKNSDKYFRWRFNLLQKNVSM
SEQ ID NO: 419
PWA44525.1|A0A2U1L675
MVVATQINPIHVEKNQEMTIPTIDLTGDESKVSKLIVKACEEYGFFKVINHGVDYHII
KTIENESFEFFNKPLVEKQRAELAKPFGYGNKNIGLSGDTGELEYLLLQANQDFIDNT
SMLISNDPSSFSCRVNSYVKAVKGVACNILELMAKGIGGVPHSVFTTLLTEDDSDLLL
RLNHYPCVTHPFQHDANPIGFGEHSDPQILTLLASNGVPGLQVSLGDSRQWLPVTPDP
RAFFVIVGDLLKAMTNGRFISVRHRAMANTSTTESRLSMVFFGGPPPHATITCPPKLV
KRNTPLVYKPFTWAEYKSHTYAHRLGEARLDHFKLS
SEQ ID NO: 420
PWA71750.1|A0A2U1NE72
MVVATQINPIHVEKNQEMTIPTIDLTGDESKVSKLIVKACEEYGFFKVINHGVDYHII
KTIENESFEFFNKPLVEKQRAELAKPFGYGNKNIGLSGDTGELEYLLLQANQDFIDNT
SMLISNDPSSFSCRVNSYVKAVKGVACNILELMAKGIGGVPHSVFTTLLTEDDSDLLL
RLNHYPCVTHPFQHDANPIGFGEHSDPQILTLLASNGVPGLQVSLGDSRQWLTVTPDP
RAFFVIVGDLLKAMTNGRFISVRHRAMANTSTTESRLSMVFFGGPPPHATIACPPKLV
KRNTPLVYKPFTWAEYKSHTYAHRLGEARLDHFKLS
SEQ ID NO: 421
PWA78998.1|A0A2U1NZT5
MENMVTKWCTTVKSVPQDYVFPEGIRPGDQVVPIGKNFQVIDLENKAVFGSQKDVVQQ
IIQASQDSGFFQVINHGISEDLIHEAMEVIKELYNMPDEDIAKIYSLDPNKSCRLYTS
SFNYVNESTHVWRDNLKHPCHPLEDWVHLWPEKPSKYRQMAKXYPPCPEPGLTIGISK
HRDPNLLTILYQGNASGLQVLKDGQWYGVEPYANAFVVNIGQQLKVITNGKFESSEHR
VVTNSKESRYTIASFINPQSNAVIGPEKSLVDKDNGRPLYKPFHFKDFRLSFQEHMGE
GEATIGMFKIDG
SEQ ID NO: 422
PWA71549.1|A0A2U1NDF4
MVTMGAPVIDMQKVDGLAEELVXISLLFGFLLIKEIISKYIKSICDLNKFLGQKLMEG
CGLTGDLFDGWCCQLRLNKYHFCPESIGLNGTPTHTDPSFLTILQDDEDVNGLQVVDK
VSGEFVPFDHVPGTLAINIGDIGKVWSNGRFCSVKHRVLCFEPKTRYSIALFVMGPND
KIVQAPSELVDSEHPRLYVPIDVEKFRHVRNTTGLRAGDALDLFSTT
SEQ ID NO: 423
PWA98439.1|A0A2U1QK91
MDILNNLRENRTKQVKAFDDTKSGVKGLIDAAASGLVDIPRIFIRPTDELAEDLELNR
TSLQVPAIDLTGVGDKGGSRRKIVEEVKHAAENWGFFQVVNHGVPIKVLQDMLNGVRE
FHEQDVEIKKEYYSRDPERMVKFNSNYDLYMSRAANWRDTLIIDLLNTYHLDPQHLPS
VCRESTVEYLNYLTKLVDTLFELLSEALGLEPNHLKLLECEKGRSLACHYYPACPKPD
QTLGLNKHTDASFITILLQDEVQGLQVMHQNQWADVEPIPGALVVNIGDLLQIISNDK
FKSVIHRVFGNVALTRTSVACFFHGVATPPKIYGPIEKVITEECPQMFREFTVIDYMM
KFQSRGLDEKSGLNYVRL
SEQ ID NO: 424
PWA98227.1|A0A2U1QJV1
MDSLEPLQSNESLQELARKLKLEVPERYIQDHLEPTFVSYDSSPLPSIPVIDMNDLIK
NLGSDMDQLKNLRSVCHEWGIFQLVNHGVDKLLVENMKKEMTEFFKIPEEEKSRYKLK
AGEYEGYGQTLLHVQDQKVDWADRFYMITNPLHRRKSHLLPNFPPSIRDTMETYLQEL
QKLAMTLFGLIGQAIDIDKREMLDVFEDGMQSVRMTYYPPCPQPDLVIGLTPHSDASG
ITILLQVSDIEGLQVKKDGTWIPVNFLPDAFIINMGDALEVMSNGVYNSIEHRVMVNA
TKERISLAMFFNPKLDADVGPAKSLLKNTGNPPIYRTLVMEQYLKEFFSAKLNGKTFL
EKMKIESGENSGT
SEQ ID NO: 425
PWA71567.1|A0A2U1NDL3
MASVVAKTSSNISGSTTMVQEKGSRNKRKFRADPPPATELKSECLGYEFSAESFVTHE
HVNGCDMCCFGRESVDPVELDLGLSCSVGGTGGLSNGNRAEIEVSDEFHDADWSDLTE
SQLEELVLANLDTIFNLMILVEDNNNWRMILDNSDEVGNNCILVFSLLDKPMVVDDNI
RFGVIIFMNSTKIYDISSACRDWGAFHVVNHGVEKKLLDEIRRNGLGFFEEMSMEEKM
KYGCEAGSAASQGYGNDISSACRDWGAFHVVNHGVEKKLLDEIRRNGLGFFEEMSMEE
KMKYGCEAGSAASQGYGSKMLVADGGNDGVLDWRDYFDHHTFPLSRRDPSRWPHFPNS
YREVVAEYSDQMKILAQKLLGLISTSLGLSSSFIEDAMGELYQNITISYYPSCPQPEL
TLGLQSHSDMGFITLLIQDDVAGLQVLKDDQWVTVDPVSHAILVILGDQTEIITNGIY
KSSVHRAITNANKARLSVATFHDPAKTTTVSPAFKPPRYNQVVYGDYVKSWYTKGPNE
KGSRNKRKFRADPPPATELKSECLGYEFSAESFVTHEHVNGCDMCCFGRESVDPVELD
LGLSCSVGGTGGLSNGNRAEIEVSDEFHDADWSDLTESQLEELVLANLDTIFKSAIKK
IVSFGYTKDVATKAVLRSGLCYGCKDAVSNIVNNTLVFLRNGQEVDSLREHHFEDLHQ
MEKYILAELVCVVREVRPFFSTGDAMWCLLVSDMNVSHACTMDSEALSSLVGDGPSNG
CVSDTNGIPQKSNHSSLCAHSSSLEPHVTSSITPVTPSKTEVSHPKPKAPFVLNKFAS
EKENHVSTHRIVSKSFSPSSQNHEEKVGGRKITGINKRESILRQKSVHLEKSYRTYGS
KGVSRSGKLTNFGGLLLDKKLKSVSESTGINLKNISLKMSKGSITDNGPTQIGLAPSA
VFKTEIIDNSISALPKKNTPVISPSVDNPPVLLASETELSLSHPPKPNVTPVPEVSTP
SFSAKPYDKSFQQRISHAKKDETIMKLVPRVHELQNQLQEWTEWANQKVMQAARRLGK
DKAELKTLRQEKEEVERLKKEKQTLEDNTMKKLSEMENALCKASGQVERANSAVRQLK
VENNHLRHEMEAANVRAAESAASCEEVSKREKKTLMQFQSWEKLKSLFQEELIAEKRK
LTQLEQDLEVAKEQRDQLETRWKQEEKAKEDLVSQATSYRIGRLEAESSAKLREDLTR
LKADKSLQRYKQDIEKLEKEISLMSLKSDSLKIAALRGGIDGSYASKLTDIKTTPSQQ
ESQAPLIPKMADNFYGGVKRERECVMCLSEEMSVVFLPCAHQVVCKTCNELHEKQGMK
DCPSCRGPIQRRICVRYARS
SEQ ID NO: 426
PWA94844.1|A0A2U1QA13
MQTTPDYERKAELKAFDDTKKGVKGLVDAGITNVPRIFHVPTPQNVKNSIQPSLSQTC
LPTINLKGINKDMIRRKEVVKEVKDALETWGFFQMVNHGIPNSMLEEVKKGVKAFFEQ
DDEVKKQWYTRGLDGGKRKVVYNSNFDLYAAPVTNWRDSIFCIMAPDPPQSHELPEPC
RDIWPEYSSQVMKLGICVLELISEALELDPNYLLDMGCAEGLGVLSNYYPSCPQPELT
IGITNHADITFITILLQDQVGGLQVFYQDQWTDVPPIPGALVINAGDLLQLITNDKFV
SAQHKVLANKIGPRVSVASFFSTSTIPTNKVFGPIKELLSEDNPPKYRGTTIKEYVEY
FTGKGLDGISTLSHFKI
SEQ ID NO: 427
PWA53652.1|A0A2U1LXB9
MPSQEKLPVITFNTQTLNPSTDAWLSTSQAVRRALEDYGCFVVSTDKIPSDLHDTIFE
LSKDLFHLPLETKIKNTSDMLGFGYGNFSSYPFWEYFSIEYGDTLEANRSFTDLMFPS
GNNAFCENALKYMKILSEIDQCVMRMVYDSYGIDTKQCDQFIASMFYLARFIKYRAPS
EDKRAIAIDHPMTEKNFISILGDNNVKGLEIQMRNGEWIYHKPSPLTFVVIAGEPFMA
WSNGRIYAPMHQVAMRTPEEEATRYTLGIFSFTRETVKVPKELVEDSENYLRFKPFNH
LNFLKYVITEELQGYKCSIESYCGVTTTAPAKADY
SEQ ID NO: 428
PWA46655.1|A0A2U1LCC4
MTSEHRYDRLKEVKEFDSSKLSVKGLLDTGIKTIPRFFHQPPENLPSTQPKKKQPQLT
VPIIDMSQDRSKVVEEVRRSSSTLGFFQIVNHGVPLALIDCVVNSMKSFYEQPNEYKM
KFYHREVGKGASYSTNFDLFHSRGASWRDTFQVRMSPMEPAWDLVPEMCRASIADWDK
AVVDLAEELMSILCEGLGIKSDKLKELSCLEGRLCASHYYPHCPQPELTIGITSHTDP
CILTVLVQNEVGDLLQVKCGEEWADVEAVPGAIVINIGDLLQMMSNDEYKSVEHRVLA
NPVEGARVSVAVFFKPSNQEGLYAPFPELISAEKPAVYQEFLLADYTRRFYARELDGK
ALTSLYRVDNTKD
SEQ ID NO: 429
PWA85944.1|A0A2U1PJL3
MIVVGQVWLSPMEPAWKMVPEMCRKALADWDKAITGLVEELMSILCEGLGIKRDKLKE
WSCLEGRLSISHYYPQCPQPELTVGLTAHTDPTVLTVLVQNEIGGLLQVKCGEQWVEV
EPVPGAIVVNIGDLLQMMSNYIYRSVEHRVLANNVEGARVSVAHLFNPSNREKLFGPF
PELISAEKPAVYHEFLHEDFMRRFLSKEIVGKSKIDFYRINNTKDE
SEQ ID NO: 430
PWA64785.1|A0A2U1MU44
MESGDCDENKAFSIGEPPQENGLSHVPRSYEISSSNRPSLNPEIANVAVVDLVGLNDP
DQRPMVVKNIGSACRESGFFQIINHGISQKVLDEALGTAFAFFDLPTTEKEKYMSNDV
HKPVRYGTSIKDEVDKIQYRRGFLKHYAHPLNEWIQEWPDKPHDYRDKMGSYVMKARR
LSIEIMGAITESLGIGRKYLENKMEDGMQVMAVNCYPKCPEPHLALGVPPHSDHTCIT
IVLQSSCGLEIIDAADGTWRLVPELQGALEVHVGDHVEVLSNGLYKSMVHRVTLNNER
TRISIVSLHSMGLDEKMATAQELVSDENPKKYKESSFRDFLNFLTRNEGKSYIESLKM
SEQ ID NO: 431
PWA89797.1|A0A2U1PVR2
MADKCTIPVIDLHNLPNQLPELISACENWGCFRLINHHEILSTKLMLEMKKVVRSLLD
LPVEIKRRSSDAIAGSGYMAPSAKNPLYETLALYDMARSGDVERFCQELDATHDQREI
IMRYAEAVHELFMRIAKKLAEGLGVKRGDIGFENWPCEFRFNKYNFIPESVGSPGVQL
HTDSAFLTILQDDESVGGLEVMDKTGKFITINPWPDTLLVNLGDMATVRL
SEQ ID NO: 432
PWA73279.1|A0A2U1NIE3
MDGSDIYKAANSIRLGSLRVGSSSRASSLRNGSTSVWRNSGMDAFSKSVREEDDEEAL
KWASLEKLPTFDRLKKGLLFGSTGPSNEVDVDNLGPDDRRRILDRLVKTADEDNEGFL
LKLRNRLDRVGIELPTIEVKYEHVTVEADVNTGSRALPSFINFHIELVEVLLGLFHLL
PNSKRHITILDDVSGVVKPKRMTLLLGPPSSGKTSLLLAMAGTLAKELKCSGKVTYNG
HELHEFVPERTSAYISQNDVHIGEMTVRETLAFSARCQGVGSRYDMLAELSRRERDAN
IKPDPDLDIFMKAAATEGQEANVVTDYTLKLLGLDICADTMVGDQMIRGISGGQRKRV
TTGEMIVGPSKVLLMDEISTGLDSSTTFQIVKSLKQFIHILEGTAVISLLQPAPETYD
LFDDIVLLTDGKIVYQGPREHVLEFFESVGFKCPERKGVADFLQEVTSKKDQQQYWMR
RDMPYRFVTAKEFAEAYQSFHVGRKLATDLATPYDKSKSHPAALTTEKYGLNKKELLK
ACTDREILLMKRNSFVYFFKLFQLIVMAFISMTVFFRTEMHKRTLTDGGFYNGAAFFG
VTLLMFNGMSEISMTIAKLPVFYKQRNFLFYPSWAYAIPSWIMKIPVSFVEAAVWTIL
TYYVMGFDPNVTRFFKYFFILMLVNQMSSGLFRFIGALGRNMIVANTFGSFGLLLVFA
LGGFILSRDDIKGWWIWGYWASPMIENVLAYNKLVAELEEMVTRMVFETYGVEKYLDA
HRKMVTYLCRGMKYRPPVENETNMGFVPHTDKDFITVLHQNGVNGLEVKARDGEWFSV
ELVPSSYIVMSGDAAMAWSNERLYSPFHRVTMNGKESRYSIAQFSFLEGIIETPKEFV
DEEHPLLYKPFDHLKYLDFYNKEENRRLECAIRTYCGV
SEQ ID NO: 433
PWA43210.1|A0A2U1L2L8
MVLDSTSTIQPDYDRKAELIAFDETKTGVKGLVDAGITQVPRIFHVPSPQILRNSVEP
SFSKPSLPTIDLEGIDEDPKRRKEVIKEVKDALETWGFFQMVNHGIPNSMLEEVKKAV
KGFFEQDDEVKKQWYTRDFSGKRKVVYNSNFDLYTGTVTNWRDSFFCPMAPNPPQPHE
LPHPCRDIWPEYSSQVMKLGIFMLEIISEALGLDSNHLLDMGCAEGLCLLANYYPSCP
QPELTVGTTNHADIGFITILLQDHVGGLQVFYQDQWIDVPPITGALVLNAGDILQMIT
NDKFVSAQHKVLANKIGPRVSVASFFRTDSVPTLKVFEPIKELLSEHNPSKYRGTTMQ
EYADHFRAKGLDGTSPLLHFKI
SEQ ID NO: 434
PWA33902.1|A0A2U1KAW5
MESGDCDENKAFSIGEPPQENGLSHVPRSYEISSSNRPSLNPEIANVAVVDLVGLNDP
DQRPMVVKNIGSACRESGFFQIINHGISQKVLDEALGTAFAFFDLPTTEKEKYMSNDV
HKPVRYGTSIKDEVDKIQYRRGFLKHYAHPLNEWIQEWPDKPHDYRDKMGSYVMKARR
LSIEIMGAITESLGIGRKYLENKMEDGMQVMAVNCYPKCPEPHLALGVPPHSDHTCIT
IVLQSSCGLEIIDAADGTWRLVPELQGALEVHVGDHVEVLSNGLYKSMVHRVTLNNER
TRISIVSLHSMGLDEKMATAQELVSDENPKKYKESSFRDFLNFLTRNEGKSYIESLKM
DKMGSYVMKARRLSIEIMGAITESLGIGRKYLENKMEDGMQVMAVNCYPKCPEPHLAL
GVPPHSDHTCITIVLQSSCGLEIIDAADGTWRLVPELQGALEVHVGDHVEVLSNGLYK
SMVHRVTLNNERTRISIVSLHSMGLDEKMATAQELVSDENPKKYKESSFRDFLNFLTR
NEGKSYIESLKM
SEQ ID NO: 435
PWA78631.1|A0A2U1NYQ3
MGLTQDDFDRESELKAFDETKGGVKGLVDSGITKVPQIFIQPPDDFPQANNKFDLPNI
DLHGFDSNPVRRNEIVKEIKEASRTWGFFQVINHEIPVSVLREMRDGALRFFNMDHEV
KREWYVTDSTKKIFYNSNVDLSSTLPVRWRDSFHCRMAPDPPNPQELPPLCRDILIEY
SKQVTKLGSSLFELISEALGLRSSYLKDIGCADGLATICHYYPASPQPELTIGARKHA
DNDFLTVLLQDHIGGLQFLHKNQWVNVPFVPGALVVNIGDLLQLMSNDKVMSAEHRVV
SSCAGPRVSVACFFTTGMVETGKVFEPIKELLSEENPAKYRPTTMKAFVQHSHSKVFD
KSSMSHFRI
SEQ ID NO: 436
PWA91413.1|A0A2U1Q0F1
MAINHYPPCPDPSLAMGIGGHTDPNLITFLQQDHYGLQILKDGQWMGIEPIPNAFVVN
LGCQLQIISNGKLKSAEHRGVLNSTASRTSIVTFLGPDSTLPLVVGPAKELVTSTTPQ
VFKSYLYNEFLAEYLAFIRKPDPRNGTPLDPYRI
SEQ ID NO: 437
PWA56815.1|A0A2U1M6C8
MGLTQDDFDRESELKAFDETKGGVKGLVDSGITKVPQIFIQPPDDFPQANNKFDLPNI
DLHGFDSNPVRRNEIVKEIKEASRTWGFFQVINHEIPVSVLREMRDGALRFFNMDHEV
KKEWYVTDSTKKIFYNSNVDLSSTLPVRWRDSFHCRMAPDPPNPQELPPLCRDILIEY
SKQVTKLGSSLFELISEALGLRSSYLKDIGCADGLAAICHYYPASPQPELTIGARKHA
DNDFLTVLLQDHIGGLQFLHKNQWVNVPFVPGALVVNIGDLLQLMSNDKVMSAEHRVV
SSCAGPRVSVACFFTTGMVETGKVFEPIKELLSEENPAKYRPTTMKAFVQHSHSKVFD
KSSMSHFRI
SEQ ID NO: 438
PWA77334.1|A0A2U1NV27
MTSAVGSNYDRGSELKAFDDTKTGVKGLVDAGLSKVPSIFIHDQPITNKAHSSTTQTR
VNIPVIDLNGTNVRNEIIENVRDACENWGFFQIINHGIPKRVLDEMIDGVLKFHEMET
EEKIQYYSRDFRKRFFCVSNFHLFTDDAATWSDSFLSVMAPDCPQFEEMPSVCRDILI
EYSNHVMRVGLTLLELFSKSLGLEPNHLKNLGCAEGLFLLGHYYPACPEPELTLGANC
HTDAGFFTIILQDLLGGLQVLHQNDWVDVKPLSGALVVNVADLLQASLRYYEKFMSTI
V
SEQ ID NO: 439
PWA33873.1|A0A2U1KB46
MERGDCDEKKAFSIGETPQEKGLSHVPRSYEISLSHRPSLNPEIANVAVVDLVGLNDP
NQRSMVVKNIGYACRESGVFQIINHGISQKVLDEALGTAFGFFDLPTTEKAKYMSNDV
HKPVRYGTSIKDGDDKIQYWRMFLKHYAHPLNEWIQQWPDKPHDYRDKMGSYVMKARR
LAIEIMGAITESLGIGPKYLENKMEDGMQVIAVNCYPKCPEPHLALGLPPHSDYSCIT
IVLHSSVGLEIMDAADGTWHLVPELHGALLVHVGDHVEVLSNGQYKSMVHRVTLNNER
TRISIASLHSMGIDEKMETAQELVSDENPKKYKESSFRDFLDFLTQNDISEGKSFIES
LKMK
SEQ ID NO: 440
PWA58886.1|A0A2U1MCE6
MSFVPIIDYSLLTSGTPNERSKVIQQLGNACKDWGCYMLVNHGIPETLIKEIMDVSDE
FFNLPKEEKLEFEAFGVFDPIRFSSGFNAVEQNKDTLWREVLRLIAHPDFHCPHKPSG
FSEIASDYVKRTQVIVNELLKGVSESLGFEASYMNKELNLDSSFRLLAVNCYPFLPDF
DLARGLMPHTDHGVFTLLYENDVPGLEVFHNGKWVVMSGVPNAFLVLSADHLEIFSNG
VYKSKLHRAVVKDECKRITLVNTNGPSLDTVVGPSPRLVDEQDRPAGYLPMKYGEYLE
LQTKLTTAGKHAFDIVRIQN
SEQ ID NO: 441
PWA56435.1|A0A2U1M5A9
MEHGEISVGDFTSAMTLTHDHERGENIPERYILPPLQRPNPNLIDHPLTSLPVIDLSL
LNDPLLRSRATNEIHAACNNLGFFQVVNHGIPISVMQDALDVATEFFHLPSEEKMQFA
SANVREPVRYGTSMNHMVDKVLCWRDFIKHYANPIQEWIHLWPSTPSSYREKMGTYAK
AVHTLQKQLMAIVLENLGLNANYLHEDIEAGCQVMAVNCYPPCPKPDLALGMLPHSDY
GTITILNQNQQGLEIMDNDNTWHSVPFIQGALIVQLGDQVELISNGRHKSTIHRATVN
TERKRLSIASVHSLPIVKKVGPAPQLVDEQHPIAYKEGSFSEFLDYISVNCLLEARYI
DTLKIQ
SEQ ID NO: 442
PWA64784.1|A0A2U1MUA5
MDSGDSNENKAFPIGETPQERGFSNVPRCYEISSSDRPSLNPKIADVVVVDLSGLNDP
NQRPMIVKNIGNVCRESGFFQIINHGIPQKVLDEALGTAFEFFDLPTSEKAKYMSNDV
HKPVRYGTSIKDGVDKIQFWRVFLKHYAHPLNEWIQQWPDKPQDYRQYLFHQKIFLYL
SYVHCRDKMGDYVMKARHLAIEIIGAITESLGIGPKYLENKMEDGMQVMAVNCYPKCP
EPQLALGLPPHSDYSCITIVLHSSCGLEIMDASDGTWHLVPELHGALQVHVGDHVEVL
SNGLYKSMVHRVTLNNERTRISIASLHSMGMDEKMATAA
SEQ ID NO: 443
PWA71241.1|A0A2U1NCN5
MDISNSNDDRTKQLKAFDDTKCGVKGFLDTSAGGVVDVPQIFIRPPEEVAEDLELGRT
SLRVPAIDLSGVGDKGSCTREKIVEQVKLASEQWGFFQVVNHGIPVKVLEAMLNGVRE
FNEQDVELKKEYYSRDPERMVKLNTSFDFYMAKSASWRDTLSVDMLYSDHLDPQDLPS
PCRNATVDYLNQVMELADVLYELLSEALGLEPNYLKQLECEKGRTLACNYFPPCPKPD
LTLGVNKHTDASFITILLQDEVGGLQVLHQNKWADVGPIPGALVVNTGDLLQSMKSGF
LDSGGGGEKKKKKKDDSLAGRPILDTRTSRTGNLDLHSNLPKLSECWHRKFAIRKLGR
WNRRQAA
SEQ ID NO: 444
PWA91703.1|A0A2U1Q186
MSEMKMPREGVPVIDMQKFDTLSMEIVKASEEWGCFRIVNHGISMDLMAEMKTVVASL
FDQPEEIKMRTVHTEVGKGYVKRNLAGPCFEGFSIDDISLPGEFCDRINASVHQRKII
NKYIKAIRDLARLLGLKLMEGSGLNGDLFDGWCCQLRMNKYHFCPKSIGSTGVPIHTD
PTFLTILQDDENVNGLQLIDKNSGQFIPVDPVPGTLAVNIGDIGKLFSQIFKEIMCI
SEQ ID NO: 445
PWA96246.1|A0A2U1QE13
MIKILLKITLFPPMDAYIYLTKLSICSSSNNNKLAKITKKMEIPMIDFSKLDGEDRSK
TMALLHHACEKWGFFQIENHGVDKKLMDKVKKLVNQHYEENMKKSFYSSEIVKKVEDH
NKTTDIDWESTFFLWHRPDNKINEYTNLTEELRETVDEYINQLIKVAETLSELMCENL
GLDKDYIKEAFTGTKGPSVGTKVAKYPKCPHPELVRGLREHTDAGGIILLLQDDQVPG
LEFFKDDQWVNIPPSKNNTIFVNTGDQVEVLSNGLYKSTVHRVMTDKDGSRLSIATFY
NPAGDAVISPAQKLLYPNRYTFQDYLKLYSSTKFEDKGPRFESMKKTVNGY
SEQ ID NO: 446
PWA71818.1|A0A2U1NEA5
MEIPMIDFSKLDGADRSKTMALLHHACEKWGFFQIENHGVDKKLMDKVKKLVNQHYEE
NMKKSFYSSEIVKKVEDHNKTTDIDWESTFFLWHRPDNKINEYTNLSEELRETVDEYI
NQLIKVAETLSELMCENLGLDKDYIKEAFTGTKGPSIGTKVAKYPKCPHPELVRGLRE
HTDAGGIILLLQDDQVPGLEFFKDDQWVNIPPSKNNTIFVNTGDQVEVLSNGLYKSTV
HRVMTDKDGSRLSIATFYNPAGDAVISPAQKLLYPNRYTFQDYLKLYSSTKFEDKGPR
FESMKKTMCSQQLQITLPYEPSTKEHTYXLVNRRPDQETVDEYINQLIKVAETLSELM
CENLGLDKDYIKEAFTGTKGPSIGTKVAKYPKCPHPELVRGLREHTDAGGIILLLQDD
QVPGLEFFKDDQWVNIPPSKNNTIFVNTGDQVEVLSNGLYKSTVHRVMTDKDGSRLSI
ATFYNPAGDAVISPAQKLLYPNRYTFQDYLKLYSSTKFEDKGPRFESMKKTVNGY
SEQ ID NO: 447
PWA86280.1|A0A2U1PKJ2
MGSLDEGWPEPVVCVQSLSDSGELSIPDRYIKPQPERPSLSVNDMNIPIIDFNGLVTS
PEATMKQISKACREWGFFQLVNHGVRTDLVDGVRETWREFFHEPMEVKQKYANSPKTY
EGFGSRLGTEKGAILDWSDYYYLHYRPPCNTKWPDQQPSLREMMEEYTSEILRLGAAL
LKVFSVTLGLKEDSFQSAFGGNDFGACLRANFYPKCPQPDLTLGLSSHSDPGGMAILL
PDEQVCGLQVRKDDQWVTVKPEPHAITVNIGDQIQVSLFLSRVF
SEQ ID NO: 448
PWA43884.1|A0A2U1L4J8
MNSIISSRDTLEAYSRELKNIAIKTLLYIAKALRMESKDMIVLFEEGMQAMRMNYYPP
CPQPEQVIGLTPHSDAGGITFLLELNEVPGLQIRKDGIWIPVKPLPNAFIVNIGDTLE
IVTNGQYKSVEHRAIVNSKKERLSIATFLSPKLDGDLGPAPSLITPKTPPKFTRVTVV
DFFKNLFSRELIKKTNLEQYYV
SEQ ID NO: 449
PWA68692.1|A0A2U1N5A1
MDAYQNEMKLLTQKLLHLMLQTLNVTQEEMNWAISAQDSQTALQLNSYPSCPNPSNAI
GLAPHTDSLLLTLLHQGGANGLEIFVEGLGWSPVNPVDGAFVVNIGDLLHILSNATFQ
AVNHRARVNQAKQRISVAYFHGPPVESVVAPSSSFENPCFRSMLVKEFLSLKAKNFHK
ALSMIRK
SEQ ID NO: 450
PWA72871.1|A0A2U1NHC5
MEVLTSNWSNVQTLPDNYIFPPDRRPGTKKILSSKDIPIINLERINGPERDQIIQQII
KSSEDFGLFQVINHGVSRDLTDQTMAVFKEFFASPAELKAKFYSNDLNSSCRLYTSTL
NYENEEVHYWRDNFTHRCHPLEDHIKHWPEKPVNYREVVGKYSIEVRRFLMKILGLIC
DGMGLETGYLEGEMSKNQLISVNHHIPCPDPSLTLGMPEHSDPNLISMIQQGDICGLQ
ALKDGQWIGIEPIPGAFVVIPGLQLRVISNGKLASIVHRVVTDSKESRTTIGTFLTPS
NDILIKPADGLLGSMAPMYRGYTYEEFFSIFIGNNCVAERALECLKL
SEQ ID NO: 451
PWA85943.1|A0A2U1PJL1
MPPNDSAWKAVPEMCRAAFADWDKAVVGLAEELMSILCEGMGIKSDKLKEELCLERRL
SLSHYYPQCPQPELILGLTSHTDPCVLTVLVQNEVGGLLQIKCGEDWVNVDPVPGAIV
INIGDLLQIMSNDEYKSVEHRVLANTEEGVRVSIAVLFTPSNLEKIYGPFPELISDEK
PSVYNDFIYSEFIRRFEN
SEQ ID NO: 452
PWA50406.1|A0A2U1LN30
MAIPVIDFSKLNGEERAKTMAQIANGCEEWGFFQLVNHGIPEELLERVKKVSSECYKR
EREEEFFKNSTPVKLLRELVDKKSNEKLENIDWEDVFLLSDDNEWPPKTLGFKETMTE
YRAELKKLAEELMVVMDENLGIPKGYIKNAFNGGEGQKAFFGTKASSCSSRMKRWPDG
LQILKGGEWIDVQPLPNSIVINTGDQIEVLSNGRYKSVWHRVNSKRDGTRRSIASFYN
PSYDATIAPASQLVTKENEDINTFGYPKFVFGDYMTVYSEQKFLPKEPRFHAVKTV
SEQ ID NO: 453
PWA58900.1|A0A2U1MCB6
MLPVIDLSSSSEDRISTAHSIRQACTDYGFFYLINHGVDEQLLQNVFEQSRLFFSLPL
EDKMKCSRNIGGDFGFSPLYAENLDSSSSSKTKGDSKETFHIGPLQNQLNQWPSQDLL
PSWRSVMETYYTSLLSIGKKLSSLIALALNLDDHFFHSIGALDKPYAFLRLLHYPGEM
GDSDEDGVVYGASAHSDYGMITLLATDGVPGLQVCREKLKHPRTWENVNHVKGAFIVN
LGDLMERWTNCLFR
SEQ ID NO: 454
PWA85674.1|A0A2U1PIT8
MPPNDSAWKAVPEMCRAAFADWDKAVVGLAEELMSILCEGMGIKSDKLKEELCLERRL
SLSHYYPQCPQPELILGLTSHTDPCVLTVLVQNEVGGLLQIKCGEDWVNVDPVPGAIV
INIGDLLQIMSNDEYKSVEHRVLANTEEGVRVSIAVLFTPSNLEKIYGPFPELISDEK
PSVYNDFIYSEFIRRFEIKELAGKRKTDFCRIANTNKR
SEQ ID NO: 455
PWA73822.1|A0A2U1NJZ4
MVLDNTSLTKPNYDRKAELKAFDETKTGVKGLVDAGITQVPRIFHVPSPQNLNSNQPC
PKPSLPIIDLEGINEDPIKREKVVKEVNDALESWGFFQIVNHGISNSTLEEVKKGVRG
FFEQDDEVKKEWYTRDSSGNRRVVYNSNFDLYVAPVTNWRDTFICRMAPNPPEPHELP
QPCRDIWPKYSSQVLKLGGCVFELISEALGLNPNHLLDMGCAEGLAVLGHYYPSCPQP
ELAIGTPNHADNSFITILLQDHVGGLQVFYQDQWTDVPPIPGALVVNAGDLLQASLFP
SLITNDKFVSAQHKVLANKIGPRVSVASFFSTNTIPTLKVFEPIKELLSEDNPPKYRG
TTVKEYVDYYRGKGLDGTSALLHFKI
SEQ ID NO: 456
PWA46813.1|A0A2U1LCT1
MVLLSTSPFPQKYDRKAELTAFDEAKTGVKGLVEAGITEVPRIFHMPSPQNLNSNQES
HSKLCPPTIDLEGIHEDPIKRKEVIKQVKDAFETWGCFQMVNHGIPLSILEEMMEGVK
RFHEDDVEVRKQWYKRNGGGKRRVVYNTNFDLYTAPMANWRDTFYVTMAPNPPQPDEL
PPQCRDILLVYSSQMMKLSSCMFELISEALGLDPNHLGDMGCAEGLAVLGHYYPSCPQ
PELTLGTSPHSDTSLITILLQDHVGALQTLYQNQWTDIPPIPGALVVNAGDILQLITN
DQFISAEHKVLAKKVGPRISVASNISTGLVETGKIFEPIKELLSEDNPAKYRGTTVTE
YVNYYRKKGLDGISSLLHFKI
SEQ ID NO: 457
PWA34131.1|A0A2U1KBL6
MLTQYRSCLIVDLLPFYARDTNEEYAQNLIGVANKLLGLLSKGLGLEEDEVKQALGGE
DLVYMLKINYYPPCPCPELALGVAPHTDMSSITILVPNEVQGLQVFKDGDWYDVAYIP
NALIIHIGDQIEILSNGKYKSVYHRTTVNKEKTRMSWPVFLEPPPEFEVGPIPKLVNK
DDPPKYKTKKYKDYVYCKLNKLPQ
SEQ ID NO: 458
PWA65959.1|A0A2U1MXK0
MSINEEYHGSHFHEATFINQNGELQTSKIPVVQELAREGLTNHFPKRFISLPQEGQSI
DTIILETPPVIDIAKLKHNDTRKMELIKLAEAAKEWGVFLIKNHGVDDTVLDDVKDVV
KGFFALSFEEKKVNVGTYRSVDNMGQSIEKYVEKSRKVLDVLLEDLAESLSLDKNAFL
QYFEPQQSEIKVRVNYYPPCPRPDLAIGILPHSDPSGLTLLLEFGATGSLQVHKDTCW
ATLQWPNNNNLLVNIGDLLEIMSNGMLKSAWHRVRTQLNAERISLAYFYNPPPQCEIG
PLVNSDSMEEIYKKVVVEDYVSHLYKISPTTSKEAIMYAKVK
SEQ ID NO: 459
PWA80841.1|A0A2U1P4Z7
MANKKIEGMETEFAQFRTEINTRFAEINDKFAAMQQENKAMQQENMAMQEANTKRLDD
VMKALAALTTSTSKIQPKNEKKNTGPQYDDFEFLMNHRNLNETFDPKKKVEGVVFQDK
NSSVFRVGSGSNGIENHYTGGSKRGSFEADFRFRKLKMPIFEGEDAHGWIYRMERYFD
IQEIQEMDQLWAAVLCMEGPPYHGIVGVREGLRLGLGKGIAQDDTGREYVALFERLAC
QLVGISEPVLQGTFINGLKPELRASVHVMQPEVLNHAMKLSISIDENKTCYNVLWGAI
PKPPVELFFTKSSKQNNNHHAKDFITIPIIDLSKPVSKHLLVKARQDFGFFKAGPPNP
FGYGNKRLGKNGDFGWVEYLLLDTKLEHDHKNPIFPIFEDNPKKFKCVVDDYVSVVKK
MACVILVFLADELKLPQNDESKMLMDEQSDSVFRVNYYPPCPDVQELEMKGKKLIGFG
EHTDPQIISFLQSNNTTGLEILLKDGSWMCVPPDPNSFFVNVGDCLQVQALIHEKVMT
NGRFKSVKHRVVANSLKSRLSMIYFGGPPLSQKIAPLPSLMEGDDVILYKEFTWFEYK
KSAFSSRLSDNRLSLYEKDHKVLS
SEQ ID NO: 460
PWA66042.1|A0A2U1MXQ3
MDAYQNEMKLLTQKLLHLMLQTLNVTQEEMNWAISARDSQTKLQLNSYPPCPNPSNSI
GLAPHTDTLLLTLLHQGGANGLEIFVEGLGWSPVNPVDGAFVVNIGDLLHILSNATFQ
AVNHRVRVNKAKQRISVAYFHGPPVESVVAPSSSFENPCFRSMLLVEALKSTVMEPLL
ESVNSIIGCQKIDLYFEADCNYIEKACVLVLEYIAAKDHKSANDVRNEDDDHKTTKFR
RLVSVNPIKVALITGITGQDGSYLTELLLSKGYSVHGLIRRSSNENTQRINHIYIDPH
NAHKARMKLHYADLTDASSLRRWIDTIAPDEIYNLAAQSHVAVSFEIPDYTADVVATG
SLRLLEALRSHISATGRKGVKYYQAGSSEMFGSTPPPQAEDTPFHPRSPYAASKVAAH
WYTVNYREAYGIYACNGILFNHESPRRGENFVTRKITRAVGRIKIGLQNKLFLGNLQA
SRDWGFAGDYVEAMWLMLQQEKPDDYVVATEESHTVEEFLEKAFGYVGLNWKDHVEID
KRYFRPTEVDNLKGDSTKARKVLGWKPKVGFEQLVKMMVDEDIELAKREKVLVDAGYM
DAQQQP
SEQ ID NO: 461
PWA72581.1|A0A2U1NGH4
MRMNYYPPCPQPEQVIGLTPHSDAVGITFLLELNEVPGLQIRKNDIWIPVKPLPNAFI
VNIGEILEIVINGQYKSVEHRAIVNSEKERLSIATFLNPKLDGDLGPAPSLITPKTPP
KFTRVAVVDFFKNLFSRELNRKTNLEQYYV
SEQ ID NO: 462
PWA77333.1|A0A2U1NUX3
MLLNTSPIQNYDRKAELKEFDETKTGVKGLVDAGITQVPRIFHVPSPQNHRNSIEPSL
SKPSLPTIDLEGIDEDPIKRKKVIKEVNDALESWGFFQIVNHGISNGTLEKVKKGVRG
FFEQDDEVKKEWYTRDYLSGNRRVVYNSNFDLYAAPVTNWRDTFFCSMAPNPPEPHEL
PQPCRDIWPKYSSQVLKLGGCVFELISEALGLKPSHLLDMGCAEGLAVLGHYYPSCPQ
PELTIGAPNHADNSFITILLQDHVGGLQVFYQDQWTDVPPIPGALVVNAGDLLQASLY
LPLSLSHYTISFCWTLITNDKFVSAQHKVLANKIGPRVSVASFFSTSAIPTLKVFEPI
KELLSEDNPPKYRGTTVKEYVDYYRGKGLDATLNANVTIFGQLFHVSIWVEFPTDRQN
IHPATDIKEWPQWPQPMSISVVMQARPKVSVPLCQALGKLLRKYEGLEVRFKQTQEIL
KVEIDVSEEEEKNKEIEAKEDEDVIEVLDVEKHRKKKAKAYQTLRARATAVPLFEVMR
NFPVSEIPTKLACFMINSLQTDSMNLMLPVENICITPETMKVMFWHTDGSLVVKYQVL
KNVLEDDDVQDIDWLTYISKCARYSKYDWNKLKKNKEVVYYGPVTFLMLVYLHYTKFE
NMEVWRKVPAFKSWNANLMKRREMLVMQRNNFGLVELIGDINEEKENVQKEELYIEDK
IENILSEKEGLSNDDDDDDDDDDDDDDDDDDAQHANQYVEQCNENA
SEQ ID NO: 463
PWA65030.1|A0A2U1MUX0
MSSITILVPNEVQGLQVFKDGDWYDVAYIPNALIIHIGDQIEILSNGKYKSVHHRTTV
NKEKTRMSWPVFLEPPPEFEVGPIPKLVNKDDPPKYKTKNKLTAQKLRLLYSHFQNTS
VYNAKCKVNLTYFTSSLLSNTFFFSKCTILHVYRDTNEEYAQSLIGVANKLLGLLSKG
LGLEEDEVKQALGGEDLVYMLKINYYPPCPCPELALGVAPHTDMSSITILVPNEVQGL
QVFKDGDWYDVAYIPNALIIHIGDQIEILSNGKYKSVYHRTTVNKEKTRMSWPVFLEP
PPEFEVGPIPKLVNKDDPPKYKTKKYKDYVYCKLNKLPQ
SEQ ID NO: 464
PWA93644.1|A0A2U1Q6K3
MASPNLLSQSDRSVQELAKTFKHDVPHRYIQEHREPTFVSNNATSLPSIPVVDMNDFI
NILGSENDQLKNLRSVCQEWGIFQLVNHGVDKLLVDKMKNEVVKFFDMPVEEKLRYEL
KAGDYQGYGQTILHAQDQKIDWADRFYMITNPHHRRKPNLLPEFPPSLRDTMENYLQE
LQKLAMTLFGLVGQAVDIDNQEMIDVFDDGMQSVRMTYYPPCPQPDLVIGLTPHSDAA
GITILLQVNGVEGLQVKRDGVWIPVNFLPDAFVVNVGDILEVRPPPHFLKLKKKL
SEQ ID NO: 465
PWA83200.1|A0A2U1PBU4
MRNSPKVYFKSIKHGVPLALIKKLRVLESRKAFCTPTDEKHKVLRSANGVTGYGTASI
SKFFDKCMWHEGFGIMGSCVEDAKILWPYDYKNFCNTMDAYQNEMKLLTQKLLLLILQ
TLNVTQEEMNWALSAQDSQTKLQLNSYPSCPNPSNAIGLAPHTDTLLLTLLHQGGANG
LEIFVEGLGWSPVNPVDGAFVVNIGDLLHILSNAIFQAVNHRVRVNQAKQRISVAYFH
GPPVESVVAPSSSF
SEQ ID NO: 466
PWA86689.1|A0A2U1PLR8
MYPKFLKELQKKSRGVADSRERQLPVIDLSFKKVNSSSSSWVATSAKVRHALEEYGCF
IALYGVSKDLHDAAFIASQEVHDLPVEVKVRNVLDATGYGYSGEKLAKMPLYERLSIE
DAATAEGAERFTKLMWPSRNDTLWDKSFMTVIHQREEGKSLEIQAKDGEWIQVDLSPS
SFIVMAGDVCMAWSNGRIQAPYHKVMMQGNKDRYSLVLSSFIRDLKIEVPQELIDEDH
PLQFKAFDHYEYIQYVDSNTIQGVRLQDAIKSYCGI
SEQ ID NO: 467
PWA36712.1|A0A2U1KJ04
MIALFEEGWQAMRMNYYPPCPQPEQVIGLTPHSDAAGITFLLELNQVPGLQIRKDDIW
IPVKPLPNAFIVNIGEILEIVTNVQYKSIEHRAIVNSEKERLSIATFLNPKLDGDLGP
APSLITRKTPPRFTRVTVVDFFKNLLSRELNRKTNLEQYYV
SEQ ID NO: 468
PWA45387.1|A0A2U1L8R1
MSNGVFKSRVHRALVNPKYERMTLAMFCMPHTEKGIGPFLEKVRESSILIFRSSMKDK
KKCLRAEDDFEGYGNDVILLDQQTLDWVDRLISLLDQNTNKSFNFGLKILLTSVFGLK
PHADGTAITVLLQDKEIEGLQLLKDGQWVGVPIVRDALTINVGDQIEIMSNGVFKSRV
HRALVNPKYERMTLAMFCMPHTEKGIGPVDELITDETPRLYKNVKYSLDFYFEH
SEQ ID NO: 469
PWA66895.1|A0A2U1N055
MAEVVPFDLVEQILLRLGVKDLLRCKSVCKLWQSLILSYRFVKYHMNHANNKDNNNKE
IGHRRILMPEIYRMNMAYDMWYYMNSWQIVGSSNGLVCVTPVDAQVYVANPSIREVKD
LPTPPICIRNEEDRKDLCWGFGYDSSIDDYKVVIGVVVVVEGKYLTRFQLLTLKSNTW
KFIGDLKYTSFSTFASLCNGSLHWIMKDSITKKWAIISFNLSQEKFKEISQPDDSRYA
FDSTDISRKLGIVEGCLCIFNVETVPNNTWVMKNYNVSSSWELLPYDCLYNTRKYIEQ
DDHMYRCSYIRTMYDIAYKPIFNIQPNDVFYNDKDIYLSKTTAHMGDPIFVESLVSPH
VNGRPKQVYLGLEKTTNGPRRVVESVRARLCINKWRHPWDLTTTSFVFSHHRNATIEF
KIRHLAWMEANYGKISSNGYNSFCLILREVVRKIARLIALALDLEANFFDRPEFLGRP
IAVLRLLHYEGQVSDPTKGMYGAGAHSDYGLVTLLATDCVSGLQICKDKDAKPQVWED
VKPLKGAFVVNLGDMLERWSNRIFRSTLHRVLVNGQERYSIPFFVEPSHDCVVECLPT
CQSKLNPPKIPFYLMSIACLVGVVYISTVLGFLLSSAKSTFFRDIGIPMLI
SEQ ID NO: 470
PWA96718.1| A0A2U1QFF9
MHHFILRTMITQLVLKFLSNSTTGDSKETFHIGPLDGEESLQNQWPSKELLPSWRFVM
EKYYKMLLSTGKRLSSLIALALNLEEDFFEKIGAVDRPSAFLRLLHYPGDMGVSDEVV
YGASAHSDYGMMTLLATDAVCREKNKHPRTWENVTHVKGAFIVNLGDMMERWTNCLFR
STLHRVMPTGKERYSVAFFMDPNPNCIVECLKSCCSESSPPRFPPILSGDYLRERIHD
AYSNVKTQSKCTKGCNLALASYYVAQGSNLTYISRIFSLSITEILKYNSQIPRGDSIE
TGTRINVPFSCLCLNGDFLGHTFLYQTQVGDTYGKIARDVFANLTDEYWVQRVNSFAP
AFIPDFAYINVTVNCTCGNKHVSKDYGLFVTYPLQPGEDLQSLTSESGVQTTLLEQFN
PSSNFSGGSGLVFVPAKALCASVASTIDRHKM
SEQ ID NO: 471
PWA39945.1|A0A2U1KT63
MNTEEANELVNHGISHELLDRVERMTKEHYKKCMEVRFKENGGSQRIRRISRGKKAFY
GSKSTAPNFGTKVSNYPPFPTPDLIKGLRAHTAAGGIILLFQDDKVSGLQLLKDVQWI
NEPAMRHTIVINLGDQIEVITNGKCKSVLHRVIAQTDGTKMSFASFYNPGGDAVIYPA
KILMMEHT
SEQ ID NO: 472
PWA91410.1|A0A2U1Q060
MAASSASSETLFSMTAFNHMMHMKLSSSNYLVWREQMLLVLDFHTLSAHVAADATPPP
ALITVAGKSSPNPDAAAWFDKDQKAVLLIKSSLTEEAAAEVLGLKGARDIWTALEQAY
SNASVERIHSLRDSLRLIKKGRGSSRGRGRGQQQRRPPHCQLCRTNGHYASACPELHS
FASKAPSDESLVKAFHAQCHVTDDSPDWRADSGATDYMVPPTDSVHNSTPYKGNNCVV
FGNGSSQHIDLSSLVVTSFLDEPSFVSTVEHHRSPSTTSMPSSDTNMLPPFNFFEAES
NAPAQQHVSETTSPVHEPTMSTDPTPVSGPENSSPNTATSSGPASASVLDQSTSAHPM
QTRSKADVIGTYSLEVRKLSLRMLELICEGLGLERGYFEGDLTMGQLLSINHYLPCPD
PGLGLGLPKHADPNIITVLFQENIYGLQVFKDQQWLGVEPLPHTFAVNIGHQLYVINH
GVSQDLMDNTMKVAKDFFNMPNEENVKLYSNDPTKSCRLSTASYHYDKEHFWRDNLTN
VFKDGQWLGVEPLPHAFVVNIGYQLEIISNGKLRSVAHRAVTNSKEDRTSIVTSIDPC
QDTIIEPAKSMIDTGIPLYRPFRNRDFRKFFTEKKGDTEALMAAYKFKP
SEQ ID NO: 473
PWA45388.1|A0A2U1L8Q5
MNMQLQQISSDFDQLPARYVHKEYKDFSGIGLSHVPSVDVPVIDFSLLTSSPFELDKL
KSAISTWGCFQAINHGTEGSFLEKVREISVLFFRSSMKDKKKCLRAEDDFEGYGNDVI
LLDQQTLDWVDRLISLEVLDEYTSKIELINKVVLKALARSLNLEDNCFLNQYGTTAKM
HARFNYYPPCQWANNPHADGTAITVLLQDKEIEGLQLLKDGQWVGVPIVRDPLTINVG
DQIEC
SEQ ID NO: 474
PWA53658.1|A0A2U1LXD3
MSNSLDFRAPPPSPVGPGRRSTVANDDILTDFLHQTLIVPDLVLPDRVFPRQNPNIQS
LPKLDFKKLSFSDDFKLEDVIEVIAQTGCFELVNHGISRKLFRDVKECGKRVFGLEDE
KKRVILRSNERLYGFVEVNGDDKDLIKSMSNSLDFRAPPPSPVGPGRRSTVANDDILT
DFLHQTLIVPDLVLPDRVFPRQNPNIQSLPKLDFKKLSFSDDFKLEDVIEVIAQTGCF
ELVNHGISRKLFRDVKECGKRVFGLEDEKKRVILRSNERLYGFVEVNGDDKDVSEEFV
WCRDESLRSEMEGVWMSYTDFSEKMENLMSKIENISESLLKLFLDPYTPKSRVDEEST
RDKETMGSMCYLYKHSKNTDNLSNDDEYMDSLSYDVIRMLIRGSEHKHTLCFHVCDGS
SEFHVYSKKGWVSFSPDKNALVVTIGDQLQTWSEGKYKHVIGRPIFKGELEDCISMAF
LYSPPISKGQEDKTISLVNQILLALFLTFIYNLFSYIST
SEQ ID NO: 475
PWA53947.1|A0A2U1LY85
MGSLDEGWPEPVVCVQSLSDSGELSIPDRYIKPQPERPSLSVLNDMDIPIIDFNGLVT
SPEATMKQISKACREWGFFQLVNHGVRTDLVDGVRETWREFFHEPMEVKQKYANSPKT
YEGFGSRLGTEKGAILDWSDYYYLHYRPPCNTKWPDQPPSLSLLRAISTQTCPLTRLE
LGVDLSFLVSSGAKWPSYPVSKSVDSNVRKMNHMVLSNAIYKSVEHRVVVNPNKERVS
LAYFYNPKSDMLIQPISELVTSKKPALYLPMTFEEYRRFIRTKGPQGKFQVESLKSPR
SEQ ID NO: 476
PWA68466.1|A0A2U1N4P5
MIIEPMETVAAEIKHASQFTTKETTEMKDISEFMLAEPKETPEMKDFSEFTLAEVTKV
EPEWLVEIATHYYERKNLKYFRAPPPSPVGPGRRSTVANDDILTDFLHQTLIVPDLVL
PDRVFPRQNPNIQSLPKLDFEKLGFSDDFKLEDVIEVIAQTGCFELVNHGISRKLLRD
VKECGKGVFGLEDEKKRVVLRSNERLYGFVEMSXEVIAQTGCFELVNHGISRKLLRDV
KECGKGVFGLEDEKKRVVLRSNERLYGFVEVNGDDKDVSEEFVWCRDESLRSEMEGVW
SNYSDFSEKMENLMSKIENISESLLKLFLDPYTPKSRVDEESTRDKETMGSMCYLYKH
SKNTDNLSNDDEYMDSLSYDVIRMLIRGSEHKHTLCFHVCDGSSEFHVYSKKGWVSFS
PDKNALVVTIGDQLQTWSEGKYKHVIGRPIFKGELEDCISMAFLNSPPTSKGQEDNTI
SIGNQILLALFLTFIYNLFSYFST
SEQ ID NO: 477
PWA68465.1|A0A2U1N4N9
MIIEPMETVAAEIKHASQFTTNETTEMNLDVPEFTLPEPKETPEMKDFSEFTLAEVTK
VEPEWLVEIATHYYERKNLKYFRAPPPSPVGPGRRSTVANDDILTDFLHQTLIVPDLV
LPDRVFPRQNPNIQSLPKLDFEKLGFSDDFKLEDVIEVIAQTGCFELVNHGISRKLLR
DVKECGKGVFGLEDEKKRVVLRSNERLYGFVEMSXEVIAQTGCFELVNHGISRKLLRD
VKECGKGVFGLEDEKKRVVLRSNERLYGFVEVNGDDKDVSEEFVWCRDESLRSEMEGV
WSNYSDFSEKMENLMSKIENISESLLKLFLDPYTPKSRVDEESTRDKETMGSMCYLYK
HSKNTDNLSNDDEYMDSLSYDVIRMLIRGSEHKHTLCFHVCDGSSEFHVYSKKGWVSF
SPDKNALVVTIGDQLQTWSEGKYKHVIGRPIFKGELEDCISMAFLNSPPTSKGQEDNT
ISLGNQILLALFLTFIYNLFSYFST
SEQ ID NO: 478
PWA80687.1|A0A2U1P4S9
MASLTAEQNYDHLKEVTQFDESKLGAKGLLDSGITTIPRIFHQPPENLPGPQPKTRPQ
LTVPVIDLSQDRSKVVEEIRRCASTLGFFQIVNHSIPVTLIGSVLNDTKKFYEQSTEY
KMQFYGRETENGATYATNLDLYQSKAASWRDTIEVWMSPKEPKWEAVPEMCWTALKDW
DKAVVGLAEELMAILCEGLGNEIGGLLQAKCGEDWVVVEAVPGAVVINIGDMLQMMSN
DAYRSVEHRVLANNAEVAPLSFAVFFEPSNREGLYGPFPELISAEKPVIYHKFMFEDF
CRRFLSKELDGKSKLDYYRIDNTNKA
SEQ ID NO: 479
PWA36538.1|A0A2U1KIJ0
MKTHKIICQICSSRETGFESLRPTVALLYEDGSPVEAKGVGRRVLVLILKVFLLVMML
LVYQQQVCREKLKHPRTWENVNHVKGAFIVNLGDMMERWTNCLFRSTLHRVMPTGKER
YSAHVSRMQSIARRLARDSSAYGPAIRSIYPISDQYYGGYGYDRRYASTVPAKGVGHL
RLGMLVLIDVEVSDLSKCEIGIVATVFGATGFLGRYLVQELAKMGSQVLVPFRGSEDS
PRHLKLMGDLGQIVPMKYNPRDENSIKAVMAKANVVINLIGREYGTRNYSLEEVNHHM
AAQLAKISKEHGGIMRFIQLSCLGASKSSPSRLLQAKAAAEESILNELPEATILRPAV
MLGTEDRILNPWAQFARKYNFLPLVSGGSTKIQPVYVADVASAVVAALKDDGSSMGKL
YELGGPDIHTQRELAEIMYEVIREWPHYVNVPLPIAKAISSPREIFLNKVPFPLPSPS
IFNLDMITAYSSDKLVSEDDGKIGNLGGLGHGSNRSFKVIQEPNLRKRHLGDLQGLVY
VDAPTLKTKAYEALQSHGMDVEIPGGGESLNQLSRRCTASLQTIARERVVVVTHGGVI
RALHERASTGKRHRVGRILNVSVNVLHLTDTDKWVIKSWGDVSHLNGAGYLDSGFGCD
KTSG
SEQ ID NO: 480
PWA92255.1|A0A2U1Q2Q9
MARRDSLPVKTAQQMAMDGDQPPSKYIYSNNTSYAKFGPLETSPPFAPVPVIDIGCLL
SSCKQDEQENELAKLRSALTTWGCFQAVNHRLSDSYLDNIRQVIKQFFELSMEDKRKY
FRETGSVEGYGNDMTYSETQVQDWCDRLFLRILPEDQRKLRFWPENPSNFRETIDDYT
KKIMSISVVIFKAMAKSLELEENCFSKHFTEESDVLQGRFILYPPCPTPDKVFGLKAH
SDRSGITLLLQDPGVEGLQVLNDSKWYMVPVIPEALFINLGDQMQILSNGIFKSPVHR
VVTNSDKGRISVAMFTEPEPNKEIGPVDALVDEKRPRVYKTVKDYAVFNHECFQKGVV
ALDAVKL
SEQ ID NO: 481
PWA47829.1|A0A2U1LFQ5
MMKQTQDIAENCDQPPERYIHKQDEEYGANNVGNAAHVITAAAIPVIDFSLLNSSPSE
LVKLKSAATTLGCFQAINHGIESSFLEEVREISKLFFKLPADEKKKCLKEENDVQGYG
NETVFSDNQTLDWTDRLYLTVLPQHQRRLQFWPQNPTHFRKVLNEYSFKIESLNVVVL
KALARSLNLEEDCFMNQYGKTSKINARFNYYPPCPLPEKVLGLKPHGDGSAITFLLQD
KEVEGLQLLKDDEWVGVPVVSNALTINIGDQMEIMSNGIFKSPLHRVFVNSKNERMTL
AMFCMPQTEKNIGPVDGLITDETPRLYKDITFTLDFMHKNYQQGRRAINACKI
SEQ ID NO: 482
PWA95190.1|A0A2U1QB04
MVLSENQTLDWTDRLYLTALPQDQQRLQFWPQNPTNFREILDEYSSKIQLVNEVVLKA
LARSLNLEEDCFLNQYGTTAKMQARFNYYPPCPWPEKVLGVKPHADGSAITVLLQDKE
VEGLQLLKDDQWVGVPIVPDALTINVGDQIEIMSNGIFKSPVHRVFVNSKNERMTLAM
FCMPQTEKDIGPVDGLITNERPRLYKNVTFTLDFFFKNYQQGRRPIDACRI
SEQ ID NO: 483
PWA95191.1|A0A2U1QB13
MMFDWFQAINHGIDGSFLEKVREISKLFFKLPADEKKKYLREENDVEGYGNDMVLSEN
QTLDWTDRLYLTALPQDQQRLQFWPQNPTNFREILDEYSSKIQLVNEVVLKALARSLN
LEEDCFLNQYGTTAKMQARFNYYPPCPWPEKVLGVKPHADGSAITVLLQDKEVEGLQL
LKDDQWVGVPIVPDALTINVGDQIEIMSNGIFKSPVHRVFVNSKNERMTLAMFCMPQT
EKDIGPVDGLITNERPRLYKNVTFTLDFFFKNYQQGRRPIDACRI
SEQ ID NO: 484
PWA95189.1|A0A2U1QB08
MMFDWFQAINHGIDGSFLEKVREISKLFFKLPADEKKKYLREENDVEGYGNDMVLSEN
QTLDWTDRLYLTALPQDQQRLQFWPQNPTNFREILDEYSSKIQLVNEVVLKALARSLN
LEEDCFLNQYGTTAKMQARFNYYPPCPWPEKVLGVKPHADGSAITVLLQDKEVEGLQL
LKDDQWVGVPIVPDALTINVGDQIEIMSNGIFKSPVHRVFVNSKNERMTLAMFCMPQT
EKDIGPVDGLITNERPRLYKNVTFTLDFFFKNYQQGGCKSFAVSSSSSVPLTNLKIWK
A
SEQ ID NO: 485
PWA82193.1|A0A2U1P8W4
MMQIAKDFFSFPEEERLKSYSDDPNKATRLSTSFNIQTEKVANWRDFLRLRCYPLEDY
VHEWPTNPSMFRAHVKEYCTIVRGLALELVEAISESLGLERDYIGKQLGKHEQHMALN
YYPPCPQPDLTYAQVLSNDKYKSILHRAVVNCDKERISIPTFYCPSRDAMISPAPQLV
TKDQPAVYRPFRYGEYYDKFLNRGLATENCLDMFMATKSTRSA
SEQ ID NO: 486
PWA80743.1|A0A2U1P4P5
MTSPTTEANTYDRETELKEFDSSKLGCKGLLDTGIKTIPRFFHQPPENLPCKQTNLKK
NMQTVPVIDMSQDRSEVVEQVRKLSSTLGFFQVVNHGVDVQMIESVINGIKDFFEMDN
EYKMRFYSRDAEKGAVYSTNFDLFHSKAASWRDTLQPQRLYFNPLRSIPPNHILPLDH
AVGAYRRGGNGVHRRVHMNGLSWVVFDLNWVKQTSTVIYKETYRETGQTGSPKADFKA
YNLLELSGPVWVRVHCHKDLIRVKHSGLFREVRIAPIKPDWETVPEMCREALKDWDKA
VVGLGDELMSILCEGLGVKSDKLKELSCLEGRASVSHYYPPCPQPDLTLGISSHTDPG
VLTVLVQNEVGGLLQVKCGEDWAAVEAVPGAIVINIGDLLQYYPISQMMSNGEYKSVE
HRVLANPEEVARVSIAVFLTPGIRESLYGPFPELISAERAAVYKEFILADYLRREALK
DWDKAVVGLGDELMSILCEGLGVKSDKLKELSCLEGRASVSHYYPPCPQPDLTLGISS
HTDPGVLTVLVQNEVGGLLQVKCGEDWAAVEAVPGAIVINIGDLLQYYPISQMMSNGE
YKSVEHRVLANPEEVARVSIAVFLTPGIRESLYGPFPELISAERAAVYKEFILADYLR
RFFTKELDGKSLTNFYKIDDTKS
SEQ ID NO: 487
PWA67117.1|A0A2U1N0V5
MKTTNKKSKLGSGDGEFRSDAEITNFDDRRYQGRITTDESRSMHTEATNLSNTQWVLG
FQIAVNGPSPAADQHSETDIYVCAGEYFTIKPDWEAVPEMCRQSLKDWDKAVVSLGDD
FMSILCEGLGVKSDKLKELTCLEGRVSVSHYYPQCPQPELTLGITPHTDPGVLTVLVQ
NKVGGLLQVKYGEDWAAVEAVPGAILFQS
SEQ ID NO: 488
PWA42446.1|A0A2U1LOB9
MSTYKFFVITIIFAPVTPCISQLEGGIVQVAIEVEDRAQHSAITRVNTANDVRVTVAA
PAARGKSNNELCLSSFLHLDNIFRDRLTGAGDLLSRRNLSAGIRHELSTNRTSEDVLS
SKYVRSNMTRIESYQAPKDWDKAVVSLGDELMSILCEGLGVKSDKLKCLEGRVSVSHY
YPQCPQPELTLGITPHTDPRVLTVLVQNEVGGLLQMCPRKQEVVPTRCMKRICVYIRS
LEDVFAILEPPTKPSHDQDHGERKPILINRVYYIESEQQKRVQLFVSENGCRRSKVVA
FTSLFWKIVALSMEESGASNKVCNMALAVDGRRRLNEIAGEDNEMLLILHFSNVLSMP
SGAIRAQVGINIPFSFQALSVETTESSGTQVFEQIPKIMLDLYFSALD

What is claimed is:

1. A microbial host cell for producing artemisinic acid, dihydroartemisinic acid (DHAA) or a derivative thereof, the microbial cell expressing a biosynthetic pathway comprising:
a heterologous enzyme having an amorphadiene synthase activity (ADS), a heterologous enzyme having an amorphadiene oxidase activity (AO), and a heterologous enzyme having a double bond reductase activity (DBR).

2. The microbial cell of claim 1, wherein the ADS comprises the amino acid sequence of SEQ ID NOs: 1 or a variant thereof.

3. The microbial cell of claim 2, wherein the ADS comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO: 1.

4. The microbial cell of claim 2, wherein the ADS comprises an amino acid sequence having from 1 to 20, from 1 to 10, or from 1 to 5 amino acid modifications independently selected from substitutions, insertions, and deletions with respect to SEQ ID NO:1.

5. The microbial cell of claim 2, wherein the ADS comprises one or more amino acid substitutions at positions shown in FIG. 3 relative to SEQ ID NO: 1.

6. The microbial cell of claim 2, wherein the ADS comprises an amino acid substitution at one or more positions corresponding to the following positions of SEQ ID NO: 1: 396, 104, 162, 474, 118, 363, 322, 173, 112, 431, 151, 291, 134, 341, 230, 245, 44, 385, 100, 469, 500, 292, 471, 207, 463, 189, 340, 510, 260, 247, 211, 430, 277, 318, 275, 170, 124, 125, 145, 169, 445, 155, 152, 507, 520, 393, 447, 455, 498, 409, 204, and 261.

7. The microbial cell of claim 6, wherein the ADS comprises one or more substitutions selected from the following substitutions numbered according to SEQ ID NO: 1: V396A, S104A, D162E, Y474E, T118S, G363A, S322D, I173S, K112Q, L431I, S151H, A291V, Q134E, M341L, E230D, V245I, K44E, H385Y, G100L, N469G, I500V, V292I, N471S, A207R, S463C, I189V, F340L, Y510N, C260F, K247R, P211S, P430K, Y277F, V318I, S275V, R170H, N124K, Y125F, E145S, S169T, L445I, P155H, M152L, A507R, A520K, D393M, T447S, K455G, K498T, C409S, I204L, and L261A.

8. The microbial cell of claim 7, wherein the ADS comprises at least two, at least three, at least four, or at least five amino acid substitutions selected from V396A, S104A, D162E, Y474E, T118S, G363A, S322D, I173S, K112Q, L431I, S151H, A291V, Q134E, M341L, E230D, V245I, K44E, H385Y, G100L, N469G, I500V, V292I, N471S, A207R, S463C, I189V, F340L, Y510N, C260F, K247R, P211S, P430K, Y277F, V318I, S275V, R170H, N124K, Y125F, E145S, S169T, L445I, P155H, M152L, A507R, A520K, D393M, T447S, K455G, K498T, C409S, I204L, and L261A.

9. The microbial cell of claim 8, wherein the ADS comprises the substitutions T118S, D162E, I173S, S322D, G363A, V396A, and Y474E.

10. The microbial cell of any one of claims 2-9, wherein the ADS exhibits increased production of amorphadiene as compared to ADS comprising the amino acid sequence of SEQ ID NO: 1.

11. The microbial cell of any one of claims 1-10, wherein the AO comprises an amino acid sequence of SEQ ID NOs: 3 or a variant thereof.

12. The microbial cell of claim 11, wherein the AO comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO: 3.

13. The microbial cell of claim 11, wherein the AO comprises an amino acid sequence having from 1 to 20, from 1 to 10, or from 1 to 5 amino acid modifications independently selected from substitutions, insertions, and deletions with respect to SEQ ID NO: 3.

14. The microbial cell of claim 11, wherein the AO comprises one or more amino acid substitutions at positions shown in FIG. 5 or FIG. 7 relative to SEQ ID NO: 3.

15. The microbial cell of claim 11, wherein the AO comprises an amino acid substitution at one or more positions corresponding to the following positions of SEQ ID NO: 3: 239, 257, 408, 410, 421, 320, 130, 489, 198, 119, 102, 186, 252, 294, 314, 457, 474, 319, 322, 390, 125, 155, 251, 445, 424, 250, 387, 462, 153, 151, 243, 308, 495, 73, 103, 59, 123, 124, 146, 256, 261, 369, 469, and 64.

16. The microbial cell of claim 11, wherein the AO comprises one or more substitutions selected from the following substitutions numbered according to SEQ ID NO:3: A239R, A257D, A408P, A410E, A421I, C320N, E130D, E489D, G198K, H119G, I102L, I186T, I252L, I294V, I314M, I457L, I474L, K319R, K322R, K390R, L125F, L155I, L25II, L445F, M424K, N250R, N387A, N462D, Q153R, S151Q, S243K, S308T, S495T, S73P, T103A, T59L, V123I, V124A, V146T, V256I, V261E, V369L, V469M, and V64L.

17. The microbial cell of claim 11, wherein the AO comprises at least two, at least three, at least four, or at least five amino acid substitutions selected from A239R, A257D, A408P, A410E, A421I, C320N, E130D, E489D, G198K, H119G, I102L, I186T, I252L, I294V, I314M, I457L, I474L, K319R, K322R, K390R, L125F, L155I, L25II, L445F, M424K, N250R, N387A, N462D, Q153R, S151Q, S243K, S308T, S495T, S73P, T103A, T59L, V123I, V124A, V146T, V256I, V261E, V369L, V469M, and V64L.

18. The microbial cell of claim 17, wherein the AO comprises V64L, S73P, L155I, C320N, K322R, and V369L substitutions.

19. The microbial cell of any one of claims 11 to 18, wherein the AO exhibits increased production of artemisinic alcohol (A-OH) as compared to AO comprising the amino acid sequence of SEQ ID NO: 3.

20. The microbial cell of any one of claims 11 to 18, wherein the AO exhibits increased production of artemisinic aldehyde (A-CHO) as compared to AO comprising the amino acid sequence of SEQ ID NO: 3.

21. The microbial cell of any one of claims 11 to 18, wherein the AO exhibits increased production of artemisinic acid (AA) as compared to AO comprising the amino acid sequence of SEQ ID NO: 3.

22. The microbial cell of any one of claims 1-21, wherein the cell further comprises a cytochrome P450 reductase (CPR).

23. The microbial cell of claim 22, wherein the CPR comprises the amino acid sequence of SEQ ID NO: 5, or a variant thereof.

24. The microbial cell of any one of claims 1-23, further comprising a heterologous enzyme having an alcohol dehydrogenase (ADH) activity.

25. The microbial cell of claim 24, wherein the ADH comprises the amino acid sequence of SEQ ID NOs: 6 or a variant thereof.

26. The microbial cell of claim 25, wherein the ADH comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO: 6.

27. The microbial cell of claim 26, wherein the ADH comprises an amino acid sequence having from 1 to 20, from 1 to 10, or from 1 to 5 amino acid modifications independently selected from substitutions, insertions, and deletions with respect to SEQ ID NO: 6.

28. The microbial cell of claim 25, wherein the ADH comprises one or more amino acid substitutions at positions shown in FIG. 9 relative to SEQ ID NO: 6.

29. The microbial cell of claim 25, wherein the ADH comprises an amino acid substitution at one or more positions corresponding to the following positions of SEQ ID NO: 6: 82, 302, 155, 360, 299, 258, 304, 19, 107, 193, 263, 168, 78, 20, 253, 75, 191, 302, 80, 153, 203, 169, 229, 221, 329, 150, 6, 305, 60, 25, 310, 92, 233, 257, and 170.

30. The microbial cell of claim 29, wherein the ADH comprises one or more substitutions selected from the following substitutions numbered according to SEQ ID NO: 6: A82V, E302G, A155V, Q360N, A299V, V258I, S304E, S19A, T107S, S193E, H263D, S168P, 178V, S20G, S253P, I75V, Q191K, E302G, K80E, A153T, I203L, Y169L, Q229P, K221R, Q329R, T150S, P6G, V305I, 160E, L25I, L310F, M92I, A233E, L257V, and P170D.

31. The microbial cell of claim 30, wherein the ADH comprises at least two, at least three, at least four, or at least five amino acid substitutions selected from A82V, E302G, A155V, Q360N, A299V, V258I, S304E, S19A, T107S, S193E, H263D, S168P, 178V, S20G, S253P, I75V, Q191K, E302G, K80E, A153T, I203L, Y169L, Q229P, K221R, Q329R, T150S, P6G, V305I, I60E, L25I, L310F, M92I, A233E, L257V, and P170D.

32. The microbial cell of claim 29, wherein the ADH comprises the substitution A82V.

33. The microbial cell of any one of claims 24-32, wherein the ADH or variant thereof has increased production of artemisinic aldehyde (A-CHO) as compared to ADH comprising the amino acid sequence of SEQ ID NO: 6.

34. The microbial cell of any one of claims 1-33, further comprising a heterologous enzyme having an aldehyde dehydrogenase (ALDH) activity.

35. The microbial cell of claim 34, wherein the ALDH comprises the amino acid sequence of SEQ ID NO: 8 or a variant thereof.

36. The microbial cell of claim 35, wherein the ALDH comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO: 8.

37. The microbial cell of claim 36, wherein the ALDH comprises an amino acid sequence having from 1 to 20, from 1 to 10, or from 1 to 5 amino acid modifications independently selected substitutions, insertions, and deletions with respect to SEQ ID NO: 8.

38. The microbial cell of any one of claims 33-37, wherein the ALDH has increased production of artemisinic acid (AA) as compared to ALDH comprising the amino acid sequence of SEQ ID NO: 8.

39. The microbial cell of any one of claims 1-38, further comprising a heterologous enzyme having a double bond reductase (DBR) activity.

40. The microbial cell of claim 39, wherein the DBR comprises an amino acid sequence of SEQ ID NOs: 9, 10, or a variant thereof.

41. The microbial cell of claim 40, wherein the DBR comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NOs: 9 or 10.

42. The microbial cell of claim 41, wherein the DBR comprises an amino acid sequence having from 1 to 20, from 1 to 10, or from 1 to 5 amino acid modifications independently selected from substitutions, insertions, and deletions with respect to SEQ ID NO: 9.

43. The microbial cell of claim 41, wherein the DBR comprises an amino acid sequence having from 1 to 20, from 1 to 10, or from 1 to 5 amino acid modifications independently selected from substitutions, insertions, and deletions with respect to SEQ ID NO: 10.

44. The microbial cell of any one of claims 39-43, wherein the DBR exhibits increased production in the microbial cell of AA or DHAA as compared to DBR comprising the amino acid sequence of SEQ ID NOs: 9 or 10.

45. The microbial cell of claim 44, wherein the enzyme having DBR activity comprises an amino acid sequence with at least 80% or at least 90% sequence identity to SEQ ID NO: 9, with an amino acid substitution at position 241 with respect to SEQ ID NO: 9.

46. The microbial cell of claim 45, wherein the enzyme having DBR activity comprises a substitution of asparagine at position 241 with respect to SEQ ID NO: 9.

47. The microbial cell of any one of claims 1-46, wherein the microbial host cell further expresses a heterologous enzyme having activity for converting AA or DHAA to artemisinin, and comprising an amino acid sequence that has at least about 70% sequence identity, or at least about 80% sequence identity, or at least about 90% sequence identity, or at least about 95% sequence identity to one of SEQ ID NOs: 11 to 200.

48. The microbial cell of any one of claims 1-46, wherein the microbial host cell further expresses a heterologous enzyme having an activity for converting AA or DHAA to artemisinin and comprises an amino acid sequence that has at least about 70% sequence identity, or at least about 80% sequence identity, or at least about 90% sequence identity, or at least about 95% sequence identity to one of SEQ ID NOs: 201 to 488.

49. The microbial cell of claim 48, wherein the heterologous enzyme comprises an amino acid sequence that has at least about 70%, at least about 80%, at least about 90%, or at least about 95% sequence identity to one of SEQ ID NOS: 302, 323, 361, 366, and 369.

50. The microbial cell of claim 48, wherein the heterologous enzyme comprises an amino acid sequence that has at least about 70%, at least about 80%, at least about 90%, or at least about 95% sequence identity to one of SEQ ID NOS: 302, 361, and 369.

51. The microbial cell of any one of claims 1 to 50, wherein one or more of the heterologous enzymes are expressed from extrachromosomal elements.

52. The microbial cell of any one of claims 1 to 50, wherein one or more of the heterologous enzymes are expressed from genes that are chromosomally integrated.

53. The microbial cell of any one of claims 1 to 52, wherein the microbial host cell overexpresses one or more enzymes in the methylerythritol phosphate (MEP) or the mevalonic acid (MVA) pathway.

54. The microbial cell of any one of claims 1 to 53, wherein the microbial cell is a bacteria, optionally selected from Escherichia spp., Bacillus spp., Corynebacterium spp., Rhodobacter spp., Zymomonas spp., Vibrio spp., and Pseudomonas spp.

55. The microbial cell of claim 54, wherein the bacterial host cell is selected from Escherichia coli, Bacillus subtilis, Corynebacterium glutamicum, Rhodobacter capsulatus, Rhodobacter sphaeroides, Zymomonas mobilis, Vibrio natriegens, or Pseudomonas putida.

56. The microbial cell of any one of claims 1 to 53, wherein the microbial host cell is a yeast, optionally selected from Saccharomyces, Pichia, or Yarrowia.

57. The microbial cell of claim 55, wherein the microbial cell is Saccharomyces cerevisiae, Pichia pastoris, and Yarrowia lipolytica.

58. A method for making AA, DHAA, or artemisinin, comprising: culturing the microbial cell of any one of claims 1 to 57, and recovering the AA, DHAA, or artemisinin from the culture.

59. The method of claim 58, wherein the microbial cells are cultured with C1, C2, C3, C4, C5, and/or C6 carbon substrates.

60. The method of claim 59, wherein the carbon source is glucose, sucrose, fructose, xylose, and/or glycerol.

61. The method of any one of claims 58 to 60, wherein culture conditions are selected from aerobic, microaerobic, and anaerobic.

62. The method of claim 61, wherein the microbial cell is cultured at a temperature between 22° C. and 37° C.

63. The method of claim 58, further comprising a step of converting DHAA to artemisinin.

64. The method of claim 63, wherein the converting of DHAA to artemisinin is done photochemically.

65. The method of claim 58, further comprising a step of recovering artemisinin from the culture.

66. A method for making a pharmaceutical product, comprising, incorporating the artemisinin made according to the method of any one of claims 63 to 65 into said industrial or consumer product.

67. An ADS variant comprising an amino acid substitution at one or more positions corresponding to the following positions of SEQ ID NO: 1: 396, 104, 162, 474, 118, 363, 322, 173, 112, 431, 151, 291, 134, 341, 230, 245, 44, 385, 100, 469, 500, 292, 471, 207, 463, 189, 340, 510, 260, 247, 211, 430, 277, 318, 275, 170, 124, 125, 145, 169, 445, 155, 152, 507, 520, 393, 447, 455, 498, 409, 204, and 261 wherein the ADS variant has increased amorphadiene synthase activity as compared to SEQ ID NO: 1.

68. The ADS variant of claim 67, wherein the variant is isolated or partially purified, or is heterologously expressed in a host cell.

69. The ADS variant of claim 67, comprising the amino acid sequence of SEQ ID NO: 2.

70. The ADS variant of claim 67 comprising an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO: 1.

71. The ADS variant of claim 67, wherein the ADS comprises an amino acid sequence having from 1 to 20, from 1 to 10, or from 1 to 5 amino acid modifications independently selected from substitutions, insertions, and deletions with respect to SEQ ID NO:1.

72. The ADS variant according to claim 67, further comprising one or more amino acid substitutions at positions shown in FIG. 3 relative to SEQ ID NO: 1.

73. The ADS variant of claim 72, comprising one or more substitutions selected from the following substitutions numbered according to SEQ ID NO: 1: V396A, S104A, D162E, Y474E, T118S, G363A, S322D, I173S, K112Q, L431I, S151H, A291V, Q134E, M341L, E230D, V245I, K44E, H385Y, G100L, N469G, I500V, V292I, N471S, A207R, S463C, I189V, F340L, Y510N, C260F, K247R, P211S, P430K, Y277F, V318I, S275V, R170H, N124K, Y125F, E145S, S169T, L445I, P155H, M152L, A507R, A520K, D393M, T447S, K455G, K498T, C409S, I204L, and L261A,

74. The ADS variant of claim 73, comprising at least two, at least three, at least four, or at least five amino acid substitutions selected from V396A, S104A, D162E, Y474E, T118S, G363A, S322D, I173S, K112Q, L431I, S151H, A291V, Q134E, M341L, E230D, V245I, K44E, H385Y, G100L, N469G, I500V, V292I, N471S, A207R, S463C, I189V, F340L, Y510N, C260F, K247R, P211S, P430K, Y277F, V318I, S275V, R170H, N124K, Y125F, E145S, S169T, L445II P155H, M152L, A507R, A520K, D393M, T447S, K455G, K498T, C409S, I204L, and L261A,

75. The ADS variant of claim 74, wherein the ADS comprises the substitutions T118S, D162E, I173S, S322D, G363A, V396A, and Y474E.

76. A recombinant nucleic acid molecule comprising a nucleotide sequence encoding the ADS variant of any one of claims 67-75.

77. An AO variant comprising an amino acid substitution at one or more positions corresponding to the following positions of SEQ ID NO: 3: 239, 257, 408, 410, 421, 320, 130, 489, 198, 119, 102, 186, 252, 294, 314, 457, 474, 319, 322, 390, 125, 155, 251, 445, 424, 250, 387, 462, 153, 151, 243, 308, 495, 73, 103, 59, 123, 124, 146, 256, 261, 369, 469, and 64 wherein the AO variant has increased amorphadiene oxidase activity as compared to SEQ ID NO: 3.

78. The AO variant of claim 77, wherein the variant is isolated or partially purified, or is heterologously expressed in a host cell.

79. The AO variant of claim 77, wherein the variant comprises a leader sequence that supports expression and activity in E. coli, a linker sequence, or a CPR or derivative thereof sufficient to regenerate the AO variant.

80. The AO variant of claim 77, comprising the amino acid sequence of SEQ ID NO: 3.

81. The AO variant of claim 77, comprising an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO: 3.

82. The AO variant of claim 77, comprising an amino acid sequence having from 1 to 20, from 1 to 10, or from 1 to 5 amino acid modifications independently selected from substitutions, insertions, and deletions with respect to SEQ ID NO: 3.

83. The AO variant of claim 77, further comprising one or more amino acid substitutions at positions shown in FIG. 5 or FIG. 7 relative to SEQ ID NO: 3.

84. The AO variant of claim 77, comprising one or more substitutions selected from the following substitutions numbered according to SEQ ID NO:3: A239R, A257D, A408P, A410E, A421I, C320N, E130D, E489D, G198K, H119G, I102L, I186T, I252L, I294V, I314M, I457L, I474L, K319R, K322R, K390R, L125F, L155I, L251I, L445F, M424K, N250R, N387A, N462D, Q153R, S151Q, S243K, S308T, S495T, S73P, T103A, T59L, V123I, V124A, V146T, V256I, V261E, V369L, V469M, and V64L.

85. The AO variant of claim 84, comprising at least two, at least three, at least four, or at least five amino acid substitutions selected from A239R, A257D, A408P, A410E, A421I, C320N, E130D, E489D, G198K, H119G, I102L, I186T, I252L, I294V, I314M, I457L, I474L, K319R, K322R, K390R, L125F, L155I, L25II, L445F, M424K, N250R, N387A, N462D, Q153R, S151Q, S243K, S308T, S495T, S73P, T103A, T59L, V123I, V124A, V146T, V256I, V261E, V369L, V469M, and V64L.

86. The AO variant of claim 85, comprising V64L, S73P, L155I, C320N, K322R, and V369L substitutions.

87. The AO variant of any one of claims 77-86, wherein the AO exhibits increased production of artemisinic alcohol (A-OH) as compared to AO comprising the amino acid sequence of SEQ ID NO: 3.

88. The AO variant of any one of claims 77-86, wherein the AO exhibits increased production of artemisinic aldehyde (A-CHO) as compared to AO comprising the amino acid sequence of SEQ ID NO: 3.

89. The AO variant of any one of claims 77-86, wherein the AO exhibits increased production of artemisinic acid (AA) as compared to AO comprising the amino acid sequence of SEQ ID NO: 3.

90. A recombinant nucleic acid molecule comprising a nucleotide sequence encoding the AO variant of any one of claims 77-89.

91. An ADH variant comprising an amino acid substitution at one or more positions corresponding to the following positions of SEQ ID NO: 6: 82, 302, 155, 360, 299, 258, 304, 19, 107, 193, 263, 168, 78, 20, 253, 75, 191, 302, 80, 153, 203, 169, 229, 221, 329, 150, 6, 305, 60, 25, 310, 92, 233, 257, and 170 wherein the ADH variant has increased alcohol dehydrogenase activity.

92. The ADH variant of claim 91, wherein the variant is isolated or partially purified, or is heterologously expressed in a host cell.

93. The ADH variant of claim 91, comprising the amino acid sequence of SEQ ID NO: 7.

94. The ADH variant of claim 91, comprising an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to SEQ ID NO: 6.

95. The ADH variant of claim 91, comprising an amino acid sequence having from 1 to 20, from 1 to 10, or from 1 to 5 amino acid modifications independently selected from substitutions, insertions, and deletions with respect to SEQ ID NO: 6.

96. The ADH variant of claim 91, further comprising one or more amino acid substitutions at positions shown in FIG. 9 relative to SEQ ID NO: 6.

97. The ADH variant of claim 91, comprising one or more substitutions selected from the following substitutions numbered according to SEQ ID NO: 6: A82V, E302G, A155V, Q360N, A299V, V258I, S304E, S19A, T107S, S193E, H263D, S168P, 178V, S20G, S253P, I75V, Q191K, E302G, K80E, A153T, I203L, Y169L, Q229P, K221R, Q329R, T150S, P6G, V305I, I60E, L25I, L310F, M92I, A233E, L257V, and P170D.

98. The ADH variant of claim 97, comprising at least two, at least three, at least four, or at least five amino acid substitutions selected from A82V, E302G, A155V, Q360N, A299V, V258I, S304E, S19A, T107S, S193E, H263D, S168P, 178V, S20G, S253P, 175V, Q191K, E302G, K80E, A153T, I203L, Y169L, Q229P, K221R, Q329R, T150S, P6G, V305I, I60E, L25I, L310F, M92I, A233E, L257V, and P170D.

99. The ADH variant of claim 98, comprising the substitution A82V.

100. A recombinant nucleic acid molecule comprising a nucleotide sequence encoding the ADH variant of any one of claims 91-99.

101. A method for producing artemisinin, comprising:
contacting one or more enzymes comprising an amino acid sequence having at least 70%, or at least about 80%, at least about 90%, or at least about 95% sequence identity to one of SEQ ID NOs: 11 to 488, with a substrate selected from one or more of artemisinic acid (AA) and dihydroartemisinic acid (DHAA), and
recovering artemisinin.

102. The method of claim 101, wherein the one or more enzymes comprises an amino acid sequence that has at least 70%, at least about 80%, or at least about 90%, or at least about 95% sequence identity to one of SEQ ID NOs: 302, 323, 361, 366, and 369.

103. The method of claim 102, wherein the one or more enzymes comprises an amino acid sequence that has at least 70%, at least about 80%, at least about 90%, or at least about 95% sequence identity to one of SEQ ID NOS: 302, 361, and 369.

104. The method of any one of claims 101 to 103, wherein the one or more enzymes are expressed in a host cell, and the substrate is fed to a culture comprising the host cell.

105. The method of any one of claims 101 to 103, wherein the one or more enzymes are expressed in a host cell, and the substrate is contacted with a lysate or partially purified lysate of the host cell under suitable reaction conditions.

106. The method of any one of claims 101 to 103, wherein the one or more enzymes are recombinantly produced, and the substrate is contacted with the enzyme under suitable reaction conditions.

107. The method of any one of claims 101 to 106, wherein the microbial cell is a bacteria, optionally selected from Escherichia spp., Bacillus spp., Corynebacterium spp., Rhodobacter spp., Zymomonas spp., Vibrio spp., and Pseudomonas spp.

108. The method of claim 107, wherein the bacterial host cell is selected from Escherichia coli, Bacillus subtilis, Corynebacterium glutamicum, Rhodobacter capsulatus, Rhodobacter sphaeroides, Zymomonas mobilis, Vibrio natriegens, or Pseudomonas putida.

109. The method of any one of claims 101 to 106, wherein the microbial host cell is a yeast, optionally selected from Saccharomyces, Pichia, or Yarrowia.

110. The method of claim 109, wherein the microbial cell is Saccharomyces cerevisiae, Pichia pastoris, and Yarrowia lipolytica.
